(19) United States
(12) Patent Application Publication
(10) Pub. No.: US 2026/0217855 A1
(43) Pub. Date: Jul. 30, 2026

(54) Engineered T Cells

(71) Applicant: Expression Therapeutics, LLC, Tucker, GA (US)

(72) Inventors: Harrison BROWN, Atlanta, GA (US); Brian PETRICH, Tucker, GA (US)

(21) Appl. No.: 19/145,404

(22) PCT Filed: Jan. 10, 2024
(86) PCT No.: PCT/US2024/011116

§ 371 Date: Jul. 2, 2025

Related U.S. Application Data
(60) Provisional application No. 63/438,181, filed on Jan. 10, 2023.
(60) Provisional application No. 63/601,999, filed on Nov. 22, 2023.

(51) Int. Cl. C07K 16/30; A61K 38/00; C07K 16/28; C07K 16/32; C12N 5/0783; C12N 15/86

(52) U.S. Cl. CPC C07K 16/30; A61K 38/00; C07K 16/2809; C07K 16/32; C12N 5/0636; C12N 15/86; C07K 2317/622; C07K 2319/035; C12N 2510/00

(57) ABSTRACT

A therapeutic molecule (single chain-based antibody or ligand-based) optimized for expression and secretion from engineered T cells, which may be gamma delta (gd) T cells. When expressed from engineered gdT cells, the STAR will be secreted and mediate engagement between gdT cells and antigen/receptor on target cells. Binding mediates the formation of a cytolytic synapse between the gdT cell and the target cell leading to activation the gdT cells to release proteolytic enzymes that kill target cells.

### CROSS REFERENCE TO RELATED APPLICATION

This application claims priority to and the benefit of U.S. Provisional Patent Application No. 63/601,999, filed Nov. 22, 2023, and U.S. Provisional Patent Application No. 63/428,181, filed Jan. 10, 2023, the entirety of which is incorporated herein by reference.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “240110_103-3002PCT3_Seq_Listing.xml” (~252 kb), which was created on Jan. 10, 2024 which is incorporated by reference herein.

### BRIEF SUMMARY

Novel T-cell activating bispecific antibody therapeutics. In some variations the bispecific antibodies may be used to engaged cytotoxic T cells against tumor cells. Engineered gamma delta T cells secreting bispecific therapeutics (antibody-based and/or ligand-based) for enhanced cytotoxicity towards various tumor antigens.

### BRIEF DESCRIPTION OF THE DRAWINGS

FIG. 1 provides an exemplary STAR framework.

FIG. 2 provides a schematic representing variable elements of a generic STAR design and exemplary specific element identities.

FIG. 3 demonstrates further schematics representing variable elements of STAR designs.

FIG. 4 demonstrates a schematic of an alternative STAR design capable of binding gamma delta T cells.

FIG. 5 is a LentET STAR schematic.

FIG. 6 provides a schematic of methods of manufacturing and genetically engineered gamma delta T cells.

FIG. 7 is a schematic of STARS Mechanism of action.

FIG. 8 is a flow diagram of a method for gdT cell production.

FIG. 9 is an overview of gdT cell expansion process.

FIG. 10 shows identification of donors with acceptable ex vivo expansion of gdT cells from peripheral blood mononuclear cells (PBMCs).

FIG. 11 shows screening of ex vivo expanded gdT cells to identify donors that generate gdT cells with high cytotoxicity toward K562 human cancer cells.

FIG. 12 is chart of GFP expression in LentET transduced gdT cells.

FIG. 13 is a chart of GFP MFI in LentET transduced gdT cells.

FIG. 14 is a chart of data showing cytotoxicity of secreted media from PTK7 and GD2 STAR expressing 293T cells.

FIG. 15 is data related to mRNA transfected gdT-CMK cytotoxicity.

FIG. 16 is a Western blot analysis of the designated STAR proteins.

FIG. 17 is quantitation of STAR secretion.

FIG. 18 is a characterization of secretion with the albumin linker.

FIG. 19 is a characterization of gdT cells transduced with STAR-encoding lentivirus gain cytotoxic potential against target cells.

FIG. 20. is data characterizing Integrin aV B3 CD3 STAR.

FIG. 21 is data characterizing IL2 CD19 CD3 STAR.

FIG. 22 is data characterizing mSA PTK7 CD3 STAR.

FIG. 23 is data characterizing mSA PTK7 CD3 STAR.

FIG. 24 is data characterizing mSA and native signal peptide hSCF CD3 STARs.

FIG. 25 shows gdT cells (effector [E]) were transfected with mRNA encoding the mSA and native signal peptide versions of the hSCF CD3 STAR and mixed with IMR5 cells (target [T]).

FIG. 26 is data characterizing mSA and IL2 GD2 CD3 STARs.

FIG. 27 is data characterizing mSA and IL2 GD2 CD3 STARs.

FIG. 28 demonstrates IL2 SSTR HL and LH CD3 STAR.

FIG. 29 demonstrates IL2 SSTR HL and LH CD3 STAR.

FIG. 30 demonstrates a humanized/deimmunized version of the CD3 scFv directs gdT mediated killing.

FIG. 31 demonstrates a humanized/deimmunized version of the CD3 scFv directs gdT mediated killing.

FIG. 32 demonstrates Lentiviral delivery of shRNA knocks down HLA Class I and II surface expression.

FIG. 33 demonstrates Lentiviral delivery of shRNA knocks down HLA Class I and II surface expression.

FIG. 34 demonstrates alternative gdT targeting moieties direct gdT mediated cytoxicity.

FIG. 35 demonstrates alternative gdT targeting moieties direct gdT mediated cytoxicity.

FIG. 36 demonstrates Somatostain ligand gdT mediated cytoxicity toward NET cells.

FIG. 37 demonstrates Somatostain ligand gdT mediated cytoxicity toward NET cells.

FIG. 38 demonstrates IL2 TPO BR CD3 STAR expression.

FIG. 39 demonstrates mRNA mediated protein expression correlates with mRNA free energy.

FIG. 40 demonstrates flow cytometry data representing percent killing activity at 1:1 and 5:1 ratios, comparing killing activity of PTK7-14, SSTR2-3, and SSTR2-8.

FIG. 41 is a Western Blot evidencing that the desired proteins were expressed from the plasmid DNA.

FIG. 42 demonstrates flow cytometry data representing percent killing activity of target cells comparing untreated versus GD2-3.

FIG. 43 demonstrates STAR-specific IMR5 death (7AAD+) results of transient transfection of 293T cells as quantified by ELISA. Cytotoxicity was determined by flow cytometry.

FIG. 44 demonstrates STAR-specific IMR5 killing (7AAD+ and/or Annexin V+) results of transient transfection of 293T cells as quantified by ELISA. Cytotoxicity was determined by flow cytometry.

FIG. 45 shows cytotoxicity of various PTK7-STAR proteins (x-axis) at 1:1 ratio of effector (gamma delta T cells) to target (IMR5 cells).

FIG. 46 shows cytotoxicity of various PTK7-STAR proteins (x-axis) at 5:1 ratio of effector (gamma delta T cells) to target (IMR5 cells).

FIG. 47 shows cytotoxicity of various SSTR-STAR proteins (x-axis) at 1:1 ratio of effector (gamma delta T cells) to target (IMR5 cells).

FIG. 48 shows cytotoxicity of various SSTR-STAR proteins (x-axis) at 5:1 ratio of effector (gamma delta T cells) to target (IMR5 cells).

FIG. 49 shows cytotoxicity of additional SSTR-STAR proteins (x-axis) at 1:1 and 5:1 ratios of effector (gamma delta T cells) to target (IMR5 cells).

### DETAILED DESCRIPTION

The claimed subject matter is now described with reference to the drawings, wherein like reference numerals are generally used to refer to like elements throughout. In the following description, for the purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

The following description of the drawings and the various system, method, and apparatus is not intended to limit the inventive system, methods and apparatus disclosed herein to one variation, but rather to enable any person skilled in the art of project management and/or software development to make and use the inventive system, method and apparatus.

The present disclosure relates to peptides, proteins, nucleic acids and cells for use in immunotherapeutic methods. In a variation, we disclose hematopoietic cells capable of secreting one or more synthetic fusion proteins and/or therapeutics. In particular, the present disclosure relates to the immunotherapy of cancer, including, e.g., B cell malignancies, neuroblastoma, osteosarcoma, neuroendocrine tumors (NETs), and acute myeloid leukemia (AML). The present disclosure furthermore relates to target cell cytotoxicity and secreted T cell actuators (referred to herein as “STARs”).

“STARs” is an umbrella term to describe the proteins genetically engineered to be expressed from gamma delta T cells. The disclosed STARs provide a unique advantage over existing soluble immune-oncology therapies, e.g., cytokines, monoclonal antibodies, and bispecific immune cell engagers. The STARs disclosed herein provide a solution to the side effects encountered by existing soluble immune-oncology therapies (i.e., side effects related to dosing, pharmacokinetics, and pharmacodynamics). The STARs disclosed herein are ECO optimized using a proprietary method of codon optimization. STARs are novel ECO optimized secreted T cell Actuators which are secreted from gamma delta T cells following gene transfer (e.g., viral vector transduction or mRNA electroporation).

We disclose herein novel gamma delta T cells (referred to herein as gdT cells or gdT cells) engineered to secrete proteins that can affect cancer. In one example, gdT cells are engineered to secrete proteins that act to alter the growth, expansion, and viability of a T cell population. In a variation, the gamma delta T cells, secrete bi-specific T-cell actuators. In the past, bi-specific T cell engagers were injected directly into patients via bolus therapy of Fc containing bi-specific antibodies (bsAbs), or continuous infusion of Fc-free bsAbs. We disclose herein a novel method of introducing STARs, T cell actuators and/or other bispecific molecules, and/or other secreted proteins by delivering to a patient gdT cells capable of secreting therapeutic agents of interest. In some variations, gdT cells delivered to the patients can secrete the proteins of interest. In a variation, a gdT T cell expressing STARs, a STAR (e.g., a bi-specific T cell engager) is inserted into a patient.

The present disclosure includes STAR designs that target gdT cells to somatostatin receptor 2 (SSTR2)+ tumor cells. A monoclonal antibody targeting SSTR2 was adapted by converting it into several single chain variable fragment (scFv) designs and used them to direct the STARs/gdT cells to the SSTR2+ tumor.

In the present disclosure, the STAR designs can be secreted in vivo from ex vivo modified gdT cells. However, the protein designs have use as recombinant proteins injected directly. The gdT cells can be modified to express the STARs by a number of methods, including lentiviral transduction, AAV transduction, mRNA electroporation, mRNA transfection, and non-viral gene transfer technologies, CRISPR knock in, etc.

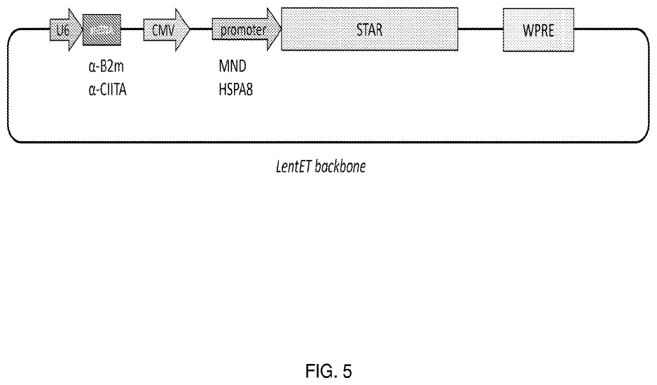

Codon optimization is an approach in genetic engineering to improve gene expression by changing synonymous codons based on an organism's codon bias. Disclosed herein are amino acid sequences, non-optimized DNA sequences, and Expression Codon Optimized (ECO) for gamma delta T-cell (ECOg) sequences. Expression codon optimized for gamma delta T-cell expression is using an algorithm with novel codon usage indices generated from target cell, in this case, gamma delta T-cell, expression data. Where the sequences are optimized for expression in or by gamma delta T-cells, they may be referred to alternately a ECOg or gamma delta T-cell optimized. This means that the sequence has been codon optimized for improved expression from gamma delta T-cells as compared to the wild type or non-optimized sequences. Additionally disclosed are two unique codon optimization sequence for the IL2 signal peptide which were uniquely ECO optimized for enhanced translation initiation to improve protein expressivity. Optimization of the IL2 signal peptide used a unique method optimization of the scFv-containing domains of the STAR. Further, these sequences in our LentET lentiviral backbone use two different promoters active in gdT cells (see FIG. 5). The first is the synthetic MND promoter; (SEQ ID NO: 152); the second is the human genome derived Heat shock 70 kDa protein 8 promoter HSPA8 (SEQ ID NO: 153). The latter promoter was found to have high activity in gamma delta T cells. Use of this promoter to drive gene expression in gamma delta T-cells, especially from a lentivector, is a novel use of this sequence.

Other systems, methods, features, and advantages of the present disclosure will be, or will become, apparent to one with skill in the art upon examination of the figures and detailed description. It is intended that all such additional systems, methods, features and advantages be included within this description, be within the scope of the present disclosure, and be protected by the following claims.

We disclose T cells secreting a therapeutic, e.g., a STAR, which results in eradication of cancer cells by various routes. A further advantage of this method is that a STAR disclosed can also recruit the patient's T cells to also fight the cancer.

As an additional or alternative technique, molecules can be added which enhance T cell function, for example but not limited to, gamma delta T cell function. Molecules can also be added which improve expansion and survival of T cells in vivo. Some examples of additional molecules are IL-2, IL-15. In some examples, the STAR is a bi-specific T cell actuator. In other variations, the STAR operates without engaging a T cell to a cancer cell. In some variations, the STAR mediates the expansion of T cells.

We disclose a STAR has a unique property of being a protein secreted from gdT cells. Secretion from gdT cells has not been demonstrated before. In fact, secretion from gdT cells required extensive optimization of the expression construct. To achieve the disclosed construct capable of expression from gdT cells, we optimized the system at several points in the protein expression chain, which will be discussed further below.

A STAR (e.g., in a single chain-based antibody and/or ligand-based format) optimized for expression and secretion from engineered gamma delta (“(gd) T cells” or “gdT”). When expressed from engineered gdT cells, the STAR will be secreted and mediate engagement between gdT cells and antigen/receptor on target cells. Binding mediates the formation of a cytolytic synapse between the gdT cell and the target cell leading to activation the gdT cells to release proteolytic enzymes that kill target cells.

We disclose STARs (e.g., in a scFv-based antibody and/or ligand-based format) optimized for gdT cell expression and secretion (IL-2 signal peptide sequence or another signal peptide).

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 11 recognized serotypes of AAV (AAV1-11).

Administration/Administer: To provide or give a subject an agent, such as a therapeutic agent (e.g., a recombinant AAV, recombinant lentivirus, STAR, vector expressing a star, modified gdT cell capable of expressing a STAR), by any effective route. Exemplary routes of administration include, but are not limited to, injection (such as subcutaneous, intramuscular, intradermal, intraperitoneal, and intravenous), oral, intraductal, sublingual, rectal, transdermal, intranasal, vaginal and inhalation routes.

Antigen Binding Moiety: As used herein, the term “antigen binding moiety” refers to a polypeptide molecule that specifically binds to an antigenic determinant. In one embodiment, an antigen binding moiety is able to direct the entity to which it is attached (e.g. a second antigen binding moiety) to a target site, for example to a specific type of tumor cell bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties comprise antibody constant regions as further defined herein and known in the art. Useful heavy chain constant regions include any of the five isotypes: α, δ, ε, γ, or μ. Useful light chain constant regions include any of the two isotypes: K and A.

Antigenic Determinant: As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

Specific Binding: By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art. In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antibody comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³M).

Affinity: “Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities are capable of comprising different rate constants, as long as the ratio of the rate constants remains the same. Affinity can be measured by well established methods known in the art, including those described herein.

As used herein, the terms “first”, “second” or “third” with respect to Fab molecules etc., are used for convenience of distinguishing when there is more than one of each type of moiety. Use of these terms is not intended to confer a specific order or orientation of the bispecific antibody unless explicitly so stated.

Valent: The term “valent” as used herein denotes the presence of a specified number of antigen binding sites in an antibody. As such, the term “monovalent binding to an antigen” denotes the presence of one (and not more than one) antigen binding site specific for the antigen in the antibody.

Antibody: The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure.

Antibody Fragment: An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. Single-domain antibodies are antibody fragments comprising all or a portion of the heavy chain variable domain or all or a portion of the light chain variable domain of an antibody. In certain embodiments, a single-domain antibody is a human single-domain antibody. Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kuby Immunology, 6^{th} ed., W.H. Freeman and Co., page 91 (2007). A single VH or VL domain may be sufficient to confer antigen-binding specificity.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1 (L1)-FR2-H2 (L2)-FR3-H3 (L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N-to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (2), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present.

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “fused” is meant that the components (e.g. a Fab molecule and an Fc domain subunit) are linked by peptide bonds, either directly or via one or more peptide linkers.

Gamma delta T cells (γδ T cells) or (gd T) are T cells that have a distinctive T cell receptor (TCR) on their surface. Most T cells are αβ (alpha beta) T cells with TCR composed of two glycoprotein chains called α (alpha) and β (beta) TCR chains. In contrast, gamma delta (γδ) T cells have a TCR that is made up of one γ (gamma) chain and one δ (delta) chain. This group of T cells is usually less common than αβ T cells.

Hematopoietic cells are cells capable of developing into blood cells through hematopoiesis.

Human peripheral blood mononuclear cells (PBMCs) are immune cells with a single nucleus. PBMCs originate in bone marrow. PBMCs are secreted into peripheral circulation. PBMCs are involved in both humoral and cell-mediated immunity. PBMCs include lymphocytes (T cells, B cells, NK cells) and monocytes.

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one embodiment, CD3 is human CD3, particularly the epsilon subunit of human CD3 (CD3c). The amino acid sequence of human CD3ε is shown in UniProt (www.uniprot.org) accession no. P07766 (version 144), or NCBI (www.ncbi.nlm.nih.gov/) RefSeq NP_000724.1.

The BiTE format, also known as a tandem scFv or (scFv) 2, is a small-sized Fc-free molecule composed of two scFvs connected by a flexible linker on a single polypeptide. The in vivo transfer of bsAb-encoding genetic information might be performed using viral and nonviral vectors.

Bispecific: The term “bispecific” (including bi-specific and bsAb) means that the antibody is able to specifically bind to at least two distinct antigenic determinants. Typically, a bispecific antibody comprises two antigen binding sites, each of which is specific for a different antigenic determinant. In certain embodiments the bispecific antibody is capable of simultaneously binding two antigenic determinants, particularly two antigenic determinants expressed on two distinct cells.

Bispecific antibodies include at least one or more antigen binding domains; multimerization core that forms a homo- or hetero-mulitmer; and linkers connecting the elements. The antigen-binding domain may be an antibody fragment, such as a Fab, single-chain garment variable (scFv), or single domain antibody (sdAb), or alternatively, an antibody mimetic. Another approach is the use of extracellular domains of natural receptors or ligands for the design of bsAbs. The multitargeting concept that bsAbs make possible is particularly appealing from a therapeutic point of view because many diseases are multifactorial, involving multiple receptors, ligands, and signaling cascades. T-cell engaging bsAbs (TCE) are designed to simultaneously bind to a selected tumor-associated antigen (TAA) on the tumor cell surface and one of the extracellular CD3 subunits (most commonly CD3e) on the T-cell surface.

cDNA (complementary DNA): A piece of DNA lacking internal, non-coding segments (introns) and regulatory sequences that determine transcription. cDNA is synthesized in the laboratory by reverse transcription from messenger RNA extracted from cells. cDNA can also contain untranslated regions (UTRs) that are responsible for translational control in the corresponding RNA molecule.

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

CAI: “CAI” is the codon adaptation index. CAI is used as a quantitative method of predicting the level of expression of a gene based on its codon sequence.

Control: A reference standard. In some embodiments, the control is a negative control sample obtained from a healthy patient. In other embodiments, the control is a positive control sample obtained from a patient diagnosed with cancer. In still other embodiments, the control is a historical control or standard reference value or range of values (such as a previously tested control sample, such as a group of cancer patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine (A), guanine (G), cytosine (C), and thymine (T) bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. For instance, it is appropriate to generate probes or primers from the reverse complement sequence of the disclosed nucleic acid molecules.

Enhancer: A nucleic acid sequence that increases the rate of transcription by increasing the activity of a promoter.

Flanking: Near or next to, also, including adjoining, for instance in a linear or circular polynucleotide, such as a DNA molecule.

Gene: A nucleic acid sequence, typically a DNA sequence, that comprises control and coding sequences necessary for the transcription of an RNA, whether an mRNA or otherwise. For instance, a gene may comprise a promoter, one or more enhancers or silencers, a nucleic acid sequence that encodes an RNA and/or a polypeptide, downstream regulatory sequences and, possibly, other nucleic acid sequences involved in regulation of the expression of an mRNA.

As is well known in the art, most eukaryotic genes contain both exons and introns. The term “exon” refers to a nucleic acid sequence found in genomic DNA that is bioinformatically predicted and/or experimentally confirmed to contribute a contiguous sequence to a mature mRNA transcript. The term “intron” refers to a nucleic acid sequence found in genomic DNA that is predicted and/or confirmed not to contribute to a mature mRNA transcript, but rather to be “spliced out” during processing of the transcript.

Gene therapy: The introduction of a heterologous nucleic acid molecule into one or more recipient cells, wherein expression of the heterologous nucleic acid in the recipient cell affects the cell's function and results in a therapeutic effect in a subject. For example, the heterologous nucleic acid molecule may encode a protein, which affects a function of the recipient cell.

Hybridizes: Hybridization assays for the characterization of nucleic acids with a certain level of identity to the nucleic acid sequences as provided herein are well known in the art; see e.g. Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989). The term “hybridization” or “hybridizes” as used herein may relate to hybridizations under stringent or non-stringentconditions. If not further specified, the conditions are preferably non-stringent. Said hybridization conditions may be established according to conventional protocols described, e.g., in Sambrook (2001) loc. cit.; Ausubel (1989) loc. cit., or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as, for example, the highly stringent hybridization conditions of 0.1×SSC, 0.1% SDS at 65° C. or 2×SSC, 60° C., 0.1% SDS. Low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single and double stranded forms of DNA. A polynucleotide may include either or both naturally occurring and modified nucleotides linked together by naturally occurring and/or non naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Nucleotide: This term includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

ORF (open reading frame): A series of nucleotide triplets (codons) coding for amino acids. These sequences are usually translatable into a peptide.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed vectors.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as vector compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Preventing, treating or ameliorating a disease: “Preventing” a disease (such as cancer) refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A region of DNA that directs/initiates transcription of a nucleic acid (e.g., a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription. A tissue-specific promoter is a promoter that directs/initiated transcription primarily in a single type of tissue or cell.

Protein: A biological molecule expressed by a gene or other encoding nucleic acid (e.g., a cDNA) and comprised of amino acids.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

A recombinant virus is one that includes a genome that includes a recombinant nucleic acid molecule. As used herein, “recombinant AAV” refers to an AAV particle in which a recombinant nucleic acid molecule has been packaged.

A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Response element (RE): A DNA sequence included in a promoter to which one or more transcription factors can bind to and confer an aspect of control of gene expression.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Additionally or alternatively, percent (%) amino acid sequence identity with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. amino acid identity is given in the output alignment header.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

As used herein, reference to “at least 90% identity” refers to “at least 90%, at least 91%, at least 92%, at least 93%, at least 94%, at least 95%, at least 96%, at least 97%, at least 98%, at least 99%, or even 100% identity” to a specified reference sequence.

Subject: Living multi-cellular vertebrate organisms, a category that includes human and non-human mammals.

Synthetic: Produced by artificial means in a laboratory, for example a synthetic nucleic acid can be chemically synthesized in a laboratory.

TATA box: A DNA sequence found in the promoter region of a gene that can be bound by TATA binding protein and transcription factor II D during DNA unwinding and binding by RNA polymerase II. A TATA box sequence typically includes a TATAAA sequence and often includes additional 3′ adenine nucleotides.

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Transcription factor (TF): A protein that binds to specific DNA sequences and thereby controls the transfer (or transcription) of genetic information from DNA to RNA. TFs perform this function alone or with other proteins in a complex, by promoting (as an activator), or blocking (as a repressor) the recruitment of RNA polymerase (the enzyme that performs the transcription of genetic information from DNA to RNA) to specific genes. The specific DNA sequences to which a TF binds is known as a response element (RE) or regulatory element. Other names include cis-element and cis-acting transcriptional regulatory element.

Transcription factors interact with their binding sites using a combination of electrostatic (of which hydrogen bonds are a special case) and Van der Waals forces. Due to the nature of these chemical interactions, most transcription factors bind DNA in a sequence specific manner. However, not all bases in the transcription factor-binding site may actually interact with the transcription factor. In addition, some of these interactions may be weaker than others. Thus, many transcription factors do not bind just one sequence but are capable of binding a subset of closely related sequences, each with a different strength of interaction.

For example, although the consensus binding site for the TATA-binding protein (TBP) is TATAAAA; however, the TBP transcription factor can also bind similar sequences such as TATATAT or TATATAA.

Transcription factors (TFs) are classified based on many aspects. For example, the secondary, tertiary and quaternary structures of the protein structures DNA-binding sequence and properties, the interaction with the double helix of the DNA, and the metal and other binding characteristics. The JASPAR database and TRANSFAC (TRANSFAC® 7.0 Public 2005) are two web-based transcription factor databases, their experimentally-proven binding sites, and regulated genes.

Transcription Start Site: The location where transcription starts at the 5′ end of a gene sequence.

Therapeutically effective amount: The amount of agent, such as a recombinant AAV vector, that is sufficient to prevent, treat (including prophylaxis), reduce and/or ameliorate the symptoms and/or underlying causes of a disorder or disease, for example to prevent, inhibit, and/or treat cancer. For instance, this can be the amount necessary to inhibit or prevent viral replication or to measurably alter outward symptoms of the disease or condition.

For example, administration of a therapeutically effective amount of a vector as disclosed herein can decrease a symptom by a desired amount, for example by at least 20%, at least 30%, at least 40%, at least 50%, at least 60%, at least 70%, at least 80%, at least 90%, at least 95%, at least 98%, at least 100% or more, as compared to a suitable control.

It is understood that to obtain a therapeutic response to the disease or condition can require multiple administrations of the agent. Thus, a therapeutically effective amount encompasses a fractional dose that contributes in combination with previous or subsequent administrations to attaining a therapeutic outcome in the patient. For example, a therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an adeno-associated virus (AAV) vector. In some embodiments, the vector is a gamma-retroviral vector, a lentiviral vector, or an adenoviral vector.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Overview

According to the present disclosure, the term STARs refers to secreted T cell Actuators. The term “actuator” is used to encompass T cell engagers and/or activators. The term “actuator” indicates secreted molecules capable of making the gamma delta T cells capable of performing therapeutic functions. STARs may be composed of STAR (scFv-based antibody or ligand-based) on one end and a T cell actuator molecule (scFv-based antibody or ligand-based) on the other end. Each of the components of a STAR construct may be sequence optimized for gamma delta T cell expression and secretion.

In a variation, STARs are a a bispecific and/or a bi-active therapeutic molecule secreted from the engineered gdT cell. STARs mediate binding to CD3 on gdT cells and ligand molecule on target cell to form an immunological synapse which in turn activates gdT cell cytotoxicity.

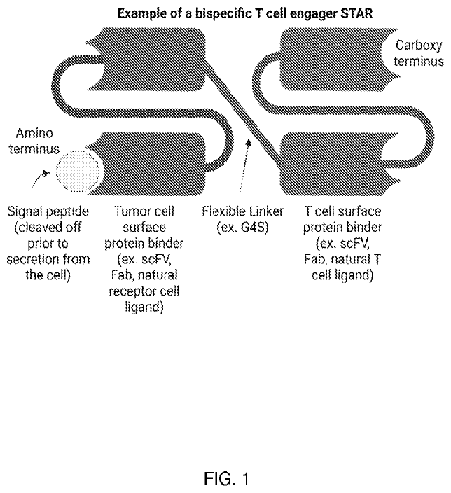

FIG. 1 provides a schematic of an exemplary STAR framework. The STAR may include at least one of the following elements: Starting on the amino terminus and traveling to the carboxy terminus, the STAR may include: (a) a signal peptide, which signal peptide may be cleaved off prior to secretion from the cell (b) a tumor cell surface protein binding (examples follow), (c) a flexible linker (optional) (also referred to herein as “Central linker”), (d) a T cell surface protein binder.

Examples of signal peptides according to the disclosure include but are not limited to IL2, mSA (modified serum albumin) (SEQ ID NO: 3) (SEQ ID NO: 5), and hSCF (human stem cell factor) (SEQ ID NO: 4) (SEQ ID NO: 6).

In a variation, the tumor cell surface binding protein may be one or more of, e.g., an scFv, Fab, or natural cell ligand. The tumor cell surface binding protein may target cancer cell surface protein targets, e.g., CD19, SSTR2, GD2, PTK7, CD5, CD20, CD22, CD110, CD117, CD19 LH scFv, PTK7 HL scFv, GD2 HL scFv, GD2 LH scFv, Integrin aVB3 HL ScFv, SSTR2 HL scFv, SSTR2 LH scFv, 2×SST28 3×G4S, 2×SST28 4×G2s, TPO ligand, hSCF ligand (SEQ ID NO: 4). (Note: HL indicates a heavy chain-light chain orientation and LH indicates a light chain-heavy chain orientation). The tumor cell binding domain may be a single chain antibody variable domain fragment or a tumor cell receptor ligand that binds one selected from the group consisting of SSTR2, PTK7, GD2, SSTR5, CD19, aVB3, CD110, and CD5. The tumor cell binding domain may be SSTR2 scFv (SEQ ID NO: 52-58) (either LH or HL), PTK7 HL scFv (SEQ ID NO: 17-23), SSTR2 HL scFv, CD19 scFv (SEQ ID NO: 7-11) (LH or HL orientation), GD2 scFv (SEQ ID NOS. 38-44) (in LH or HL orientations), Integrin aVB3 ScFv (SEQ ID NOS: 45-51) (in LH or HL orientations), 2×SST28 3×G4S (SEQ ID NOS: 59-65), 2×SST28, 4×G2s (SEQ ID NOS: 66-72), TPO (ligand) (SEQ ID NOS: 80-86), and SCF (ligand) (SEQ ID NOS: 87-93), SSTR2-8 (SEQ ID NOS: 156-160), GD2-3 (SEQ ID NOS: 161-165), PTK7-14 (SEQ ID NOS: 166-170).

In a variation, the flexible linker may be, e.g., G4S (SED ID NO: 155), albumin (SEQ ID NOS: 31-37), Fc (SEQ ID NOS: 87-93). As referenced herein, the linkers may be combined, e.g, G4S refers to AA Sequence (GGGGS) (SEQ ID NO: 155); G4S-albumin-G4S (e.g., (based on AA Seq) SEQ ID NO: 155-SEQ ID NO: 31-SEQ ID NO: 155); G4S-Fc-G4S (e.g., based on AA Seq.) SEQ ID NO: 155-SEQ ID NO: 87-SEQ ID NO: 155).

In a variation, the gamma delta T cell surface protein binding may be one or more of, e.g., scFV, Fab, and/or natural gamma delta T cell ligand. In a variation, an example of gamma delta T cell surface antigen proteins targets disclosed herein include, e.g., CD3DL subunits, ITCR (T cell receptor) subunits, CD16, NKG2D, FasL, TRAIL. The gamma delta T cell surface antigen protein actuators, engagers and/or activators may be at least one of CD3 HL scFv, CD3 (Hum2) HL scFv, JAML HL scFv, CDXAR ligand, gd-c (V1) HL scFv, gd-c (V6) HL scFv.

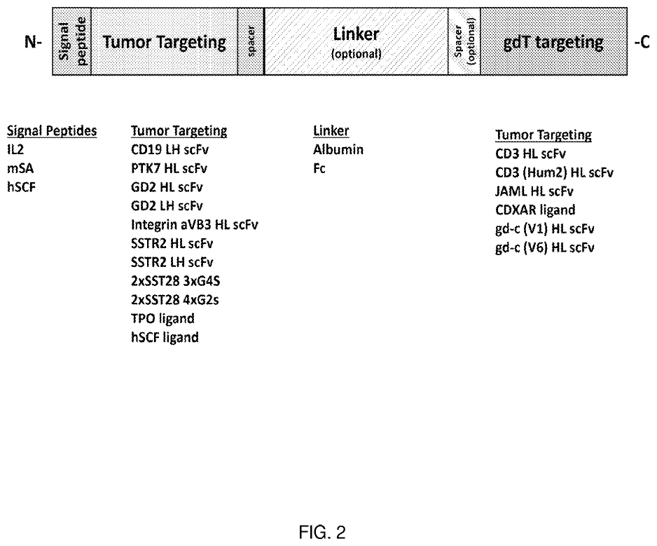

FIG. 2 provides a schematic representing variable elements of a generic STAR design and exemplary specific element identities.

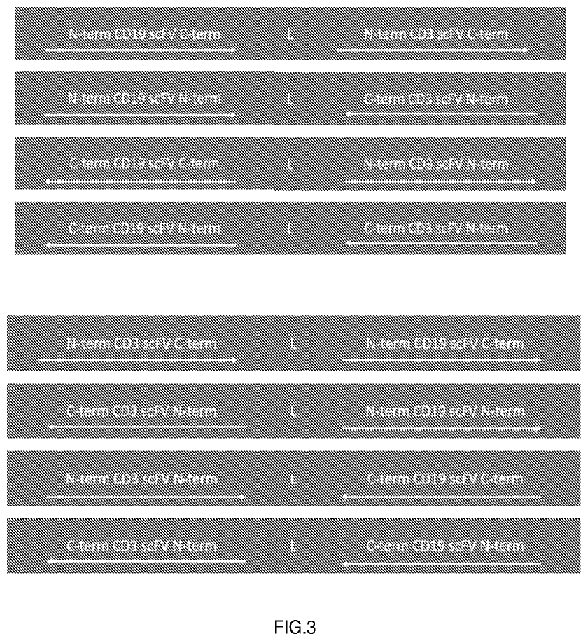

FIG. 3 demonstrates further schematics representing variable elements of STAR designs. In the present disclosure, the N terminus vs C terminus arrangement of the elements can be arbitrary. For instance, an exemplary CD19/CD3 molecule can be arranged with CD19 scFV at/toward the N terminus and CD3 at/toward the C terminus. However, the CD3 could likely be located at the N terminus and the CD19 at the C terminus. The IL2 signal sequence (or alternative) might always be at the N terminus. This general scheme may apply to all the described molecules according to the present disclosure. In addition, it is possible that the protein sequences may be reversed relative to their location within the protein.

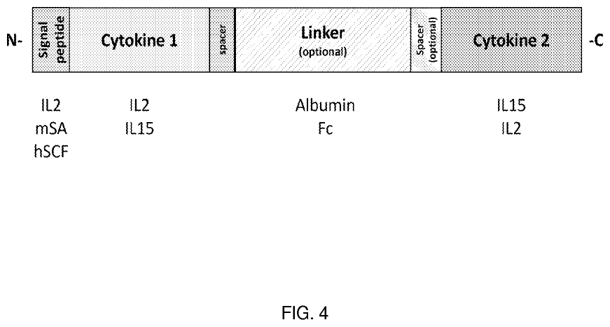

FIG. 4 demonstrates a schematic of an alternative STAR design capable of binding gamma delta T cells. The ligand based STAR is shown with cytokind actuators on each end of the construct. In this exemplary STAR, the signal peptide can be, e.g., IL2, mSA (SEQ ID NO: 3), or hSCF (SEQ ID NO: 4). Cytokine 1 may be IL2 or IL15. The optional linker (e.g., the flexible linker or Central linker) may be albumin (e.g., SEQ ID NOS: 31-37) or Fc (SEQ ID NOS: 87-93), Cytokine 2 may be IL 15 or IL2.

FIG. 5 is a LentET STAR schematic. It shows the elements that may be variably present in the transgene design on a lentiviral cassette for the expression of a STAR from a target cell. The transgene may include one or more of an (optional) shRNA cassette, internal promoter (e.g., MND or HSPA8), and WPRE sequence (optional). LentET is a lentiviral packaging system. In a variation, the transgene packaging plasmid is driven by an external CMV promoter which drives the expression of a transgene cassette RNA including one or more of: 1) the cis-viral elements necessary for viral assembly and packaging, 2) an internal promoter sufficient for gene expression in the target tissue (e.g, the MND promoter (SEQ ID NO: 152) or HSPA8 promoter (SEQ ID NO: 153)), 3) an ECO optimized STAR-encoding cDNA sequence, 4) optionally a mutated version of the Woodchuck Hepatitis Virus Posttranscriptional Regulatory element (WPREmut) and 5) optionally an shRNA expression cassette driving the expression of on shRNA directed against a component of the major histocompatibility class (MHC) I or II complexes.

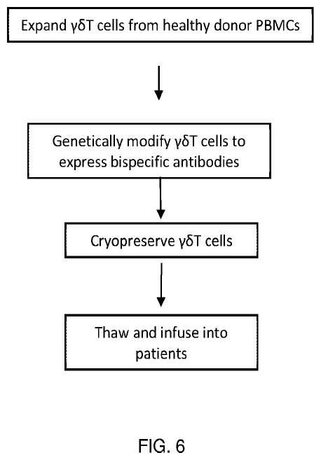

FIG. 6 provides a schematic of methods of manufacturing and genetically engineered gamma delta T cells. Donor peripheral blood mononuclear cells (PBMCs) of autologous or allogenic sources that can be pre-screened for disease-specific profiles may be used as starting material for the expansion, genetic engineering, and purification of genetically modified gamma delta T cells. Final products can include various compositions of gamma delta T cells which may include, e.g., gamma delta 1+ and gamma delta 2+ T cells, at various ratios for the adoptive transfer and treatment of various blood cancers and solid tumors.

Also disclosed are methods and compositions of gamma delta T cells which may include, e.g., gamma delta 1+ and gamma delta 2+ T cells genetically engineered to express and/or secret therapeutics against tumor antigens for the treatment of various blood cancers and solid tumors. Gamma delta T cells can be expanded from autologous or allogeneic donors under serum-free conditions. Donors can be selected from a set of screening criteria that include, but are not limited to, disease-specific/target specific profiles such as cytotoxicity assay in the presence or absence of other drugs and/or immunotherapies. Expansion of gamma delta T cells can be performed under serum-free conditions in a two-phase expansion procedure using PBMCs of autologous or allogenic source as starting material. Briefly, in one variation, PBMCs may be divided into two cultures for 1) gamma delta 2+ T cell expansion in the presence of zoledronic acid and IL-2 and 2) gamma delta 1+ T cell expansion by delta 1 monoclonal antibody-based activation in the presence of IL-2. Both expansion procedures may include two-phases: Phase 1 in the presence of the indicated supplements followed by an alpha beta T cell depletion; and Phase 2 in the presence of only IL-2. Alternatively, gamma delta 1+ T cell expansion can also be performed by exposure to concanavalin A (Con A) or phytohaemagglutinin (PHA) stimulation in the presence of IL-2.

Genetic modification may be performed during Phase 1 or Phase 2 of expansion. Phase 1 modifications can be achieved using modalities that heritably modify chromosomal DNA using, for example, viral or non-viral approaches. These approaches include but are not limited to lentivirus, gamma retrovirus, CRISPR, TALENs, etc. Modification of the genome at this phase can pass the modification to all daughter cells produced during further expansion. Phase 2 modifications can be achieved using non-integrating approaches such as AAV or mRNA which are not passed to daughter cells.

In the present disclosure, STARs may be secreted from genetically engineered gamma delta T cell. Actuation may occur through several mechanisms occurring alone or in combination. STARs may mediate binding to CD3 (or other T cell ligands) on gamma delta T cells and ligand molecules on target cells to form an immunological synapse which in turn activates T cell cytotoxicity. The secreted STARs can also mediate engagement between non-genetically modified gamma delta T cells and ligand molecules on target cells to form an immunological synapse which in turn activates T cell cytotoxicity.

In the present disclosure, genetic modification can take place with STARs and/or chimeric antigen receptors (CARs) in gamma delta 1+ and gamma delta 2+ T cells in combination or separately.

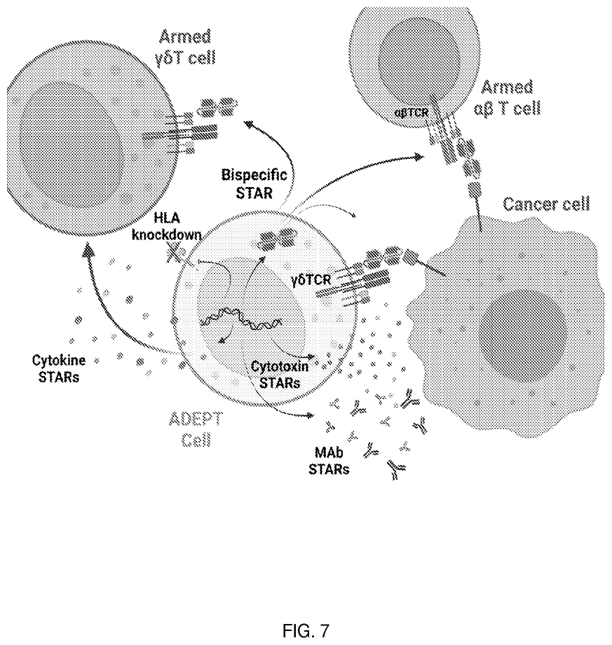

FIG. 7 is a schematic of STARS Mechanism of action. The mechanism of action can include STARs expression and secretion from genetically engineered T cells which mediates engagement between T cells and antigen/receptors on target cells. The formation of a cytolytic synapse between the T cell and the target cell by the STARs leads to T cell activation and the release of proteolytic enzymes that mediates cytotoxicity of target cells. Secretion of STARs from genetically modified T cells also leads to the engagement of non-modified T cells with target cells, resulting in enhanced cytotoxicity. Genetic modification of T cells with STARs can be combined with other immunotherapeutic approaches (such as, but not limited to, chimeric antigen receptors or CARs, monoclonal antibodies, and/or cytotoxic enhancing molecules). The present molecules could also be produced and purified from in vitro expression systems and delivered as a recombinant protein product.

By generating a final drug product of various gamma delta 1+/gamma delta 2+ T cell ratios, enhanced tumor specific targeting and cytotoxicity is mediated by the unique features of each gamma delta T cell type as summarized in Table 1:

TABLE 1
Features of human gd T cells for Immunotherapy

| Feature | Vd1+ T cells | Vd2+ T cells |
|---|---|---|
| Tumor Migration | Enhanced | Only CCR5 |
| | (CCR5 and CCR2 | expression |
| | expression) | |
| Activation-Induced | Lower | — |
| Cell Death (AICD) | susceptibility | |
| Persistence | Greater persistence | Less persistence |
| | in vivo | in vivo |
| MHC Unrestricted | Yes | Yes |
| TCR | | |
| Anti-Tumor | Broad | Broad |
| Toxicity | | |
| Peripheral | Stable with age | Decreases with |
| Circulation | | age |

In aspects of the present disclosure, gd T cell codon optimized scFv and ligand nucleic acid sequences are provided as SEQ ID NOS: 1-185. In the present disclosure, T cells may be genetically engineered via plasmid, mRNA, AAV, lentivirus, retrovirus. According to the present disclosure, the engineered T cells may provide endogenous expression and secretion of STARs for enhanced cytotoxicity and be used for adoptive cell transfer.

According to the present disclosure, the production of recombinant STARs can be used as a therapeutic agent for cancers and solid tumors, including neuroendocrine tumors (NETs) and neuroblastoma. The therapeutic mechanism may include sequence optimized expression and secretion of STARs from the engineered T cells for autocrine/paracrine engagement between T cells and target tumor cells.

According to the present disclosure, the STARs may be a recombinant protein product or a recombinant purified molecule for direct use as a therapeutic agent. The present disclosure may include the endogenous expression of STARs and/or adoptive cell transfer of T cells to enhance cytotoxic function of not only adoptively transferred cells but also endogenous T cells.

According to the present disclosure, there can be provided IL-2 and other leader sequences for secretion from genetically modified T cells.

According to the present disclosure, the treatment of cancers and tumors expressing a target antigen can use T cell therapeutics expressing and secreting STARs which can be combined with other immunotherapeutic agents. The present disclosure can be a recombinant protein that can be delivered directly, and which can be combined with other immunotherapeutic agents.

According to the present disclosure, a product can include one or more autologous or allogeneic derived T cells, STARs, and/or other immunotherapeutic agents expressed from genetically modified gamma delta T cells or co-administered with genetically modified gamma delta T cells.

We disclose herein an engineered T cell, specifically, gamma delta T cells, secreting a STAR agent.

We disclose herein a gamma delta T cells (gdT cells) cell secreting one or more synthetic fusion proteins.

We disclose herein an engineered gamma delta T cells (gdT cells) secreting one or more synthetic fusion proteins targeted for secretion by inclusion of a modified serum albumin (mSA) signal peptide or stem cell factor signal peptide to enhance fusion protein production.

We disclose herein an engineered gamma delta T cells (gdT cells) cell secreting one or more synthetic fusion proteins that have been expression cassette optimized.

We disclose herein an engineered gamma delta T cells (gdT cells) secreting one or more synthetic fusion proteins that have been expression cassette optimized for expression in gamma delta T cells (gdT cells).

We disclose herein an engineered gamma delta T cells (gdT cells) secreting one or more synthetic fusion proteins that have been expression cassette optimized for expression in gamma delta T cells possessing gamma 9 and delta 2 T cell receptor subunits.

We disclose herein an engineered gamma delta T cells (gdT cells) secreting a STAR agent, where the therapeutic agent is a bispecific T cell actuator.

We disclose herein gamma delta T cells (gdT cells) secreting one or more synthetic fusion proteins, where the synthetic fusion proteins are bispecific T cell actuators.

We disclose herein an engineered gamma delta T cells (gdT cells) secreting one or more synthetic fusion proteins where the synthetic fusion proteins are bispecific T cell actuators including an anti-CD3 scFv fused to an scFv capable of binding at least one of CD19, PTK7, GD2, SSTR2, and/or alpha-V beta-3 integrin.

We disclose an engineered gamma delta T cells (gdT cells) secreting one or more synthetic fusion proteins where the synthetic fusion proteins are bispecific T cell actuators comprised of an anti-CD3 scFv fused to a cognate receptor ligand domain.

We disclose an engineered gamma delta T cells (gdT cells) secreting synthetic fusion proteins where the synthetic fusion proteins are bispecific T cell actuators including an anti-CD3 scFv fused to the receptor ligand domain from stem cell factor (SCF), thrombopoietin (TPO), SSTR2, or SSTR5.

We disclose an engineered gamma delta T cells (gdT cells) secreting synthetic fusion proteins are bispecific T cell actuators including an anti-CD3 scFv fused to two or more copies of the receptor ligand SSTR2 and/or SSTR5.

We disclose an engineered gamma delta T cells (gdT cells) secreting synthetic fusion proteins are bispecific T cell actuators including an anti-gamma delta TCR scFv fused to an scFv that binds at least one of CD19, PTK7, GD2, SSTR2, and/or alpha-V beta-3 integrin.

We disclose an engineered gamma delta T cells secreting synthetic fusion proteins are bispecific T cell actuators including an anti-gamma delta TCR scFv fused to a cognate receptor ligand domain.

We disclose an engineered gamma delta T cells secreting synthetic fusion proteins are bispecific T cell actuators an anti-gamma delta TCR scFv fused to the receptor ligand domain from stem cell factor (SCF), thrombopoietin (TPO), SSTR2, or SSTR5.

We disclose an engineered gamma delta T cells secreting synthetic fusion proteins are bispecific T cell actuators including an anti-JAML scFv fused to an scFv that binds CD19, PTK7, GD2, SSTR2, or alpha-V beta-3 integrin.

We disclose an engineered T cell secreting one or more synthetic fusion proteins where the synthetic fusion proteins are bispecific T cell actuators including an anti-JAML scFv fused to a cognate receptor ligand domain.

We disclose an engineered T cell secreting one or more synthetic fusion proteins where the synthetic fusion proteins are bispecific T cell actuators including an anti-JAML scFv fused to the receptor ligand domain from stem cell factor (SCF), thrombopoietin (TPO), SSTR2, or SSTR5.

We disclose an engineered T cell secreting one or more synthetic fusion proteins where the synthetic fusion proteins are bispecific T cell actuators including an anti-JAML scFv fused to two or more copies of the receptor ligand SSTR2 or SSTR5.

We disclose an engineered T cell secreting one or more synthetic fusion proteins where the synthetic fusion proteins are bispecific T cell actuators including an a cognate JAML ligand fused to an scFv that binds CD19, PTK7, GD2, SSTR2, or alpha-V beta-3 integrin.

We disclose an engineered T cell secreting one or more synthetic fusion proteins where the synthetic fusion proteins are bispecific T cell actuators including a cognate JAML ligand fused to a cognate receptor ligand domain.

We disclose an engineered T cell secreting one or more synthetic fusion proteins where the synthetic fusion proteins are bispecific T cell actuators including a cognate JAML ligand fused to the receptor ligand domain from stem cell factor (SCF), thrombopoietin (TPO), SSTR2, or SSTR5.

We disclose an engineered T cell secreting one or more synthetic fusion proteins where the synthetic fusion proteins are bispecific T cell actuators including a cognate JAML ligand fused to two or more copies of the receptor ligand of SSTR2 or SSTR5.

We disclose herein an engineered T cell secreting a STARagent, where the therapeutic agent is a cytokine.

We disclose an engineered T cell secreting one or more synthetic fusion proteins where the synthetic fusion proteins include a dual cytokine.

We disclose an engineered T cell secreting one or more synthetic fusion proteins where the synthetic fusion proteins include a dual cytokine, the dual cytokine being one or more of IL2 and/or IL15 (e.g., IL2-IL2, IL2-IL15, IL15-IL15).

We disclose an engineered T cell secreting one or more synthetic fusion proteins where the engineered T cell is a gamma delta T cell.

We disclose an engineered T cell secreting one or more synthetic fusion proteins where the engineered T cell is a gamma delta T cell and the gamma delta T cell has gamma 9 and delta 2 T cell receptor subunits.

We disclose an engineered T cell secreting one or more synthetic fusion proteins where the engineered T cell is a gamma delta T cell with gamma 9 and delta 2 T cell receptor subunits produced by ex vivo expansion of health donor peripheral blood mononuclear cells using a using a two-stage culture method.

We disclose an engineered T cell secreting one or more synthetic fusion proteins where the engineered T cell, which may be a gamma delta T cell, and which may further be with a gamma delta T cell gamma 9 and delta 2 T cell receptor subunits, is modified for synthetic protein production by lentiviral or Retroviral vector transduction or mRNA transfection.

We disclose herein an engineered hematopoietic cell secreting one or more synthetic fusion proteins that have been expression cassette optimized

We disclose optimized expression and secretion of a bispecific scFv-based antibody or soluble ligand from engineered gdT cells for autocrine/paracrine engagement between gdT cells and target expression tumor cells.

We disclose localized expression and secretion of a bispecific scFv-based antibody or soluble ligand from engineered gdT cells.

We disclose Endogenous expression bispecific scFv-based antibody or soluble ligand from gdT cells and adoptive cell transfer of gdT cells to increase T cell cytotoxic function.

We disclose unique features include optimized sequence for gdT cell expression, and IL-2 leader sequence for secretion from genetically modified gdT cells as seen in the attached figures.

We disclose treatment of target positive tumors using off-the-shelf gdT cell therapeutics expressing a bispecific scFv-based antibody or soluble ligand.

We disclose a recombinant vector encoding one or more synthetic fusion proteins disclosed herein.

We disclose a recombinant vector, which may be any vector known by one of skill in the art including but not limited to a recombinant lentiviral or recombinant retroviral vector, encoding one or more synthetic fusion proteins as disclosed herein. A recombinant vector (e.g., lentiviral or retroviral) may include an internal promoter. The internal promoter may be a e.g., MND or HSPA8 promoter.

We disclose a recombinant lentiviral or retroviral vector encoding one or more fusion proteins as disclosed herein and additionally encoding short hairpin RNAs targeting beta 2 microglobulin (B2M) (SEQ ID NO: 150) and/or class II transcriptional transactivator (CIITA) (SEQ ID NO: 151). The vector may optionally include the internal promoter MND or HSPA8.

We disclose a method of preparing an engineered T cell secreting one or more synthetic fusion proteins including the steps of (a) expansion of healthy donor immune cells (e.g., g9d2 T cells) (See FIG. 6).

The disclosed engineered cells may be applied as a cancer therapeutic agent capable of treating cancers including but not limited to B cell malignancies, neuroblastoma, osteosarcoma, neuroendocrine tumors (NETs), and acute myeloid leukemia (AML).

We disclose an expression vector for an engineered cell expressing a therapeutic agent or synthetic fusion protein disclosed herein.

We disclose a method of treating cancers including the steps of (a) third-party, healthy donor PBMCs are culture and expanded ex vivo in the presence of IL-2 and zoledronate to enrich for gdT cells, (b) enriched gdT cells are genetically modified ex vivo for STAR expression and secretion. Genetically modified gdT cells are qualified and can be frozen to generate a bank for subsequent use or immediately transfused into recipient to control and/or reduce tumor growth.

We disclose a therapeutic agent comprising a single chain antibody variable domain that binds SSTR2, wherein the single chain antibody variable region that binds SSTR2 has an amino acid sequence at least 96% identical to SEQ ID NO: 156.

We disclose a therapeutic agent comprising a single chain antibody variable domain that binds GD2-3, wherein the single chain antibody variable region that binds GD2-3 has an amino acid sequence at least 96% identical to SEQ ID NO: 161.

We disclose a therapeutic agent having, from N-terminus to C-terminus, a gamma delta T cell optimized signal peptide that is cleaved off prior to secretion, a linker that is SEQ ID NO: 155 (G4S) and a T cell binding protein that is SEQ ID NO: 94 (HUM2).

We disclose a therapeutic where the single chain antibody variable domain that binds SSTR2 is encoded by a nucleic acid sequence at least 96% identical to SEQ ID NOS: 157, 158, 159, or 160.

We disclose a therapeutic agent where the single chain antibody variable domain that binds GD2-3 is encoded by a nucleic acid sequence at least 96% identical to SEQ ID NO: 162, 163, 164, or 165.

We disclose an engineered gamma delta T cell capable of secreting at least one therapeutic protein, the therapeutic protein, from N-terminus to C-terminus, including a gamma delta T cell optimized signal peptide, a tumor cell binding domain, a linker, and a T cell binding domain, where the tumor cell binding domain has an amino acid sequence at least 96% identical to SEQ ID NO: 156 or SEQ ID NO: 161. The engineered gamma delta T cell optionally includes a linker with the sequence of SEQ ID NO: 155 (G4S). The engineered gamma delta T cell optionally includes a T cell binding protein with the sequence of SEQ ID NO: 94 (HUM2).

In a variation, the engineered gamma delta T cell has a tumor cell binding domain that is encoded by a nucleic acid sequence at least 96% identical to SEQ ID NO: 157, 158, 159, or 160.

In a variation, the engineered gamma delta T cell has a tumor cell binding domain that is encoded by a nucleic acid sequence at least 96% identical to SEQ ID NO: 162, 163, 164, or 165.

We disclose a recombinant viral vector encoding a therapeutic protein capable of biosynthesis and secretion by a gamma delta T cell, the therapeutic protein comprising, from N-terminus to C-terminus, a gamma delta T cell optimized signal peptide that is cleaved off prior to secretion, a tumor cell-binding protein domain, a linker, and a T cell binding protein, wherein the tumor cell-binding protein domain binds either of SSTR2 or GD2-3, where the tumor cell-binding protein domain that binds SSTR2 has an amino acid sequence at least 96% identical to SEQ ID NO: 156, or the tumor cell-binding protein domain that binds GD2-3 and has an amino acid sequence at least 96% identical to SEQ ID NO: 161. In a variation, the linker is SEQ ID NO: 155. In a further variation, the T cell binding protein is SEQ ID NO: 94.

In a variation, the tumor cell-binding protein domain that binds SSTR2 is encoded by a nucleic acid sequence at least 96% identical to at least one of SEQ ID NO: 157, 158, 159, or 160.

In a variation, the tumor cell-binding protein domain that binds GD2-3 is encoded by a nucleic acid sequence at least 96% identical to at least one of SEQ ID NO: 162, 163, 164, or 165.

### Experimental Data

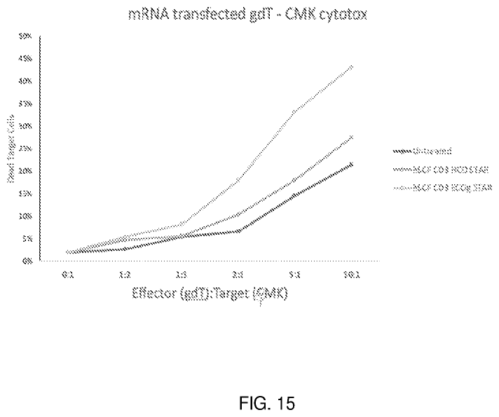

We provide optimized STAR contructs capable of improved expression from gamma delta T cells. We have shown that our proprietary ECOg optimized molecules offer improved expression in gamma delta T cells over standard human codon optimization. FIG. 15. Improved expression applied to a gamma delta T cell is reflected in improved or increased cytotoxicity. ECOg offers improvement over standard human codon optimization in gamma delta mediated gene expression. Here, gdT cells were transfected with mRNA carrying hSCF-coding STARs that had been ECOg or human (HCO) optimized. 4 hours post transfection gdT cells (effector) were co-incubated with CMK cells (target) and the cytotoxic potential of the transfected gdTs against CMK cells was measured.

This ECOg optimization was applied to optimize elements of the STAR relating to multiple steps along the expression pathway to provide highly efficient therapeutic effect through improved cytotoxicity over non-optimized cells. FIG. 1 provides a schematic of an exemplary STAR framework. The STARs disclosed by construct and through the Sequence Listings provided below, are optimized for improved expression over corresponding, human codon optimized and/or non-optimized constructs. The STAR constructs provided were optimized specifically for improved expression in gamma delta T cells. This optimization improved the ability of gamma delta T cells to more efficiently produce and express therapeutic proteins, as demonstrated by improved cytotoxicity.

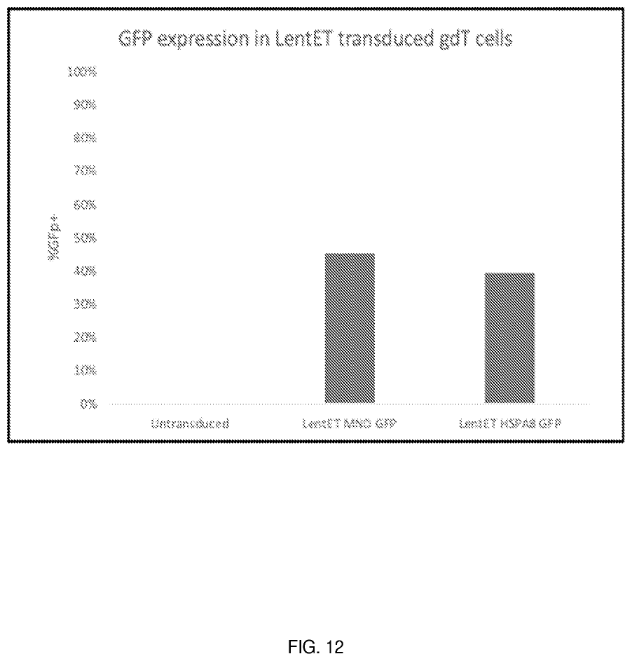

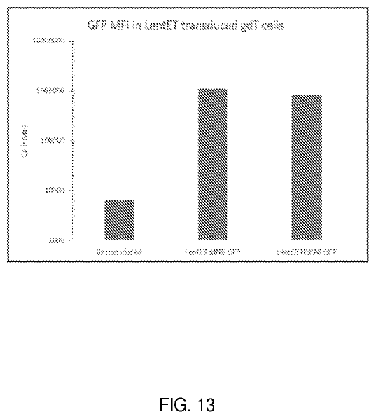

The optimizations targeted each step of the protein production pathway to increase translation and expression in the gamma delta T cell environment. For example, the promoter element of the STAR has been optimized for improved gamma delta T cell expression of therapeutic molecules. For example, we provide an ECO optimized MND promoter (SEQ ID NO: 152). In a variation, we provide a non-viral promoter, the HSP8 promoter (SEQ ID NO: 153), which has been ECO optimized. See FIG. 12 and FIG. 13 for a data demonstrating the retained expression of GFP in cells driven by the novel disclosed HSP8 promoters when compared to the MND promoters. MND and HSPA8 GFP expression in lentivirally transduced gdT cells. Fresh gdT cells were transduced with lentiviral particles carrying a GFP expression cassette driven by either the MND (myeloproliferative sarcoma virus MPSV enhancer, negative control region NCR deletion) or HSPA8 (Heat shock 70 kDa protein 8) (FIG. 12) The percent of GFP+ cells was measured at day 6 of gdT expansion. (FIG. 13) The mean fluorescent intensity (MFI) of all cells was measured at day 6 of gdT expansion.

We disclose herein the novel HSPA8 promoter (SEQ ID NO: 153) which was optimized to provide a non-viral promoter capable of reaching expression levels comparable to that of the viral promoter MND. With the HSPA8 promoter, we disclose a STAR with a preferable composition which, by using a non-viral promoter, reduces known problems created by the MND viral promoter. As shown in FIG. 12 and FIG. 13, the novel HSPA8 promoter was optimized to reach expression levels comparable to the MND promoter while providing a desirable non-viral promoter.

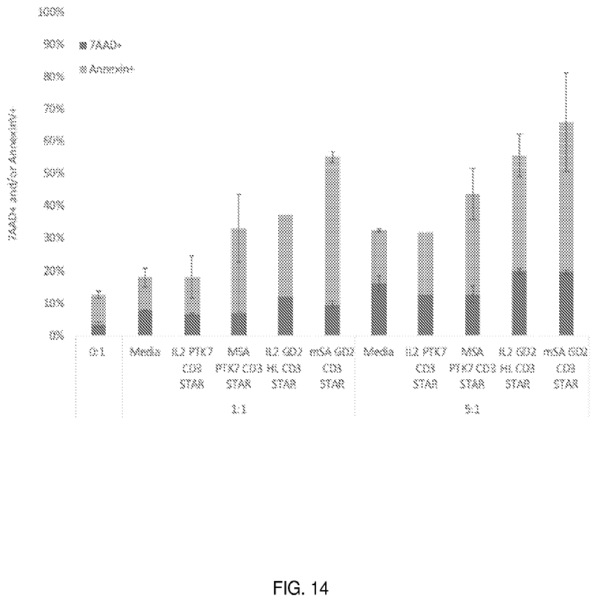

STARs were specialized for expression in gamma delta T cells through optimization of the signal peptide components. The increased expression generated by the optimized signal peptide components were demonstrated by improved cytotoxicity of the STAR expressing cells. In a variation, the mSA signal peptide (SEQ ID NO: 3) was optimized for gamma delta T cell expression. The disclosed signal peptides improved cytotoxicity of the gamma delta T cells through increased expression. FIG. 14 shows that the mSA signal peptide improves cytotoxicity of secreted media from PTK7 and GD2 STAR expressing 293T cells. Here, 293T cells were transfected with the respective STARs and conditioned media was collected 48 hours post-transfection. Gamma delta cells (effector) and IMR5 cells (target) were co-incubated in the presence of the conditioned media and the percent killing of IMR5 cells was measured after 4 hours. Ratios of effector: target cells are shown on the X axis label.

In a variation of the STAR provides expression optimized linkers (e.g, flexible linkers or Central linkers). The linkers were optimized specifically for improved ability to be secreted from gamma delta T cells. We provide two optimized linkers, albumin (SEQ ID NO:SEQ ID NOS; 31-37) and Fc (SEQ NOS: 87-93).

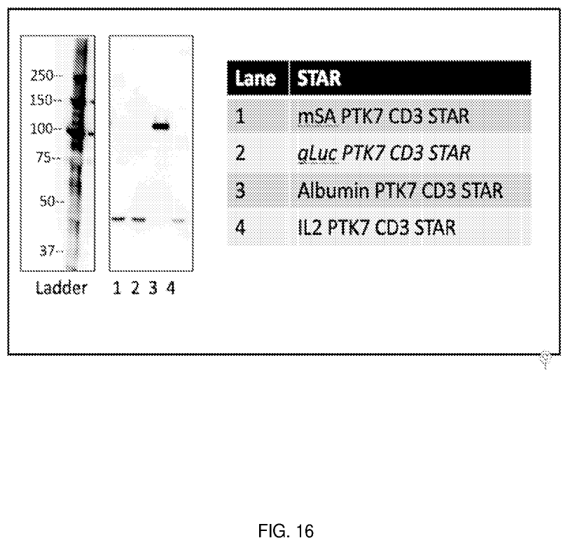

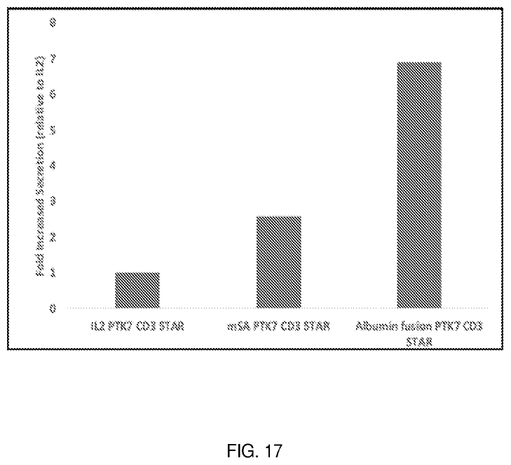

FIG. 16 and FIG. 17 demonstrate the improvements provided by the disclosed linkers. Here, albumin fusion and mSA signal peptide increase STAR secretion over IL2 design. Various STAR designs were transfected into 293T cells and conditioned media was collected 48 hours post transfection. FIG. 16 provides Western blot analysis of the designated STAR proteins. FIG. 17 shows quantitation of STAR secretion in the media normalized to the IL2 PTK7 CD3 STAR design.

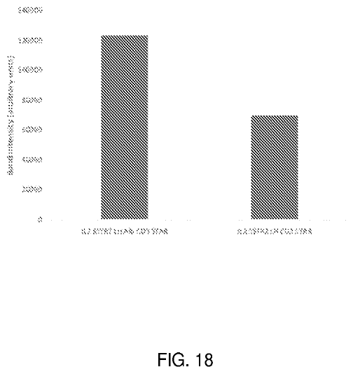

FIG. 18 further characterizes improvements provided by the disclosed optimized albumin linker, referred to as albumin fusion. FIG. 18 demonstrates that the central albumin fusion improves STAR secretion. To remedy the potential for steric hinderance of an N-terminus albumin fusion on STAR proteins, the albumin fusion was tested positioned between the tumor targeting end gdT targeting end of the STAR. 293T cells were transfected with the IL2 SSTR2 LH STAR with and without a centrally located albumin molecule. Conditioned media was collected 48 hours post-transfection and subjected to quantitative western blot.

The following data demonstrate the cytotoxic activity of the disclosed STAR variations.

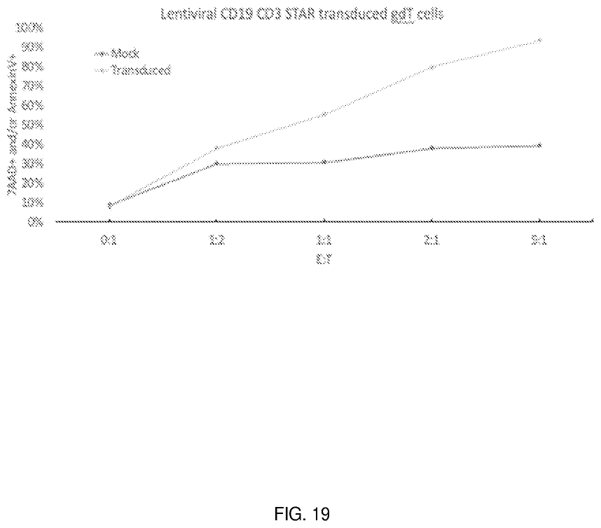

FIG. 19. gdT cells transduced with STAR-encoding lentivirus gain cytotoxic potential against target cells. Here, Fresh gdT cells were transduced with lentiviral particles carrying the IL2 CD19 LH CD3 STAR (e.g., SEQ ID NOS: 1-2). After several days of expansion the transduced and mock-transduced gdT cells (Effector, [E]) were coincubated with 697 cells (Target [T]) and toxicity toward the 697 cells was measured. Ratios of E: T cells are shown on the X axis. This demonstrates that gdT cells transduced with STAR-encoding lentivirus gain cytotoxic potential against target cells.

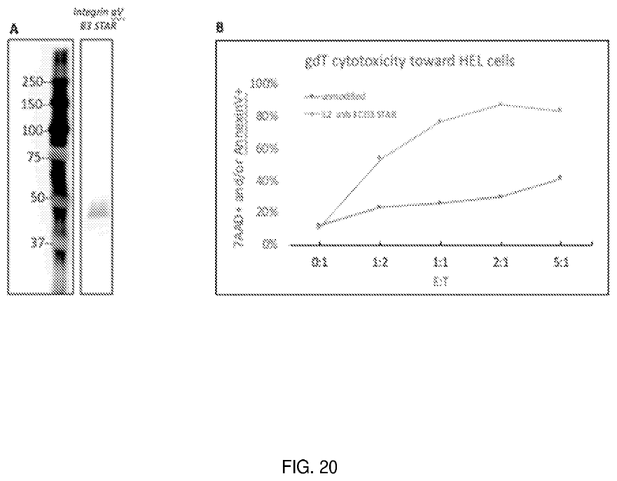

FIG. 20. Integrin aV B3 CD3 STAR (e.g., SEQ ID NOS: 45-51) promotes killing of target cells. (A) Plasmid expressing the integrin aV B3 CD3 STAR was transfected into 293T cells and conditioned supernatant was collected 48 hours post transfection. Western blot of the conditioned media shows the correct size of the integrin aV B3 STAR. (B) Fresh gdT cells were transfected with RNA encoding the integrin aV B3 CD3 STAR. gdT (effector, [E]) cells were mixed with Human ErythroLeukemia (HEL) cells (target, [T]) and cytotoxicity against the HEL cells was measured. Ratios of effector to target cells are shown along the X axis.

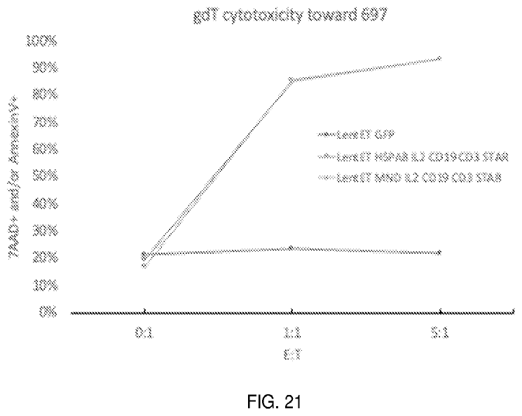

FIG. 21. IL2 CD19 CD3 STAR (e.g., SEQ ID NOS: 1-2) promotes killing of target cells. Here, 293T cells were transduced with lentivirus encoding GFP or the IL2 CD19 CD3 STAR (e.g., SEQ ID NOS: 1-2) driven by either the HSPA8 or MND promoters. Conditioned media was collected 48 hours after transduction. gdT cells (effector [E]) and 697 cells (target [T]) were mixed and conditioned media was added and cytotoxicity against the target cells was measured. Effector to target ratios are shown along the X axis. This figure shows both the efficacy of the IL2 CD19 CD3 STAR (e.g., SEQ ID NOS: 1-2) variations and demonstrates that the novel HSPA8 promoter provides a comparable non-viral option to the MND promoter.

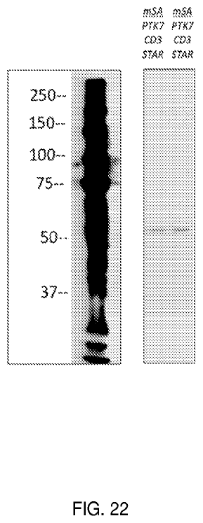

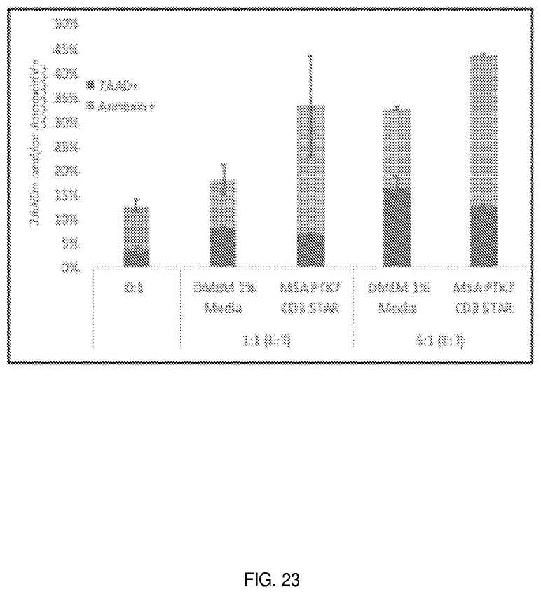

FIG. 22 and FIG. 23 demonstrate mSA PTK7 CD3 STAR (e.g., SEQ ID NO: 3, SEQ ID NOS: 17-23) promotes killing of target cells. Here, 293T cells were transfected with plasmid expressing the mSA PTK7 CD3 STAR (FIG. 22). Conditioned media was collected 48 hours after transfection. Western blot of the conditioned media shows a product of the expected size. In FIG. 23 gdT cells (effector [E]) and IMR5 cells (target [T]) were mixed and conditioned media was added and cytotoxicity against the target cells was measured. Effector to target ratios are shown along the X axis.

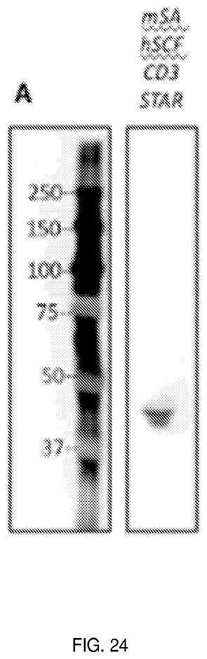

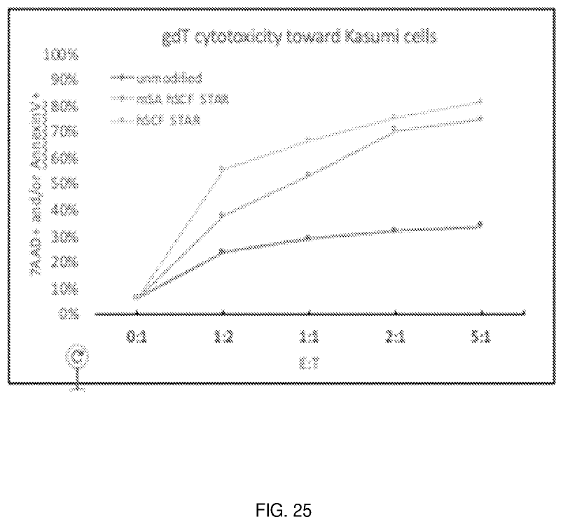

FIG. 24 and FIG. 25 show mSA (SEQ ID NO: 3) and native signal peptide hSCF CD3 STARs promote killing of target cells. FIG. 24 shows 293T cells were transfected with plasmid expressing the SCF CD3 STAR using the native SCF signal peptide (hSCF STAR). Conditioned media was collected 48 hours after transfection. Western blot of the conditioned media shows a product of the expected size. FIG. 25 shows gdT cells (effector [E]) were transfected with mRNA encoding the mSA (SEQ ID NO: 3) and native signal peptide versions of the hSCF CD3 STAR and mixed with IMR5 cells (target [T]). Cytotoxicity against the target cells was measured. Effector to target ratios are shown along the X axis.

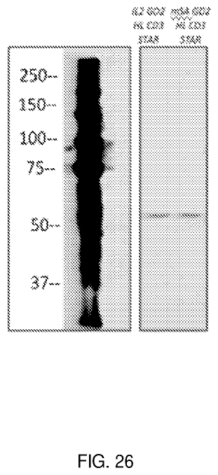

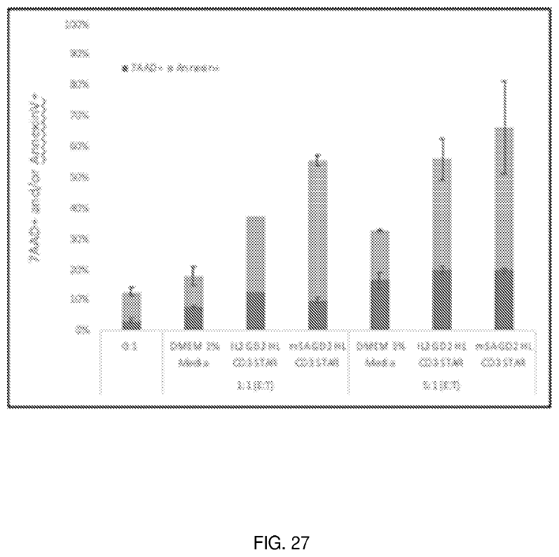

FIG. 26 and FIG. 27 demonstrate mSA and IL2 GD2 (e.g., SEQ ID NOS: 38-44) CD3 STARs promote killing of target cells. In FIG. 26, 293T cells were transfected with plasmid expressing the mSA or IL2 GD2 HL CD3 STAR. Conditioned media was collected 48 hours after transfection. Western blot of the conditioned media shows a product of the expected size. In FIG. 27, gdT cells (effector [E]) and IMR5 cells (target [T]) were mixed and conditioned media was added and cytotoxicity against the target cells was measured. Effector to target ratios are shown along the X axis.

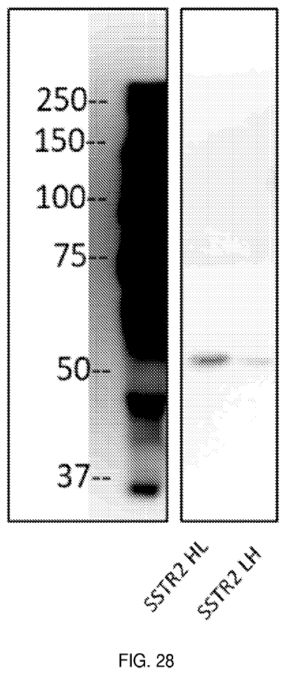

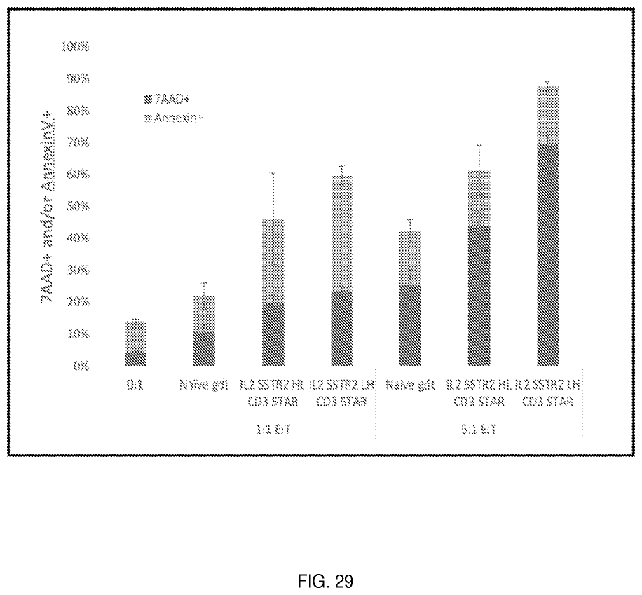

FIG. 28 and FIG. 29 demonstrate IL2 SSTR HL and LH CD3 STAR promote killing of target cells. In FIG. 28, Plasmids expressing the heavy/light (HL) and light/heavy (LH) arrangement of the SSTR2 scFv in the IL2 SSTR2 CD3 STAR chassis were transfected into 293T cells. Conditioned media was collected 48 hours post transfection where western blot detected bands of the appropriate size. In FIG. 29, mRNA encoding the IL2 SSTR2 HL or LH CD3 STAR were transfected into gdT cells. The transfected cells gdT cells (effectors [E]) were mixed with IMR5 cells (target [T] and the resulting cytotoxicity against the target cells were measured.

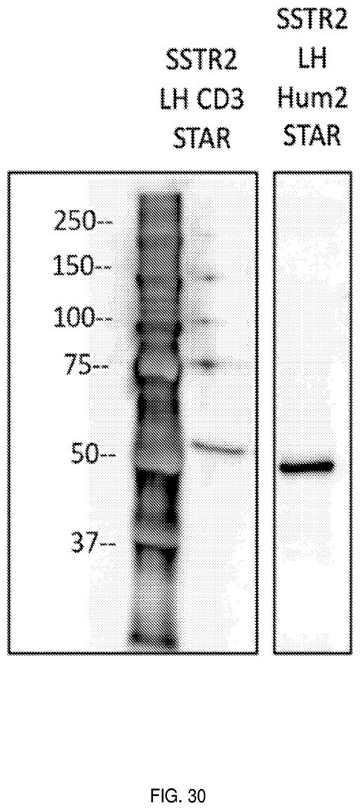

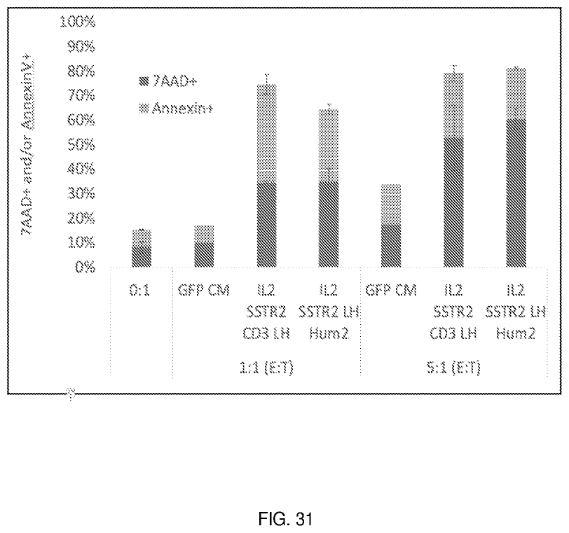

FIG. 30 and FIG. 31 demonstrate a humanized/deimmunized version of the CD3 scFv directs gdT mediated killing. In FIG. 30, 293T cells were transfected with plasmid expressing the CD3 (control) and Hum2 scFV (e.g., SEQ ID NOS: 94-100) (humanized/deimmunized) versions of the IL2 SSTR2 LH STAR. Conditioned media was collected 48 hours after transfection. Western blot of the conditioned media shows products of the expected sizes. In FIG. 31, gdT cells (effector [E]) were co-incubated with IMR5 cells (target [T]) in the presence of the conditioned media. Cytotoxicity against the target cells was measured. Effector to target ratios are shown along the X axis.

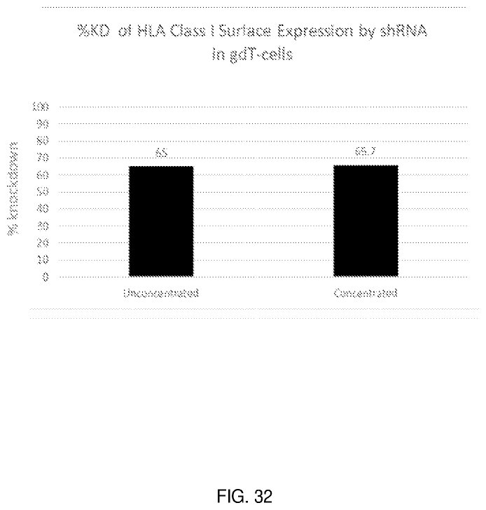

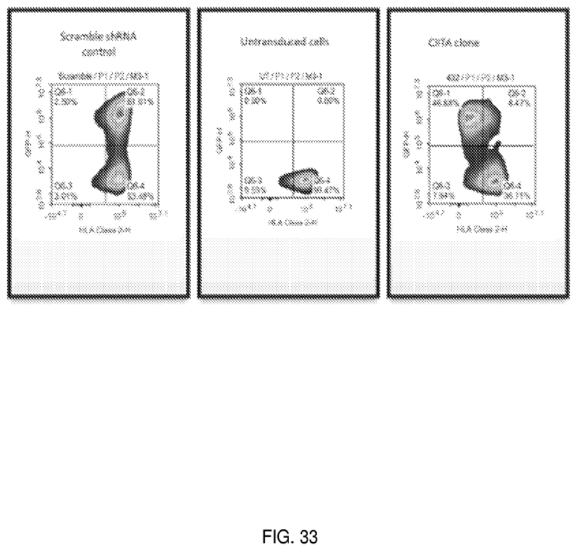

FIG. 32 and FIG. 33 demonstrate Lentiviral delivery of shRNA knocks down HLA Class I and II surface expression. In FIG. 32, gdT cells were transduced with concentrated and unconcentrated LentET vector carrying a cassette driving the expression of an anti B2m shRNA (B2m shRNA1) and percent knockdown of B2m (HLA Class I) was measured by flow cytometry. In FIG. 33, gdT cells were transduced with LentET vector carrying a cassette driving the expression of an anti CIITA shRNA (CIITA shRNA7) and knockdown of CIITA (HLA Class II) was measured by flow cytometry.

Our disclosed STARs are uniquely modified to preemptively address and avoid HLA mismatch to improve chances of graft survival. In a variation, our disclosed gdT-cell product (STAR) is allogeneic, meaning they are derived from non-donor PBMCs. It is generally observed that graft vs host disease (GVHD) is not a major concern for gdT cells as they kill in an MHC-independent manner. However, in an abundance of caution we preemptively try to circumvent immunogenicity ever being an issue my making our cell product more “universal” given that these gdT-cell therapies will probably be administered to patients who are already severely immunosuppressed.

In current literature, reduction in immunogenicity has been accomplished by reducing (siRNA/shRNA) or eliminating (CRISPR knockout) the presence of HLA I & II complexes on the donor cell surface. This poses a unique hurdle as there are multiple HLA genes, most of which are highly polymorphic. There are, however, non-polymorphic protein targets whose expression, if abrogated, would result in loss/reduction of expression of HLA at the cell's surface. For HLA I, we are using a lentiviral vector expressing an shRNA targeting the non-polymorphic beta chain of all HLA class I surface complexes called beta-2-microglobulin. By reducing levels of B2M, an essential component of the HLA I complexes, we are also reducing HLA I expression on the cell surface. Reduction of HLA class II expression is not as straightforward. There is no common structural element to all HLA II complexes to provide a target for knockdown, but the literature has shown that by targeting the “Class II Major Histocompatibility Complex Transcriptional Transactivator” (CIITA), it is possible to universally decrease or eliminate expression of HLA II on the cell surface. CIITA is necessary for transcription of the HLA II genes, and without it, transcription cannot take place. As with HLA I, we are using a lentiviral vector encoding an shRNA against CIITA to reduce HLA II surface expression. Ultimately, we would want to have a lentiviral vector that produces a STAR as well as both shRNAs against B2M and CIITA so that we can reduce HLA I & II levels on our gdT cell surface resulting in a safer and more effective therapeutic, siLentET.

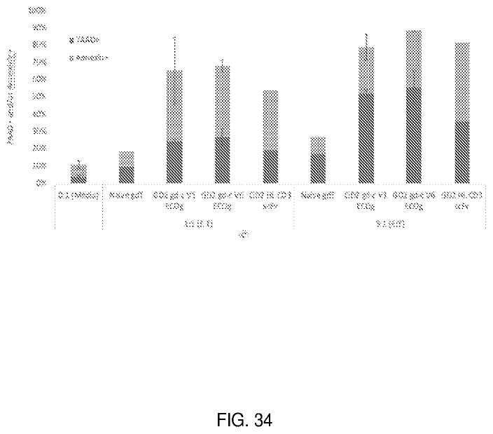

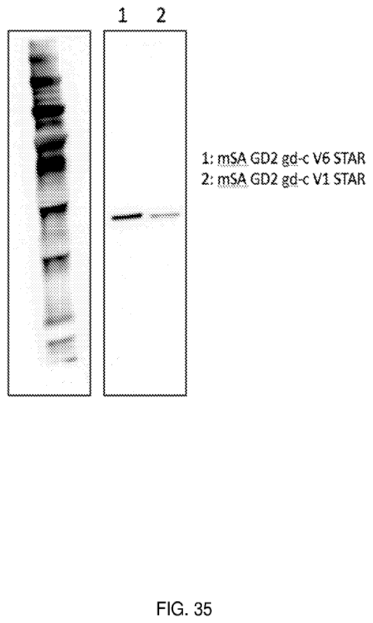

FIG. 34 and FIG. 35 demonstrate alternative gdT targeting moieties direct gdT mediated cytoxicity. In FIG. 34, shows 293T cells were transfected with plasmid expressing STARs directed toward target cells with an anti GD2 scFv and directed toward gdT cells with anti-gdT TCR scFv (gd-c V1 and gc-c V6) or anti CD3 scFv. Conditioned media was collected 48 hours after transfection. In FIG. 35, gdT cells (effector [E]) were co-incubated with IMR5 cells (target [T]) in the presence of the conditioned media. Cytotoxicity against the target cells was measured. Effector to target ratios are shown along the X axis.

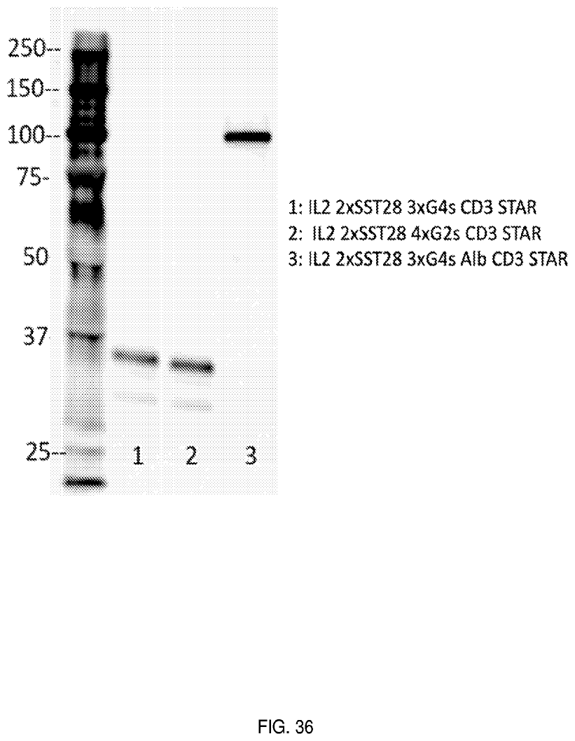

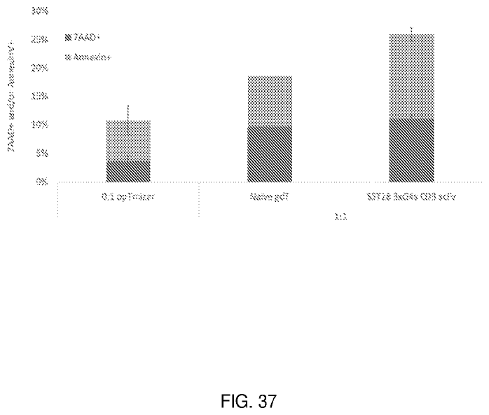

FIG. 36 and FIG. 37 demonstrates Somatostain ligand gdT mediated cytoxicity toward NET cells. In FIG. 36, 293T cells were transfected with plasmid expressing STARs directed toward target cells with somatostain ligand (SST28) and directed toward gdT cells with anti CD3 scFv. Conditioned media was collected 48 hours after transfection. In FIG. 37, gdT cells (effector [E]) were co-incubated with IMR5 cells (target [T]) in the presence of the conditioned media. Cytotoxicity against the target cells was measured. Effector to target ratios are shown along the X axis.

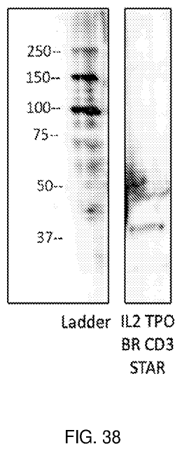

FIG. 38 demonstrates IL2 TPO BR CD3 STAR expression. Here, plasmid carrying the IL2 TPO BR CD3 STAR transfected into 293T cells express the expected protein product as detected by western blot.

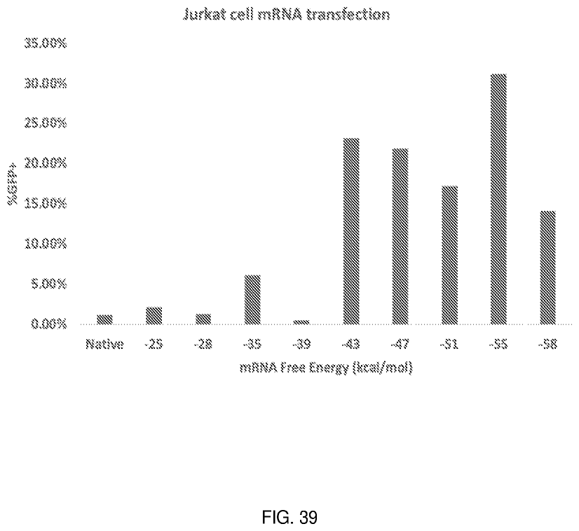

FIG. 39 demonstrates mRNA mediated protein expression correlates with mRNA free energy. Here, Jurkat cells were transfected with a panel of ECOg optimized mRNA constructs encoding varying from low to high mRNA free energy. GFP expression was measured 8 hours post transfection.

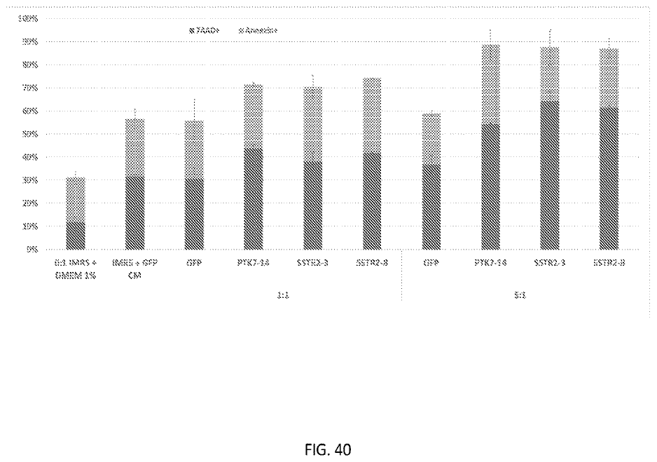

In FIG. 40, PTK7-14 and SSTR2-8 STAR plasmids were transfected into 293T-17 cells and conditioned media was harvested 48 hours after transfection. IMR5 tumor target model cells were co-incubated with the gamma delta T cells at ratios of 1:1 or 5:1 in the presence of the STAR-containing conditioned media. Percent killing of target cells was determined by flow cytometry. 7AAD refers to 7-aminoactinomycin D, a dye used to assess cell death. Annexin is use to measure apoptosis or cell death.

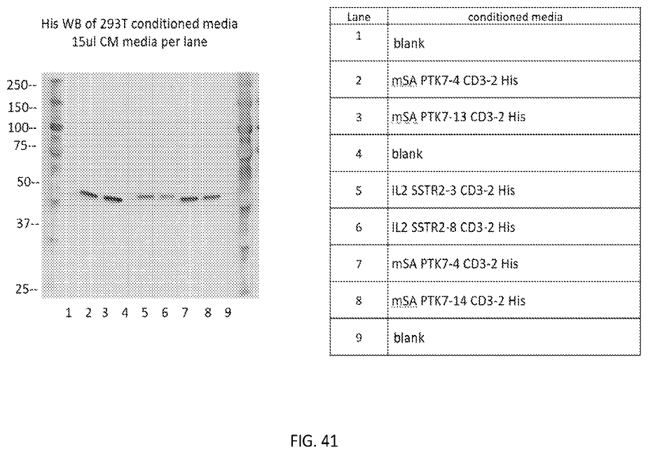

In FIG. 41, 293T-17 cells were transfected with STAR-expressing plasmid DNA. Conditioned media was harvested 48 hours post transfection and analyzed for STAR protein presence by anti-his detection western blot assay. Row 1 is blank. Row 2 is mSA PTK7-4 CD3-2 His. Row 3 is mSA PTK7-13 CD302 His. Row 4 is blank. Row 5 is IL2 SSTR2-3 CD3-2 His. Row 6 is IL2 SSTR2-8 CD3-2 His. Row 7 is mSA PTK7-4 CD3-2 His. Row 8 is mSA PTK7-14 CD3-2 His. Row 9 is blank.

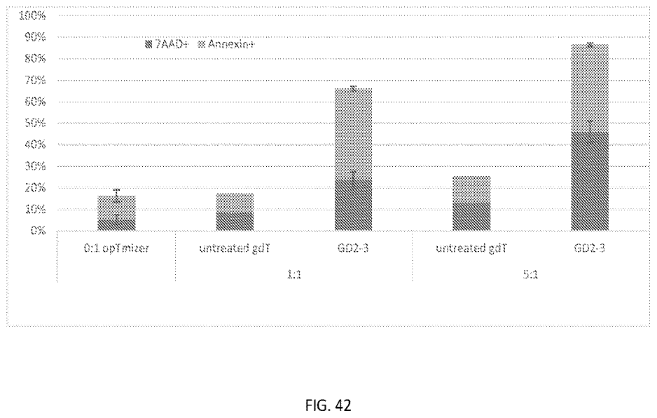

In FIG. 42, gd2-3 plasmid was transfected into 293T-17 cells and conditioned media was harvested 48 hours after transfection. IMR5 tumor target model cells were co-incubated with the gamma delta T cells at ratios of 1:1 or 5:1 in the presence of the STAR-containing conditioned media. Percent killing of target cells was determined by flow cytometry.

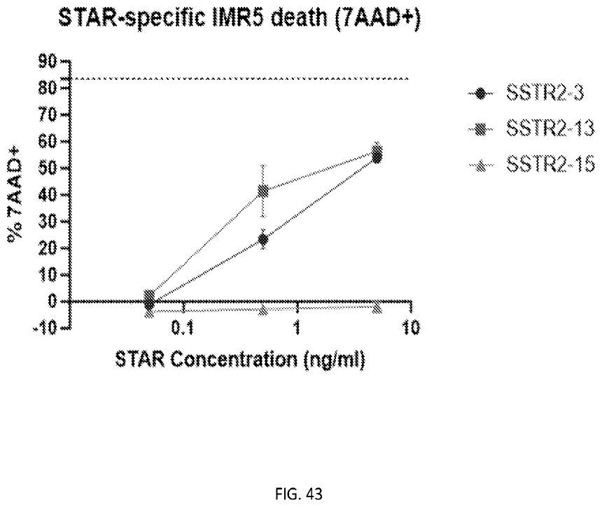

In FIG. 43, SSTR-STAR conditioned media was produced by transient transfection in 293T cells and quantified by ELISA. Gamma delta T cells and IMR5 cells were co-incubated in the presence of varying concentrations of STAR protein. Cytotoxicity was determined by flow cytometry.

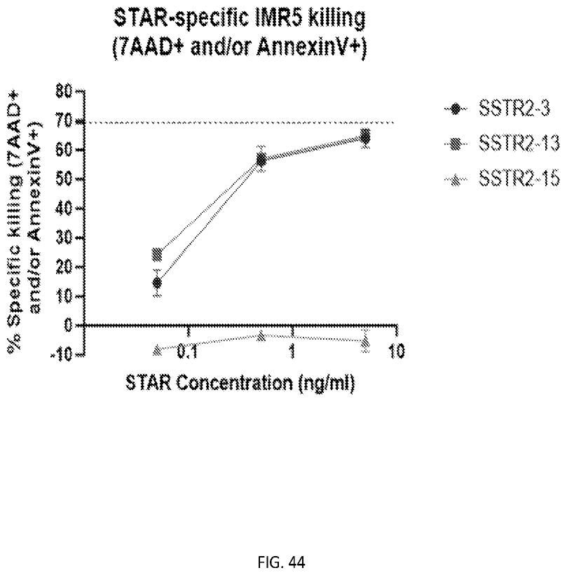

In FIG. 44, SSTR-STAR conditioned media was produced by transient transfection in 293T cells and quantified by ELISA. Gamma delta T cells and IMR5 cells were co-incubated in the presence of varying concentrations of STAR protein. Cytotoxicity was determined by flow cytometry.

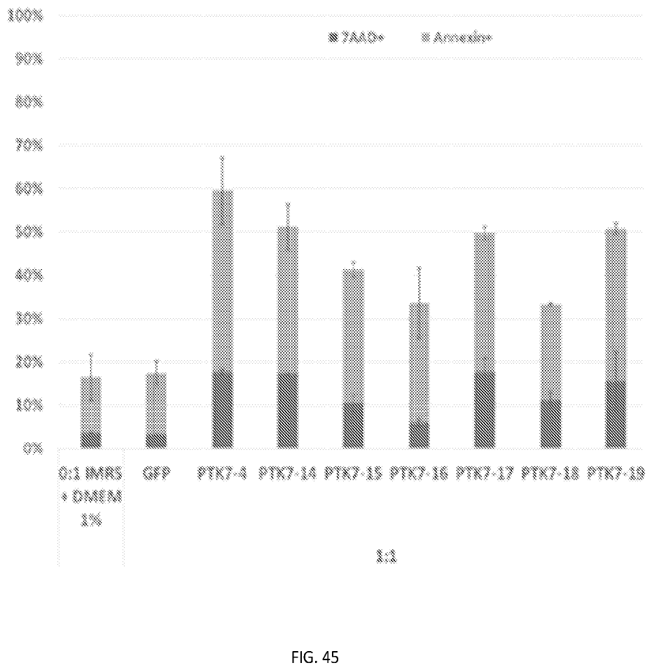

FIG. 45, shows cytotoxicity of various PTK7-STAR proteins (x-axis) at 1:1 ratio of effector (gamma delta T cells) to target (IMR5 cells). STAR conditioned media was produced by transient transfection in 293T cells. Gamma delta T cells and IMR5 cells were co-incubated in the presence of STAR protein at varying ratios of effector (gamma delta T cells) to target (IMR5 cells). Cytotoxicity was determined by flow cytometry.

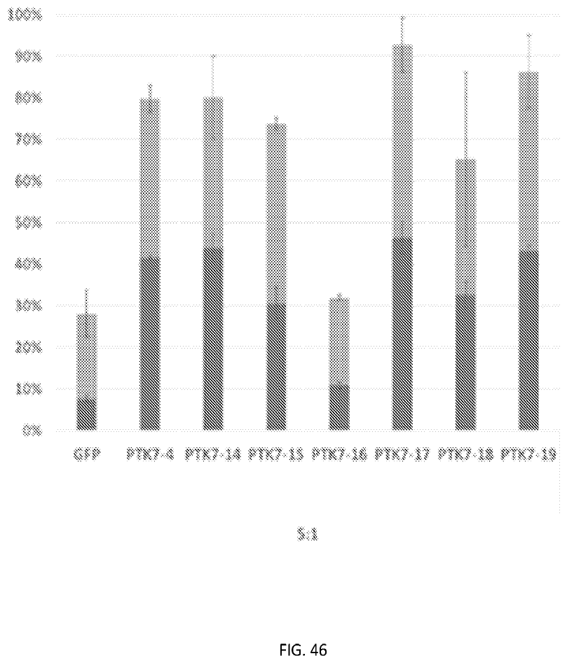

FIG. 46 shows cytotoxicity of various PTK7-STAR proteins (x-axis) at 5:1 ratio of effector (gamma delta T cells) to target (IMR5 cells). STAR conditioned media was produced by transient transfection in 293T cells. Gamma delta T cells and IMR5 cells were co-incubated in the presence of STAR protein at varying ratios of effector (gamma delta T cells) to target (IMR5 cells).

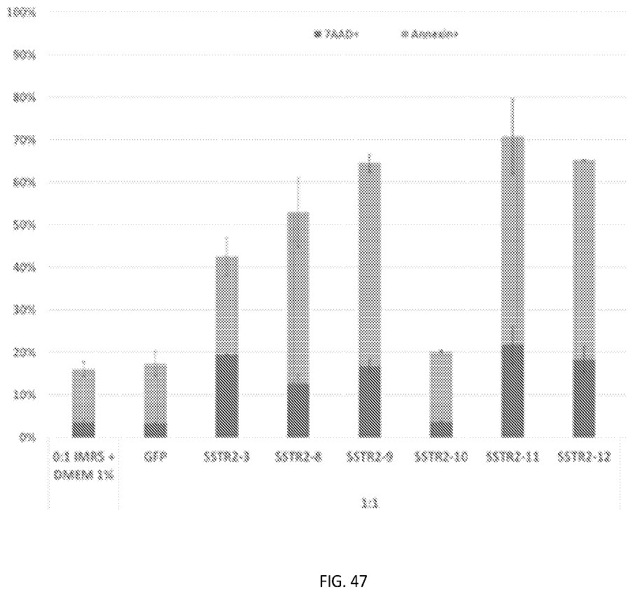

FIG. 47 shows cytotoxicity of various SSTR-STAR proteins (x-axis) at 1:1 ratio of effector (gamma delta T cells) to target (IMR5 cells). SSTR-STAR conditioned media was produced by transient transfection in 293T cells. Gamma delta T cells and IMR5 cells were co-incubated in the presence of STAR protein at varying ratios of effector (gamma delta T cells) to target (IMR5 cells). Cytotoxicity was determined by flow cytometry.

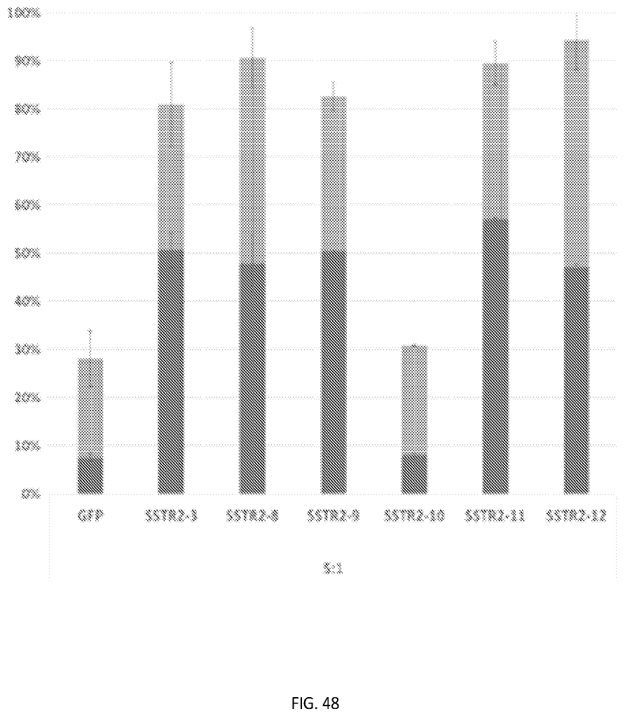

FIG. 48 shows cytotoxicity of various SSTR-STAR proteins (x-axis) at 5:1 ratio of effector (gamma delta T cells) to target (IMR5 cells). SSTR-STAR conditioned media was produced by transient transfection in 293T cells. Gamma delta T cells and IMR5 cells were co-incubated in the presence of STAR protein at varying ratios of effector (gamma delta T cells) to target (IMR5 cells). Cytotoxicity was determined by flow cytometry.

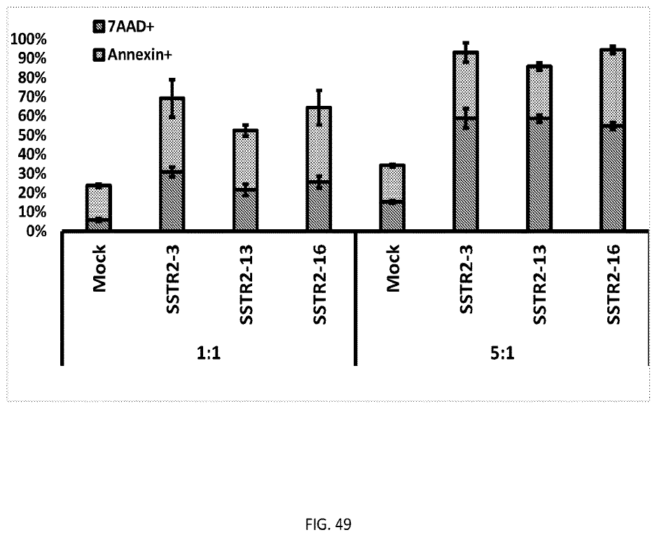

FIG. 49 shows cytotoxicity of additional SSTR-STAR proteins (x-axis) at 1:1 and 5:1 ratios of effector (gamma delta T cells) to target (IMR5 cells). SSTR2 cytotoxicity assay-293T cells were transfected with STAR encoding plasmids. Conditioned media was harvested 48 hours after transfection. Gamma delta T cells (effector) were co-incubated with IMR5 cells (target) at 1:1 and 1:5 effector: target ratios. Cytotoxicity was determined by flow cytometry.

### Methods

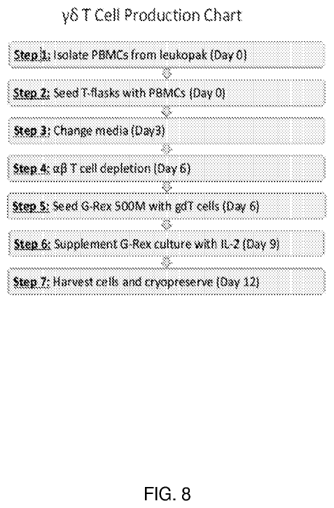

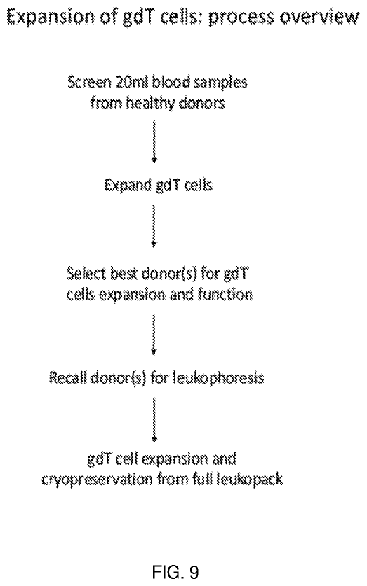

In the present disclosure, methods and compositions of gamma delta T cells genetically engineered to express and secret STARs for the treatment of various cancers and solid tumors, are provided. Turning to FIG. 9. Gamma delta T cells can be expanded from autologous or allogeneic donors under serum-free conditions. Donors may be selected from a set of screening criteria that include, but are not limited to, disease-specific/target specific profiles such as cytotoxicity assay in the presence or absence of other drugs and/or immunotherapies. (FIG. 8) Expansion of gamma delta T cells can be performed under serum-free conditions in a two-phase expansion procedure consisting of Days 0-6 in IL-2 and zoledronic acid in T-flask based culture (Phase 1) and Days 6-12 in IL-2 in Bioreactor based culture (Phase 2). An alpha beta T cell depletion step can be performed at Day 6 of culture, prior to Bioreactor based culture. Viral based genetic modification (lentiviral and/or gamma retroviral) can be performed on Phase 1 or Phase 2 of gamma delta T cell expansion. Alternatively, AAV-based or mRNA based genetic modification can be performed on Phase 2 of gamma delta T cell expansion for expression and secretion of STARs and/or other immunomodulators such as IL2-IL5 bispecific molecules.

In the present disclosure, the manufacturing of genetically engineered gamma delta T cells from autologous or allogeneic donor PBMCs under serum-free conditions can be in a two-phased expansion procedure. Genetic modification can take place during either phase 1 or phase 2 of gamma delta T cell expansion.

Donor pre-screening may be performed under a set of criteria to allow for optimal expansion, genetic modification, and cytotoxicity. Donor pre-screening may be based on disease-specific selection criteria such as disease/target specific cytotoxicity assay in the presence or absence of other drugs and/or immunotherapies.

According to the present disclosure, the manufacturing method can solve the problems of: serum-free expansion conditions; screening of donors for optimal expansion, genetic modification, and cytotoxicity towards disease specific target. According to the present disclosure, a two-phase expansion procedure can include an alpha beta depletion step for optimal product safety profile. Genetic modification may occur either in phase 1 or phase 2 or a combination thereof for optimal STARs secretion and/or other immunomodulators such as IL2-IL5 bispecific molecules secretion.

In aspects of the present disclosure, the expansion and genetic modification of gamma delta T cells can be capable of expression and secretion of STARs and/or other immunomodulators for disease-specific enhanced efficacy.

According to the present disclosure, a method can include: serum-free expansion conditions in a two-phased expansion method, genetic modification in either phase of expansion for combinatorial engineering with STARs and/or immunomodulators, expression and secretion of immunomodulators that promote gamma delta T cell expansion and viability both in vitro and in vivo.

An autologous or allogeneic gamma delta T cell genetically engineered with STARs harboring target tumor antigen for the treatment of cancers and solid tumors can be provided according to the present disclosure.

According to the present disclosure, the following can be provided: two phased expansions; genetic modification in either or both phases of the expansion; serum-free expansion conditions; and IL2-IL5 bispecific molecule expressed and secreted endogenously to enhance expansion, viability, and function.

In FIG. 8, a method according to the present disclosure is disclosed and summarized below.
Step 1: Isolate PBMCs from leukopak (Day 0)
Step 2: Seed T-flasks with PBMCs (Day 0)
Step 3: Change media (Day3)
Step 4: aß T cell depletion (Day 6)
Step 5: Seed Bioreactor with gdT cells (Day 6)
Step 6: IL-2 supplementation (Day 9)
Step 7: Harvest cells and cryopreserve (Day 12)

Full leukopak from American Red Cross, typically 1-1.5e10 total nucleated cells per leukopak and typically 150-400 ml total volume.

### Procedure:

### Day 0

Step 1: Isolate PBMCs from leukopak (Day 0). This step includes the following protocol: Obtain 22 ml whole blood, Add 18 ml of Ficoll-Paque to 50 ml conical tubes (2 per donor), Remove blood from collection tubes and pipette into a 50 mL conical tube for each donor, Record the starting volume of blood. This volume does not include the PBS washing volume in the next step. Wash collection tubes with 4 ml PBS to remove residual blood and cells off the sides of the tube, then put into 50 mL conical tube with the rest of the blood. Add additional PBS to make final PBS: Blood 1:1 ratio by volume. Total diluted blood volume/donor=44 ml. Mix the blood gently by inverting the 50 ml conical tube. Carefully add 22 ml of diluted blood to the top of the 18 ml of Ficoll-Paque media. (do not mix). Spin the conicals, 400×g, 35 min 20 C. Remove the top layer (plasma) with a pipette. Collect PBMC layer with a pipette and move into a new 50 ml conical tube pooling samples from the same donor. Wash the cells by resuspending the pellet in 3 volumes, ~45 ml PBS, then spin 500×g 10 min 20 C. Resuspend with 10 ml PBS to wash again. spin, 200×g 5 min 20 C. Resuspend with 10 ml PBS to wash again. Spin, 200×g 5 min 20 C.

Step 2: Seed T-flasks with PBMCs (Day 0). This step involves the following protocol: Resuspend cell pellet in 10 mL OpTmizer media. Count cells. Remove 1 ml (500K) cells for immunotyping CD3/gdTCR. Include gdTCR FMO control from pooled samples. Use ~100K cells/flow tube. Bring final cell density to 1.5×10⁶ cells/mL in OpTmizer. Add IL2 and zoledronate to make concentrations of these 500 IU/mL IL-2 and 5 μmol/L zoledronic acid. Culture cells at 37 C/5% CO₂ tilting flask (propped) to concentrate cell culture to bottom half of T-75 flask.

Day 3. Step 3: Change media (Day3). This step includes the following protocol: Transfer cells to 50 mL conical and gently pipette up/down with a 10 ml pipette to break up cell aggregates. Centrifuge cells at 250×g 10 min RT. Resuspend cells in OpTmizer media. Count cells. Bring final cell density to 1.5×10⁶ cells/mL in complete OpTmizer supplemented with 500 IU/mL of IL-2 and 5 μmol/L zoledronic acid and replating in the same T-75 flask. Culture cells at 37 C/5% CO₂ tilting flask (propped) to concentrate cell culture to bottom half of T-75 flask. Lentiviral/Retroviral transduction. After Step 4, bring cells to a final density of 1.5×10⁶ cells/mL in complete OpTmizer supplemented with 500 IU/mL of IL-2 and 5 μmol/L zoledronic acid and lentivirus/retrovirus at desired TU/mL in the presence of transduction enhancers and replate in the same T-75 flask. Culture cells at 37 C/5% CO₂ tilting flask (propped) to concentrate cell culture to bottom half of T-75 flask.

Day 4. This step includes the following protocol. Perform a second round of transduction at same TU/mL by removing half of the cells and transferring to a 50 ml conical tube. Centrifuge cells at 250×g 10 min RT. Bring cells to a final density of 1.5×10⁶ cells/mL in complete OpTmizer supplemented with 500 IU/mL of IL-2 and 5 μmol/L zoledronic acid and lentivirus/retrovirus at desired TU/mL in the presence of transduction enhancers and replate in the same T-75 flask. Culture cells at 37 C/5% CO₂ tilting flask (propped) to concentrate cell culture to bottom half of T-75 flask.

Day 5. This step includes the following protocol. Transfer cells to 50 ml conical tube. Centrifuge cells at 250×g 10 min RT. Resuspend cells in complete OpTmizer supplemented with 500 IU/mL of IL-2 and 5 μmol/L zoledronic acid and replating in the same T-75 flask. Culture cells at 37 C/5% CO₂ tilting flask (propped) to concentrate cell culture to bottom half of T-75 flask.

Day 6. Step 4: αβ T cell depletion (Day 6). This step includes the following protocol. Transfer cells to sterile container. Count cells. Remove 5e6 cells and save for flow immunotyping. Proceed with CliniMACS Plus depletion of αβ T cells. Step 5: Seed Bioreactor 500M-CS with gdT cells (Day 6). This step includes the following protocol. Resuspend cells in OpTmizer media. Count cells. Add 2e9 total live cells to each Bioreactor 500M-CS device containing 2.5 L of complete OpTmizer media supplemented 1000 IU/mL of IL-2. Culture cells at 37 C/5% CO2.

Day 9 Step 6: IL-2 supplementation (Day 9). This step includes the following protocol. Add 1000 U/ml IL-2 to each Bioreactor device through the needle port/tubing on the top of the device. Return GRex devices to 37 C/5% CO₂ incubator.

Day 11. AAV Transduction. This step includes the following protocol. Reduce media in GRex devices by ½ to ¾ of culture volume. Add desired AAV VG/cell supplemented with desired transduction enhancers directly into the GRex. Return GRex devices to 37 C/5% CO2 incubator.

Day 12. Step 7: Harvest cells and cryopreserve (Day 12). This step includes the following protocol. Remove top 2 L (80%) of media from each Bioreactor device without disturbing the cell layer on the Bioreactor membrane using the Gather-Rex pump. Recover cells in remaining 500 ml media and transfer cells to a 500 ml centrifuge bottle. Remove a sample of the cells for immunotyping analysis and count live cell numbers and determine viability by trypan blue. Centrifuge cells at 300×g 20 min RT. Resuspend cells in cryopreservation solution (PBS+5% HSA+10% DMSO) at 10e6 cells/ml. Distribute cells into cryobags. Load bags into control rate freezer and run specified freezing program. When freezing program is complete move cryovials to liquid nitrogen for storage.

mRNA electroporation. This step includes the following protocol. After Step 7 #4 (centrifugation), resuspend cells in electroporation buffer and add mRNA. Electroporate cells using appropriate instrument settings. Cells are either returned to complete media for 2 hrs for incubation at 37 C/5% CO₂ prior to, or immediately resuspended in cryomedia for freezing as described in Step 7 #5.

### EXAMPLES

### Example 1. Gamma Delta T Cells were Thawed. Cells were Incubated for 2 Hours at

37 C in complete media. Cells (1e7) were electroporated with 15 ug mRNA. 24 hours later, set up 697 cell cytotoxicity assay with gdT cells+50% conditioned media in assay tube.

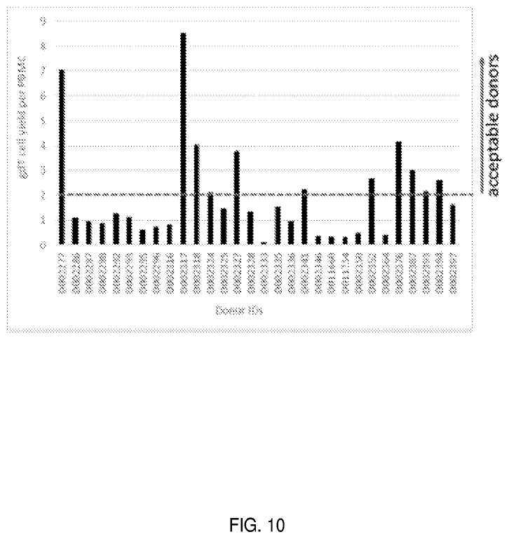

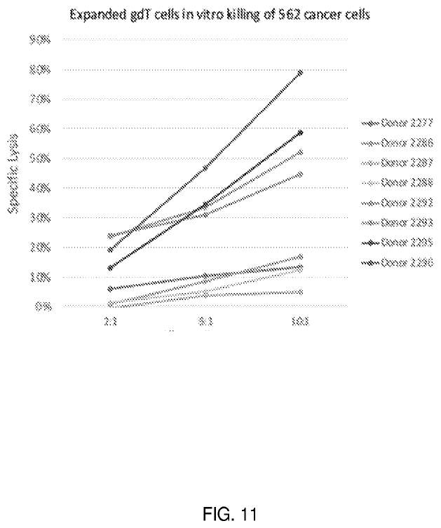

FIGS. 9-11 show experimental results. FIG. 9 is an overview of gdT cell expansion process. FIG. 10 shows identification of donors with acceptable ex vivo expansion of gdT cells from peripheral blood mononuclear cells (PBMCs). FIG. 11 shows screening of ex vivo expanded gdT cells to identify donors that generate gdT cells with high cytotoxicity toward K562 human cancer cells.

### Protocol Includes:

Purpose: Test the cytotoxicity of gdT cells transfected with CD19-CD3 STAR mRNA toward 697 cells, Nalm6 cells and 697-CD19KO cells.
Experimental Outline: Thawed gdT cells transfected with CD19-CD3 STAR mRNA, ~24 hrs later cytotox with 697 and Nalm6 cells

### Materials

Thawed day 12 gdT cells from 2 ARC2387 20e6 vials

HSA (25%) from Grifols Sterile PBS

Nalm6 cells

697 L cells

CD19_CD3 STAR mRNA CTS OpTmizer T cell expansion kit, Life Technologies, Cat #A1048501

** Need ~25 ml complete gdT media (OpTmizer+supplement+glutamine+1000 IU/ml IL-2)

Combine 40 mL of OpTmizer T-cell basal expansion medium with 1.106 mL of OpTmizer supplement and add 400 μL of 200 mM L-Glutamine (Gibco, Cat. #16777-162).

Prepare freshly and store for only 1 week at 4 degrees.

IL-2 (1000 U/ul), stored at −80

Reconstitute in 250 μg of IL-2 (Peprotech, Cat. #AF-200-02) in 100 mM acetic acid, add 16.25 mL of sterile 1% BSA. Aliquot and store at −80 degrees for long-term storage (up to 1 year) and store at −20 degrees for short-term storage during expansion.

For 100 mM acetic acid, dissolve 14.4 μL of glacial acetic acid (Sigma, Cat. #A6283) in 2.486 mL of PBS (Gibco, cat. #14190250).

For 1% BSA, dissolve 200 mg of BSA (Sigma, Cat. #A9647) to a final volume of 20 mL in PBS.

CD19/CD3 bispecific antibody BPS Biosciences, item 100441-1, 0.82 mg/ml, for this experiment the ab was taken from a 10 ul aliquot frozen at −80. Dilute this stock 1:100 in OpTmizer media, Add 2.4 ul of ab to each 200 ul reaction tube to give a final concentration of 100 ng/ml.

OpTiMem media (Fisher cat #31-985-062)

4 mm cuvette Violet Proliferation Dye (VPD450, BD Cat. 562158)

### Procedure:

1 vial of 25e6 cells thawed into 5 ml of 5% HSA/PBS:

Prepare thawing media: 2 ml of 25% HSA into 8 ml PBS. Warm to 37 C.

take vial directly from −80 C to 37 C water bath to thaw

thaw until just a small chunk of ice remains.

Transfer cells to 15 ml conical tube.

Add 1 ml of prewarmed thawing media to the cells slowly (dropwise over 30 sec) while mixing.

Add an additional 3 ml of thawing media

spin 250×g 5 min RT

Resuspended cells in 8 ml OpTmizer+15.1 ug/ml IL2 in a T25 flask.

Incubated in TC incubator 2 hrs.

2 hrs after thaw collect cells and disperse into single cells by pipetting up/down.

Count cells and determine viability

### Electroporation:

Prewarm and equilibrate 3 ml Optimizer complete media containing 1000 IU/ml IL-2 in each well of a 6 well plate.

Centrifuge gdT cells counted in step 8 above 250×g 5 min RT.

Resuspend cells to 10e6/ml in Optimem media

Add 1 ml of cells (=10e6 cells) to each of 2 1.5 ml epi tubes.

Centrifuged epi tubes noted above 250×g 5 min, RT

Remove supernate completely

Resuspend cells in 100 ul Optimem. Pipette cells up/down 3-5 times to break up cell pellet.

For the CD19-CD3 STAR group-Add 10 ul mRNA (=15 ug). Mix and put the cell/mRNA mixture into a 4 mm cuvette. (loaded the entire mixture, ~130 ul)

Electroporate using the following settings:
Square wave
500V
5 ms pulse length
1 pulse
4 mm Cuvette

Add ~500 ul warmed media (from 6 well plate) to cuvette and transfer media/cells back to 6 well plate.

For untransfected group, used 3 ml media from 6 well to resuspend cells in epi tube and transferred to 6 well plate.

TC Incubator o/n.

### Cytotoxicity Assay

### Staining Target Cells (697 Cells and Nalm6 Cells)

Place 5e6 target cells into a 15 ml conical tube and spin down.

Wash cells 2× with PBS to remove any serum proteins. Remove ⅕th of cells for unstained control cells.

Prepare VPD450: Add 1 μL of 1 mM VPD450 stock to 1 ml PBS and mix.

Resuspend cells with 500 ul VPD450/PBS. Resuspend unstained control cells in PBS.

Incubate for 10 min in 37° C. water bath

Add 9 ml PBS and spin cells down 300×g 5 min.

Resuspend in 10 ml PBS and spin cells down 300×g 5 min.

Resuspend cells in 2 ml complete ptimizer+1000 U/ml IL2.

Count live cells using trypan blue.

Add enough additional OpTmizer+IL2 to bring cell concentration to 1e6 cells/ml.

Incubate target and effector cells in a total volume of 200 μL in flow tubes per table below:

### Preparation of gdT Cells

Collect gdT cells and conditioned media from the multiwell plate and put into a 15 ml conical tube.

Wash flask with PBS and collect this in a separate 15 ml conical tube.

Spin 300×g 5 min RT.

Remove the conditioned media from the cell pellet and transfer to fresh conical tube.

Spin the conditioned media 500×g for 10 min to remove and cell debri.

Filter conditioned media with a 0.45 μm filter attached to a 5 ml syringe.

Store the filtered conditioned media at −20 C.

Resuspend and pool the cell pellets (from the cell pellet under the conditioned media and the PBS wash of the well). Resuspend in 300 ul of OpTmizer (a volume small enough to ensure >5e6 cells/ml)

break up cell aggregates by pipetting up/down with a P1000 pipette.

Take 10 μl of the cells and dilute 1:10 by adding to 90 ul of PBS.

Count this 1:10 dilution of cells with trypan blue

Add enough OpTmizer media to bring the cell concentration of each group to 5e6 cells/ml.

### Flow Cytometry Analysis of Cell Killing

Incubate target and effector cells for 4 hours at 37 degrees in flow tubes covered in aluminum foil.

Setup comp tubes! See below.

Wash cells with 1 mL of FACS buffer.

Wash cells 1× with 500 ul AnnexinV binding buffer. resuspend in 100 μL of 1× ANNEXIN V BINDING BUFFER containing 2.5 ul

Annexin V-APC for 20 min at RT.

Add 3 ul 7-ADD per tube and incubate 10 min longer.

Add 1 ml Annexin V binding buffer per rxn to wash.

spin down 300×g 5 min.

Decant and resuspend in 200 ul AnnexinV binding buffer (NOT FACS buffer!)

Run flow to determine % apoptotic cells and dead target cells by gating on VPD450 positive cells.

### Compensation Tubes:

Comp1: Unstained-target cells that have not been stained with VPD450.

Comp2: VPD450 stained target cells (mix of 1:1 stained and unstained target cells).

Comp3: 7-ADD stained target cells (mix 1:1 of live and dead target cells).

Comp4: Annexin V-APC stained target cells (1:1 of live and dead target cells).

### Example 2, Lentivirus Transduction

This example supports FIGS. 12, 13, 15, 19, 25, and 26.

### Day 1—Transduction #1

(1) Thaw frozen Day 12 gdTCs cells, (2) Thaw lentivirus on ice, (3) Thaw LentiBoost and IL-2, (4) Warm Optimizer at 37 degrees C., (5) Count gdTCs, (6) Aliquot 750,000 cells per 1.5 mL tube, (7) Spin gdT cells down at 300 g for 5 minutes, (8) Carefully pipette off the media from the gdTCs, (9) Replace with 500 microliters of the designated or complete OpTmizer media for the Mock transduced cells, (10) Add 10.5 microliters of the LentiBoost+IL-2 master mix, (11) Pipette up and down three times to mix, then dispense into a labeled well in a 48-well plate, (12) Add PBS to all surrounding wells to prevent the plate from drying out, (13) Incubate at 37 degrees C. overnight.

### Day 2—Transduction #2

(1) Pipette all samples from wells into 1.5 mL Eppendorf tubes, (2) Spin down at 300 g for 5 minutes at RT. (3) Carefully pipette off the media from the gdTCs. (4) Replace with 500 microliters of the designated LV. (5) Add 10.5 microliters of the LentiBoost+IL-2 master mix. (6) Pipette up and down three times to mix, then dispense into a labeled well in a 48-well plate. (7) Add PBS to all surrounding wells to prevent the plate from drying out. (8) Incubate at 37 degrees C. overnight.

### Day 3—Media Change

(1) Thaw IL-2 to thaw. (2) Warm Optimizer media in 37 degree the water bath. (3) Gently pipette cells into labeled 1.5 mL tubes. (4) Spin down at 300 g for 5 minutes at RT. (5) Prepare IL-2 supplemented complete Optimizer media. (6) Carefully pipette off the media from the gdTCs. (7) Replace with 500 microliters of complete Optimizer. (8) vPipette gently up and down once and then distribute into labeled wells in a 48-well plate. (9) Add PBS to all surrounding wells to prevent the plate from drying out. (10) Incubate at 37 degrees C.

### Day 6—Analysis

Remove cells from wells and analyze with appropriate assay

See FIG. 12 and FIG. 13 for a data demonstrating the retained expression of GFP in cells driven by the novel disclosed HSP8 promoters when compared to the MND promoters. MND and HSPA8 GFP expression in lentivirally transduced gdT cells. Fresh gdT cells were transduced with lentiviral particles carrying a GFP expression cassette driven by either the MND (myeloproliferative sarcoma virus MPSV enhancer, negative control region NCR deletion) or HSPA8 (Heat shock 70 kDa protein 8) (FIG. 12) The percent of GFP+ cells was measured at day 6 of gdT expansion. (FIG. 13) The mean fluorescent intensity (MFI) of all cells was measured at day 6 of gdT expansion.

FIG. 25 shows gdT cells (effector [E]) were transfected with mRNA encoding the mSA (SEQ ID NO: 3) and native signal peptide versions of the hSCF CD3 STAR and mixed with IMR5 cells (target [T]). Cytotoxicity against the target cells was measured. Effector to target ratios are shown along the X axis.

### Example 3, 293T Conditioned Media Production

This example supports FIGS. 14, 16-18, 20-24, 27-31, 34-37, and 40-42.

Day 1—Producer cell plating

(1) Trypsinize 293T-17 cells (2) Aspirate media from flask (3) Wash with 10 mL PBS (4) Add 10 mL of TrypLe then immediately aspirate off (5) Incubate dry flask for ~10 minutes (6) Resuspend cells in 10 mL DMEM/F12+10% FBS and transfer to 50 ml conical (7) Add 10 more mL DMEM/F12+10% FBS to dilute out cells (8) Dilute cells to 1e6 cells/mL (9) Plate 2 mL/well into 6 well plates (10) Incubate cells overnight

### Day 2—Transfection (Per Well)

(1) Add 84.66 μL of Optimem to a 1.5 mL tube (2) Add 2.5 ug of plasmid to the tube of Optimem. (3) Add 2.5 μL PEI to Optimem/DNA mix and immediately vortex (4) Vortex every 5 minutes for 15 minutes (5) Add full volume (84.88 μL) dropwise to each well then rock gently to mix (6) Place in incubator overnight

### Day 3—Media Change

Change media on transfected cells to 1 mL DMEM/F12+1% FBS Aspirate media from cells and added 1 mL media to each well

### Day 4—Harvest

Harvest supernatant and clarify by spinning at 3000×g for 10 minutes retubing

### Example 4, Adherent Cytotoxicity Assay

This example supports FIGS. 14, 15, 23, 27, 29, 31, 34, 36, 37, 40 and 42.

### Staining Target Cells (CMK or IMR5 Cells)

Place 5e6 target cells into a 15 ml conical tube and spin down.

Wash cells 2 times with PBS to remove any serum proteins. Remove ⅕th of cells for unstained control cells.

Add 1 μL of 1 mM VPD450 stock to 1 ml PBS and mix.

Resuspend cells with 500 ul VPD450/PBS. Resuspend unstained control cells in PBS.

Incubate for 10 min in 37° C. water bath

Add 9 ml PBS and spin cells down 300×g 5 min.

Resuspend in 10 ml PBS and spin cells down 300×g 5 min.

Resuspend cells in 2 ml complete opTmizer+1000 U/ml IL2.

Count live cells using trypan blue.

Add enough additional OpTmizer+IL2 to bring cell concentration to 1e6 cells/ml.

Incubate target and effector cells in a total volume of 200 μL in flow tubes.

### Preparation of gdT Cells

Collect gdT cells and conditioned media from the multiwell plate and put into a 15 ml conical tube.

Wash flask with PBS and collect this in a separate 15 ml conical tube.

Spin 300×g 5 min RT.

Remove the conditioned media from the cell pellet and transfer to fresh conical tube.

Spin the conditioned media 500×g for 10 min to remove and cell debris.

Store the filtered conditioned media at −20 C.

Resuspend and pool the cell in 300 ul of OpTmizer

Break up cell aggregates by pipetting up/down with a P1000 pipette.

Take 10 μl of the cells and dilute 1:10 by adding to 90 ul of PBS.

Count this 1:10 dilution of cells with trypan blue.

Add enough OpTmizer media to bring the cell concentration of each group of gdT cells to 5e6 cells/ml.

### AnnexinV/7AAD Staining

Incubate target and effector cells for 4 hours at 37 degrees in flow tubes covered in aluminum foil.

Wash cells with 1 mL of FACS buffer.

Wash cells 1 time with 500 ul AnnexinV binding buffer.

Resuspend in 100 μL of 1× annexin binding buffer containing 2.5 ul Annexin V-APC and 3 ul 7AAD for 15 min at RT.

Add 1 ml Annexin V binding buffer per reaction to wash.

Spin down 300×g 5 min.

Decant and resuspend in 200 ul AnnexinV binding buffer.

Run flow cytometry to determine % apoptotic cells and dead target cells by gating on VPD450 p

### Example 5, Western Blot

This example supports FIGS. 16-18, 20, 22, 24, 26, 28, 30, 35, 38 and 41.

### SDS-PAGE

Prepare 500 ml 1×SDS Running buffer.

50 ml 10× Tris-Glycine Running Buffer

450 ml dH20

500 ml total

Combine: 4× Sample buffer (4×LDS Sample buffer+β-Mercapthenol (12 ul per 100 ul sample buffer)

+H20

+protein sample

Heat at 95° C. for 5 min in a heat block.

Cool on ice 1 min.

While heating samples, prepare gel and gel box apparatus

Remove gel from package

Rinse gel with H20

Remove tape from bottom of gel

Assemble gel apparatus and fill inner chamber

fill inner chamber with 200 ml 1×RB+anti-ox.

Fill outer chamber with 300 ml remaining 1× RB

carefully remove comb from gel

rinse wells with running buffer using a transfer pipette

Load samples onto gel, include a lane loaded w/5 μL protein standard.

Run gel at ~80-125V (which will result in ~20-40 mA current initially and ~10 mA current at the end of the run)

### Transfer of Proteins to Membrane

Prepare 800 ml Transfer buffer (TB):

32 ml 25× Transfer Buffer (Invitrogen)

160 ml methanol

H20 to 800 ml

Pour approximately 600 ml of TB into a glass dish.

Soak a gel-sized piece of nitrocellulose membrane in TB containing dish, soak in TB for at least 5 min.

Disassemble the gel tank.

Rinse the gel cassette with water.

Prepare the gel/membrane “sandwich” as picture in FIG. 1. The assembly can be made with either 1 or 2 (as pictured) blotting pads on each side of the sandwich. During the assembly of this sandwich, take care not to introduce air bubbles between any of the materials.

With the wells facing up, open the plastic gel cassette with the gel knife and discard the top piece of plastic.

Cut off the top part of the wells and place a TB-wetted piece of filter paper on top of the gel.

Flip the gel over and push the gel away from the plastic by placing the gel knife through the slot at the bottom of the gel.

Cut off the foot of the gel and place the pre-soaked membrane on the gel.

Place a TB wetted piece of filter paper on top of the membrane.

Place a pre-soaked blotting pad in the transfer module.

Place the filter paper/gel/membrane/filter paper in the same orientation on top of the transfer pads place another pre-soaked transfer pad on top of the filter paper.

After assembly of the module, place it into the gel box and clamp it in place.

Fill the inside of the module with TB so that there is just enough to cover the top of the pads. Pour water in the outside chamber (in the gel box).

Set the voltage to 15V (constant) and transfer for 1-2 hrs at RT. The starting current should be approximately 100 mA with an a current at the end of the transfer of approximately ~20 mA.

### Antibody Incubations

### Blocking

After transfer, rinse the membrane with 1× Tris Buffered Saline (TBS).

Block by incubating in 2% milk/TBST for 60 minutes RT while shaking (e.g. orbital shaker or rocker)

Wash several times in TBST.

Primary antibody incubations

Incubate 1-2 hr RT or overnight 4° rocking in 1° antibody in appropriate buffer.

Wash 5×5 minutes in TBST

Secondary antibody incubations

Incubate in 2° Ab in 2% milk/TBST, 1 hour RT

Wash 5×5 minutes in TBST

### Imaging

While washing post secondary ab incubation, turn on FujiFilm imaging system

Combine ECL solution part A and B at 1:1 ratio, 1 ml part A+1 ml part B is sufficient for 1 membrane (Dilute with MilliQ water 1:1 if necessary)

Transfer the membrane on to a clear plastic sheet protector

Wick away excess buffer with a paper towel

Promptly add ECL solution.

Incubate membrane with ECL for 1 min.

Drain excess of solution off the membrane and wick away excess ECL reagent with a paper towel.

Sandwich membrane between two pieces of sheet protector.

Imagine using FUJIFILM imager

### Example 6, Electroporation

Prewarm and equilibrate 3 ml Optimizer complete media containing 1000 IU/ml IL-2 in each well of 6-well plate.

Centrifuge gdT cells at 250×g for 5 min at room temperature.

Resuspend cells to 10e6/ml in Optimem media

Add 1 mL of cells at 10e6 cells/mL to each of 2×1.5 ml epi tubes.

Centrifuge tubes at 250×g 5 min at room temperature.

Remove supernatant completely, first with P1000 pipette, then with P200 pipette tips

Resuspend cells with 100 ul Optimem, pipette cells up/down 3-5 times to break up cell pellet.

Add 15 ug mRNA. Mix and put the cell/mRNA mixture into a 4 mm cuvette.

Electroporate using the following settings:
Settings
500V
Square Wave
5 ms pulse length
1 pulse
4 mm Cuvette
Pulse cells

Use singly wrapped sterile pipette, to add ~500 ul warmed media (from 6 well plate) to cuvette and carefully transfer media/cells back to 6 well plate.

Incubator cells overnight.

### Example 7, CD19 Cytotox Assay

This example supports FIGS. 19 and 21.

### Staining Target 697 Cells

Place 5e6 target cells into a 15 ml conical tube and spin down.

Wash cells 2× with PBS to remove any serum proteins.

Prepare VPD450: Add 1 μL of 1 mM VPD450 stock to 1 ml PBS and mix.

Resuspend cells with 500 ul VPD450/PBS. Resuspend unstained control cells in PBS.

Incubate for 10 min in 37° C. water bath

Add 9 ml PBS and spin cells down 300×g 5 min.

Resuspend in 10 ml PBS and spin cells down 300×g 5 min.

Resuspend cells in 2 ml complete Optimizer+1000 U/ml IL2.

Count live cells using trypan blue.

Add enough additional Optimizer+IL2 to bring the target cell concentration to 1e6 cells/ml.

Incubate target and effector cells in a total volume of 200 μL in 48 well plate table below:

### Preparation of gdT Cells

Collect gdT cells and conditioned media and put into a 15 ml conical tube.

Wash flask with PBS and collect this in a separate 15 ml conical tube.

Spin 300×g 5 min RT.

Remove the conditioned media from the cell pellet and transfer to fresh conical tube.

Spin the conditioned media 500×g for 10 min to remove and cell debris.

Store the filtered conditioned media at −20 C.

Resuspend and pool the cell pellets (from the cell pellet under the conditioned media and the PBS wash of the well). Resuspend in 300 ul of Optmizer (a volume small enough to ensure >5e6 cells/ml)

Break up cell aggregates by pipetting up/down with a P1000 pipette.

Take 10 μl of the cells and dilute 1:10 by adding to 90 ul of PBS.

Count the 1:10 dilution of cells with trypan blue.

Add enough Optmizer media to bring the cell concentration of gdT cells to 5e6 cells/ml.

Incubate target and effector cells for 18 hr at 37 degrees in 48-well plate.

Before Annexin V/7AAD staining, transfer assay samples from wells to well-labelled flow tubes. Include a tube of unlabeled target cells in the washing steps

Wash cells with 1 mL of flow buffer.

Wash cells 1× with 500 ul Annexin V binding buffer.

Resuspend unstained target cells in AnnexinV binding buffer. Transfer half of the cells to a new 1.5 ml tube and heat-kill them on a 100 C heat block for 2 min. Cool down the heated tube on ice and combine dead cells with live cells. Use these cells for compensation.

Resuspend in 100 μL of 1× annexin binding buffer containing 2.5 ul Annexin V-APC and 3 ul 7AAD for 15 min at RT.

Add 1 ml Annexin V binding buffer per reaction to wash.

spin down 300×g 5 min.

Decant and resuspend in 200 ul AnnexinV binding buffer (NOT FACS buffer!)

Run flow to determine % apoptotic cells and dead target cells by gating on VPD450 positive cells.

### Example 8, Cytotox Assay (HEL and Kasumi Cells)

This example supports FIGS. 20 and 25.

### VPD Labelling Target Cells (Kasumi1 or HEL Cells)

Place 5e6 target cells into a 15 ml conical tube and spin down.

Wash cells 2× with PBS to remove any serum proteins.

Prepare VPD450: Add 1 μL of 1 mM VPD450 stock to 1 ml PBS and mix.

Resuspend cells with 500 ul VPD450/PBS. Resuspend unstained control cells in PBS.

Incubate for 10 min in 37° C. water bath Add 9 ml PBS and spin cells down 300×g 5 min.

Resuspend in 10 ml PBS and spin cells down 300×g 5 min.

Resuspend cells in 2 ml complete Optimizer+1000 U/ml IL2.

Count live cells using trypan blue.

Add enough additional OpTmizer+IL2 to bring cell concentration to 1e6 cells/ml.

Incubate target and effector cells in a total volume of 200 μL in flow tubes

### Preparation of gdT Cells

Collect gdT cells and conditioned media from the multiwell plate and put into a 15 ml conical tube.

Wash flask with PBS and collect this in a separate 15 ml conical tube.

Spin 300×g 5 min RT.

Resuspend in 150 ul of Optimizer.

Break up cell aggregates by pipetting up/down with a P1000 pipette.

Take 10 μl of the cells and dilute 1:10 by adding to 90 ul of PBS.

Count this 1:10 dilution of cells with trypan blue.

Add enough Optimizer media to bring the cell concentration of each group of gdT cells to 5e6 cells/ml.

Analyze cells by flow cytometry

### Example 9, HLA Class I and Class II shRNA Knockdown and Surface Expression

This example supports FIGS. 32 and 33.

Day 0—Transduce gdT cells:

Thaw gdT frozen gdT cells

Take vial directly from −80 C to 37 C water bath to thaw.

Move the contents of the cryovial to a 15 ml conical tube.

Add 2 ml of thawing media to the cells in the conical tube dropwise.

Rinse cryovial out with 1 ml thawing media and add this wash to the 15 ml conical tube, bringing total volume in conical tube to 4 ml.

Spin cells at 300×g 5 min RT

Resuspended cells in 37 C complete Optimizer+IL2 to a density of 3e6 cells/ml.

Place cell suspension into flask of appropriate size in 37 C TC incubator.

Spin cells down at 250 G for 5 minutes in 1.5 mL tubes

Resuspend cells in unconcentrated lentivirus and plus Optimizer media

Plate cells onto a well of a 96-well plate per condition and transduced overnight at 37 degrees/5% CO₂.

### Day 1—Media Change:

Remove cells from wells via pipette

Spun cells down at 300 g for 5 minutes and resuspended in 150 ul of complete

Optimizer+IL2

Plate cells and incubate for 72 hours

Day 4—Prepare cells for flow cytometry:

Spin transduced gdT cells at 350×g for 10 minutes Decant tubes that were spinning and add 2.5 ul of anti-human B2m antibody+97.5 uL

FACS mix to each tube and mix.

Place samples in a light-shielded location for 10 minutes at room temperature.

After 10 minutes has passed, add 3 ul of 7AAD to each sample and mix.

Put the samples back in a dark place and incubate at room temperature for 5 minutes

After 5 minutes, add 500 ul of FACS buffer to each sample and spin down at 300 g for 5 minutes

Resuspend cells in 200 ul of FACS buffer and run flow cytometry.

### Example 10, Jurkat Electroporation

This example supports FIG. 39

The day prior to electroporation split cells to ~300K/ml

On day of transfection, add 3 ml of culture media to each needed well of 6 well plate

Collected cells in 15 ml conical tube and counted-15 ml volume, 8.6e5/ml=12.9e6 cells

Spin down 200×g 10 min

Resuspend cells in 4.3 ml Optimem (cells at 3e6/ml)

Add 1 ml of cells suspension to each 1.5 ml epi tubes.

Spin down 160×g for 10 min

Resuspend cells in 100 ul Optimem.

Add 5 ul (=5 ug) GFP mRNA to tube. Mix by pipetting up/down several times and move contents into cuvette.

Zap cells using the following parameters:

Voltage: 140V

Length of pulse: 5 ms

number of pulses: 2

Pulse interval: 0.1 second

cuvette gap: 2 mm

Transfer cells into 6 well plate.

culture overnight in TC incubator

Cells analyzed ~24 hrs after transfection by flow cytometry

### Sequence Product Listings

In the following section, the sequence listings are followed by descriptions of the sequences. For each we provide the region representing the Signal Peptide, followed by the residues; Target scFv, which in the following paragraphs is short hand for the residues of the sequence making up the scFv portion of the targeting scFv, followed by the residues; Target VL, which in the following is short hand for the residues of the sequence making of the VL portion of the targeting scFv, followed by the residues; Target linker, which in the following is short hand for the residues of the sequence making up the linker portion: followed by the residues; Target VH, which in the following is short hand for the residues of the sequence making up the VH of the targeting scFv: followed by the residues; Central Linker, which is shorthand for what is referred to herein also as a flexible linker, see, e.g., FIG. 1: followed by the residues; gdT scFv; gdT VH; gdT Linker; and gdT VL.

We also provide, where applicable, the free energy, the gdT CAI, and the ORF count.

SEQ ID NO: 1 is the sequence name for IL2 CD19 CD3 STAR. It is a Complete STAR construct. It is an Amino Acid sequence. The sequence is:

MYRMQLLSCIALSLALVTNSDIQLTQSPASLAVSLGQRATISCKASQSVD
YDGDSYLNWYQQIPGQPPKLLIYDASNLVSGIPPRFSGSGSGTDFTLNIH
PVEKVDAATYHCQQSTEDPWTFGGGTKLEIKGGGGSGGGGSGGGGSQVQL
QQSGAELVRPGSSVKISCKASGYAFSSYWMNWVKQRPGQGLEWIGQIWPG
DGDTNYNGKFKGKATLTADESSSTAYMQLSSLASEDSAVYFCARRETTTV
GRYYYAMDYWGQGTTVTVSSGGGGSDIKLQQSGAELARPGASVKMSCKTS
GYTFTRYTMHWVKQRPGQGLEWIGYINPSRGYTNYNQKFKDKATLTTDKS
SSTAYMQLSSLTSEDSAVYYCARYYDDHYCLDYWGQGTTLTVSSVEGGSG
GSGGSGGSGGVDDIQLTQSPAIMSASPGEKVTMTCRASSSVSYMNWYQQK
SGTSPKRWIYDTSKVASGVPYRFSGSGSGTSYSLTISSMEAEDAATYYCQ
QWSSNPLTFGAGTKLELK.

SEQ ID NO: 1 includes Signal Peptide: 1-20; Target scFv: 21-270; Target VL: 21-131; Target linker: 132-146; Target VH: 147-270; Central Linker: 271-275; gdT scFv: 276-518; gdT VH: 397-410; gdT Linker: 397-410; gdT VL: 411-518.

SEQ ID NO: 2 is the sequence name for IL2 CD19 CD3 STAR. It is a Complete STAR construct. It is a DNA sequence. The sequence is:

ATGTACAGGATGCAGCTGCTGTCCTGCATTGCCCTGTCCCTGGCCCTGGT
GACCAACTCCGACATTCAATTGACACAAAGCCCGGCAAGCCTGGCTGTGT
CCCTGGGCCAGCGGGCGACTATTTCATGTAAAGCAAGTCAGAGCGTAGAC
TACGATGGTGATAGCTATCTGAACTGGTATCAGCAGATTCCAGGACAACC
TCCCAAATTGCTGATTTACGACGCCTCGAACCTGGTCAGCGGGATTCCAC
CACGATTCTCTGGAAGCGGCAGTGGAACCGATTTTACGCTGAATATACAC
CCAGTGGAAAAGGTGGACGCTGCGACTTATCATTGTCAGCAGTCTACCGA
GGACCCATGGACCTTCGGCGGGGGAACAAAGCTGGAGATCAAGGGTGGTG
GTGGAAGCGGTGGTGGTGGATCTGGCGGTGGTGGAAGTCAGGTGCAGCTT
CAGCAGTCCGGAGCCGAACTGGTGCGCCCCGGCAGTAGCGTGAAGATAAG
CTGCAAGGCTAGCGGATACGCTTTCTCTTCCTACTGGATGAACTGGGTGA
AGCAGCGCCCTGGCCAAGGGCTTGAGTGGATCGGCCAGATATGGCCTGGT
GATGGTGACACCAACTATAATGGCAAGTTTAAGGGAAAGGCTACACTCAC
TGCCGATGAAAGTTCATCCACTGCCTACATGCAGCTCTCCAGTCTGGCAA
GTGAAGACTCTGCTGTCTATTTCTGCGCTAGACGAGAGACTACAACTGTC
GGGAGGTACTACTATGCAATGGATTACTGGGGTCAGGGAACCACAGTGAC
AGTGAGCTCAGGAGGTGGTGGGTCTGACATTAAGCTACAACAGTCTGGCG
CCGAGTTGGCCAGGCCTGGGGCCAGCGTGAAGATGTCTTGCAAGACCTCA
GGTTATACCTTCACGAGATACACCATGCATTGGGTAAAACAGAGACCTGG
GCAAGGCTTGGAATGGATCGGCTACATCAACCCCAGTCGCGGATACACAA
ACTACAATCAGAAATTTAAAGACAAGGCGACTCTCACCACAGATAAGTCC
TCCTCTACCGCCTACATGCAGCTGTCATCTCTCACAAGCGAAGACTCTGC
CGTGTATTATTGCGCAAGGTATTACGATGACCACTATTGTTTAGATTATT
GGGGGCAAGGAACTACACTCACTGTCAGCTCAGTTGAGGGTGGAAGTGGG
GGATCTGGTGGTTCAGGCGGATCTGGTGGGGTCGACGACATCCAGCTGAC
CCAGAGTCCCGCCATCATGTCAGCTAGTCCCGGGGAGAAAGTGACTATGA
CATGCAGGGCATCTAGCAGCGTTTCGTACATGAATTGGTATCAGCAGAAA
TCAGGTACCTCCCCGAAACGTTGGATTTATGACACGAGCAAGGTTGCAAG
CGGTGTTCCATACCGGTTTTCGGGCTCCGGTTCCGGCACCTCCTACTCTC
TCACGATCTCCAGTATGGAAGCCGAGGATGCCGCAACCTATTACTGTCAG
CAATGGTCATCCAATCCCTTAACCTTCGGAGCCGGAACAAAACTGGAGCT
CAAA.

SEQ ID NO: 2 includes Signal Peptide: 1-60; Target scFv: 61-810; Target VL: 61-393; Target linker: 394-438; Target VH: 439-810; Central Linker: 811-825; gdT scFv: 826-1544; gdT VH: 1189-1230; gdT Linker: 1189-1230; gdT VL: 1231-1544.

SEQ ID NO: 3 is the sequence name for modified serum albumin (mSA). It is a Signal Peptide construct. It is an AA sequence. The sequence is: MKWVTFISLLFLFSSSSRA. SEQ ID NO: 3 includes Signal Peptide: 1-19.

SEQ ID NO: 4 is the sequence name for human stem cell factor (hSCF). It is a Signal Peptide construct. It is an AA sequence. The sequence is: MKKTQTWILTCIYLQLLLFNPLVKT.

SEQ ID NO: 4 includes Signal Peptide: 1-25.

SEQ ID NO: 5 is the sequence name for modified serum albumin (mSA). It is a Signal Peptide construct. It is a DNA sequence. The sequence is:

ATGAAATGGGTTACTTTTATTAGTTTATTATTCCTGTTCAGCTCCAGCTC
CAGGGCC.

SEQ ID NO: 5 includes Signal Peptide: 1-57.

SEQ ID NO: 6 is the sequence name for human stem cell factor (hSCF). It is a Signal

Peptide construct. It is a DNA sequence. The sequence is:

ATGAAGAAAACTCAAACTTGGATACTAACTTGCATCTACCTGCAGCTGCT
GCTCTTCAACCCCTTGGTGAAGACG.

SEQ ID NO: 6 includes Signal Peptide: 1-75.

SEQ ID NO: 7 is the sequence name for CD19 scFv ECOg (154). It is a Tumor Targeting scFv construct. It is a DNA sequence. The sequence is:

GACATCCAGCTCACCCAGAGCCCCGCATCCCTGGCCGTGTCCCTGGGGCA
GCGCGCAACCATCTCCTGCAAGGCTTCCCAGTCCGTGGACTACGACGGGG
ACTCCTACCTGAACTGGTACCAGCAGATCCCCGGGCAGCCACCGAAGCTG
CTGATCTACGACGCCTCCAACCTGGTGTCCGGGATTCCTCCGCGGTTCTC
CGGGAGCGGGTCCGGGACCGACTTCACCCTGAACATCCATCCCGTGGAGA
AGGTGGACGCCGCCACCTACCACTGCCAGCAGTCCACCGAGGACCCCTGG
ACCTTCGGCGGCGGCACCAAGCTGGAGATCAAAGGTGGCGGAGGCTCCGG
TGGCGGAGGTTCCGGTGGCGGCGGCTCCCAGGTGCAGCTGCAGCAGTCAG
GGGCCGAGCTGGTGAGGCCCGGGAGCTCCGTGAAGATCTCCTGCAAGGCC
TCCGGGTACGCCTTCTCCTCCTACTGGATGAACTGGGTGAAGCAGAGGCC
CGGGCAGGGGCTGGAGTGGATCGGGCAGATCTGGCCCGGGGACGGGGACA
CCAACTACAACGGGAAGTTCAAGGGGAAGGCTACCCTCACCGCCGACGAG
AGCTCCTCCACCGCCTACATGCAGCTGAGCTCCCTCGCCTCCGAGGACTC
CGCCGTGTACTTCTGCGCCCGGCGCGAGACCACCACCGTGGGGCGCTACT
ACTACGCCATGGACTACTGGGGCCAGGGGACCACCGTGACCGTGAGCTC
C.

SEQ ID NO: 7 includes Target scFv: 1-750; Target VL: 1-333; Target linker: 334-378; Target VH: 379-750; Free energy: −345.2; gdT CAI: 0.89467859593316; ORF count: 1.

SEQ ID NO: 8 is the sequence name for CD19 scFv ECOg (94). It is a Tumor Targeting scFv construct. It is a DNA sequence. The sequence is:

GACATCCAGCTGACCCAGTCGCCCGCTAGCCTGGCGGTCTCGCTGGGCCA
GCGAGCCACCATCAGCTGCAAGGCCAGCCAGTCGGTCGACTACGACGGCG
ACAGCTACCTCAACTGGTACCAGCAGATCCCCGGCCAGCCACCGAAGCTG
CTGATCTACGACGCCAGCAACCTGGTGAGCGGGATACCACCGCGGTTCTC
GGGGTCGGGGTCGGGGACCGACTTCACCCTCAACATCCATCCCGTCGAGA
AGGTCGACGCCGCCACCTACCACTGCCAGCAGTCGACCGAGGACCCCTGG
ACCTTCGGCGGCGGCACCAAGCTGGAGATCAAAGGTGGCGGTGGCTCCGG
TGGCGGTGGTAGCGGTGGCGGCGGCTCCCAGGTCCAGCTCCAGCAGTCTG
GGGCCGAGCTGGTGCGACCCGGGAGCTCGGTCAAGATCAGCTGCAAGGCC
AGCGGCTACGCCTTCTCCAGCTACTGGATGAACTGGGTCAAGCAGCGACC
CGGCCAGGGGCTGGAGTGGATCGGCCAGATCTGGCCCGGCGACGGCGACA
CCAACTACAACGGCAAGTTCAAGGGCAAGGCCACGCTGACCGCCGACGAG
TCGAGCTCGACCGCCTACATGCAGCTGAGCTCGCTCGCCAGCGAGGACTC
CGCGGTCTACTTCTGCGCCCGGCGCGAGACCACCACCGTGGGGCGCTACT
ACTACGCGATGGACTACTGGGGCCAGGGGACCACGGTGACCGTGAGCTCG

SEQ ID NO: 8 includes Target scFv: 1-750; Target VL: 1-333; Target linker: 334-378; Target VH: 379-750; Free energy: −360.7; gdT CAI: 0.767359962199379; ORF count: 2.

SEQ ID NO: 9 is the sequence name for CD19 scFv ECOg (139). It is a Tumor Targeting scFv construct. It is a DNA sequence. The sequence is:

GACATCCAGCTGACGCAGTCGCCCGCGTCGCTCGCCGTGTCGCTCGGGCA
ACGCGCGACGATCTCGTGCAAGGCGTCGCAGTCCGTCGACTACGACGGCG
ACTCGTACCTGAACTGGTACCAGCAGATCCCCGGGCAGCCGCCGAAGCTG
CTGATCTACGACGCGAGCAACCTCGTGTCCGGCATTCCGCCGCGGTTCAG
CGGCAGCGGCAGCGGCACCGACTTCACGCTGAACATTCACCCCGTCGAGA
AGGTCGACGCCGCGACGTACCACTGCCAGCAGTCCACCGAGGACCCGTGG
ACGTTCGGCGGCGGCACGAAGCTGGAGATCAAAGGTGGCGGCGGTTCCGG
TGGCGGTGGTTCCGGTGGCGGCGGCTCGCAGGTGCAGCTGCAGCAGTCTG
GCGCCGAGCTCGTGCGACCCGGGTCGTCCGTGAAGATCTCGTGCAAGGCG
TCCGGGTACGCATTCTCGTCGTACTGGATGAACTGGGTGAAGCAGCGACC
CGGGCAGGGGCTGGAGTGGATCGGGCAGATCTGGCCCGGCGACGGCGACA
CGAACTACAACGGCAAGTTCAAGGGCAAGGCGACGCTGACCGCCGACGAG
TCGTCGTCCACCGCGTACATGCAGCTGTCGTCGCTCGCGAGCGAGGACAG
CGCCGTGTACTTCTGCGCGCGGCGCGAGACGACGACCGTCGGGCGCTACT
ACTACGCGATGGACTACTGGGGCCAGGGCACGACCGTGACCGTGTCCAG
C.

SEQ ID NO: 9 includes Target scFv: 1-750; Target VL: 1-333; Target linker: 334-378; Target VH: 379-750; Free energy: −357.7; gdT CAI: 0.724573751544555; ORF count: 1.

SEQ ID NO: 10 is the sequence name for CD19 scFv ECOg (195). It is a Tumor Targeting scFv construct. It is a DNA sequence. The sequence is:

GACATCCAGCTGACCCAGAGCCCCGCGAGCCTGGCCGTGAGCCTGGGGCA
GAGGGCCACCATCAGCTGCAAGGCGTCCCAGAGCGTGGACTACGACGGGG
ACAGCTACCTGAACTGGTACCAGCAGATCCCCGGGCAGCCGCCGAAGCTG
CTGATCTACGACGCCTCCAACCTGGTGTCCGGGATACCGCCGCGGTTCTC
CGGGTCCGGGTCCGGGACCGACTTCACCCTGAACATTCACCCCGTGGAGA
AGGTGGACGCCGCCACCTACCACTGCCAGCAGAGCACCGAGGACCCCTGG
ACCTTCGGCGGCGGCACCAAGCTGGAGATCAAAGGTGGCGGAGGCTCCGG
TGGCGGTGGTAGCGGTGGCGGCGGCTCCCAGGTGCAGCTGCAGCAGTCTG
GGGCCGAGCTGGTGCGACCCGGGTCCAGCGTGAAGATCAGCTGCAAGGCC
AGCGGGTACGCCTTCAGCTCCTACTGGATGAACTGGGTGAAGCAGAGGCC
CGGGCAGGGGCTGGAGTGGATCGGGCAGATCTGGCCCGGGGACGGGGACA
CCAACTACAACGGGAAGTTCAAGGGGAAGGCCACGCTGACCGCCGACGAG
AGCAGCTCCACCGCCTACATGCAGCTGTCCAGCCTGGCCTCCGAGGACAG
CGCCGTGTACTTCTGCGCCCGGCGCGAGACCACCACCGTGGGGCGCTACT
ACTACGCCATGGACTACTGGGGCCAGGGGACCACCGTGACCGTGAGCTC
C.

SEQ ID NO: 10 includes Target scFv: 1-750; Target VL: 1-333; Target linker: 334-378; Target VH: 379-750; Free energy: −356; gdT CAI: 0.858340915819879; ORF count: 2.

SEQ ID NO: 11 is the sequence name for CD19 scFv ECOg (160). It is a Tumor Targeting scFv construct. It is a DNA sequence. The sequence is:

GACATCCAGCTGACCCAGTCACCCGCTAGCCTGGCCGTGTCCCTGGGCCA
GCGAGCCACGATCTCCTGCAAGGCCAGCCAGTCCGTGGACTACGACGGGG
ACTCCTACCTCAACTGGTACCAGCAGATCCCCGGCCAGCCACCGAAGCTG
CTGATCTACGACGCCTCCAACCTGGTGAGCGGGATTCCGCCGCGGTTCAG
CGGGTCCGGGTCCGGGACCGACTTCACCCTCAACATCCATCCCGTGGAGA
AGGTGGACGCCGCCACCTACCACTGCCAGCAGTCCACCGAGGACCCCTGG
ACCTTCGGCGGCGGCACCAAGCTGGAGATCAAAGGCGGTGGTGGCTCCGG
AGGCGGTGGCTCTGGTGGCGGCGGCTCCCAGGTGCAGCTCCAGCAGAGCG
GGGCTGAGCTGGTGAGGCCCGGGTCCTCCGTGAAGATCTCCTGCAAGGCC
TCCGGGTACGCCTTCTCCAGCTACTGGATGAACTGGGTGAAGCAGAGGCC
CGGGCAGGGGCTGGAGTGGATCGGGCAGATCTGGCCCGGGGACGGGGACA
CCAACTACAACGGGAAGTTCAAGGGGAAGGCCACGCTGACCGCCGACGAG
AGCAGCTCCACCGCCTACATGCAGCTGTCCAGCCTGGCCTCCGAGGACTC
CGCCGTGTACTTCTGCGCCCGGCGCGAGACCACCACCGTGGGGCGCTACT
ACTACGCCATGGACTACTGGGGCCAGGGGACCACCGTGACCGTGTCCAG
C.

SEQ ID NO: 11 includes Target scFv: 1-750; Target VL: 1-333; Target linker: 334-378; Target VH: 379-750; Free energy: −354.8; gdT CAI: 0.876843948859594; ORF count: 2.

SEQ ID NO: 12 is the sequence name for CD3 scFv ECOg (70). It is a gdT Targeting scFv construct. It is a DNA sequence. The sequence is:

GATATCAAGCTCCAGCAGTCCGGGGCTGAGCTGGCTAGGCCCGGGGCCTC
CGTGAAGATGTCCTGCAAGACCTCCGGGTACACCTTCACCAGGTACACCA
TGCACTGGGTGAAGCAGAGGCCCGGGCAGGGGCTGGAGTGGATCGGGTAC
ATCAACCCCAGCCGCGGGTACACCAACTACAACCAGAAGTTCAAGGACAA
GGCCACCTTGACCACCGACAAGTCCTCCTCCACCGCCTACATGCAGCTGA
GCTCCCTGACCTCCGAGGACTCCGCCGTCTACTACTGCGCCAGGTACTAC
GACGACCACTACTGCCTGGACTACTGGGGCCAGGGGACTACCCTGACCGT
GAGCTCCGTGGAAGGTGGCTCCGGCGGCTCCGGAGGTTCCGGAGGCTCCG
GCGGCGTGGACGACATCCAGCTGACCCAGTCCCCAGCTATCATGTCCGCC
AGCCCCGGGGAGAAGGTGACCATGACCTGCCGGGCCTCCTCCTCCGTGAG
CTACATGAACTGGTACCAGCAGAAGTCCGGGACCTCTCCCAAGAGGTGGA
TCTACGACACCTCCAAGGTGGCCTCCGGGGTCCCCTACAGGTTCTCCGGG
TCCGGGTCCGGGACCTCCTACTCCCTGACCATCTCCTCCATGGAGGCCGA
GGACGCCGCCACCTACTACTGCCAGCAGTGGAGCTCCAATCCCCTGACCT
TCGGGGCCGGGACCAAGCTGGAGCTGAAG.

SEQ ID NO: 12 includes gdT scFv: 1-729; gdT VH: 364-405; gdT Linker: 364-405; gdT VL: 406-729; Free energy: −320.1; gdT CAI: 0.924743; ORF count: 1.

SEQ ID NO: 13 is the sequence name for CD3 scFv ECOg (197). It is a gdT Targeting scFv construct. It is a DNA sequence. The sequence is:

GACATCAAGCTCCAGCAGTCCGGGGCTGAGCTTGCTCGCCCCGGGGCCAG
CGTGAAGATGTCCTGCAAGACCTCGGGGTACACCTTCACCAGGTACACCA
TGCACTGGGTGAAGCAGCGCCCAGGGCAGGGCCTGGAGTGGATAGGGTAC
ATCAACCCCAGCCGCGGGTACACAAACTACAACCAGAAGTTCAAGGACAA
GGCCACGCTGACCACGGACAAGTCCTCGTCCACGGCGTACATGCAGCTGT
CCTCGCTGACCTCCGAGGACAGCGCGGTGTACTACTGCGCGCGGTACTAC
GACGACCACTACTGCCTGGACTACTGGGGCCAGGGGACAACGCTGACCGT
GAGCAGCGTGGAAGGCGGCAGCGGCGGCAGCGGAGGTAGCGGAGGCTCCG
GCGGCGTGGACGACATCCAGCTCACCCAGAGCCCGGCGATCATGTCCGCC
AGCCCCGGGGAGAAGGTGACCATGACCTGCCGGGCGTCGTCCTCGGTGAG
CTACATGAACTGGTACCAGCAGAAGTCCGGGACCTCGCCCAAGCGCTGGA
TCTACGACACCAGCAAGGTGGCCTCCGGGGTGCCCTACCGCTTCTCCGGG
TCGGGGTCGGGGACATCGTACTCCCTGACCATCTCCAGCATGGAGGCCGA
GGACGCCGCGACGTACTACTGCCAGCAGTGGTCGTCCAACCCGCTGACCT
TCGGCGCGGGGACAAAGCTGGAGCTTAAG.

SEQ ID NO: 13 includes gdT scFv: 1-729; gdT VH: 364-405; gdT Linker: 364-405; gdT VL: 406-729; Free energy: −341.1; gdT CAI: 0.803206301402276; ORF count: 2.

SEQ ID NO: 14 is the sequence name for CD3 scFv ECOg (109). It is a gdT Targeting scFv construct. It is a DNA sequence. The sequence is:

GACATCAAGCTGCAGCAGAGCGGCGCTGAGCTCGCGCGACCAGGCGCGAG
CGTGAAGATGAGCTGCAAGACGAGCGGCTACACGTTCACGCGCTACACGA
TGCACTGGGTGAAGCAGCGTCCCGGGCAGGGGCTGGAGTGGATCGGCTAC
ATCAACCCGTCGCGCGGCTACACGAACTACAACCAGAAGTTCAAGGACAA
GGCGACGCTGACGACCGACAAGAGCAGCAGCACCGCGTACATGCAGCTGA
GCTCGCTGACGAGCGAGGACAGCGCCGTGTACTACTGCGCGCGCTACTAC
GACGACCACTACTGCCTCGACTACTGGGGCCAGGGCACGACGCTGACCGT
GAGCAGCGTCGAAGGCGGCAGCGGCGGCAGCGGTGGAAGCGGTGGCAGCG
GCGGCGTCGACGACATCCAGCTGACGCAGTCGCCCGCGATCATGAGCGCG
TCGCCCGGCGAGAAGGTGACGATGACGTGCCGCGCGAGCAGCAGCGTGTC
GTACATGAACTGGTACCAGCAGAAGAGCGGCACGTCGCCGAAGCGCTGGA
TCTACGACACGTCGAAGGTCGCGAGCGGCGTGCCGTACCGCTTCAGCGGC
AGCGGCAGCGGCACGTCGTACTCGCTGACGATCAGCAGCATGGAGGCCGA
GGACGCCGCGACGTACTACTGCCAGCAGTGGAGCTCGAACCCGCTGACGT
TCGGCGCCGGCACGAAGCTGGAGCTGAAG.

SEQ ID NO: 14 includes gdT scFv: 1-729; gdT VH: 364-405; gdT Linker: 364-405; gdT VL: 406-729; Free energy: −334.7; gdT CAI: 0.70167404462703; ORF count: 1.

SEQ ID NO: 15 is the sequence name for CD3 scFv ECOg (119). It is a gdT Targeting scFv construct. It is a DNA sequence. The sequence is:

GACATCAAGCTGCAGCAGAGCGGCGCGGAATTAGCGCGCCCCGGGGCGTC
CGTCAAGATGTCCTGCAAGACCTCCGGGTACACCTTCACGCGGTACACCA
TGCACTGGGTGAAGCAACGCCCCGGGCAGGGCCTGGAGTGGATCGGGTAC
ATCAACCCGTCGCGGGGCTACACGAACTACAACCAGAAGTTCAAGGACAA
GGCGACCCTCACGACCGACAAGTCGAGCAGCACGGCGTACATGCAGCTCT
CCTCGCTGACCAGCGAGGACTCCGCGGTGTACTACTGCGCGCGGTACTAC
GACGACCACTACTGCCTGGACTACTGGGGCCAGGGCACCACGCTGACCGT
CAGCTCGGTGGAAGGCGGCTCCGGCGGCTCCGGAGGTTCCGGAGGTTCCG
GCGGAGTGGACGACATCCAGCTCACCCAGAGCCCCGCGATTATGAGCGCC
TCGCCCGGCGAGAAGGTCACCATGACCTGCCGGGCGAGCAGCTCGGTGAG
CTACATGAACTGGTACCAGCAGAAGAGCGGCACGTCGCCCAAGCGGTGGA
TCTACGATACCAGCAAGGTGGCGAGCGGCGTGCCGTACCGCTTCTCGGGG
AGCGGGTCCGGCACGTCGTACAGCCTGACGATCAGCAGCATGGAGGCGGA
GGACGCCGCGACGTACTACTGCCAGCAGTGGAGCAGCAACCCGCTGACCT
TCGGGGCCGGCACGAAGCTCGAACTGAAG.

SEQ ID NO: 15 includes gdT scFv: 1-729; gdT VH: 364-405; gdT Linker: 364-405; gdT VL: 406-729; Free energy: −328.1; gdT CAI: 0.739432850740138; ORF count: 0.

SEQ ID NO: 16 is the sequence name for CD3 scFv ECOg (45). It is a gdT Targeting scFv construct. It is a DNA sequence. The sequence is:

GACATCAAGCTGCAGCAGTCCGGAGCCGAGTTGGCACGGCCCGGGGCCTC
CGTGAAGATGTCCTGCAAGACGTCCGGGTACACGTTCACCCGGTACACCA
TGCACTGGGTGAAGCAGCGGCCCGGGCAGGGGCTGGAATGGATCGGGTAC
ATCAACCCCAGCCGCGGGTACACCAACTACAACCAGAAGTTCAAGGACAA
GGCCACGCTGACCACCGACAAGTCGTCGTCGACCGCCTACATGCAGCTGA
GCTCGCTGACCAGCGAGGACAGCGCCGTCTACTACTGCGCCCGCTACTAC
GACGACCACTACTGCCTGGACTACTGGGGCCAGGGGACCACGCTGACAGT
GAGCAGCGTGGAAGGCGGCTCCGGCGGCTCCGGAGGTTCCGGAGGCAGCG
GCGGCGTCGACGACATCCAGCTCACCCAGTCCCCGGCCATCATGTCGGCG
AGCCCCGGCGAGAAGGTGACCATGACGTGCCGGGCCTCCAGCTCGGTGTC
CTACATGAACTGGTACCAGCAGAAGTCGGGGACCAGCCCCAAGCGGTGGA
TCTACGACACCAGCAAGGTGGCCAGCGGGGTCCCCTACCGCTTCTCGGGG
TCCGGGTCCGGGACCTCGTACTCGCTGACCATCTCCAGCATGGAGGCCGA
GGACGCCGCCACCTACTACTGCCAGCAGTGGTCCTCGAACCCGCTCACCT
TCGGGGCCGGCACCAAGCTGGAGCTGAAG.

SEQ ID NO: 16 includes gdT scFv: 1-729; gdT VH: 364-405; gdT Linker: 364-405; gdT VL: 406-729; Free energy: −323.7; gdT CAI: 0.817020513172323; ORF count: 0.

SEQ ID NO: 17 is the sequence name for PTK7 scFv. It is a Tumor Targeting scFv construct. It is an AA sequence. The sequence is:

EVQLVQSGGGLVHPGGSLRLSCAGSGFTFSTYLMYWVRQAPGKTLEWVSA
IGSGGDTYYADSVKGRFTISRDNAKNSLYLQMNSLRAEDMAVYYCARGLG
YWGQGTLVTVSSGGGGSGGGGSGGGGSEIVLTQSPGTLSLSPGERATLSC
RASQSVSSSYLAWYQQKPGQAPRLLIYGASSRATGIPDRFSGSGSGTDFT
LTISRLEPEDFAVYYCQQYGSSPMYTFGQGTKLEIK.

SEQ ID NO: 17 includes Target scFv: 1-236; Target VL: 128-236; Target linker: 113-127; Target VH: 1-236.

SEQ ID NO: 18 is the sequence name for PTK7 scFv. It is a Tumor Targeting scFv construct. It is a DNA sequence. The sequence is:

GAGGTGCAGCTGGTGCAGTCCGGAGGAGGTTTAGTTCACCCAGGAGGCT
CGCTGCGACTGTCCTGTGCAGGGAGCGGCTTTACCTTCAGCACATATCT
GATGTACTGGGTGAGACAGGCCCCCGGCAAGACGCTGGAGTGGGTCTCA
GCTATCGGATCAGGTGGCGACACCTATTACGCTGATAGCGTGAAGGGCC
GGTTTACCATAAGCCGCGACAACGCCAAAAATAGTCTTTACCTGCAGAT
GAACAGCCTCCGAGCAGAGGATATGGCCGTCTATTACTGCGCTCGGGGA
CTCGGGTATTGGGGGCAGGGCACCTTGGTGACTGTCTCTTCAGGGGGAG
GTGGTTCAGGTGGTGGAGGTTCTGGCGGCGGCGGTTCCGAAATTGTACT
AACCCAATCTCCTGGCACACTTAGTCTGTCTCCTGGAGAACGTGCAACG
TTGAGCTGCCGCGCTAGCCAGTCCGTGTCTTCCTCGTACCTCGCCTGGT
ACCAGCAAAAGCCAGGGCAAGCTCCCAGGTTGCTGATTTACGGAGCCTC
AAGTAGGGCGACTGGAATCCCTGACAGATTCAGTGGGAGCGGGTCCGGG
ACTGATTTTACTCTCACCATTTCTAGACTTGAGCCCGAAGACTTCGCCG
TTTATTACTGTCAGCAGTATGGCTCTTCCCCGATGTACACATTCGGCCA
GGGCACAAAACTGGAAATCAAG.

SEQ ID NO: 18 includes Target scFv: 1-708; Target VL: 382-708; Target linker: 337-381; Target VH: 1-336; Free energy: −268.3; gdT CAI: 0.722372746155158; ORF count: 6.

SEQ ID NO: 19 is the sequence name for PTK7 ECOg (74). It is a Tumor Targeting scFv construct. It is a DNA sequence. The sequence is:

GAGGTGCAGCTCGTGCAGTCCGGCGGAGGCCTCGTGCACCCAGGCGGCT
CCCTGCGCCTGTCCTGCGCCGGGTCAGGCTTCACCTTCTCCACGTACCT
CATGTACTGGGTGCGGCAGGCCCCAGGGAAGACCCTGGAGTGGGTGTCC
GCCATCGGGTCCGGCGGCGACACGTACTACGCCGACTCCGTGAAGGGGC
GCTTCACCATCTCCCGGGACAACGCCAAGAACTCCCTGTACCTGCAGAT
GAACTCCCTGCGGGCCGAGGACATGGCCGTGTACTACTGCGCCAGGGGC
CTGGGGTACTGGGGCCAGGGGACCCTCGTGACCGTGTCCTCCGGCGGCG
GAGGCTCAGGAGGAGGAGGCTCAGGAGGCGGCGGCAGCGAGATCGTCCT
GACGCAGAGCCCCGGGACCCTGTCCCTGAGCCCCGGGGAGAGGGCGACC
CTGTCCTGCCGGGCCAGCCAGTCCGTGTCCTCCTCCTACCTGGCCTGGT
ACCAGCAGAAGCCCGGGCAAGCCCCACGCCTCCTCATCTACGGGGCTTC
CTCCAGGGCCACGGGCATCCCCGACCGCTTCTCCGGGTCCGGGTCCGGG
ACGGACTTCACCCTGACCATCTCCCGGCTGGAGCCCGAGGACTTCGCCG
TGTACTACTGCCAGCAGTACGGGTCCTCGCCCATGTACACCTTCGGGCA
GGGGACGAAGCTGGAGATCAAG.

SEQ ID NO: 19 includes Target scFv: 1-708; Target VL: 382-708; Target linker: 337-381; Target VH: 1-336; Free energy: −336.8; gdT CAI: 0.861158; ORF count: 0.

SEQ ID NO: 20 is the sequence name for PTK7 ECOg (18). It is a Tumor Targeting scFv construct. It is a DNA sequence. The sequence is:

GAGGTCCAGCTGGTGCAGTCCGGCGGTGGCCTGGTGCACCCAGGCGGCA
GCCTGCGGCTGTCGTGCGCCGGCAGTGGGTTCACCTTCTCCACCTACCT
GATGTACTGGGTGCGCCAGGCCCCAGGGAAGACCCTGGAGTGGGTGTCC
GCCATCGGCTCCGGCGGCGACACCTACTACGCCGATAGCGTCAAGGGGC
GCTTCACCATCTCCCGCGACAACGCCAAGAACTCCCTCTACCTCCAGAT
GAACTCCCTGCGGGCCGAGGACATGGCCGTCTACTACTGCGCCAGGGGC
CTGGGCTACTGGGGCCAGGGGACCCTGGTGACCGTGTCCTCCGGCGGCG
GTGGCTCTGGTGGTGGTGGCTCTGGTGGCGGCGGCAGCGAGATCGTGCT
GACCCAGTCGCCCGGGACCCTGTCGCTGTCGCCCGGCGAGCGAGCGACC
CTCAGCTGCCGCGCGAGCCAGAGCGTGAGCTCCAGCTACCTCGCGTGGT
ACCAGCAGAAGCCCGGGCAAGCCCCACGGCTGCTGATCTACGGCGCTAG
CTCCAGGGCCACCGGCATCCCCGATCGCTTCAGCGGCAGCGGCAGCGGG
ACCGACTTCACCCTGACGATCTCCCGGCTGGAGCCCGAGGACTTCGCCG
TCTACTACTGCCAGCAGTACGGCTCCAGCCCCATGTACACCTTCGGCCA
GGGGACCAAGCTGGAGATCAAG.

SEQ ID NO: 20 includes Target scFv: 1-708; Target VL: 382-708; Target linker: 337-381; Target VH: 1-336; Free energy: −356; gdT CAI: 0.803927502492027; ORF count: 4.

SEQ ID NO: 21 is the sequence name for PTK7 ECOg (70). It is a Tumor Targeting scFv construct. It is a DNA sequence. The sequence is:

GAGGTGCAGCTGGTGCAGAGCGGCGGAGGCCTGGTGCACCCAGGCGGCA
GCCTGCGGCTGAGCTGCGCTGGCTCCGGCTTCACCTTCAGCACCTACCT
GATGTACTGGGTGCGGCAGGCCCCAGGCAAGACCCTGGAGTGGGTGTCC
GCCATCGGCAGCGGCGGCGACACCTACTACGCCGACAGCGTGAAGGGGC
GCTTCACCATCAGCCGGGACAACGCCAAGAACAGCCTGTACCTGCAGAT
GAACAGCCTGCGGGCCGAGGACATGGCCGTGTACTACTGCGCCAGGGGC
CTGGGCTACTGGGGCCAGGGCACCCTGGTGACCGTGAGCAGCGGCGGCG
GTGGCTCTGGTGGAGGAGGCTCTGGTGGCGGCGGCTCCGAGATCGTGCT
GACCCAGAGCCCCGGCACCCTGAGCCTGAGCCCCGGGGAGCGGGCTACC
CTGAGCTGCCGGGCTTCCCAGAGCGTGAGCAGCTCCTACCTGGCCTGGT
ACCAGCAGAAGCCCGGGCAGGCACCACGGCTGCTGATCTACGGGGCTAG
CAGCCGGGCTACCGGCATCCCCGACCGCTTCTCCGGCAGCGGCAGCGGC
ACCGACTTCACCCTGACCATCTCCAGGCTGGAGCCCGAGGACTTCGCCG
TGTACTACTGCCAGCAGTACGGCAGCTCTCCCATGTACACCTTCGGGCA
GGGCACCAAGCTGGAGATCAAG.

SEQ ID NO: 21 includes Target scFv: 1-708; Target VL: 382-708; Target linker: 337-381; Target VH: 1-336; Free energy: −355.6; gdT CAI: 0.830356865758289; ORF count: 3.

SEQ ID NO: 22 is the sequence name for PTK7 ECOg (68). It is a Tumor Targeting scFv construct. It is a DNA sequence. The sequence is:

GAGGTCCAGCTGGTCCAGTCCGGCGGCGGCCTGGTTCATCCCGGCGGCA
GCCTGCGGCTGAGCTGCGCCGGGTCTGGCTTCACCTTCAGCACCTACCT
GATGTACTGGGTCCGGCAGGCGCCCGGCAAGACCCTGGAGTGGGTGTCG
GCCATCGGGTCCGGCGGCGACACCTACTACGCCGACTCGGTCAAGGGCC
GGTTCACCATCAGCCGGGACAACGCCAAGAACAGCCTGTACCTGCAGAT
GAACAGCCTGCGGGCCGAGGACATGGCCGTCTACTACTGCGCCCGCGGG
CTGGGTTACTGGGGCCAGGGGACCCTGGTGACCGTCAGCTCCGGCGGCG
GCGGATCTGGTGGTGGCGGATCTGGTGGCGGCGGCTCCGAGATCGTGCT
GACCCAGTCGCCCGGGACCCTGAGCCTGAGCCCCGGCGAGCGGGCTACC
CTGAGCTGCCGGGCCAGCCAGTCGGTCAGCTCCAGCTACCTGGCCTGGT
ACCAGCAGAAACCCGGCCAGGCTCCCCGGCTGCTGATCTACGGGGCTAG
TAGCCGGGCCACCGGCATCCCCGATCGGTTCAGCGGGTCCGGGTCCGGG
ACCGACTTCACCCTGACCATCAGCCGGCTGGAGCCCGAGGACTTCGCCG
TCTACTACTGCCAGCAGTACGGGTCCAGCCCGATGTACACCTTCGGCCA
GGGGACCAAGCTGGAGATCAAG.

SEQ ID NO: 22 includes Target scFv: 1-708; Target VL: 382-708; Target linker: 337-381; Target VH: 1-336; Free energy: −355.5; gdT CAI: 0.811107619741586; ORF count: 2.

SEQ ID NO: 23 is the sequence name for PTK7 ECOg (2). It is a Tumor Targeting scFv construct. It is a DNA sequence. The sequence is:

GAGGTGCAGCTGGTGCAGAGCGGCGGAGGCCTGGTGCACCCAGGCGGCT
CCCTGCGGCTCAGCTGCGCCGGTTCCGGGTTCACCTTTTCCACGTATCT
GATGTACTGGGTGCGCCAGGCCCCGGGGAAGACCCTGGAATGGGTCTCC
GCCATCGGCTCCGGCGGCGACACCTACTACGCCGACTCCGTGAAGGGGC
GCTTCACCATTTCCCGGGACAACGCGAAGAATTCCCTGTACCTGCAGAT
GAACAGTCTCCGCGCCGAGGACATGGCCGTGTACTACTGTGCCCGGGGC
CTCGGATACTGGGGCCAGGGGACCCTGGTCACCGTGTCCAGCGGCGGCG
GAGGCTCAGGAGGAGGAGGCTCAGGAGGCGGCGGCTCCGAGATCGTGTT
GACCCAGAGCCCCGGGACCCTGTCCCTGAGCCCCGGGGAACGGGCCACG
CTGAGCTGCCGCGCCTCGCAGAGCGTCTCCTCCTCGTACCTGGCCTGGT
ACCAGCAGAAGCCGGGGCAGGCTCCTCGGCTCCTCATCTACGGGGCCTC
TTCCCGGGCCACGGGTATCCCCGACAGGTTCAGCGGCTCCGGGTCCGGG
ACCGATTTCACCTTGACCATTAGCCGCCTGGAACCCGAGGACTTCGCCG
TGTACTATTGTCAGCAGTACGGGTCCTCGCCCATGTACACCTTTGGCCA
GGGGACCAAGCTGGAGATCAAG.

SEQ ID NO: 23 includes Target scFv: 1-708; Target VL: 382-708; Target linker: 337-381; Target VH: 1-336; Free energy: −350.2; gdT CAI: 0.803914913406133; ORF count: 2.

SEQ ID NO: 24 is the sequence name for hSCF ligand. It is a Tumor Targeting Ligand construct. It is an AA sequence. The sequence is:

EGICRNRVTNNVKDVTKLVANLPKDYMITLKYVPGMDVLPSHCWISEMV
VQLSDSLTDLLDKFSNISEGLSNYSIIDKLVNIVDDLVECVKENSSKDL
KKSFKSPEPRLFTPEEFFRIFNRSIDAFKDFVVASETSDCVVSS.

SEQ ID NO: 24 includes Target Ligand: 1-142.

SEQ ID NO: 25 is the sequence name for hSCF ligand. It is a Tumor Targeting Ligand construct. It is a DNA sequence. The sequence is:

GAGGGGATCTGCAGGAACAGGGTGACCAACAATGTGAAGGATGTGACCA
AGCTGGTGGCCAACCTGCCCAAGGACTACATGATCACCCTGAAGTATGT
GCCAGGGATGGATGTGCTGCCCAGCCACTGCTGGATCTCTGAGATGGTG
GTGCAGCTGTCTGACTCCCTGACAGACCTGCTGGACAAGTTCTCCAACA
TCTCAGAGGGGCTGTCCAACTACTCCATCATTGACAAGCTGGTGAACAT
AGTGGATGACCTGGTGGAGTGTGTGAAGGAGAACTCCTCCAAGGACCTG
AAGAAGTCCTTCAAGTCCCCTGAGCCCAGGCTGTTCACCCCTGAGGAGT
TCTTCAGGATCTTCAACAGGTCCATTGATGCCTTCAAGGACTTTGTGGT
GGCCTCTGAGACCTCTGACTGTGTGGTGTCCTCA.

SEQ ID NO: 25 includes Target Ligand: 1-426; Free energy: −157.8; gdT CAI: 0.937866445503855; ORF count: 3.

SEQ ID NO: 26 is the sequence name for hSCF ligand ECOg (87). It is a Tumor Targeting Ligand construct. It is a DNA sequence. The sequence is:

GAGGGGATCTGCCGGAACCGGGTGACCAACAACGTGAAGGACGTGACCA
AGCTGGTGGCCAACCTGCCCAAGGACTACATGATCACCCTGAAGTACGT
GCCCGGGATGGACGTGCTGCCCAGCCACTGCTGGATCAGCGAGATGGTG
GTGCAGCTGTCCGACTCCCTGACCGACCTGCTGGACAAGTTCTCCAACA
TCAGCGAGGGGCTGAGCAACTACTCCATCATCGACAAGCTGGTGAACAT
CGTGGACGACCTGGTGGAGTGCGTGAAGGAGAACAGCAGCAAGGACCTG
AAGAAGTCCTTCAAGAGCCCCGAGCCCCGGCTGTTCACGCCCGAGGAGT
TCTTCCGGATCTTCAACCGGAGCATCGACGCCTTCAAGGACTTCGTGGT
GGCCAGCGAGACCAGCGACTGCGTGGTGTCCTCC.

SEQ ID NO: 26 includes Target Ligand: 1-426; Free energy: −160.7; gdT CAI: 0.928424; ORF count: 0.

SEQ ID NO: 27 is the sequence name for hSCF ligand ECOg (62). It is a Tumor Targeting Ligand construct. It is a DNA sequence. The sequence is:

GAGGGGATCTGCCGGAACCGGGTGACCAACAATGTGAAGGACGTCACAA
AGTTGGTCGCCAATCTGCCGAAGGACTACATGATTACGCTGAAGTACGT
CCCCGGAATGGATGTGCTGCCCAGCCACTGCTGGATTTCGGAGATGGTG
GTGCAGCTGTCCGACAGTCTGACCGATCTGCTGGACAAGTTCAGCAACA
TCTCCGAAGGGCTGTCCAACTACAGCATCATCGATAAGCTGGTCAACAT
CGTCGACGATCTGGTGGAGTGCGTCAAAGAGAACAGCAGCAAAGATCTG
AAGAAGTCGTTCAAATCGCCGGAGCCGCGGCTGTTCACACCGGAGGAGT
TCTTCCGGATCTTCAATCGGTCGATCGACGCCTTCAAAGATTTTGTGGT
GGCCAGCGAAACCAGCGACTGCGTCGTCAGCAGC.

SEQ ID NO: 27 includes Target Ligand: 1-426; Free energy: −164.6; gdT CAI: 0.805174371566857; ORF count: 2.

SEQ ID NO: 28 is the sequence name for hSCF ligand ECOg (61). It is a Tumor Targeting Ligand construct. It is a DNA sequence. The sequence is:

GAGGGGATCTGCCGGAACCGCGTGACGAACAACGTGAAGGACGTCACGA
AGCTCGTCGCGAACCTGCCGAAGGACTACATGATCACCCTGAAGTACGT
CCCCGGGATGGACGTGCTCCCCTCGCACTGCTGGATCTCCGAGATGGTC
GTCCAGCTGTCCGACTCCCTGACGGACCTCCTCGACAAGTTCTCCAACA
TCTCCGAGGGGCTCTCCAACTACTCGATCATCGACAAGCTGGTGAACAT
CGTGGACGACCTCGTGGAGTGCGTCAAGGAGAACTCCTCGAAGGACCTC
AAGAAGAGCTTCAAGTCGCCCGAGCCGCGGCTCTTCACGCCCGAGGAGT
TCTTCCGGATCTTCAACCGGAGCATCGACGCCTTCAAGGACTTCGTGGT
GGCCTCCGAGACGTCCGACTGCGTCGTGTCGAGC.

SEQ ID NO: 28 includes Target Ligand: 1-426; Free energy: −163.9; gdT CAI: 0.83416441300645; ORF count: 0.

SEQ ID NO: 29 is the sequence name for hSCF ligand ECOg (2). It is a Tumor Targeting Ligand construct. It is a DNA sequence. The sequence is:

GAGGGGATCTGCCGGAACCGGGTGACCAACAACGTGAAGGACGTCACCA
AGCTGGTCGCCAACCTGCCCAAGGACTACATGATCACCTTGAAGTACGT
GCCCGGCATGGACGTCCTGCCCAGCCACTGCTGGATCTCCGAGATGGTC
GTCCAGCTCAGCGACTCCCTGACCGACCTCCTCGACAAGTTCTCCAACA
TCTCCGAGGGGCTCAGCAACTACTCCATCATCGACAAGCTCGTGAACAT
AGTGGATGACCTCGTGGAGTGCGTGAAGGAGAACAGCTCCAAGGACTTG
AAGAAGTCCTTCAAGTCCCCGGAGCCCAGGCTGTTCACGCCCGAGGAGT
TCTTCAGGATCTTCAACCGATCCATTGACGCCTTCAAGGACTTCGTGGT
GGCCTCCGAGACCAGCGACTGCGTGGTGTCCTCC.

SEQ ID NO: 29 includes Target Ligand: 1-426; Free energy: −163.7; gdT CAI: 0.886419970682739; ORF count: 2.

SEQ ID NO: 30 is the sequence name for hSCF ligand ECOg (48). It is a Tumor Targeting Ligand construct. It is a DNA sequence. The sequence is:

GAGGGGATCTGCCGCAACCGCGTGACGAACAACGTGAAGGACGTGACGA
AGCTCGTGGCGAACCTGCCCAAGGACTACATGATCACCCTGAAGTACGT
CCCCGGGATGGACGTGCTGCCCAGCCACTGCTGGATCTCCGAGATGGTG
GTGCAGCTGAGCGACAGCCTGACGGACCTGCTGGACAAGTTCAGCAACA
TCTCCGAGGGGCTGAGCAACTACAGCATCATCGACAAGCTGGTGAACAT
CGTGGACGACCTGGTGGAGTGCGTGAAGGAGAACAGCTCCAAGGACCTG
AAGAAGAGCTTCAAGTCGCCCGAGCCCCGGCTGTTCACGCCCGAGGAGT
TCTTCCGGATCTTCAACCGGAGCATCGACGCCTTCAAGGACTTCGTCGT
GGCGAGCGAGACGTCCGACTGCGTCGTGTCCTCC.

SEQ ID NO: 30 includes Target Ligand: 1-426; Free energy: −163.5; gdT CAI: 0.876702550243938; ORF count: 0.

SEQ ID NO: 31 is the sequence name for Albumin. It is a Fusion Moiety construct. It is an AA sequence. The sequence is:

DAHKSEVAHRFKDLGEENFKALVLIAFAQYLQQCPFEDHVKLVNEVTEF
AKTCVADESAENCDKSLHTLFGDKLCTVATLRETYGEMADCCAKQEPER
NECFLQHKDDNPNLPRLVRPEVDVMCTAFHDNEETFLKKYLYEIARRHP
YFYAPELLFFAKRYKAAFTECCQAADKAACLLPKLDELRDEGKASSAKQ
RLKCASLQKFGERAFKAWAVARLSQRFPKAEFAEVSKLVTDLTKVHTEC
CHGDLLECADDRADLAKYICENQDSISSKLKECCEKPLLEKSHCIAEVE
NDEMPADLPSLAADFVESKDVCKNYAEAKDVFLGMFLYEYARRHPDYSV
VLLLRLAKTYETTLEKCCAAADPHECYAKVFDEFKPLVEEPQNLIKQNC
ELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSA
LEVDETYVPKEFNAETFTFHADICTLSEKERQIKKQTALVELVKHKPKA
TKEQLKAVMDDFAAFVEKCCKADDKETCFAEEGKKLVAASQAALGLGGG
GS.

SEQ ID NO: 31 includes Fusion Moeity: 1-590.

SEQ ID NO: 32 is the sequence name for Albumin. It is a Fusion Moiety construct. It is a DNA sequence. The sequence is:

GACGCTCATAAGTCCGAGGTGGCCCATCGCTTTAAGGATCTGGGTGAGG
AAAATTTTAAGGCGCTGGTGTTGATTGCATTTGCGCAGTATTTGCAGCA
GTGTCCATTCGAGGATCACGTGAAGTTAGTGAACGAGGTGACTGAGTTC
GCAAAGACCTGTGTCGCGGATGAGAGCGCTGAGAACTGTGATAAATCCC
TTCACACCCTGTTCGGCGATAAACTTTGCACTGTGGCCACATTGCGCGA
AACTTACGGAGAGATGGCCGACTGCTGCGCTAAACAGGAGCCCGAGAGG
AACGAGTGCTTCTTACAGCATAAGGATGACAACCCCAATCTGCCAAGAC
TGGTCAGGCCTGAAGTGGACGTCATGTGTACAGCCTTCCATGACAACGA
GGAGACGTTCCTCAAGAAATACTTGTACGAGATCGCTCGACGTCACCCT
TATTTCTACGCACCTGAATTGCTGTTTTTTGCCAAGCGGTATAAAGCAG
CTTTCACAGAGTGCTGCCAGGCTGCAGACAAGGCTGCCTGCTTGCTTCC
AAAATTGGACGAGCTGAGAGATGAGGGCAAGGCATCCTCAGCTAAACAG
CGGCTGAAATGTGCCTCTCTGCAAAAGTTCGGGGAGCGGGCTTTCAAGG
CCTGGGCCGTTGCGCGGCTGAGCCAGAGATTTCCCAAGGCCGAGTTCGC
TGAAGTTTCAAAGCTCGTCACCGACCTTACCAAGGTTCATACTGAATGT
TGCCATGGCGACCTGCTAGAATGCGCAGACGACAGGGCGGATCTGGCCA
AGTACATCTGTGAGAATCAAGACTCTATTAGTAGCAAGCTCAAAGAGTG
TTGCGAGAAGCCATTACTCGAAAAGTCCCATTGCATCGCAGAGGTAGAA
AACGATGAAATGCCTGCCGACCTTCCCAGTCTCGCCGCCGACTTTGTGG
AGAGCAAAGATGTGTGCAAAAATTACGCAGAGGCCAAGGATGTATTCCT
GGGGATGTTTCTCTACGAGTATGCCAGAAGACACCCCGATTATAGTGTG
GTGCTCCTTCTGCGCTTAGCCAAAACATACGAGACCACCCTGGAGAAGT
GTTGTGCAGCCGCCGATCCACACGAGTGCTATGCTAAGGTCTTTGATGA
ATTTAAACCACTAGTCGAAGAGCCCCAGAATCTCATAAAGCAGAATTGC
GAACTTTTTGAACAGCTGGGCGAATATAAATTCCAAAATGCTCTGCTAG
TACGATATACCAAGAAAGTGCCCCAAGTAAGTACTCCTACGTTGGTTGA
GGTGAGCCGTAACCTGGGGAAGGTGGGATCAAAGTGCTGTAAACACCCT
GAAGCGAAAAGGATGCCGTGCGCAGAAGATTACCTTTCTGTGGTACTCA
ACCAGCTCTGCGTGTTACACGAGAAGACTCCGGTGTCTGACCGAGTTAC
AAAATGTTGCACCGAGTCACTCGTCAATCGCCGGCCCTGCTTCAGCGCT
CTAGAAGTCGACGAAACCTACGTGCCTAAGGAATTCAACGCCGAGACGT
TCACCTTTCACGCCGACATCTGTACTCTCTCCGAGAAGGAGAGGCAGAT
TAAGAAACAAACAGCACTGGTGGAACTGGTTAAACACAAACCAAAGGCT
ACAAAGGAACAGCTGAAAGCGGTTATGGATGACTTCGCCGCCTTTGTCG
AAAAGTGTTGTAAAGCAGACGATAAAGAAACATGTTTTGCTGAAGAAGG
TAAGAAACTGGTCGCCGCCTCGCAGGCAGCTCTGGGACTGGGAGGTGGT
GGGTCT.

SEQ ID NO: 32 includes Fusion Moeity: 1-1770; Free energy: −557.6; gdT CAI: 0.787786351918606; ORF count: 21.

SEQ ID NO: 33 is the sequence name for Albumin ECOg (8). It is a Fusion Moiety construct. It is a DNA sequence. The sequence is:

GACGCCCACAAGTCCGAGGTGGCCCACAGGTTCAAGGACCTGGGCGAGGAGAACTTCAA
GGCCCTGGTGCTGATCGCCTTCGCCCAGTACCTGCAGCAGTGCCCCTTCGAGGACCACGT
GAAGTTGGTGAACGAGGTGACCGAGTTCGCCAAGACCTGCGTGGCCGACGAGAGCGCCG
AGAACTGCGACAAGTCCCTGCACACCCTGTTCGGGGACAAGCTGTGCACCGTGGCCACG
CTGCGGGAGACCTACGGGGAGATGGCCGACTGCTGCGCCAAGCAGGAGCCCGAGCGGA
ACGAGTGCTTCCTGCAGCACAAGGACGACAACCCCAACCTGCCCCGACTGGTCCGGCCC
GAGGTGGACGTGATGTGCACCGCCTTCCACGACAACGAGGAGACCTTCCTGAAGAAGTA
CCTGTACGAGATCGCCCGGCGGCACCCCTACTTCTACGCGCCCGAGCTGCTGTTCTTCGC
CAAGCGGTACAAGGCCGCCTTCACCGAGTGCTGCCAGGCCGCCGACAAGGCCGCCTGCC
TGCTGCCCAAGCTGGACGAGCTGCGGGACGAGGGGAAGGCCTCCAGCGCCAAGCAGCG
GCTGAAGTGCGCGTCCCTGCAGAAGTTCGGGGAGCGGGCCTTCAAGGCTTGGGCCGTGG
CCCGGCTGTCCCAACGGTTCCCCAAGGCCGAGTTCGCCGAGGTGAGCAAGTTGGTGACC
GACCTGACCAAGGTGCACACCGAGTGCTGCCACGGGGACCTGCTGGAGTGCGCCGACGA
CCGGGCCGATCTGGCCAAGTACATCTGCGAGAACCAGGACAGCATCAGCTCCAAGCTGA
AGGAGTGCTGCGAGAAGCCCCTGCTGGAGAAGTCCCACTGCATCGCCGAGGTGGAGAAC
GACGAGATGCCGGCCGATCTGCCCTCGCTGGCCGCCGACTTCGTGGAGAGCAAGGACGT
GTGCAAGAACTACGCCGAGGCCAAGGACGTGTTCCTGGGGATGTTCCTGTACGAGTACG
CCCGGCGGCACCCCGACTACTCCGTGGTGCTGCTGCTGCGGCTGGCCAAGACCTACGAG
ACCACCTTGGAGAAGTGCTGCGCCGCGGCCGATCCGCACGAGTGCTACGCCAAGGTGTT
CGACGAGTTCAAGCCCCTGGTGGAGGAGCCCCAGAACCTGATCAAGCAGAACTGCGAGC
TGTTCGAGCAGCTCGGGGAGTACAAGTTCCAGAACGCCCTGCTGGTGCGGTACACCAAG
AAGGTGCCCCAGGTGAGCACCCCGACCCTGGTGGAGGTGAGCCGGAACCTGGGGAAGG
TCGGGTCCAAGTGCTGCAAGCACCCCGAGGCCAAGCGGATGCCCTGCGCCGAGGACTAC
CTGTCCGTGGTGCTGAACCAGCTGTGCGTGCTGCACGAGAAGACGCCCGTGTCCGACCG
GGTGACCAAGTGCTGCACCGAGAGCTTGGTGAACCGGCGGCCCTGCTTCAGCGCCCTGG
AGGTGGACGAGACCTACGTGCCCAAGGAGTTCAACGCCGAGACCTTCACCTTCCACGCC
GACATCTGCACCCTGAGCGAGAAGGAGCGGCAGATCAAGAAGCAGACGGCCCTGGTGG
AGCTGGTGAAGCACAAGCCCAAGGCCACCAAGGAGCAGCTGAAGGCCGTGATGGACGA
CTTCGCCGCCTTCGTGGAGAAGTGCTGCAAGGCCGACGACAAGGAGACCTGCTTCGCCG
AAGAGGGCAAGAAGTTGGTGGCCGCGTCCCAGGCCGCACTGGGTCTGGGAGGTGGTGG
GTCT.

SEQ ID NO: 33 includes Fusion Moeity: 1-1770; Free energy: −755.4; gdT CAI: 0.932865573516391; ORF count: 0.

SEQ ID NO: 34 is the sequence name for Albumin ECOg (60). It is a Fusion Moiety construct. It is a DNA sequence. The sequence is:

GACGCCCACAAGAGCGAGGTGGCGCACCGCTTCAAGGACCTGGGCGAGGAGAACTTCA
AGGCGCTGGTGCTGATCGCGTTCGCCCAGTACCTCCAGCAGTGCCCCTTCGAGGACCACG
TGAAGCTGGTGAACGAGGTGACCGAGTTCGCCAAGACCTGCGTGGCCGACGAGAGCGCC
GAGAACTGCGACAAGAGCCTCCACACCCTGTTCGGGGACAAGCTGTGCACCGTGGCCAC
GCTGCGCGAGACCTACGGGGAGATGGCCGACTGCTGCGCCAAGCAGGAGCCCGAGCGC
AACGAGTGCTTCCTCCAGCACAAGGACGACAACCCCAACCTACCCCGTCTGGTGCGCCC
CGAGGTGGACGTGATGTGCACCGCGTTCCACGACAACGAGGAGACCTTCCTCAAGAAGT
ACCTCTACGAGATCGCGCGGCGGCACCCCTACTTCTACGCGCCCGAGCTGCTGTTCTTCG
CCAAGCGCTACAAGGCCGCGTTCACCGAGTGCTGCCAGGCCGCCGACAAGGCCGCGTGC
CTGCTCCCCAAGCTCGACGAGCTGCGCGACGAGGGGAAGGCGTCGAGCGCCAAGCAGA
GGCTCAAGTGCGCCTCGCTCCAGAAGTTCGGGGAGCGCGCGTTCAAGGCGTGGGCCGTG
GCGAGGCTCTCCCAGCGGTTCCCCAAGGCCGAGTTCGCCGAGGTGAGCAAGCTGGTGAC
CGACCTCACCAAGGTGCACACCGAGTGCTGCCACGGGGACCTGCTGGAGTGCGCCGACG
ACCGCGCCGATCTCGCCAAGTACATCTGCGAGAACCAGGACAGCATCTCCTCCAAGCTC
AAGGAGTGCTGCGAGAAGCCCCTGCTGGAGAAGAGCCACTGCATCGCCGAGGTGGAGA
ACGACGAGATGCCAGCCGATCTCCCCTCGCTCGCCGCCGACTTCGTGGAGAGCAAGGAC
GTGTGCAAGAACTACGCCGAGGCCAAGGACGTGTTCCTGGGGATGTTCCTCTACGAGTA
CGCGCGGCGGCACCCCGACTACAGCGTGGTGCTGCTGCTGCGGCTCGCCAAGACCTACG
AGACCACGCTGGAGAAGTGCTGCGCCGCCGCCGATCCCCACGAGTGCTACGCCAAGGTG
TTCGACGAGTTCAAGCCCCTGGTGGAGGAGCCCCAGAACCTGATCAAGCAGAACTGCGA
GCTGTTCGAGCAGCTCGGGGAGTACAAGTTCCAGAACGCGCTGCTGGTGCGCTACACCA
AGAAGGTGCCCCAGGTGAGCACCCCGACCCTGGTGGAGGTGAGCCGCAACCTGGGGAA
GGTCGGGAGCAAGTGCTGCAAGCACCCCGAGGCCAAGCGGATGCCCTGCGCCGAGGACT
ACCTCTCGGTGGTGCTCAACCAGCTGTGCGTGCTCCACGAGAAGACCCCGGTGAGCGAC
AGGGTGACCAAGTGCTGCACCGAGTCGCTGGTGAACCGGAGGCCCTGCTTCTCCGCGCT
GGAGGTGGACGAGACCTACGTGCCCAAGGAGTTCAACGCCGAGACCTTCACCTTCCACG
CCGACATCTGCACCCTCTCCGAGAAGGAGCGCCAGATCAAGAAGCAGACCGCGCTGGTG
GAGCTGGTGAAGCACAAGCCCAAGGCCACCAAGGAGCAGCTCAAGGCGGTGATGGACG
ACTTCGCCGCGTTCGTGGAGAAGTGCTGCAAGGCCGACGACAAGGAGACCTGCTTCGCC
GAAGAGGGGAAGAAGCTGGTGGCCGCGAGCCAAGCCGCTCTGGGGCTGGGAGGTGGTG
GGTCT.

SEQ ID NO: 34 includes Fusion Moeity: 1-1770; Free energy: −753.1; gdT CAI: 0.893692649517376; ORF count: 0.

SEQ ID NO: 35 is the sequence name for Albumin ECOg (91). It is a Fusion Moiety construct. It is a DNA sequence. The sequence is:

GACGCCCACAAGTCTGAGGTCGCCCATCGGTTCAAGGATCTCGGCGAGGAGAACTTCAA
GGCCCTCGTGCTGATCGCCTTCGCGCAGTACCTCCAGCAGTGCCCCTTCGAGGACCACGT
GAAGCTGGTCAACGAGGTGACCGAGTTCGCCAAGACCTGCGTGGCCGACGAGAGCGCCG
AGAACTGCGATAAGAGCCTGCACACCCTGTTCGGCGACAAGCTGTGCACCGTGGCGACC
CTCAGGGAGACCTACGGGGAGATGGCCGACTGCTGCGCCAAGCAGGAGCCCGAGCGGA
ACGAGTGCTTCCTGCAGCACAAGGATGACAACCCCAATCTGCCCCGCTTGGTGCGCCCC
GAGGTGGACGTGATGTGCACCGCGTTCCACGACAACGAGGAGACCTTCCTGAAGAAGTA
CCTCTACGAGATCGCCAGGCGGCACCCCTACTTCTACGCTCCCGAGCTGCTGTTCTTCGC
CAAGCGGTACAAGGCGGCCTTCACGGAGTGCTGCCAGGCGGCCGACAAGGCGGCCTGCC
TGCTGCCCAAGCTGGACGAGCTGCGCGACGAGGGGAAGGCCAGCAGCGCCAAGCAGAG
ACTGAAGTGCGCCTCTCTGCAGAAGTTCGGCGAGCGGGCCTTCAAGGCGTGGGCCGTCG
CTCGGCTCTCCCAGCGGTTCCCCAAGGCGGAGTTCGCCGAGGTGTCCAAGCTGGTGACC
GACCTGACCAAGGTGCACACCGAGTGCTGCCACGGGGACCTCCTGGAGTGCGCCGACGA
TCGGGCCGACCTGGCGAAGTACATCTGCGAGAACCAGGATAGCATCAGCTCCAAGCTGA
AGGAGTGCTGCGAGAAGCCCCTGCTGGAGAAGAGCCACTGCATCGCCGAGGTGGAGAA
CGACGAGATGCCCGCAGATCTGCCCAGCCTGGCGGCCGACTTCGTGGAGAGCAAGGATG
TCTGCAAGAATTACGCCGAGGCGAAGGACGTGTTCCTCGGGATGTTCCTCTACGAGTAC
GCCCGGCGGCACCCCGATTACAGCGTGGTGCTCCTCCTGCGGCTCGCCAAGACCTACGA
GACAACCCTGGAGAAGTGCTGCGCTGCCGCCGATCCCCACGAGTGCTACGCCAAGGTGT
TCGACGAGTTCAAGCCCCTCGTGGAGGAGCCCCAGAATCTCATCAAGCAGAACTGCGAG
CTGTTCGAGCAGCTCGGGGAGTACAAGTTCCAGAACGCCCTGCTGGTGAGGTACACCAA
GAAGGTGCCCCAGGTGAGCACCCCGACCCTCGTGGAGGTGAGCCGCAACCTGGGGAAG
GTCGGCTCTAAGTGCTGCAAGCACCCCGAGGCCAAGCGGATGCCCTGCGCGGAGGACTA
CCTGTCCGTGGTGCTCAACCAGCTGTGCGTGCTGCACGAGAAGACACCCGTGTCCGACC
GGGTGACCAAGTGCTGCACGGAGTCCCTGGTGAACCGGCGGCCCTGCTTCTCCGCTCTGG
AGGTCGACGAGACCTACGTGCCCAAGGAGTTCAACGCCGAGACGTTCACCTTCCACGCG
GATATCTGCACGCTGAGCGAGAAGGAGAGGCAGATCAAGAAGCAGACGGCCCTCGTGG
AGCTCGTGAAGCACAAGCCCAAGGCCACCAAGGAGCAGCTGAAGGCCGTGATGGACGA
CTTCGCGGCCTTCGTGGAGAAGTGCTGCAAGGCGGACGACAAGGAGACCTGCTTCGCCG
AGGAAGGCAAGAAGCTGGTGGCCGCCAGTCAGGCTGCCCTGGGGCTCGGAGGTGGTGG
GTCT.

SEQ ID NO: 35 includes Fusion Moeity: 1-1770; Free energy: −750.9; gdT CAI: 0.909026600521571; ORF count: 7.

SEQ ID NO: 36 is the sequence name for Albumin ECOg (51). It is a Fusion Moiety construct. It is a DNA sequence. The sequence is:

GACGCCCACAAGTCCGAGGTGGCGCACCGCTTCAAGGACCTCGGGGAAGAGAACTTCAA
GGCCCTGGTGCTGATCGCCTTCGCGCAGTACCTGCAGCAGTGCCCCTTCGAGGACCACGT
GAAGCTCGTGAACGAGGTCACGGAGTTCGCCAAGACCTGCGTCGCCGACGAGAGCGCCG
AGAACTGCGACAAGAGCCTCCACACCCTGTTCGGGGACAAGCTCTGCACCGTGGCGACG
CTGCGGGAGACCTACGGCGAGATGGCGGACTGCTGCGCCAAGCAGGAGCCCGAGCGCA
ACGAGTGCTTCCTGCAGCACAAGGACGACAACCCCAACCTGCCGCGGCTCGTGAGGCCC
GAGGTCGACGTGATGTGCACCGCGTTCCACGACAACGAGGAGACGTTCCTGAAGAAGTA
CCTCTACGAGATCGCGAGGCGGCACCCCTACTTCTACGCCCCGGAGCTGCTGTTCTTCGC
GAAGCGCTACAAGGCCGCCTTCACGGAGTGCTGCCAGGCCGCCGACAAGGCCGCGTGCC
TGCTGCCGAAGCTCGACGAGCTCCGGGACGAGGGCAAGGCCTCCAGCGCCAAGCAGCGC
CTCAAGTGCGCCTCGCTGCAGAAGTTCGGGGAGCGGGCCTTCAAGGCCTGGGCCGTGGC
GCGGCTGTCACAGCGGTTCCCCAAGGCCGAGTTCGCCGAGGTCTCGAAGCTCGTGACGG
ACCTCACCAAGGTCCACACCGAGTGCTGCCACGGGGACCTCCTGGAGTGCGCCGACGAC
AGGGCCGACCTCGCGAAGTACATCTGCGAGAACCAGGACTCCATCAGCTCCAAGCTCAA
GGAGTGCTGCGAGAAGCCCCTGCTGGAGAAGTCCCACTGCATCGCCGAGGTCGAGAACG
ACGAGATGCCCGCGGATCTCCCGAGCCTCGCCGCGGACTTCGTGGAGTCCAAGGACGTC
TGCAAGAACTACGCCGAGGCCAAGGACGTGTTCCTGGGGATGTTCCTGTACGAGTACGC
GAGGCGGCACCCCGACTACTCCGTGGTGCTGCTGCTGCGGCTCGCCAAGACCTACGAGA
CGACCCTGGAGAAGTGCTGCGCCGCCGCAGATCCCCACGAGTGCTACGCCAAGGTCTTC
GACGAGTTCAAGCCCCTGGTGGAGGAGCCCCAGAACCTCATCAAGCAGAACTGCGAGCT
CTTCGAGCAGCTCGGGGAGTACAAGTTCCAGAACGCGCTGCTGGTGCGGTACACCAAGA
AGGTGCCCCAGGTCTCGACCCCGACCCTGGTGGAGGTGTCCCGGAACCTCGGGAAGGTC
GGGTCCAAGTGCTGCAAGCACCCCGAGGCGAAGCGGATGCCCTGCGCCGAGGACTACCT
CTCCGTGGTGCTGAACCAGCTCTGCGTGCTGCACGAGAAGACCCCGGTGTCGGACCGCG
TCACGAAGTGCTGCACCGAGTCCCTGGTGAACCGGAGGCCCTGCTTCAGCGCCCTGGAG
GTCGACGAGACCTACGTCCCCAAGGAGTTCAACGCCGAGACCTTCACGTTCCACGCCGA
CATCTGCACCCTGTCCGAGAAGGAGCGCCAGATCAAGAAGCAGACGGCCCTGGTGGAGC
TCGTGAAGCACAAGCCCAAGGCCACCAAGGAGCAGCTCAAGGCCGTGATGGACGACTTC
GCGGCCTTCGTGGAGAAGTGCTGCAAGGCCGACGACAAGGAGACCTGCTTCGCCGAGGA
AGGCAAGAAGCTCGTGGCGGCGTCCCAGGCGGCTCTGGGACTCGGAGGTGGTGGGTCT.

SEQ ID NO: 36 includes Fusion Moeity: 1-1770; Free energy: −749.1; gdT CAI: 0.879685715389261; ORF count: 0.

SEQ ID NO: 37 is the sequence name for Albumin ECOg (62). It is a Fusion Moiety construct. It is a DNA sequence. The sequence is:

GACGCCCACAAGTCCGAGGTGGCGCACCGCTTCAAGGACCTGGGCGAGGAGAACTTCAA
GGCCCTGGTGCTGATCGCCTTCGCCCAGTACCTGCAGCAGTGCCCCTTCGAGGACCACGT
GAAGCTGGTGAACGAGGTGACCGAGTTCGCCAAGACCTGCGTGGCCGACGAGTCCGCCG
AGAACTGCGACAAGTCCCTGCACACCCTGTTCGGGGACAAGCTGTGCACCGTGGCCACG
CTGCGGGAGACCTACGGGGAGATGGCCGACTGCTGCGCCAAGCAGGAGCCCGAGCGGA
ACGAGTGCTTCCTGCAGCACAAGGACGACAACCCCAACCTGCCCAGGCTGGTGCGGCCC
GAGGTGGACGTGATGTGCACCGCCTTCCACGACAACGAGGAGACCTTCCTGAAGAAGTA
CCTGTACGAGATCGCCCGGCGGCACCCCTACTTCTACGCGCCCGAGCTGCTGTTCTTCGC
CAAGCGGTACAAGGCCGCCTTCACCGAGTGCTGCCAGGCCGCCGACAAGGCCGCCTGCC
TGCTGCCCAAGCTGGACGAGCTGCGGGACGAGGGGAAGGCCTCCTCCGCCAAGCAGCGG
CTGAAGTGCGCCAGCCTGCAGAAGTTCGGGGAGCGGGCCTTCAAGGCTTGGGCCGTGGC
CCGGCTGTCCCAGAGGTTCCCCAAGGCCGAGTTCGCCGAGGTGTCCAAGCTGGTGACCG
ACCTGACCAAGGTGCACACCGAGTGCTGCCACGGGGACCTGCTGGAGTGCGCCGACGAC
CGGGCTGACCTGGCCAAGTACATCTGCGAGAACCAGGACTCCATCTCCTCCAAGCTGAA
GGAGTGCTGCGAGAAGCCCCTGCTGGAGAAGTCCCACTGCATCGCCGAGGTGGAGAACG
ACGAGATGCCGGCTGACCTGCCCAGCCTGGCCGCCGACTTCGTGGAGTCCAAGGACGTG
TGCAAGAACTACGCCGAGGCCAAGGACGTGTTCCTGGGGATGTTCCTGTACGAGTACGC
CCGGCGGCACCCCGACTACTCCGTGGTGCTGCTGCTGCGGCTGGCCAAGACCTACGAGA
CGACCCTGGAGAAGTGCTGCGCCGCCGCCGATCCCCACGAGTGCTACGCCAAGGTGTTC
GACGAGTTCAAGCCCCTGGTGGAGGAGCCCCAGAACCTGATCAAGCAGAACTGCGAGCT
GTTCGAGCAGCTGGGCGAGTACAAGTTCCAGAACGCCCTGCTGGTGCGGTACACCAAGA
AGGTGCCCCAGGTGAGCACCCCGACCCTGGTGGAGGTGTCCCGGAACCTGGGGAAGGTC
GGGTCCAAGTGCTGCAAGCACCCCGAGGCCAAGCGGATGCCCTGCGCCGAGGACTACCT
GTCCGTGGTGCTGAACCAGCTGTGCGTGCTGCACGAGAAGACGCCCGTGTCCGACCGGG
TGACCAAGTGCTGCACCGAGTCCCTGGTGAACCGGCGGCCCTGCTTCAGCGCCCTGGAG
GTGGACGAGACCTACGTGCCCAAGGAGTTCAACGCCGAGACCTTCACCTTCCACGCCGA
CATCTGCACCCTGTCCGAGAAGGAGCGGCAGATCAAGAAGCAGACGGCCCTGGTGGAGC
TGGTGAAGCACAAGCCCAAGGCCACCAAGGAGCAGCTGAAGGCCGTGATGGACGACTT
CGCCGCCTTCGTGGAGAAGTGCTGCAAGGCCGACGACAAGGAGACCTGCTTCGCCGAAG
AGGGGAAGAAGCTGGTGGCCGCCAGCCAGGCTGCCCTGGGACTGGGAGGTGGTGGGTCT

SEQ ID NO: 37 includes Fusion Moeity: 1-1770; Free energy: −745.2; gdT CAI: 0.955655916201255; ORF count: 4.

SEQ ID NO: 38 is the sequence name for GD2 scFv. It is a Tumor targeting scFv construct. It is an AA sequence. The sequence is:

EVQLLQSGPELEKPGASVMISCKASGSSFTGYNMNWVRQNIGKSLEWIGAIDPYYGGTSYNQ
KFKGRATLTVDKSSSTAYMHLKSLTSEDSAVYYCVSGMKYWGQGTSVTVSSGGGGSGGGG
SGGGGSDVVMTQTPLSLPVSLGDQASISCRSSQSLVHRNGNTYLHWYLQKPGQSPKLLIHKV
SNRFSGVPDRFSGSGSGTDFTLKISRVEAEDLGVYFCSQSTHVPPLTFGAGTKLELKRAD.

SEQ ID NO: 38 includes Target scFv: 1-244; Target VL: 129-244; Target linker: 114-128; Target VH: 1-113.

SEQ ID NO: 39 is the sequence name for GD2 scFv. It is a Tumor targeting scFv construct. It is a DNA sequence. The sequence is:

GAAGTGCAGTTGCTGCAGTCTGGACCCGAGCTGGAGAAGCCTGGGGCTTCAGTGATGAT
AAGTTGTAAAGCGAGCGGCTCTTCGTTCACGGGATACAACATGAATTGGGTAAGACAGA
ACATCGGAAAGTCCCTCGAATGGATTGGTGCAATCGATCCATACTATGGGGGCACAAGC
TATAATCAGAAGTTTAAAGGCAGGGCCACTCTGACCGTCGACAAATCCTCATCCACCGCT
TATATGCACTTAAAGAGTCTTACTTCTGAAGACAGCGCCGTTTATTACTGCGTGTCAGGC
ATGAAGTACTGGGGTCAAGGGACAAGCGTGACCGTCAGTTCCGGTGGTGGTGGAAGCGG
TGGTGGTGGATCTGGCGGTGGTGGAAGTGATGTAGTGATGACCCAGACTCCTCTGAGTCT
GCCCGTTAGTTTAGGTGACCAGGCCAGCATCAGCTGCAGGTCCAGCCAGTCATTGGTTCA
TAGAAACGGCAATACCTACCTGCACTGGTACCTGCAAAAACCCGGGCAATCTCCTAAAC
TTCTCATACACAAGGTCTCTAACCGTTTCTCGGGGGTCCCGGATCGGTTTAGCGGATCAG
GCTCCGGAACAGATTTTACGTTGAAGATTTCTCGCGTGGAGGCCGAGGACCTTGGTGTGT
ATTTCTGTTCCCAGTCCACTCATGTGCCACCACTGACATTCGGCGCTGGGACCAAACTCG
AACTAAAGCGAGCAGAC.

SEQ ID NO: 39 includes Target scFv: 1-732; Target VL: 385-732; Target linker: 340-384; Target VH: 1-339; Free energy: −243.8; gdT CAI: 0.70651634854966; ORF count: 9.

SEQ ID NO: 40 is the sequence name for GD2 scFv ECOg (18). It is a Tumor

GAGGTCCAGCTCCTCCAGTCCGGGCCCGAGCTGGAGAAGCCCGGGGCCTCCGTGATGAT
CTCCTGCAAGGCCTCCGGGTCCTCCTTCACCGGGTACAACATGAACTGGGTCCGCCAGAA
CATCGGGAAGAGCCTGGAGTGGATCGGGGCCATCGACCCCTACTACGGAGGCACCTCCT
ACAACCAGAAGTTCAAGGGCCGGGCCACTCTGACCGTGGACAAGTCCTCCTCCACCGCC
TACATGCACCTGAAGAGCCTGACCTCCGAGGACTCCGCCGTCTACTACTGCGTCTCCGGG
ATGAAGTACTGGGGCCAGGGGACCTCCGTGACCGTCTCCTCCGGAGGAGGAGGCTCCGG
AGGAGGAGGCTCCGGAGGAGGAGGCTCCGACGTGGTGATGACCCAGACCCCTCTGAGCC
TGCCCGTGAGCCTCGGGGACCAGGCCTCCATCAGCTGCAGGAGCTCCCAGAGCCTGGTC
CACAGGAACGGGAACACCTACCTCCACTGGTACCTCCAGAAGCCCGGGCAGAGCCCCAA
GCTCCTGATCCACAAGGTCTCCAACAGGTTCTCCGGGGTCCCCGACCGCTTCAGCGGGTC
CGGGTCCGGGACCGACTTCACCCTGAAGATCTCCAGAGTGGAGGCCGAGGACCTCGGGG
TCTACTTCTGCTCCCAGAGCACCCACGTGCCTCCTCTGACCTTCGGGGCCGGGACCAAGC
TGGAGCTCAAGAGGGCCGAC.

SEQ ID NO: 40 includes Target scFv: 1-732; Target VL: 385-732; Target linker: 340-384; Target VH: 1-339; Free energy: −328.1; gdT CAI: 0.877232; ORF count: 0.

SEQ ID NO: 41 is the sequence name for GD2 scFv ECOg (84). It is a Tumor targeting scFv construct. It is a DNA sequence. The sequence is:

GAGGTCCAGCTGCTGCAGTCGGGGCCCGAGCTGGAGAAGCCCGGGGCCTCCGTCATGAT
CTCGTGCAAGGCCTCCGGGTCCTCGTTCACCGGGTACAACATGAACTGGGTGCGCCAGA
ACATCGGGAAGTCGCTGGAGTGGATCGGGGCCATCGACCCCTACTACGGCGGCACCAGC
TACAACCAGAAGTTCAAGGGGCGCGCCACGCTGACGGTGGACAAGTCGTCGTCGACCGC
CTACATGCACCTGAAGTCGCTGACGTCGGAGGACTCCGCCGTCTACTACTGCGTCAGCGG
GATGAAGTACTGGGGCCAGGGGACCTCGGTCACCGTGTCCTCCGGCGGCGGAGGAAGCG
GAGGAGGAGGCTCCGGCGGAGGAGGCTCCGACGTCGTGATGACGCAGACCCCGCTGTCG
CTCCCGGTGTCCCTCGGGGACCAGGCCTCCATCAGCTGCCGGAGCTCGCAGTCCCTGGTG
CACCGGAACGGGAACACCTACCTCCACTGGTACCTGCAGAAGCCGGGGCAGTCGCCCAA
GCTGCTGATCCACAAGGTGTCCAACAGGTTCTCCGGGGTCCCCGACCGCTTCAGCGGGA
GCGGGAGCGGGACCGACTTCACCCTGAAGATCTCCCGCGTGGAGGCCGAGGACCTCGGG
GTCTACTTCTGCAGCCAGAGCACCCACGTGCCACCGCTGACCTTCGGCGCCGGCACCAA
GCTGGAGCTCAAGCGGGCCGAC.

SEQ ID NO: 41 includes Target scFv: 1-732; Target VL: 385-732; Target linker: 340-384; Target VH: 1-339; Free energy: −351.8; gdT CAI: 0.797734389193656; ORF count: 0.

SEQ ID NO: 42 is the sequence name for GD2 scFv ECOg (88). It is a Tumor

GAGGTCCAGCTGCTCCAGTCCGGGCCCGAGCTGGAGAAGCCCGGGGCCTCGGTGATGAT
CAGCTGCAAGGCCTCCGGGTCGAGCTTCACCGGGTACAACATGAACTGGGTCCGGCAGA
ACATCGGGAAGTCGCTGGAGTGGATCGGGGCGATCGACCCCTACTACGGCGGCACCAGC
TACAACCAGAAGTTCAAGGGGCGCGCGACCCTGACCGTCGACAAGTCGAGCTCGACCGC
CTACATGCACCTGAAGTCGCTGACCTCCGAGGACTCCGCGGTCTACTACTGCGTGAGCGG
GATGAAGTACTGGGGCCAGGGGACCTCGGTGACCGTGAGCTCCGGCGGCGGCGGATCTG
GTGGTGGCGGTTCCGGCGGTGGCGGTTCCGACGTGGTGATGACCCAGACCCCGCTCTCG
CTCCCGGTCTCGCTCGGGGACCAGGCCTCGATCAGCTGCCGGAGCTCCCAGTCGCTGGTC
CACCGGAACGGGAACACCTACCTCCACTGGTACCTCCAGAAGCCCGGGCAGTCGCCCAA
GCTGCTGATCCACAAGGTGAGCAACCGCTTCTCCGGGGTCCCCGACCGCTTCTCCGGGTC
CGGGTCCGGGACCGACTTCACCCTGAAGATCTCCCGGGTCGAGGCCGAGGACCTCGGGG
TCTACTTCTGCTCCCAGTCGACCCACGTGCCGCCACTGACCTTCGGGGCCGGGACCAAGC
TGGAGCTGAAGCGGGCCGAC.

SEQ ID NO: 42 includes Target scFv: 1-732; Target VL: 385-732; Target linker: 340-384; Target VH: 1-339; Free energy: −340; gdT CAI: 0.799370908165938; ORF count: 1.

SEQ ID NO: 43 is the sequence name for GD2 scFv ECOg (2). It is a Tumor targeting scFv construct. It is a DNA sequence. The sequence is:

GAGGTGCAGCTGCTGCAGTCCGGCCCCGAGCTGGAGAAGCCCGGGGCCAGCGTGATGAT
CAGCTGCAAGGCCAGCGGGTCCAGCTTCACGGGGTACAACATGAACTGGGTGCGGCAGA
ACATCGGGAAGAGCCTGGAGTGGATCGGGGCCATCGACCCGTACTACGGCGGCACGTCG
TACAACCAGAAGTTCAAGGGGCGCGCCACGCTGACCGTGGACAAGTCCAGCAGCACGGC
CTACATGCACCTCAAGTCGCTGACCAGCGAGGACAGCGCGGTGTACTACTGCGTGTCCG
GCATGAAGTACTGGGGCCAGGGGACCAGCGTGACGGTGAGCAGCGGCGGTGGTGGCAG
CGGTGGTGGCGGTAGCGGCGGTGGTGGCAGCGACGTGGTGATGACCCAGACGCCCCTCA
GCCTGCCGGTCAGCCTGGGCGACCAGGCCAGCATTTCGTGCCGCAGCTCGCAGAGCCTG
GTGCACCGCAACGGGAACACCTACCTGCACTGGTACCTGCAGAAGCCCGGGCAGAGCCC
CAAGCTGCTGATCCACAAGGTGTCCAACCGCTTCAGCGGGGTGCCCGACCGCTTCAGCG
GGTCGGGCAGCGGGACGGACTTCACCCTGAAGATCAGCCGCGTGGAGGCCGAGGACCTC
GGGGTGTACTTCTGCTCGCAGAGCACCCACGTGCCGCCGCTTACCTTCGGGGCCGGCACC
AAGCTGGAGCTCAAGCGGGCCGAC.

SEQ ID NO: 43 includes Target scFv: 1-732; Target VL: 385-732; Target linker: 340-384; Target VH: 1-339; Free energy: −339.9; gdT CAI: 0.78785010580307; ORF count: 2.

SEQ ID NO: 44 is the sequence name for GD2 scFv ECOg (86). It is a Tumor

GAGGTCCAGCTCCTCCAGTCCGGGCCCGAGCTGGAGAAGCCCGGGGCCTCCGTGATGAT
CTCCTGCAAGGCCTCCGGGTCCTCGTTCACCGGGTACAACATGAACTGGGTGAGGCAGA
ACATCGGGAAGAGCCTGGAGTGGATCGGGGCCATCGACCCCTACTACGGCGGAACCTCG
TACAACCAGAAGTTCAAGGGGAGAGCCACGCTGACCGTGGACAAGTCCTCGTCCACCGC
GTACATGCACCTGAAGAGCCTGACCTCCGAGGACTCCGCCGTCTACTACTGCGTCTCCGG
GATGAAGTACTGGGGACAGGGGACCTCCGTGACCGTCTCCTCCGGCGGAGGAGGCTCAG
GCGGAGGAGGCTCCGGCGGAGGAGGCTCCGACGTGGTGATGACCCAGACTCCCCTCTCC
CTGCCCGTCTCCCTCGGCGACCAGGCGAGCATCTCCTGCCGGTCCTCTCAGTCCCTCGTC
CACCGGAACGGGAACACCTACCTCCACTGGTACCTGCAGAAGCCCGGGCAGTCGCCCAA
GCTGCTCATCCACAAGGTGAGCAACCGCTTCTCCGGGGTCCCCGACCGCTTCTCCGGGTC
CGGGTCCGGGACCGACTTCACCCTGAAGATCTCCCGGGTCGAGGCCGAGGACCTCGGCG
TCTACTTCTGCTCCCAGTCTACCCACGTTCCTCCGCTGACCTTCGGCGCGGGGACGAAGC
TGGAGCTGAAGAGGGCCGAT.

SEQ ID NO: 44 includes Target scFv: 1-732; Target VL: 385-732; Target linker: 340-384; Target VH: 1-339; Free energy: −336.5; gdT CAI: 0.849533769168508; ORF count: 0.

SEQ ID NO: 45 is the sequence name for integrin aVb3 scFv. It is a Tumor targeting scFv construct. It is an AA sequence. The sequence is:

EVQLEESGGGLVKPGGSLKLSCAASGFAFSSYDMSWVRQIPEKRLEWVAKVSSGGGSTYYL
DTVQGRFTISRDNAKNTLYLQMSSLNSEDTAMYYCARHNYGSFAYWGQGTLVTVSAAKGG
GGSGGGGSGGGGSELVMTQTPATLSVTPGDSVSLSCRASQSISNHLHWYQQKSHESPRLLIK
YASQSISGIPSRFSGSGSGTDFTLSINSVETEDFGMYFCQQSNSWPHTFGGGTKLEIK.

SEQ ID NO: 45 includes Target scFv: 1-241; Target VL: 135-241; Target linker: 120-134; Target VH: 1-119.

SEQ ID NO: 46 is the sequence name for integrin aVb3 scFv. It is a Tumor targeting scFv construct. It is a DNA sequence. The sequence is:

GAGGTGCAGCTGGAGGAGTCTGGAGGTGGCCTGGTGAAGCCAGGAGGCAGCCTGAAGC
TGAGCTGTGCTGCCTCTGGGTTTGCCTTCAGCTCCTATGACATGAGCTGGGTGAGGCAGA
TCCCTGAGAAGAGGCTGGAGTGGGTAGCTAAGGTGAGCTCTGGAGGTGGCAGCACCTAC
TACCTGGACACAGTGCAGGGCAGGTTCACCATCAGCAGGGACAATGCTAAGAACACCCT
GTACCTGCAGATGAGCAGCCTGAACTCTGAGGACACAGCTATGTACTACTGTGCCAGGC
ACAACTATGGGTCCTTTGCCTACTGGGGCCAGGGGACCCTGGTGACAGTGTCTGCAGCTA
AAGGTGGAGGTGGCTCTGGAGGTGGAGGCTCTGGAGGTGGAGGCTCTGAGCTGGTGATG
ACCCAGACCCCAGCTACCCTGAGTGTGACCCCAGGGGACTCTGTGTCCCTGAGCTGCAG
GGCCAGCCAGTCCATCTCCAACCACCTGCACTGGTACCAGCAGAAGTCCCATGAGAGCC
CCAGGCTGCTGATCAAGTATGCCTCTCAGTCCATCTCTGGCATCCCCAGCAGGTTCTCAG
GGTCAGGGTCAGGGACAGACTTCACCCTGAGCATCAACTCTGTGGAGACAGAGGACTTT
GGGATGTACTTCTGCCAGCAGTCCAACTCCTGGCCCCACACCTTTGGAGGTGGCACCAAG
CTGGAGATCAAG.

SEQ ID NO: 46 includes Target scFv: 1-723; Target VL: 403-723; Target linker: 358-402; Target VH: 1-357; Free energy: −326.8; gdT CAI: 0.836939021276497; ORF count: 8.

SEQ ID NO: 47 is the sequence name for integrin a Vb3 scFv ECOg (62). It is a Tumor targeting scFv construct. It is a DNA sequence. The sequence is:

GAGGTGCAGCTGGAGGAGTCCGGCGGCGGCTTGGTGAAGCCCGGCGGCTCCCTGAAGCT
GTCCTGCGCCGCCTCCGGGTTCGCCTTCTCCAGCTACGACATGTCCTGGGTGCGGCAGAT
CCCCGAGAAGCGGCTGGAGTGGGTCGCCAAGGTGTCCTCCGGCGGCGGCTCCACCTACT
ACCTGGACACCGTGCAGGGGCGCTTCACCATCTCCCGGGACAACGCCAAGAACACCCTG
TACCTGCAGATGAGCTCCCTGAACAGCGAGGACACCGCCATGTACTACTGCGCCCGGCA
CAACTACGGGTCCTTCGCCTACTGGGGCCAGGGGACCCTGGTGACCGTGTCCGCCGCCA
AAGGAGGCGGCGGCTCCGGAGGAGGTGGCTCAGGCGGCGGAGGCAGCGAGCTGGTGAT
GACCCAGACCCCGGCCACGCTGTCCGTGACGCCCGGGGACTCCGTGTCCCTGAGCTGCC
GGGCCAGCCAGTCCATCAGCAACCACCTGCACTGGTACCAGCAGAAGTCCCACGAGAGC
CCGCGGCTGCTGATCAAGTACGCCAGCCAGTCCATCTCCGGCATCCCCAGCCGGTTCTCC
GGGTCCGGGTCCGGGACCGACTTCACCCTGTCCATCAACTCCGTGGAGACCGAGGACTT
CGGGATGTACTTCTGCCAGCAGAGCAACAGCTGGCCCCACACCTTCGGCGGCGGCACCA
AGCTGGAGATCAAG.

SEQ ID NO: 47 includes Target scFv: 1-723; Target VL: 403-723; Target linker: 358-402; Target VH: 1-357; Free energy: −324.4; gdT CAI: 0.868908; ORF count: 1.

SEQ ID NO: 48 is the sequence name for integrin aVb3 scFv ECOg (26). It is a Tumor targeting scFv construct. It is a DNA sequence. The sequence is:

GAAGTCCAGCTGGAGGAGAGCGGCGGTGGCCTCGTGAAGCCCGGCGGTTCGCTCAAGCT
GAGCTGCGCGGCCAGCGGGTTCGCCTTCAGCTCGTACGACATGAGCTGGGTGCGGCAGA
TCCCCGAGAAGCGCCTGGAGTGGGTCGCCAAGGTGTCCTCCGGCGGCGGCTCGACGTAC
TACCTGGACACCGTGCAGGGGCGCTTCACGATCAGCCGGGACAACGCGAAGAACACCCT
GTACCTCCAGATGTCCTCGCTGAACTCCGAGGACACCGCGATGTACTACTGCGCGCGGC
ACAACTACGGGAGCTTCGCCTACTGGGGCCAGGGCACCCTGGTCACCGTGAGCGCGGCG
AAAGGCGGTGGCGGCAGTGGTGGCGGAGGCTCTGGCGGCGGTGGCTCCGAGCTGGTGAT
GACCCAGACCCCGGCGACGCTCTCCGTGACGCCCGGGGACTCCGTGAGCCTGTCCTGCC
GGGCCTCGCAGAGCATCTCCAACCACCTGCACTGGTACCAGCAGAAGAGCCACGAGTCG
CCCCGGCTGCTCATCAAGTACGCCTCGCAGAGCATCTCCGGGATTCCCTCGCGGTTCTCC
GGGAGCGGCTCGGGGACGGACTTCACGCTGTCCATCAACTCCGTGGAGACCGAGGACTT
CGGGATGTACTTCTGCCAGCAGAGCAACTCCTGGCCGCACACCTTCGGCGGCGGCACCA
AGCTGGAGATCAAG.

SEQ ID NO: 48 includes Target scFv: 1-723; Target VL: 403-723; Target linker: 358-402; Target VH: 1-357; Free energy: −342.6; gdT CAI: 0.773341369143768; ORF count: 1.

SEQ ID NO: 49 is the sequence name for integrin a Vb3 scFv ECOg (12). It is a Tumor targeting scFv construct. It is a DNA sequence. The sequence is:

GAGGTCCAGCTGGAGGAATCCGGCGGCGGCCTCGTGAAGCCAGGCGGCAGCCTGAAGCT
GTCGTGCGCGGCCTCCGGGTTCGCCTTCAGCTCGTACGACATGAGCTGGGTGCGCCAGAT
CCCCGAGAAGCGCCTGGAGTGGGTCGCCAAGGTGTCCTCCGGCGGCGGCAGCACGTACT
ACTTGGACACCGTGCAGGGGCGCTTCACGATCTCGCGGGACAACGCGAAGAACACGCTG
TACCTCCAGATGTCCTCGCTGAACTCGGAGGACACCGCGATGTACTACTGCGCGCGGCA
CAACTATGGGTCCTTCGCCTACTGGGGCCAGGGGACCCTGGTGACCGTGAGCGCGGCCA
AAGGTGGCGGCGGCTCAGGTGGCGGAGGCAGCGGTGGCGGAGGCAGCGAGCTCGTGAT
GACGCAGACGCCGGCGACCCTCAGTGTGACCCCAGGGGACTCCGTCAGCCTGAGCTGCC
GGGCGTCCCAGTCGATCTCGAACCACCTGCACTGGTACCAGCAGAAGTCCCACGAGAGC
CCCAGGTTGCTCATCAAGTACGCCAGCCAGTCGATCTCCGGGATTCCCTCTCGGTTCTCG
GGGTCCGGCAGCGGGACCGACTTCACGCTGTCGATCAACAGCGTGGAGACCGAGGACTT
CGGGATGTACTTCTGCCAGCAGTCCAACAGTTGGCCCCACACGTTCGGCGGCGGCACCA
AGCTGGAGATCAAG.

SEQ ID NO: 49 includes Target scFv: 1-723; Target VL: 403-723; Target linker: 358-402; Target VH: 1-357; Free energy: −339.8; gdT CAI: 0.762733907249084; ORF count: 2.

SEQ ID NO: 50 is the sequence name for integrin a Vb3 scFv ECOg (48). It is a Tumor targeting scFv construct. It is a DNA sequence. The sequence is:

GAGGTCCAGCTGGAGGAGTCCGGCGGAGGCCTGGTCAAGCCCGGCGGCAGCCTGAAGCT
GTCCTGCGCCGCCTCCGGCTTCGCCTTCAGCAGCTACGACATGTCCTGGGTCCGGCAGAT
CCCGGAGAAGCGGCTGGAGTGGGTCGCCAAGGTCAGCTCCGGCGGCGGCAGCACCTACT
ACCTGGACACCGTCCAGGGCCGGTTCACCATCAGCCGGGACAACGCCAAGAACACCCTG
TACCTGCAGATGTCCAGCCTGAACTCCGAGGACACGGCCATGTACTACTGCGCCCGGCA
CAACTACGGCAGCTTCGCCTACTGGGGCCAGGGGACCCTGGTGACCGTGTCGGCCGCCA
AAGGCGGAGGCGGCTCAGGAGGCGGAGGTTCCGGCGGCGGAGGCTCCGAGCTGGTCAT
GACCCAGACGCCGGCCACGCTGTCCGTGACGCCCGGCGACTCCGTCAGCCTGTCCTGCC
GGGCCAGCCAGTCCATCAGCAACCACCTGCACTGGTACCAGCAGAAGTCCCACGAGTCG
CCCCGGCTGCTGATCAAGTACGCCAGCCAGTCCATCTCCGGCATCCCCAGCCGGTTCTCC
GGGTCCGGGTCCGGGACCGACTTCACCCTGTCCATCAACTCCGTCGAGACCGAGGACTTC
GGCATGTACTTCTGCCAGCAGTCCAACAGCTGGCCGCACACCTTCGGCGGCGGCACCAA
GCTGGAGATCAAG.

SEQ ID NO: 50 includes Target scFv: 1-723; Target VL: 403-723; Target linker: 358-402; Target VH: 1-357; Free energy: −329.4; gdT CAI: 0.82265589760209; ORF count: 0.

SEQ ID NO: 51 is the sequence name for integrin a Vb3 scFv ECOg (97). It is a Tumor targeting scFv construct. It is a DNA sequence. The sequence is:

GAGGTCCAGCTGGAGGAGTCCGGAGGAGGACTCGTGAAGCCCGGAGGCTCCCTGAAGCT
CTCCTGCGCCGCCTCCGGGTTCGCCTTCTCCTCCTACGACATGTCCTGGGTCCGGCAGAT
CCCCGAGAAGAGGCTGGAGTGGGTCGCGAAGGTCTCCTCCGGAGGAGGCTCGACCTACT
ATCTCGACACGGTCCAGGGCCGGTTCACGATCTCCCGGGACAACGCGAAGAACACGCTC
TACCTCCAGATGTCGAGCCTGAACTCCGAGGACACCGCGATGTACTACTGCGCCCGGCA
CAACTACGGGAGCTTCGCCTACTGGGGCCAGGGGACTCTCGTGACCGTCTCCGCCGCGA
AAGGCGGAGGAGGCTCCGGAGGAGGAGGCTCCGGAGGAGGAGGCTCCGAGCTCGTGAT
GACCCAGACCCCGGCGACCCTCTCCGTGACGCCCGGGGACTCCGTCTCCCTCTCCTGTCG
CGCGAGCCAGTCGATCTCGAACCATCTCCACTGGTACCAGCAGAAGAGTCACGAGTCTC
CCCGGCTCCTGATCAAGTACGCGAGCCAGTCGATCTCCGGGATTCCCTCGCGGTTCTCCG
GGTCCGGGTCCGGGACCGACTTCACGCTCTCGATCAACTCCGTCGAGACCGAGGACTTC
GGGATGTACTTCTGCCAGCAGTCGAACTCCTGGCCCCACACGTTCGGCGGCGGCACGAA
GCTGGAGATCAAG.

SEQ ID NO: 51 includes Target scFv: 1-723; Target VL: 403-723; Target linker: 358-402; Target VH: 1-357; Free energy: −329.3; gdT CAI: 0.777965026449684; ORF count: 0.

SEQ ID NO: 52 is the sequence name for SSTR2 scFv. It is a Tumor targeting scFv construct. It is an AA sequence. The sequence is:

DIVMTQSPDSLAVSLGERATINCKSSQSLLNSRNRKNYLAWYQQKPDQSPKLLIYWASTRES
GVPDRFSGSGSGTDFTLTISSLQAEDVAVYYCKQSYYLWTFGGGTKVEIKGGGGSGGGGSG
GGGSEVQLVESGGGLVQPGGSLRLSCAASGFTFSDYGMAWFRQAPGKGLEWVSFISNLGYSI
YYADSVKGRFTISRDNAKNSLYLQMNSLRAEDTAVYYCARAPYDYDSFDPMDYWGQGTLV
TVS.

SEQ ID NO: 52 includes Target scFv: 1-248; Target VL: 1-112; Target linker: 113-127; Target VH: 128-248.

SEQ ID NO: 53 is the sequence name for SSTR2 scFv. It is a Tumor targeting scFv construct. It is a DNA sequence. The sequence is:

GACATTGTGATGACCCAGAGCCCAGACTCCCTGGCTGTGAGCCTAGGGGAGAGGGCCAC
CATCAACTGCAAGTCCTCTCAGAGCCTCCTCAACTCCAGGAACAGGAAGAACTACCTGG
CCTGGTACCAGCAGAAGCCAGACCAGAGCCCCAAGCTGCTCATCTACTGGGCCTCCACC
AGGGAGTCTGGGGTGCCTGACAGGTTCTCTGGGTCTGGGTCTGGGACTGACTTCACCCTG
ACCATCAGCTCCCTGCAGGCTGAGGATGTGGCTGTGTACTACTGCAAGCAGAGCTACTA
CCTGTGGACCTTTGGTGGAGGCACCAAGGTGGAGATCAAAGGAGGTGGAGGCTCTGGTG
GTGGAGGCTCTGGTGGTGGAGGCTCTGAGGTGCAGCTGGTGGAGTCTGGTGGTGGCCTG
GTCCAGCCAGGAGGCTCCCTGAGGCTGAGCTGTGCTGCCTCTGGGTTCACCTTCTCAGAC
TATGGGATGGCCTGGTTCAGGCAGGCCCCAGGGAAGGGCCTGGAGTGGGTGAGCTTCAT
CTCCAACCTGGGCTACTCCATCTACTATGCTGACTCTGTGAAGGGCAGGTTCACCATCAG
CAGGGACAATGCCAAGAACAGCCTGTACCTGCAGATGAACTCCCTGAGGGCTGAGGACA
CAGCTGTGTACTACTGTGCCAGGGCCCCATATGACTATGACAGCTTTGACCCCATGGACT
ACTGGGGCCAGGGGACCCTGGTGACTGTGAGC.

SEQ ID NO: 53 includes Target scFv: 1-744; Target VL: 1-336; Target linker: 337-381; Target VH: 382-744; Free energy: −344; gdT CAI: 0.841055073258909; ORF count: 7.

SEQ ID NO: 54 is the sequence name for SSTR2 svFv ECOg (72). It is a Tumor targeting scFv construct. It is a DNA sequence. The sequence is:

GACATCGTGATGACCCAGAGCCCCGACTCCCTGGCCGTGAGCCTCGGGGAGAGGGCCAC
GATCAACTGCAAGAGCTCCCAGAGCCTGCTCAACTCCCGGAACCGGAAGAACTACCTGG
CCTGGTACCAGCAGAAGCCCGACCAGTCGCCCAAGCTGCTCATCTACTGGGCCTCCACC
AGGGAGAGCGGGGTGCCCGACCGCTTCTCCGGGAGCGGGTCCGGGACCGACTTCACCCT
GACCATCTCCTCTCTGCAGGCCGAGGACGTGGCCGTGTACTACTGCAAGCAGAGCTACT
ACCTGTGGACATTCGGCGGCGGCACCAAGGTGGAGATCAAAGGCGGCGGCGGCAGTGG
TGGAGGAGGCAGCGGCGGCGGTGGCTCAGAGGTGCAGCTGGTGGAGAGCGGAGGCGGC
CTGGTGCAGCCAGGCGGCTCTCTGCGCCTGTCCTGCGCCGCCTCTGGGTTCACCTTCTCC
GACTACGGGATGGCCTGGTTCCGCCAGGCCCCAGGGAAGGGGCTGGAGTGGGTGTCCTT
CATCTCCAACCTGGGGTACAGCATCTACTACGCCGACTCCGTGAAGGGCCGGTTCACCAT
CAGCCGGGACAACGCCAAGAACAGCCTGTACCTGCAGATGAACTCCCTGCGCGCCGAGG
ACACCGCCGTGTACTACTGCGCCCGGGCTCCCTACGACTACGACAGCTTCGACCCCATGG
ACTACTGGGGCCAGGGGACACTGGTCACCGTGTCC.

SEQ ID NO: 54 includes Target scFv: 1-744; Target VL: 1-336; Target linker: 337-381; Target VH: 382-744; Free energy: −325.1; gdT CAI: 0.865568; ORF count: 1.

SEQ ID NO: 55 is the sequence name for SSTR2 svFv ECOg (64). It is a Tumor targeting scFv construct. It is a DNA sequence. The sequence is:

GACATCGTGATGACGCAGAGCCCCGACTCTTTGGCGGTGAGCCTCGGGGAGAGGGCCAC
CATCAACTGCAAGTCGTCGCAGAGCCTCCTCAACAGCCGCAACCGGAAGAACTACCTCG
CCTGGTATCAGCAGAAGCCCGACCAAAGCCCCAAGCTGCTGATCTACTGGGCCTCCACA
CGGGAGTCTGGGGTGCCCGACCGCTTCAGCGGCAGCGGCAGCGGCACCGACTTCACCCT
CACCATCAGCAGCCTGCAGGCGGAGGATGTGGCGGTGTACTACTGCAAACAGTCCTACT
ATCTGTGGACCTTCGGCGGCGGCACAAAGGTGGAGATCAAAGGCGGCGGCGGCAGTGG
AGGAGGAGGCAGCGGCGGCGGAGGCTCTGAGGTGCAGCTGGTGGAGAGCGGCGGCGGT
TTGGTGCAGCCCGGCGGCAGCCTCCGACTCAGCTGCGCCGCCTCCGGCTTCACCTTCTCC
GACTACGGGATGGCGTGGTTTCGGCAGGCCCCGGGGAAGGGGTTGGAGTGGGTGAGCTT
CATCTCCAACTTGGGGTACAGCATCTACTACGCCGACTCTGTGAAGGGGCGCTTCACCAT
CAGCCGCGACAACGCCAAAAACAGCCTCTATCTGCAGATGAACAGCCTCCGCGCTGAGG
ACACAGCGGTGTACTACTGCGCCCGCGCCCCATACGACTACGACTCCTTCGACCCCATGG
ACTACTGGGGTCAGGGGACGTTGGTGACGGTGTCG.

SEQ ID NO: 55 includes Target scFv: 1-744; Target VL: 1-336; Target linker: 337-381; Target VH: 382-744; Free energy: −333.3; gdT CAI: 0.782038165701074; ORF count: 4.

SEQ ID NO: 56 is the sequence name for SSTR2 svFv ECOg (15). It is a Tumor targeting scFv construct. It is a DNA sequence. The sequence is:

GACATCGTCATGACCCAGTCACCTGACAGCCTGGCCGTCAGCCTGGGCGAACGGGCCAC
CATCAACTGTAAGTCATCTCAGAGCCTGCTGAACAGCCGGAACCGGAAGAACTACCTGG
CCTGGTATCAGCAGAAGCCTGATCAGTCACCTAAGCTGCTGATCTACTGGGCCTCAACCA
GAGAGTCCGGCGTGCCTGACAGGTTCAGCGGGTCCGGGTCCGGGACCGACTTCACCCTG
ACCATCAGCAGCCTGCAGGCCGAGGACGTGGCCGTCTATTACTGTAAGCAGTCTTATTAC
CTGTGGACCTTCGGCGGCGGCACCAAGGTCGAGATCAAAGGCGGCGGCGGCTCAGGTGG
TGGTGGCTCCGGCGGCGGTGGCTCTGAGGTCCAGCTGGTCGAGTCCGGCGGTGGCCTGG
TCCAGCCAGGCGGCAGCCTGAGGCTGTCCTGCGCCGCCTCCGGCTTCACCTTCTCTGACT
ACGGCATGGCCTGGTTCAGACAGGCCCCAGGCAAGGGCCTGGAGTGGGTCAGCTTCATC
AGCAACCTGGGCTACAGCATCTATTACGCCGACAGCGTCAAGGGCCGGTTCACCATCAG
CCGGGACAACGCCAAGAACAGCCTGTACCTGCAGATGAACAGCCTGAGGGCCGAGGAC
ACGGCCGTCTATTACTGCGCCCGGGCCCCTTATGACTATGACAGCTTTGACCCCATGGAC
TACTGGGGCCAGGGGACCCTGGTGACCGTGTCA.

SEQ ID NO: 56 includes Target scFv: 1-744; Target VL: 1-336; Target linker: 337-381; Target VH: 382-744; Free energy: −331.6; gdT CAI: 0.812422023876491; ORF count: 8.

SEQ ID NO: 57 is the sequence name for SSTR2 svFv ECOg (50). It is a Tumor targeting scFv construct. It is a DNA sequence. The sequence is:

GACATCGTGATGACCCAGAGCCCCGACTCCCTGGCCGTGTCCCTCGGGGAGAGGGCCAC
CATCAACTGCAAGAGCTCCCAGTCCCTGCTGAACTCCCGGAACCGGAAGAACTACCTGG
CCTGGTACCAGCAGAAGCCCGACCAGAGCCCCAAGCTGCTGATCTACTGGGCCAGCACC
AGGGAGAGCGGGGTGCCCGACCGCTTCTCCGGGTCCGGGTCCGGGACCGACTTCACCCT
GACCATCAGCTCCCTGCAGGCCGAGGACGTGGCCGTGTACTACTGCAAGCAGAGCTACT
ACCTGTGGACCTTCGGCGGCGGCACCAAGGTGGAGATCAAAGGCGGCGGCGGCTCAGG
AGGAGGTGGCTCCGGCGGCGGAGGCTCTGAGGTGCAGCTGGTGGAGAGCGGCGGCGGA
CTGGTGCAGCCAGGCGGCTCCCTGCGGCTGAGCTGCGCCGCCTCTGGGTTCACCTTCTCC
GACTACGGGATGGCCTGGTTCCGGCAGGCCCCAGGGAAGGGGCTGGAGTGGGTGAGCTT
CATCTCCAACCTGGGGTACTCCATCTACTACGCCGACTCCGTGAAGGGGCGCTTCACCAT
CTCCCGGGACAACGCCAAGAACTCCCTGTACCTGCAGATGAACTCCCTGCGGGCCGAGG
ACACCGCGGTGTACTACTGCGCCCGGGCCCCGTACGACTACGACAGCTTCGACCCCATG
GACTACTGGGGCCAGGGGACCCTGGTGACCGTGAGC.

SEQ ID NO: 57 includes Target scFv: 1-744; Target VL: 1-336; Target linker: 337-381; Target VH: 382-744; Free energy: −328.9; gdT CAI: 0.878055710303915; ORF count: 3.

SEQ ID NO: 58 is the sequence name for SSTR2 svFv ECOg (42). It is a Tumor targeting scFv construct. It is a DNA sequence. The sequence is:

GATATCGTGATGACCCAGTCCCCGGACTCCCTGGCAGTGTCCCTCGGGGAGCGGGCCAC
CATCAACTGCAAGAGCTCCCAGTCCCTGCTGAACTCCCGGAACCGGAAGAACTACCTGG
CCTGGTACCAGCAGAAGCCCGACCAGTCCCCGAAGCTGCTGATCTACTGGGCCAGCACC
CGGGAATCCGGGGTGCCCGACCGCTTCTCCGGGTCCGGGTCCGGGACCGACTTCACCCT
GACCATCAGCTCCCTGCAGGCCGAGGACGTGGCAGTGTACTACTGCAAGCAGTCCTACT
ACCTGTGGACCTTCGGCGGCGGCACCAAGGTGGAGATCAAAGGCGGCGGCGGCTCTGGA
GGAGGAGGCTCCGGCGGCGGAGGTTCCGAGGTGCAGCTGGTGGAGTCCGGCGGAGGAC
TGGTGCAGCCCGGCGGCTCCCTGCGACTGTCCTGCGCCGCCTCCGGGTTCACCTTCTCCG
ACTACGGGATGGCCTGGTTCCGGCAGGCTCCCGGGAAGGGGCTGGAGTGGGTGTCCTTC
ATCTCCAACCTGGGGTACTCCATCTACTACGCCGACTCCGTGAAGGGCCGGTTCACCATC
TCCCGGGACAACGCCAAGAACTCCCTGTACCTGCAGATGAACTCCCTGCGGGCCGAGGA
CACCGCAGTGTACTACTGCGCCCGGGCCCCGTACGACTACGACTCCTTCGACCCCATGGA
CTACTGGGGCCAGGGGACCCTGGTGACCGTGTCC.

SEQ ID NO: 58 includes Target scFv: 1-744; Target VL: 1-336; Target linker: 337-381; Target VH: 382-744; Free energy: −328.6; gdT CAI: 0.886975497635559; ORF count: 1.

SEQ ID NO: 59 is the sequence name for 2×SST28 3×G4S ligand. It is a Tumor targeting ligand construct. It is an AA sequence. The sequence is:

SANSNPAMAPRERKAGCKNFFWKTFTSCGGGGSGGGGSGGGGSSANSNPAMAPRERKAGC
KNFFWKTFTSC.

SEQ ID NO: 59 includes Target Ligand: 1-71.

SEQ ID NO: 60 is the sequence name for 2×SST28 3×G4S ligand. It is a Tumor Targeting Ligand construct. It is a DNA sequence. The sequence is:

AGCGCGAACAGCAACCCGGCGATGGCGCCGCGCGAACGCAAAGCGGGCTGCAAAAACT
TTTTTTGGAAAACCTTTACCAGCTGCGGCGGCGGCGGCAGCGGCGGCGGCGGCAGCGGC
GGCGGCGGCAGCAGCGCGAACAGCAACCCGGCGATGGCGCCGCGCGAACGCAAAGCGG
GCTGCAAAAACTTTTTTTGGAAAACCTTTACCAGCTGCGGCGGCGGCGGCAGCGGCGGC
GGCGGCAGCGGCGGCGGCGGCAGC.

SEQ ID NO: 60 includes Target Ligand: 1-213; Free energy: −117; gdT CAI: 0.603084331934136; ORF count: 0.

SEQ ID NO: 61 is the sequence name for 2×SST28 3×G4S ligand ECOg (192). It is a Tumor Targeting Ligand construct. It is a DNA sequence. The sequence is:

TCTGCCAACTCCAACCCCGCTATGGCTCCCAGGGAGCGGAAGGCCGGGTGCAAGAACTT
CTTCTGGAAGACCTTCACCTCCTGCGGAGGCGGAGGCTCAGGAGGCGGAGGCTCCGGAG
GAGGCGGCTCCTCCGCCAACTCCAACCCCGCTATGGCTCCCAGGGAGCGGAAGGCCGGG
TGCAAGAACTTCTTCTGGAAGACCTTCACCTCCTGCGGAGGCGGAGGCTCCGGAGGAGG
CGGCTCAGGAGGCGGCGGCAGC.

SEQ ID NO: 61 includes Target Ligand: 1-213; Free energy: −138.2; gdT CAI: 0.800017929945784; ORF count: 0.

SEQ ID NO: 62 is the sequence name for 2×SST28 3×G4S ligand ECOg (141). It is a Tumor Targeting Ligand construct. It is a DNA sequence. The sequence is:

TCCGCCAACTCCAACCCCGCTATGGCTCCCCGGGAGCGGAAGGCTGGGTGCAAGAACTT
CTTCTGGAAGACCTTCACCTCCTGCGGAGGCGGAGGTTCCGGAGGCGGAGGTTCCGGCG
GAGGCGGCTCCTCCGCCAACTCCAACCCCGCGATGGCTCCCAGGGAGCGGAAGGCCGGG
TGCAAGAACTTCTTCTGGAAGACCTTCACCTCCTGCGGAGGCGGAGGTTCCGGCGGAGG
AGGTTCCGGAGGCGGCGGCTCC.

SEQ ID NO: 62 includes Target Ligand: 1-213; Free energy: −146.7; gdT CAI: 0.786043171489395; ORF count: 0.

SEQ ID NO: 63 is the sequence name for 2×SST28 3×G4S ligand ECOg (241). It is a Tumor Targeting Ligand construct. It is a DNA sequence. The sequence is:

AGCGCCAACTCCAACCCCGCTATGGCTCCCCGGGAGCGCAAGGCTGGGTGCAAGAACTT
CTTCTGGAAGACCTTCACCTCCTGCGGAGGCGGAGGCTCTGGAGGCGGAGGCTCTGGCG
GAGGCGGCTCCTCCGCCAACTCCAACCCCGCGATGGCTCCCAGGGAGCGCAAGGCCGGG
TGCAAGAACTTCTTCTGGAAGACCTTCACCTCCTGCGGAGGCGGAGGCTCTGGCGGAGG
AGGCTCTGGAGGCGGCGGCTCC.

SEQ ID NO: 63 includes Target Ligand: 1-213; Free energy: −142.6; gdT CAI: 0.788525759670669; ORF count: 0.

SEQ ID NO: 64 is the sequence name for 2×SST28 3×G4S ligand ECOg (172). It is a Tumor Targeting Ligand construct. It is a DNA sequence. The sequence is:

TCCGCCAACTCCAACCCGGCCATGGCTCCCCGGGAGAGGAAGGCCGGGTGCAAGAACTT
CTTCTGGAAGACCTTCACCTCCTGCGGAGGAGGAGGAAGCGGAGGAGGAGGAAGCGGA
GGAGGAGGCTCCTCGGCCAACTCCAACCCGGCCATGGCTCCCCGGGAGAGGAAGGCCGG
GTGCAAGAACTTCTTCTGGAAGACCTTCACCTCCTGCGGAGGAGGAGGAAGCGGAGGAG
GAGGAAGCGGAGGAGGAGGATCT.

SEQ ID NO: 64 includes Target Ligand: 1-213; Free energy: −140; gdT CAI: 0.719392416533176; ORF count: 0.

SEQ ID NO: 65 is the sequence name for 2×SST28 3×G4S ligand ECOg (266). It is a Tumor Targeting Ligand construct. It is a DNA sequence. The sequence is:

TCCGCCAACTCCAACCCCGCTATGGCTCCCCGGGAGCGGAAGGCTGGGTGCAAGAACTT
CTTCTGGAAGACCTTCACCTCCTGTGGCGGCGGCGGATCTGGCGGAGGAGGCTCTGGCG
GAGGCGGCTCCTCCGCCAACTCCAACCCCGCTATGGCTCCCAGGGAGCGGAAGGCCGGG
TGCAAGAACTTCTTCTGGAAGACCTTCACCTCCTGTGGCGGCGGCGGATCTGGCGGAGG
AGGCTCTGGCGGAGGCGGAAGC.

SEQ ID NO: 65 includes Target Ligand: 1-213; Free energy: −135.3; gdT CAI: 0.799023029714678; ORF count: 0.

SEQ ID NO: 66 is the sequence name for 2×SST28 2×G4S ligand. It is a Tumor Targeting Ligand construct. It is an AA sequence. The sequence is:

SANSNPAMAPRERKAGCKNFFWKTFTSCGGSGGSGGSGGSGGSANSNPAMAPRERKAGCK
NFFWKTFTSCGGSGGSGGSGGSGG.

SEQ ID NO: 66 includes Target Ligand: 1-70; Central Linker: 71-84.

SEQ ID NO: 67 is the sequence name for 2×SST28 2×G4S ligand. It is a Tumor Targeting Ligand construct. It is a DNA sequence. The sequence is:

AGCGCGAACAGCAACCCGGCGATGGCGCCGCGCGAACGCAAAGCGGGCTGCAAAAACT
TTTTTTGGAAAACCTTTACCAGCTGCGGCGGCAGCGGCGGCAGCGGCGGCAGCGGCGGC
AGCGGCGGCAGCGCGAACAGCAACCCGGCGATGGCGCCGCGCGAACGCAAAGCGGGCT
GCAAAAACTTTTTTTGGAAAACCTTTACCAGCTGCGGCGGCAGCGGCGGCAGCGGCGGC
AGCGGCGGCAGCGGCGGC.

SEQ ID NO: 67 includes Target Ligand: 1-210; Central Linker: 221-252; Free energy: −110.4; gdT CAI: 0.602525074392084; ORF count: 0.

SEQ ID NO: 68 is the sequence name for 2×SST28 2×G4S ligand ECOg (114). It is a Tumor Targeting Ligand construct. It is a DNA sequence. The sequence is:

TCCGCCAACTCCAACCCCGCAATGGCTCCCCGGGAGAGGAAGGCCGGGTGCAAGAACTT
CTTCTGGAAGACCTTCACCAGCTGCGGAGGCTCCGGAGGCTCCGGAGGCTCCGGAGGCT
CCGGAGGCTCCGCCAACTCCAACCCCGCAATGGCTCCCCGGGAGAGGAAGGCCGGGTGC
AAGAACTTCTTCTGGAAGACCTTCACCAGCTGCGGAGGCTCCGGAGGCTCCGGAGGCTC
CGGAGGCTCCGGAGGC.

SEQ ID NO: 68 includes Target Ligand: 1-210; Central Linker: 221-252; Free energy: −140.6; gdT CAI: 0.815587600211903; ORF count: 0.

SEQ ID NO: 69 is the sequence name for 2×SST28 2×G4S ligand ECOg (86). It is a Tumor Targeting Ligand construct. It is a DNA sequence. The sequence is:

TCTGCCAATTCCAACCCCGCGATGGCCCCTCGGGAGAGGAAGGCCGGGTGCAAGAACTT
CTTCTGGAAGACCTTCACCTCCTGCGGAGGCTCCGGAGGCTCCGGAGGCTCCGGAGGCT
CCGGAGGCTCTGCCAATTCCAACCCCGCGATGGCCCCTCGGGAGAGGAAGGCCGGGTGC
AAGAACTTCTTCTGGAAGACCTTCACCTCCTGCGGAGGCTCCGGAGGCTCCGGAGGCTCC
GGAGGCTCCGGAGGC.

SEQ ID NO: 69 includes Target Ligand: 1-210; Central Linker: 221-252; Free energy: −146.5; gdT CAI: 0.793448781755408; ORF count: 0.

SEQ ID NO: 70 is the sequence name for 2×SST28 2×G4S ligand ECOg (132). It is a Tumor Targeting Ligand construct. It is a DNA sequence. The sequence is:

TCCGCGAACTCCAACCCCGCGATGGCTCCCCGGGAGAGGAAGGCCGGGTGCAAGAACTT
CTTCTGGAAGACGTTCACGTCCTGCGGAGGCTCCGGAGGCTCCGGAGGCTCCGGAGGCT
CCGGAGGCTCCGCGAACTCCAACCCCGCGATGGCTCCCCGGGAGAGGAAGGCCGGGTGC
AAGAACTTCTTCTGGAAGACGTTCACGTCCTGCGGAGGCTCCGGAGGCTCCGGAGGCTC
CGGAGGCTCCGGAGGA.

SEQ ID NO: 70 includes Target Ligand: 1-210; Central Linker: 221-252; Free energy: −141.6; gdT CAI: 0.768520163043281; ORF count: 0.

SEQ ID NO: 71 is the sequence name for 2×SST28 2×G4S ligand ECOg (131). It is a Tumor Targeting Ligand construct. It is a DNA sequence. The sequence is:

AGCGCCAACTCCAACCCCGCGATGGCTCCCAGGGAGCGGAAGGCCGGGTGCAAGAACTT
CTTCTGGAAGACCTTCACCTCCTGCGGAGGCTCCGGAGGCTCCGGAGGCTCCGGAGGCT
CCGGAGGCTCCGCCAACTCCAACCCCGCGATGGCTCCCAGGGAGCGGAAGGCCGGGTGC
AAGAACTTCTTCTGGAAGACCTTCACCTCCTGCGGAGGCTCCGGAGGCTCCGGAGGCTCC
GGAGGCTCCGGTGGC.

SEQ ID NO: 71 includes Target Ligand: 1-210; Central Linker: 221-252; Free energy: −140.8; gdT CAI: 0.805786107124917; ORF count: 0.

SEQ ID NO: 72 is the sequence name for 2×SST28 2×G4S ligand ECOg (137). It is a Tumor Targeting Ligand construct. It is a DNA sequence. The sequence is:

TCCGCCAACTCCAACCCCGCTATGGCCCCTAGGGAGAGGAAGGCCGGGTGCAAGAACTT
CTTCTGGAAGACCTTCACCTCCTGCGGAGGCTCCGGAGGCTCCGGAGGCTCCGGAGGCT
CCGGAGGCTCCGCCAACTCCAACCCCGCTATGGCCCCTAGGGAGAGGAAGGCCGGGTGC
AAGAACTTCTTCTGGAAGACCTTCACCTCCTGCGGAGGCTCCGGAGGCTCCGGAGGCTCC
GGAGGCTCCGGAGGA.

SEQ ID NO: 72 includes Target Ligand: 1-210; Central Linker: 221-252; Free energy: −140; gdT CAI: 0.822267579371957; ORF count: 1.

SEQ ID NO: 73 is the sequence name for SST28 ligand. It is a Tumor Targeting Ligand construct. It is an AA sequence. The sequence is:

SANSNPAMAPRERKAGCKNFFWKTFTSC.

SEQ ID NO: 73 includes Target Ligand: 1-28.

SEQ ID NO: 74 is the sequence name for SST28 ligand. It is a Tumor Targeting Ligand construct. It is a DNA sequence. The sequence is:

TCCGCCAACTCCAACCCGGCCATGGCTCCCCGGGAGAGGAAGGCCGGGTGCAAGAACTT
CTTCTGGAAGACCTTCACCAGCTGC.

SEQ ID NO: 74 includes Target Ligand: 1-84; Free energy: −26.1; gdT CAI: 0.909139392619506; ORF count: 0.

SEQ ID NO: 75 is the sequence name for SST28 ligand ECOg (10). It is a Tumor Targeting Ligand construct. It is a DNA sequence. The sequence is:

AGCGCCAACAGCAACCCCGCTATGGCTCCCAGGGAGCGCAAGGCCGGGTGCAAGAACTT
CTTCTGGAAGACCTTCACCTCTTGC.

SEQ ID NO: 75 includes Target Ligand: 1-84; Free energy: −27.4; gdT CAI: 0.925222356313033; ORF count: 0.

SEQ ID NO: 76 is the sequence name for SST28 ligand ECOg (172). It is a Tumor Targeting Ligand construct. It is a DNA sequence. The sequence is:

AGCGCGAACAGCAACCCGGCCATGGCCCCTCGCGAGCGAAAGGCCGGGTGCAAGAACT
TCTTCTGGAAGACCTTCACCTCGTGC.

SEQ ID NO: 76 includes Target Ligand: 1-84; Free energy: −31.1; gdT CAI: 0.762381535851502; ORF count: 0.

SEQ ID NO: 77 is the sequence name for SST28 ligand ECOg (38). It is a Tumor Targeting Ligand construct. It is a DNA sequence. The sequence is:

AGCGCCAACTCCAACCCGGCTATGGCGCCCAGGGAGAGGAAGGCCGGCTGCAAGAACTT
CTTCTGGAAGACCTTCACCTCCTGC.

SEQ ID NO: 77 includes Target Ligand: 1-84; Free energy: −31; gdT CAI: 0.866223933524215; ORF count: 0.

SEQ ID NO: 78 is the sequence name for SST28 ligand ECOg (5). It is a Tumor Targeting Ligand construct. It is a DNA sequence. The sequence is:

AGTGCGAACTCGAACCCGGCCATGGCGCCCAGAGAGCGCAAGGCCGGGTGCAAGAACT
TTTTCTGGAAAACGTTCACATCGTGC.

SEQ ID NO: 78 includes Target Ligand: 1-84; Free energy: −30.7; gdT CAI: 0.714442287269154; ORF count: 0.

SEQ ID NO: 79 is the sequence name for SST28 ligand ECOg (44). It is a Tumor Targeting Ligand construct. It is a DNA sequence. The sequence is:

AGCGCGAACTCGAACCCGGCCATGGCTCCGCGCGAGCGTAAGGCCGGGTGCAAGAACTT
TTTCTGGAAAACCTTCACGAGTTGT.

SEQ ID NO: 79 includes Target Ligand: 1-84; Free energy: −30.1; gdT CAI: 0.688444421590185; ORF count: 0.

SEQ ID NO: 80 is the sequence name for TPO. It is a Tumor Targeting Ligand construct. It is an AA sequence. The sequence is:

SPAPPACDLRVLSKLLRDSHVLHSRLSQCPEVHPLPTPVLLPAVDFSLGEWKTQMEETKAQDI
LGAVTLLLEGVMAARGQLGPTCLSSLLGQLSGQVRLLLGALQSLLGTQGRTTAHKDPNAIFL
SFQHLLRGKVRFLMLVGGSTLCVRRAPPTTAVPSRTSLVLTLNELG.

SEQ ID NO: 80 includes Target Ligand: 1-171.

SEQ ID NO: 81 is the sequence name for TPO. It is a Tumor Targeting Ligand construct. It is a DNA sequence. The sequence is:

TCCCCAGCTCCACCAGCCTGTGACCTGAGGGTGCTGAGCAAGCTCCTGAGGGACTCCCAT
GTGCTGCACAGCAGGCTGAGCCAGTGCCCTGAGGTGCATCCCCTGCCAACCCCTGTGCTG
CTGCCAGCTGTGGACTTCTCCCTTGGGGAGTGGAAGACCCAGATGGAGGAGACCAAGGC
CCAGGACATCCTTGGGGCTGTGACCCTGCTGCTGGAAGGGGTGATGGCTGCCAGGGGCC
AGCTGGGGCCAACCTGCCTCAGCTCCCTGCTGGGGCAGCTGTCAGGGCAGGTGAGGCTG
CTGCTGGGAGCCCTGCAGTCCCTGCTGGGGACCCAGGGCAGGACCACAGCCCACAAGGA
CCCCAATGCCATCTTCCTGAGCTTCCAGCACCTGCTGAGGGGCAAGGTGAGGTTCCTGAT
GCTGGTTGGAGGCAGCACCCTGTGTGTCAGGAGAGCTCCACCAACCACAGCTGTGCCCA
GCAGGACCAGCCTGGTGCTGACCCTGAATGAGCTTGGA.

SEQ ID NO: 81 includes Target Ligand: 1-513; Free energy: −251.6; gdT CAI: 0.86805332586369; ORF count: 4.

SEQ ID NO: 82 is the sequence name for TPO ECOg (6). It is a Tumor Targeting Ligand construct. It is a DNA sequence. The sequence is:

AGCCCCGCACCACCTGCCTGCGACCTGCGGGTGCTGTCCAAGCTGCTGCGGGACAGCCA
CGTGCTGCACAGCAGGCTGTCCCAGTGCCCCGAGGTGCACCCACTGCCCACGCCCGTGCT
GCTGCCCGCTGTGGACTTCTCCCTGGGCGAGTGGAAGACACAGATGGAGGAGACCAAGG
CCCAGGACATCCTGGGCGCCGTGACCCTGCTGCTGGAAGGGGTGATGGCCGCCAGAGGG
CAGCTGGGGCCAACGTGCCTGTCCTCACTGCTGGGGCAGCTGTCCGGGCAGGTGCGGCT
GCTGCTGGGCGCCCTGCAGTCCCTGCTGGGCACCCAGGGGCGCACCACAGCTCACAAGG
ACCCCAACGCCATCTTCCTGTCCTTCCAGCACCTGCTGCGGGGCAAGGTGCGGTTCCTGA
TGCTGGTCGGCGGCAGCACCCTGTGCGTGCGCAGGGCACCACCGACCACAGCTGTGCCC
AGCAGGACCTCACTGGTGCTGACCCTGAACGAGCTGGGC.

SEQ ID NO: 82 includes Target Ligand: 1-513; Free energy: −258.8; gdT CAI: 0.891232089689473; ORF count: 1.

SEQ ID NO: 83 is the sequence name for TPO ECOg (42). It is a Tumor Targeting Ligand construct. It is a DNA sequence. The sequence is:

AGCCCGGCTCCTCCGGCCTGCGACCTGCGCGTGCTGAGCAAGCTCCTGCGGGACTCGCA
CGTGCTGCACTCGCGCCTGAGCCAGTGCCCCGAGGTGCATCCCCTGCCTACCCCGGTGCT
CCTGCCCGCGGTGGACTTCTCGCTCGGGGAGTGGAAGACCCAGATGGAGGAGACCAAGG
CCCAGGACATACTCGGGGCCGTGACCCTGCTCCTGGAAGGGGTCATGGCAGCTCGGGGC
CAGCTCGGGCCTACGTGCCTGAGCTCCCTGCTCGGGCAGCTGTCCGGGCAGGTCCGGCTC
CTGCTCGGGGCCCTGCAGAGCCTGCTCGGGACCCAGGGCCGGACCACGGCTCACAAGGA
CCCGAACGCGATCTTCCTGAGCTTCCAGCACCTGCTCCGGGGCAAGGTCAGGTTCCTGAT
GCTGGTCGGAGGCTCGACCCTGTGCGTGCGCAGGGCTCCTCCGACCACGGCCGTGCCCTC
GCGCACGAGCCTGGTCCTGACCCTGAACGAGCTCGGG.

SEQ ID NO: 83 includes Target Ligand: 1-513; Free energy: −273.6; gdT CAI: 0.779630301042555; ORF count: 0.

SEQ ID NO: 84 is the sequence name for TPO ECOg (19). It is a Tumor Targeting Ligand construct. It is a DNA sequence. The sequence is:

TCTCCGGCTCCGCCTGCCTGCGACCTGCGGGTGCTGTCGAAGCTGCTGCGGGACAGCCAC
GTCCTCCACAGCCGCCTGAGCCAGTGCCCGGAGGTGCACCCGCTGCCTACGCCGGTGCT
GCTGCCGGCCGTGGACTTCAGCCTCGGGGAGTGGAAGACGCAGATGGAGGAGACCAAG
GCCCAGGACATCCTCGGGGCCGTGACCCTGCTCCTGGAAGGGGTGATGGCAGCGCGAGG
GCAGCTGGGGCCTACCTGCCTCAGCTCCCTGCTGGGGCAGCTGTCGGGGCAGGTGCGGC
TGCTGCTCGGGGCCCTGCAGTCCCTGCTCGGGACCCAGGGCCGGACCACAGCCCACAAG
GACCCCAACGCCATCTTCCTCTCCTTCCAGCACCTGCTCCGGGGCAAGGTCCGGTTCCTG
ATGCTGGTCGGCGGCAGCACCCTGTGCGTGAGACGGGCTCCGCCTACCACGGCCGTGCC
CTCGCGCACGAGCCTGGTCCTGACCCTGAACGAGCTCGGG.

SEQ ID NO: 84 includes Target Ligand: 1-513; Free energy: −269.4; gdT CAI: 0.79850475511585; ORF count: 1.

SEQ ID NO: 85 is the sequence name for TPO ECOg (53). It is a Tumor Targeting Ligand construct. It is a DNA sequence. The sequence is:

AGCCCCGCGCCGCCAGCGTGCGATCTGCGCGTGCTGAGCAAGCTGCTGCGCGACTCGCA
CGTGCTGCACTCGCGGCTCTCGCAGTGCCCCGAGGTGCACCCGCTGCCCACACCCGTGCT
GCTGCCCGCGGTGGACTTCTCGCTCGGCGAGTGGAAGACGCAGATGGAGGAGACGAAA
GCGCAGGACATCCTCGGCGCGGTGACGCTGCTGCTCGAAGGCGTGATGGCTGCTCGCGG
GCAGCTCGGGCCTACGTGCCTGAGCTCGCTGCTCGGGCAGCTGAGCGGGCAGGTGCGGC
TGCTGCTCGGCGCGCTGCAGTCGCTGCTCGGCACGCAGGGGCGCACCACAGCGCACAAG
GACCCGAACGCGATCTTCCTGAGCTTCCAGCACCTGCTGCGCGGGAAGGTGCGCTTCCTG
ATGCTCGTCGGCGGCAGCACGCTGTGCGTGCGCAGAGCGCCTCCGACCACCGCGGTGCC
CTCGCGCACCTCGCTCGTGCTCACGCTGAACGAGCTCGGC.

SEQ ID NO: 85 includes Target Ligand: 1-513; Free energy: −268.8; gdT CAI: 0.737843607302898; ORF count: 1.

SEQ ID NO: 86 is the sequence name for TPO ECOg (57). It is a Tumor Targeting Ligand construct. It is a DNA sequence. The sequence is:

AGCCCCGCTCCGCCAGCCTGCGACCTGCGGGTGCTGAGCAAGCTGCTGCGGGACAGCCA
CGTGCTGCACAGCCGGCTGAGCCAGTGCCCCGAGGTGCACCCGCTGCCCACGCCCGTGC
TGCTGCCCGCTGTGGACTTCAGCCTGGGCGAGTGGAAGACCCAGATGGAGGAGACCAAG
GCCCAGGACATCCTGGGCGCCGTGACCCTGCTGCTGGAAGGGGTGATGGCCGCTAGGGG
CCAGCTGGGGCCTACCTGCCTGAGCAGCCTGCTGGGGCAGCTGAGCGGGCAGGTGCGGC
TGCTGCTGGGCGCCCTGCAGAGCCTGCTGGGCACCCAGGGGCGCACCACAGCCCACAAG
GACCCCAACGCCATCTTCCTGAGCTTCCAGCACCTGCTGCGGGGCAAGGTGCGCTTCCTG
ATGCTGGTCGGCGGCAGCACCCTGTGCGTGCGCAGGGCTCCGCCTACCACCGCCGTGCC
CAGCCGCACCAGCCTGGTGCTGACCCTGAACGAGCTCGGC.

SEQ ID NO: 86 includes Target Ligand: 1-513; Free energy: −267.9; gdT CAI: 0.853835989559969; ORF count: 2.

SEQ ID NO: 87 is the sequence name for Fc. It is a Fusion Moiety construct. It is an AA sequence. The sequence is:

DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK.

SEQ ID NO: 87 includes Fusion Moeity: 1-227.

SEQ ID NO: 88 is the sequence name for Fc. It is a Fusion Moiety construct. It is a DNA sequence. The sequence is:

GACAAGACACACACCTGCCCCCCATGTCCCGCGCCTGAACTCCTGGGGGGGCCCTCTGTC
TTCCTGTTCCCACCGAAGCCGAAAGACACCCTTATGATATCTCGGACCCCTGAGGTAACT
TGTGTAGTAGTTGATGTGTCTCACGAAGATCCAGAAGTGAAGTTTAATTGGTATGTGGAT
GGAGTGGAAGTTCACAACGCTAAGACCAAGCCCAGGGAGGAACAATACAATTCCACAT
ACCGAGTTGTTTCAGTGCTCACTGTGTTGCATCAGGACTGGCTCAACGGCAAGGAGTATA
AATGCAAAGTCAGTAACAAAGCCCTGCCCGCCCCCATCGAGAAAACCATCTCGAAGGCA
AAAGGCCAGCCTCGCGAGCCACAGGTCTACACCCTGCCTCCCTCACGTGACGAGCTGAC
AAAGAATCAGGTCAGCTTGACGTGTTTAGTGAAAGGATTTTATCCAAGCGACATTGCCGT
GGAATGGGAGAGCAATGGCCAGCCTGAGAACAATTACAAAACAACTCCACCTGTGCTCG
ACAGTGATGGGTCTTTCTTCTTATACTCCAAACTAACGGTCGATAAGAGCAGATGGCAGC
AAGGTAACGTGTTTTCCTGCTCAGTGATGCATGAGGCTCTGCATAACCACTATACTCAGA
AGTCCCTTAGTCTGAGCCCGGGTAAG.

SEQ ID NO: 88 includes Fusion Moeity: 1-681; Free energy: −199.9; gdT CAI: 0.747707161534413; ORF count: 6.

SEQ ID NO: 89 is the sequence name for FC ECOg (85). It is a Fusion Moiety construct. It is a DNA sequence. The sequence is:

GATAAGACCCACACCTGTCCGCCGTGCCCCGCACCCGAGCTTCTCGGCGGCCCCAGCGT
GTTCCTGTTTCCGCCGAAGCCCAAGGACACCCTGATGATCTCCCGGACGCCCGAGGTGAC
CTGCGTGGTGGTGGACGTGTCCCACGAGGACCCCGAGGTGAAGTTCAACTGGTACGTGG
ACGGGGTCGAGGTGCACAACGCCAAGACCAAGCCCCGCGAGGAGCAGTACAACTCCAC
CTACCGGGTCGTGTCCGTGCTGACCGTGCTGCACCAGGACTGGCTGAACGGGAAGGAGT
ACAAGTGCAAGGTGTCCAACAAGGCCCTGCCCGCGCCCATCGAGAAGACCATCTCCAAG
GCCAAGGGGCAGCCCAGGGAGCCCCAGGTGTACACCCTGCCTCCGTCCCGGGACGAGCT
GACCAAGAACCAGGTGTCCCTGACCTGCCTGGTGAAGGGGTTCTACCCCAGCGACATCG
CCGTGGAGTGGGAGTCCAACGGGCAGCCCGAGAACAACTACAAGACCACGCCGCCGGT
GCTGGACTCCGACGGGAGCTTCTTCCTGTACTCCAAGCTGACCGTGGACAAGTCCCGGTG
GCAGCAGGGGAACGTGTTCAGCTGCAGCGTGATGCACGAGGCCCTGCACAACCACTACA
CCCAGAAGTCCCTGAGCCTGAGCCCCGGGAAG.

SEQ ID NO: 89 includes Fusion Moeity: 1-681; Free energy: −269.2; gdT CAI: 0.894515513769793; ORF count: 0.

SEQ ID NO: 90 is the sequence name for FC ECOg (59). It is a Fusion Moiety construct. It is a DNA sequence. The sequence is:

GACAAGACGCACACGTGTCCGCCGTGCCCCGCACCCGAACTGCTCGGCGGCCCCAGCGT
GTTCCTGTTTCCGCCGAAGCCCAAGGACACGCTGATGATCTCGCGCACGCCCGAGGTGA
CGTGCGTCGTCGTCGACGTGTCGCACGAGGACCCCGAGGTGAAGTTCAACTGGTACGTC
GACGGCGTCGAGGTGCACAACGCCAAGACGAAGCCGCGCGAGGAGCAGTACAACAGCA
CGTACCGCGTCGTGAGCGTGCTGACCGTGCTGCACCAGGACTGGCTGAACGGCAAGGAG
TACAAGTGCAAGGTGAGCAACAAGGCGCTGCCCGCGCCGATCGAGAAGACGATCAGCA
AGGCCAAGGGGCAGCCACGCGAGCCGCAGGTGTACACGCTGCCGCCGTCGCGCGACGA
GCTGACGAAGAACCAGGTGTCGCTGACGTGCCTCGTGAAGGGCTTCTACCCCAGCGACA
TCGCCGTCGAGTGGGAGAGCAACGGGCAGCCCGAGAACAACTACAAGACGACGCCGCC
GGTGCTCGACAGCGACGGCAGCTTCTTCCTGTACAGCAAGCTGACCGTCGACAAGTCGC
GCTGGCAGCAGGGCAACGTGTTCAGCTGCAGCGTGATGCACGAGGCGCTGCACAACCAC
TACACGCAGAAGTCGCTGTCGCTGTCGCCCGGCAAG.

SEQ ID NO: 90 includes Fusion Moeity: 1-681; Free energy: −299.3; gdT CAI: 0.760430405503452; ORF count: 0.

SEQ ID NO: 91 is the sequence name for FC ECOg (5). It is a Fusion Moiety construct. It is a DNA sequence. The sequence is:

GACAAGACGCACACCTGTCCGCCGTGCCCGGCTCCCGAACTGCTCGGCGGCCCGTCCGT
CTTCCTGTTTCCGCCGAAGCCGAAGGACACCCTGATGATCAGCCGGACGCCGGAGGTGA
CCTGCGTCGTCGTCGACGTCAGCCACGAGGACCCCGAGGTCAAGTTCAACTGGTACGTC
GACGGCGTCGAGGTCCACAACGCCAAGACGAAGCCCCGCGAGGAGCAGTACAACTCGA
CGTACCGGGTCGTCTCCGTCCTGACCGTCCTCCACCAGGACTGGCTGAACGGCAAGGAG
TACAAGTGCAAGGTCAGCAACAAGGCCCTGCCGGCCCCGATCGAGAAGACGATCTCGAA
GGCCAAGGGCCAGCCTCGGGAGCCCCAGGTCTACACGCTGCCGCCGTCCCGGGACGAGC
TGACCAAGAACCAGGTCAGCCTGACCTGCCTGGTCAAGGGCTTCTACCCGTCCGACATC
GCCGTCGAGTGGGAGTCGAACGGCCAGCCCGAGAACAACTACAAGACGACGCCGCCGG
TCCTGGACTCCGACGGCAGCTTCTTCCTGTACAGCAAGCTGACCGTCGACAAGTCCCGGT
GGCAGCAGGGCAACGTCTTCAGCTGCTCCGTCATGCACGAGGCCCTGCACAACCACTAC
ACCCAGAAGTCCCTGTCCCTGTCGCCCGGCAAG.

SEQ ID NO: 91 includes Fusion Moeity: 1-681; Free energy: −278.1; gdT CAI: 0.766513451450202; ORF count: 0.

SEQ ID NO: 92 is the sequence name for FC ECOg (23). It is a Fusion Moiety construct. It is a DNA sequence. The sequence is:

GACAAGACCCATACCTGTCCGCCGTGCCCGGCTCCGGAACTGCTCGGCGGCCCGTCCGTC
TTCCTGTTTCCGCCGAAGCCGAAGGACACCCTGATGATCAGCCGGACGCCGGAGGTGAC
CTGCGTCGTCGTCGACGTCAGCCACGAGGACCCGGAGGTCAAGTTCAACTGGTACGTCG
ACGGCGTCGAGGTCCACAACGCCAAGACCAAGCCCCGGGAAGAGCAGTACAACAGCAC
CTACCGGGTCGTGTCCGTCCTGACCGTCCTGCACCAGGACTGGCTGAACGGCAAGGAGT
ACAAGTGCAAGGTCAGCAACAAGGCCCTGCCGGCCCCGATCGAGAAGACCATCAGCAA
GGCCAAGGGCCAGCCCAGGGAGCCGCAGGTCTACACCCTGCCGCCGTCCCGGGACGAGC
TGACCAAGAACCAGGTCAGCCTGACCTGCCTGGTCAAGGGCTTCTACCCGTCCGACATC
GCCGTCGAATGGGAGTCCAACGGCCAGCCGGAGAACAACTACAAGACGACGCCGCCGG
TCCTGGACTCCGACGGCAGCTTCTTCCTGTACAGCAAGCTGACCGTCGACAAGTCCCGGT
GGCAGCAGGGCAACGTCTTCAGCTGCTCCGTCATGCACGAGGCCCTGCACAACCACTAC
ACCCAGAAGTCCCTGTCCCTGTCGCCCGGCAAG.

SEQ ID NO: 92 includes Fusion Moeity: 1-681; Free energy: −276.3; gdT CAI: 0.769448727316174; ORF count: 0.

SEQ ID NO: 93 is the sequence name for FC ECOg (56). It is a Fusion Moiety construct. It is a DNA sequence. The sequence is:

GACAAGACGCACACGTGTCCGCCGTGCCCAGCCCCGGAGCTTCTCGGCG
GCCCCTCGGTGTTCCTGTTTCCGCCGAAGCCCAAGGACACGCTGATGAT
CTCCCGGACCCCGGAGGTGACCTGCGTCGTCGTCGACGTGTCCCACGAG
GACCCCGAGGTGAAGTTCAACTGGTACGTCGACGGCGTCGAGGTGCACA
ACGCGAAGACGAAGCCCCGCGAGGAGCAGTACAACTCCACGTACCGCGT
CGTCTCCGTGCTCACCGTGCTGCACCAGGACTGGCTCAACGGCAAGGAG
TACAAGTGCAAGGTGTCCAACAAGGCGCTGCCCGCGCCCATCGAGAAGA
CCATCTCCAAGGCCAAGGGGCAGCCCCGGGAACCCCAGGTGTACACGCT
GCCGCCGAGCCGCGACGAGCTGACCAAGAACCAGGTGTCCCTGACCTGC
CTCGTCAAGGGGTTCTACCCCTCGGACATCGCCGTGGAGTGGGAGTCCA
ACGGGCAGCCCGAGAACAACTACAAGACGACGCCGCCGGTGCTCGACTC
CGACGGGTCCTTCTTCCTCTACTCGAAGCTCACCGTGGACAAGTCCCGG
TGGCAGCAGGGCAACGTGTTCAGCTGCTCCGTGATGCACGAGGCGCTGC
ACAACCACTACACGCAGAAGTCCCTGTCCCTGAGCCCCGGGAAG.

SEQ ID NO: 93 includes Fusion Moeity: 1-681; Free energy: −275.4; gdT CAI: 0.816569307205555; ORF count: 0.

SEQ ID NO: 94 is the sequence name for Hum2 scFv. It is a gdT Targeting scFv construct. It is an AA sequence. The sequence is:

IQLVQSGAEVKKPGASVKVSCKASGYTFTRYTMHWVRQAPGQGLEWIGY
INPSRGYTNYNQKFKDRATLTTDKSTSTAYMELSSLRSEDTAVYYCARY
YDDHYCLDYWGQGTLVTVSSGGSGGSGGSGGSGGDIQLTQSPSSLSASV
GDRVTITCRASSSVSYMNWYQQKPGKAPKRWIYDTSKVASGAPSRFTGS
GSGTDYTLTISSLQPEDFATYYCQQWSSNPLTFGGGTKLEIK.

SEQ ID NO: 94 includes gdT scFv: 1-238; gdT VH: 119-132; gdT Linker: 119-132; gdT VL: 133-238.

SEQ ID NO: 95 is the sequence name for Hum2 scFv ECOg (0). It is a gdT Targeting scFv construct. It is a DNA sequence. The sequence is:

ATCCAGCTGGTGCAGTCCGGGGCCGAGGTGAAGAAGCCCGGGGCCTCCG
TGAAGGTGTCCTGCAAGGCCTCCGGGTACACCTTCACCCGGTACACCAT
GCACTGGGTGAGGCAGGCTCCCGGGCAGGGGCTGGAGTGGATCGGGTAC
ATCAACCCCAGCCGCGGGTACACCAACTACAACCAGAAGTTCAAGGACC
GGGCCACGCTGACCACCGACAAGTCCACCTCCACCGCCTACATGGAGCT
GAGCTCCCTGCGGTCCGAGGACACCGCCGTGTACTACTGCGCCCGGTAC
TACGACGACCACTACTGCCTGGACTACTGGGGCCAGGGGACCCTGGTGA
CCGTGTCCTCCGGCGGCTCAGGCGGCTCAGGAGGCTCAGGCGGCTCAGG
CGGCGACATCCAGCTGACCCAGAGCCCCAGCTCCCTGTCCGCCTCCGTC
GGGGACCGGGTGACCATCACCTGCCGGGCCTCCTCCTCCGTGTCCTACA
TGAACTGGTACCAGCAGAAGCCCGGGAAGGCGCCCAAGCGGTGGATCTA
CGACACCTCCAAGGTGGCCTCCGGGGCTCCCTCACGGTTCACCGGGTCC
GGGTCCGGGACCGACTACACCCTGACCATCAGCTCCCTGCAGCCCGAGG
ACTTCGCCACCTACTACTGCCAGCAGTGGTCCTCCAACCCGCTGACCTT
CGGCGGCGGCACCAAGCTGGAGATCAAG.

SEQ ID NO: 95 includes gdT scFv: 1-714; gdT VH: 355-396; gdT Linker: 355-396; gdT VL: 397-714; Free energy: −325.7; gdT CAI: 0.910798811448247; ORF count: 0.

SEQ ID NO: 96 is the sequence name for Hum2 scFv ECOg (183). It is a gdT Targeting scFv construct. It is a DNA sequence. The sequence is:

ATCCAGCTCGTGCAGAGCGGGGCCGAGGTGAAGAAGCCCGGGGCGAGCG
TGAAGGTGTCGTGCAAGGCGAGCGGGTACACCTTCACGCGCTACACCAT
GCACTGGGTGCGCCAAGCTCCCGGGCAGGGGCTGGAGTGGATCGGGTAC
ATCAACCCCTCGCGCGGGTACACCAACTACAACCAGAAGTTCAAGGACC
GCGCCACGCTCACCACCGACAAGTCCACCTCCACCGCCTACATGGAGCT
CTCCTCGCTGCGCTCCGAGGACACCGCGGTGTACTACTGCGCGCGCTAC
TACGACGACCACTACTGCCTCGACTACTGGGGCCAGGGGACCCTCGTGA
CCGTGTCGAGTGGTGGTAGTGGTGGTAGTGGTGGTAGTGGTGGTAGTGG
TGGCGACATCCAGCTCACCCAGAGCCCCAGCTCGTTGAGCGCGAGCGTC
GGGGACCGCGTGACCATCACGTGCCGCGCCTCGTCGAGCGTGAGCTACA
TGAACTGGTACCAGCAGAAGCCCGGGAAGGCCCCGAAGAGGTGGATCTA
CGACACCTCCAAGGTGGCGAGCGGGGCTCCATCGCGCTTCACGGGGAGC
GGGAGCGGGACCGACTACACCCTCACCATCTCCTCGCTCCAGCCCGAGG
ACTTCGCCACCTACTACTGCCAGCAGTGGAGCTCCAACCCGCTCACCTT
CGGCGGCGGCACCAAGTTGGAGATCAAG.

SEQ ID NO: 96 includes gdT scFv: 1-714; gdT VH: 355-396; gdT Linker: 355-396; gdT VL: 397-714; Free energy: −331.6; gdT CAI: 0.766742326650226; ORF count: 2.

SEQ ID NO: 97 is the sequence name for Hum2 scFv ECOg (6). It is a gdT Targeting scFv construct. It is a DNA sequence. The sequence is:

ATCCAGCTGGTGCAGAGCGGCGCCGAGGTGAAGAAGCCCGGGGCCTCGG
TGAAGGTCTCCTGCAAGGCCTCGGGGTACACCTTCACCCGGTACACCAT
GCACTGGGTGCGCCAAGCCCCAGGGCAGGGCCTGGAGTGGATCGGGTAC
ATCAACCCCAGCCGCGGGTACACCAACTACAACCAGAAGTTCAAGGACC
GGGCCACGCTGACCACGGACAAGTCCACGTCCACGGCGTACATGGAGCT
GAGCTCCCTGCGCTCCGAGGACACCGCGGTGTACTACTGCGCCCGGTAC
TACGACGACCACTACTGCCTGGACTACTGGGGCCAGGGGACCCTGGTCA
CCGTGTCCTCCGGAGGCTCCGGAGGCTCCGGAGGCTCCGGAGGCTCCGG
AGGCGACATCCAGCTGACCCAGAGCCCCAGCTCCCTCAGCGCCTCCGTC
GGGGACCGGGTGACCATCACCTGCCGCGCCTCGTCCTCGGTGTCCTACA
TGAACTGGTACCAGCAGAAGCCCGGGAAGGCCCCGAAGCGGTGGATCTA
CGACACGTCCAAGGTCGCCTCCGGGGCTCCGTCGAGGTTCACCGGCTCC
GGCTCCGGGACGGACTACACCCTGACCATCTCCTCGCTGCAGCCCGAGG
ACTTCGCCACGTACTACTGCCAGCAGTGGTCCTCGAATCCCCTGACCTT
CGGCGGCGGCACGAAGCTGGAGATTAAG.

SEQ ID NO: 97 includes gdT scFv: 1-714; gdT VH: 355-396; gdT Linker: 355-396; gdT VL: 397-714; Free energy: −331; gdT CAI: 0.825626192459745; ORF count: 1.

SEQ ID NO: 98 is the sequence name for Hum2 scFv ECOg (42). It is a gdT Targeting scFv construct. It is a DNA sequence. The sequence is:

ATCCAGCTGGTCCAGTCCGGGGCCGAGGTGAAGAAGCCCGGGGCCTCCG
TGAAGGTCTCCTGCAAGGCCTCCGGGTACACCTTCACCCGGTACACCAT
GCACTGGGTCCGGCAGGCTCCAGGGCAGGGGCTGGAGTGGATCGGGTAC
ATCAACCCCAGCCGCGGGTACACCAACTACAACCAGAAGTTCAAGGACC
GGGCCACGCTGACCACCGACAAGTCCACCTCCACCGCCTACATGGAGCT
GAGCTCCCTGCGGTCCGAGGACACCGCCGTCTACTACTGCGCCCGGTAC
TACGACGACCACTACTGCCTGGACTACTGGGGCCAGGGGACCCTGGTGA
CCGTCTCCTCCGGAGGCTCCGGAGGCTCCGGAGGCTCCGGAGGCTCCGG
AGGCGACATCCAGCTGACCCAGAGCCCCAGCTCCCTCTCCGCCTCCGTC
GGGGACCGGGTGACCATCACCTGCCGGGCCTCCTCCTCCGTCTCCTACA
TGAACTGGTACCAGCAGAAGCCCGGGAAGGCCCCGAAGCGGTGGATCTA
CGACACCTCCAAGGTGGCCTCCGGGGCCCCTAGCAGGTTCACCGGGTCC
GGGTCCGGGACCGACTACACCCTGACCATCTCCAGCCTCCAGCCCGAGG
ACTTCGCCACCTACTACTGCCAGCAGTGGTCCTCCAACCCGCTGACCTT
CGGCGGCGGCACCAAGCTGGAGATCAAG.

SEQ ID NO: 98 includes gdT scFv: 1-714; gdT VH: 355-396; gdT Linker: 355-396; gdT VL: 397-714; Free energy: −330; gdT CAI: 0.887375945709965; ORF count: 1.

SEQ ID NO: 99 is the sequence name for Hum2 scFv ECOg (196). It is a gdT Targeting scFv construct. It is a DNA sequence. The sequence is:

ATCCAGCTGGTGCAGTCCGGGGCCGAGGTGAAGAAGCCCGGGGCCTCCG
TGAAGGTGTCCTGCAAGGCCTCCGGGTACACCTTCACCCGGTACACCAT
GCACTGGGTGAGGCAGGCTCCCGGGCAGGGGCTGGAGTGGATCGGGTAC
ATCAACCCCTCGCGCGGGTACACCAACTACAACCAGAAGTTCAAGGACC
GGGCCACGCTGACCACCGACAAGAGCACCTCCACCGCCTACATGGAGCT
GTCCAGCCTGCGGTCCGAGGACACCGCCGTGTACTACTGCGCCCGGTAC
TACGACGACCACTACTGCCTGGACTACTGGGGCCAGGGGACCCTGGTGA
CCGTGTCCAGCGGCGGCTCTGGCGGCTCTGGAGGCTCCGGCGGCTCTGG
AGGCGACATCCAGCTGACCCAGTCCCCGTCCAGCCTGTCCGCCAGCGTC
GGGGACCGGGTGACCATCACCTGCCGGGCCTCCTCCAGCGTGTCCTACA
TGAACTGGTACCAGCAGAAGCCCGGGAAGGCGCCCAAGCGGTGGATCTA
CGACACCTCCAAGGTGGCCTCCGGGGCTCCCAGCCGTTTCACCGGGTCC
GGGTCCGGGACCGACTACACCCTGACCATCTCCAGCCTGCAGCCCGAGG
ACTTCGCCACCTACTACTGCCAGCAGTGGTCCAGCAACCCGCTGACCTT
CGGCGGCGGCACCAAGCTGGAGATCAAG.

SEQ ID NO: 99 includes gdT scFv: 1-714; gdT VH: 355-396; gdT Linker: 355-396; gdT VL: 397-714; Free energy: −324.7; gdT CAI: 0.892144940351689; ORF count: 1.

SEQ ID NO: 100 is the sequence name for Hum2 scFv ECOg (172). It is a gdT Targeting scFv construct. It is a DNA sequence. The sequence is:

ATCCAGCTCGTCCAGTCCGGGGCCGAGGTGAAGAAGCCCGGGGCGTCCG
TGAAGGTGTCCTGCAAGGCGTCCGGGTACACGTTCACCCGGTACACGAT
GCACTGGGTCCGTCAGGCTCCCGGGCAGGGGCTGGAGTGGATCGGGTAC
ATCAACCCGTCCCGCGGGTACACGAACTACAACCAGAAGTTCAAGGACC
GGGCGACCCTGACGACCGACAAGTCCACGTCCACCGCGTACATGGAGCT
GTCGTCCCTGCGGTCCGAGGACACCGCCGTGTACTACTGCGCCCGGTAC
TACGACGACCACTACTGCCTCGACTACTGGGGCCAGGGGACCCTCGTGA
CCGTGTCCTCCGGAGGTTCCGGCGGCTCCGGAGGATCTGGCGGCTCCGG
CGGCGACATCCAGCTGACCCAGTCGCCCTCGTCCCTGTCCGCGTCCGTC
GGGGACCGGGTGACGATCACGTGCCGGGCGTCCTCCTCCGTGTCCTACA
TGAACTGGTACCAGCAGAAGCCCGGGAAGGCCCCGAAGAGGTGGATCTA
CGACACGTCCAAGGTCGCGTCCGGGGCTCCATCCCGGTTCACCGGGTCC
GGGTCCGGGACCGACTACACCCTGACGATCTCCTCGCTCCAGCCCGAGG
ACTTCGCGACGTACTACTGCCAGCAGTGGTCCTCGAATCCCCTGACGTT
CGGCGGCGGCACGAAGCTGGAGATCAAG.

SEQ ID NO: 100 includes gdT scFv: 1-714; gdT VH: 355-396; gdT Linker: 355-396; gdT VL: 397-714; Free energy: −324.5; gdT CAI: 0.827859696643083; ORF count: 0.

SEQ ID NO: 101 is the sequence name for gd-c V6 scFv. It is a gdT Targeting scFv construct. It is an AA sequence. The sequence is:

QVQLQQSGPGLVKPSQTLSLTCAISGDSVSSNRAAWNWIRQSPSKGLEW
LGRTYYRSKWYNEYAASVKSRMSINPDTSKNQFSLQLNSVTPEDTALYY
CARDLWELREACDIWGQGTMVTVSSGGSGGSGGSGGSGGDIVMTQSPSF
LSTFVGDRVTITCRASQGISSYLAWYQQKPGKVPKLLIYVASTLQSGVP
SRFSGSGSGTEFTLTISSLQPEDFATYYCQQLNSYPFTFGPGTKVDIK.

SEQ ID NO: 101 includes gdT scFv: 1-244; gdT VH: 124-137; gdT Linker: 124-137; gdT VL: 138-244.

SEQ ID NO: 102 is the sequence name for gd-C V6 scFv. It is a gdT Targeting scFv construct. It is a DNA sequence. The sequence is:

CAGGTGCAGCTGCAGCAGTCCGGGCCCGGGCTGGTGAAGCCCAGCCAGA
CCCTGAGCCTGACCTGCGCCATCAGCGGGGACTCCGTCAGCTCCAACCG
GGCCGCCTGGAACTGGATCAGGCAGTCCCCGTCCAAGGGGCTGGAGTGG
CTCGGCCGGACCTACTACCGCTCCAAGTGGTACAACGAGTACGCCGCCT
CCGTGAAGTCCCGGATGAGCATCAACCCCGATACCTCCAAGAACCAGTT
CAGCCTGCAGCTGAACTCCGTGACGCCCGAGGATACGGCCCTGTACTAC
TGCGCCAGGGACCTGTGGGAGCTGCGCGAGGCCTGCGACATCTGGGGCC
AGGGGACCATGGTGACCGTGTCCTCCGGCGGCTCAGGCGGCTCAGGTGG
CTCCGGCGGATCAGGCGGCGACATCGTGATGACCCAGTCCCCATCCTTC
CTGTCCACCTTCGTCGGGGACCGGGTGACCATCACCTGCCGGGCCAGCC
AGGGGATCAGCTCCTACCTGGCCTGGTACCAGCAGAAGCCCGGGAAGGT
GCCCAAGCTGCTGATCTACGTGGCCTCAACCCTGCAGTCCGGGGTGCCC
AGCCGGTTCAGCGGGTCCGGGTCCGGGACCGAGTTCACCCTGACCATCA
GCTCCCTGCAGCCCGAGGACTTCGCCACCTACTACTGCCAGCAGCTGAA
CTCCTACCCCTTCACCTTCGGGCCCGGGACCAAGGTGGACATCAAG.

SEQ ID NO: 102 includes gdT scFv: 1-732; gdT VH: 369-411; gdT Linker: 369-411; gdT VL: 412-732; Free energy: −325.2; gdT CAI: 0.901042; ORF count: 1.

SEQ ID NO: 103 is the sequence name for gd-c V6 scFv ECOg (69). It is a gdT Targeting scFv construct. It is a DNA sequence. The sequence is:

CAGGTGCAGCTGCAGCAGTCCGGGCCCGGGCTGGTGAAGCCTTCCCAGA
CCCTCTCCCTCACCTGCGCCATCTCCGGGGATTCCGTGTCCTCCAACCG
CGCCGCCTGGAACTGGATCAGGCAGTCCCCTTCCAAGGGGCTGGAGTGG
CTGGGGCGCACGTACTACCGCTCCAAGTGGTACAACGAGTACGCCGCTT
CCGTGAAGTCCCGCATGAGCATCAACCCCGATACCTCCAAGAACCAGTT
CTCCCTGCAGCTGAACTCCGTGACCCCGGAGGATACCGCGCTGTACTAC
TGCGCCCGGGACCTGTGGGAGCTGCGGGAAGCCTGCGACATCTGGGGCC
AGGGGACCATGGTGACCGTGTCCTCCGGAGGCTCCGGAGGCTCCGGAGG
TTCCGGAGGCTCCGGCGGCGACATCGTGATGACGCAGTCCCCTTCCTTC
CTGAGCACCTTCGTAGGGGACCGCGTTACCATCACCTGCAGGGCTTCCC
AGGGGATCTCCTCCTACCTCGCATGGTACCAGCAGAAGCCCGGGAAGGT
TCCCAAGCTGCTGATCTACGTGGCTTCCACGCTGCAGTCCGGGGTGCCT
TCCCGGTTCTCCGGGAGCGGGAGCGGGACGGAGTTCACCCTCACCATCA
GCTCCCTGCAGCCCGAGGACTTCGCAACGTACTACTGCCAGCAGCTGAA
CTCCTACCCCTTCACCTTCGGGCCCGGGACGAAGGTGGACATCAAG.

SEQ ID NO: 103 includes gdT scFv: 1-732; gdT VH: 369-411; gdT Linker: 369-411; gdT VL: 412-732; Free energy: −328.2; gdT CAI: 0.885003557188278; ORF count: 0.

SEQ ID NO: 104 is the sequence name for gd-c V6 scFv ECOg (34). It is a gdT Targeting scFv construct. It is a DNA sequence. The sequence is:

CAAGTGCAGCTGCAGCAGAGCGGGCCCGGGCTGGTGAAGCCGTCGCAGA
CGCTGTCGCTGACGTGCGCCATCAGCGGCGACAGCGTGAGCAGCAACCG
CGCCGCGTGGAACTGGATCAGGCAGTCGCCCAGCAAGGGGCTGGAGTGG
CTGGGGCGCACGTACTACCGCAGCAAGTGGTACAACGAGTACGCCGCCA
GCGTGAAGAGCCGCATGAGCATCAACCCCGACACCAGCAAGAACCAGTT
CTCGCTGCAGCTGAACAGCGTGACGCCCGAGGACACCGCGCTGTACTAC
TGCGCGCGCGACTTGTGGGAGCTGCGCGAGGCGTGCGACATCTGGGGCC
AGGGCACCATGGTGACCGTGAGCAGCGGCGGCAGTGGTGGCAGCGGTGG
TAGCGGCGGTAGCGGCGGCGACATCGTGATGACGCAGTCGCCGTCGTTC
TTGAGCACGTTCGTGGGCGACCGCGTGACCATCACGTGCCGCGCGTCGC
AGGGCATCAGCAGCTACTTGGCGTGGTACCAGCAGAAGCCCGGCAAGGT
GCCCAAGCTGCTGATCTACGTGGCCAGCACGCTGCAGAGCGGCGTGCCG
TCGCGCTTCAGCGGCAGCGGCAGCGGCACCGAGTTCACGCTGACCATCA
GCAGCCTGCAGCCCGAGGACTTCGCCACGTACTACTGCCAGCAGCTGAA
CAGCTACCCGTTCACGTTCGGGCCCGGCACCAAGGTGGACATCAAG.

SEQ ID NO: 104 includes gdT scFv: 1-732; gdT VH: 369-411; gdT Linker: 369-411; gdT VL: 412-732; Free energy: −353.6; gdT CAI: 0.744822337548797; ORF count: 2.

SEQ ID NO: 105 is the sequence name for gd-c V6 scFv ECOg (55). It is a gdT Targeting scFv construct. It is a DNA sequence. The sequence is:

CAGGTGCAGCTGCAGCAGAGCGGCCCCGGGCTCGTGAAGCCGTCGCAGA
CCCTGAGCCTCACCTGCGCCATCTCCGGGGACTCCGTGTCCTCGAACCG
CGCCGCGTGGAACTGGATTCGGCAGAGCCCCAGCAAGGGCCTGGAGTGG
CTGGGGCGCACCTACTACCGCTCCAAGTGGTACAACGAGTACGCCGCCT
CGGTGAAGTCCCGGATGAGCATCAACCCCGACACCTCGAAGAACCAGTT
CTCGCTGCAGCTGAACTCCGTGACCCCGGAGGACACGGCGCTGTACTAC
TGCGCCCGGGACCTCTGGGAGCTCCGCGAGGCCTGCGACATCTGGGGCC
AGGGGACCATGGTCACCGTGTCCTCCGGAGGCTCCGGAGGCTCCGGAGG
CTCCGGAGGCTCCGGAGGCGACATCGTGATGACCCAGAGCCCCAGCTTC
CTGAGCACCTTCGTCGGGGACCGGGTCACCATCACGTGCCGCGCGTCCC
AGGGGATCTCCTCGTACCTGGCCTGGTACCAGCAGAAGCCCGGGAAGGT
GCCGAAGCTGCTGATCTACGTGGCCTCGACGCTGCAGTCCGGGGTCCCG
AGCCGCTTCAGCGGCTCCGGGTCCGGGACCGAGTTCACCCTGACCATCT
CGTCGCTGCAGCCCGAGGACTTCGCCACGTACTACTGCCAGCAGCTGAA
CAGCTACCCCTTCACCTTCGGCCCCGGGACCAAGGTGGACATCAAG.

SEQ ID NO: 105 includes gdT scFv: 1-732; gdT VH: 369-411; gdT Linker: 369-411; gdT VL: 412-732; Free energy: −351.8; gdT CAI: 0.812038056353435; ORF count: 0. SEQ ID NO: 106 is the sequence name for gd-c V6 scFv ECOg (21). It is a gdT Targeting scFv construct. It is a DNA sequence. The sequence is:

CAGGTGCAGCTGCAGCAGAGCGGGCCCGGGCTGGTGAAGCCCAGCCAGA
CCCTGAGCCTGACCTGCGCCATCTCCGGGGACAGCGTGAGCAGCAACCG
GGCCGCCTGGAACTGGATCAGGCAGAGCCCCAGCAAGGGGCTGGAGTGG
CTGGGGCGCACATACTACCGCTCCAAGTGGTACAATGAGTATGCCGCCA
GCGTGAAGAGCCGCATGAGCATCAACCCCGACACCTCCAAGAACCAGTT
CAGCCTGCAGCTGAACAGCGTGACGCCCGAGGACACTGCCCTGTACTAC
TGCGCCAGGGACCTGTGGGAGCTGCGCGAGGCCTGCGACATCTGGGGCC
AGGGCACCATGGTGACAGTGTCCTCCGGCGGCTCAGGAGGCTCCGGAGG
CTCTGGCGGCTCAGGCGGCGACATCGTGATGACCCAGAGCCCCAGCTTC
CTGAGCACCTTCGTGGGCGACAGGGTGACCATCACCTGCAGGGCCAGCC
AGGGCATCAGCAGCTACCTGGCCTGGTACCAGCAGAAGCCCGGCAAGGT
GCCCAAGCTGCTGATCTATGTGGCCAGCACCCTGCAGAGCGGGGTGCCC
AGCCGCTTCAGCGGCAGCGGCAGCGGCACAGAGTTCACCCTGACCATCA
GCAGCCTGCAGCCCGAGGACTTCGCCACATACTACTGCCAGCAGCTGAA
CAGCTACCCCTTCACCTTCGGGCCCGGCACCAAGGTGGACATCAAG.

SEQ ID NO: 106 includes gdT scFv: 1-732; gdT VH: 369-411; gdT Linker: 369-411; gdT VL: 412-732; Free energy: −338.7; gdT CAI: 0.860184134678756; ORF count: 3.

SEQ ID NO: 107 is the sequence name for gd-c V6 scFv ECOg (99). It is a gdT Targeting scFv construct. It is a DNA sequence. The sequence is:

CAGGTGCAGCTGCAGCAGAGCGGGCCCGGGCTCGTGAAGCCCTCGCAGA
CCCTCTCCCTCACGTGCGCGATCTCCGGGGACTCCGTGTCCTCCAACCG
CGCCGCGTGGAACTGGATACGGCAGAGCCCCTCGAAGGGGCTGGAGTGG
CTGGGGCGCACGTACTACCGCTCCAAGTGGTACAACGAGTACGCCGCCT
CCGTGAAGTCCCGCATGAGCATCAACCCCGACACCTCCAAGAACCAGTT
CTCCCTGCAGCTGAACTCCGTGACTCCCGAGGACACCGCGCTGTACTAC
TGCGCGCGGGACCTGTGGGAGCTGCGCGAGGCGTGCGACATCTGGGGAC
AGGGGACCATGGTGACCGTGTCCTCCGGAGGAAGCGGAGGAAGCGGAGG
AAGCGGAGGAAGCGGAGGAGACATCGTGATGACGCAGTCCCCTTCCTTC
CTCTCCACCTTCGTGGGAGACCGCGTGACCATCACGTGCCGCGCTTCCC
AGGGGATCTCCTCCTACCTCGCGTGGTACCAGCAGAAGCCCGGGAAGGT
GCCCAAGCTCCTCATCTACGTGGCCTCCACGCTGCAGAGCGGGGTGCCC
TCGCGCTTCTCCGGGAGCGGGAGCGGGACGGAGTTCACCCTCACCATCT
CTTCCCTGCAGCCCGAGGACTTCGCCACGTACTACTGCCAGCAGCTGAA
CTCCTACCCCTTCACCTTCGGGCCCGGGACGAAGGTGGACATCAAG.

SEQ ID NO: 107 includes gdT scFv: 1-732; gdT VH: 369-411; gdT Linker: 369-411; gdT VL: 412-732; Free energy: −335; gdT CAI: 0.835639819762746; ORF count: 0.

SEQ ID NO: 108 is the sequence name for gd-c V1 HL scFv. It is a gdT Targeting scFv construct. It is an AA sequence. The sequence is:

EVQLLESGGGLVKPGGSLRLSCAASRFTLSSYDMNWVRQAPGKGLEWVSSISSSSSYIYYAD
SVKGRFTISRDNAKNSLYLQMNSLRAEDTAVYYCARDRGVGGTDYYYYGLDVWGQGTTV
TVSSGGSGGSGGSGGSGGEIVMTQSPGTLSLSPGERATLSCRASQSVSSSYLAWYQQKPGQA
PRLLIYGASSRATGIPDRFSGSGSGTDFTLTISRLEPEDFAVYYCQQYGSSPPYTFGQGTKVEIK.

SEQ ID NO: 108 includes gdT scFv: 1-248; gdT VH: 126-139; gdT Linker: 126-139; gdT VL: 140-248.

SEQ ID NO: 109 is the sequence name for gd-c V1 HL scFv. It is a gdT Targeting scFv construct. It is a DNA sequence. The sequence is:

GAGGTGCAGCTGCTGGAGAGCGGCGGCGGCCTGGTGAAACCCGGCGGCTCCCTGCGGCT
GTCCTGCGCCGCCTCCAGGTTCACCCTGTCCAGCTACGACATGAACTGGGTGAGGCAGG
CTCCCGGGAAGGGGCTGGAGTGGGTGTCCTCCATCTCCTCCAGCTCCAGCTACATCTACT
ACGCCGATTCCGTGAAGGGGAGATTCACCATCTCCAGGGACAACGCCAAGAACTCCCTG
TACCTGCAGATGAACTCCCTGCGGGCCGAGGACACCGCCGTGTACTACTGCGCCAGGGA
CAGGGGCGTTGGCGGCACCGACTACTACTACTACGGGCTGGACGTGTGGGGCCAGGGGA
CCACCGTGACCGTGTCCAGCGGCGGCTCTGGCGGCTCTGGAGGCTCTGGCGGCTCTGGC
GGCGAGATCGTGATGACCCAGTCTCCCGGGACCCTGTCCCTGTCTCCCGGGGAGAGGGC
TACCCTGTCCTGCAGGGCCAGCCAGTCCGTGTCCTCCAGCTACCTGGCCTGGTACCAGCA
GAAACCCGGGCAGGCTCCCCGGCTGCTGATCTACGGGGCCTCTTCCAGGGCCACCGGCA
TCCCCGACAGGTTCTCCGGGTCCGGGTCCGGGACCGACTTCACCCTGACCATCTCCAGGC
TGGAGCCCGAGGACTTCGCCGTGTACTACTGCCAGCAGTACGGGTCCTCTCCACCTTACA
CCTTCGGGCAGGGGACCAAGGTGGAGATCAAG.

SEQ ID NO: 109 includes gdT scFv: 1-744; gdT VH: 376-417; gdT Linker: 376-417; gdT VL: 418-744; Free energy: −341.2; gdT CAI: 0.91246469035254; ORF count: 2.

SEQ ID NO: 110 is the sequence name for gd-c V1 HL scFv (63). It is a gdT Targeting scFv construct. It is a DNA sequence. The sequence is:

GAGGTCCAGCTCCTGGAGAGCGGAGGAGGCCTGGTGAAGCCCGGAGGCTCCCTGAGGCT
CTCCTGCGCCGCCTCCAGGTTCACCCTCTCCTCCTACGACATGAACTGGGTGAGGCAGGC
CCCGGGGAAGGGGCTGGAGTGGGTCTCCTCCATCTCCTCCTCCTCCTCCTACATCTACTA
CGCCGACTCCGTGAAGGGGCGCTTCACCATCTCCCGGGACAACGCCAAGAACTCCCTCT
ACCTCCAGATGAACTCCCTGAGGGCCGAGGACACGGCCGTCTACTACTGCGCCCGGGAC
CGAGGGGTAGGAGGCACCGACTACTACTACTACGGGCTGGACGTCTGGGGCCAGGGGAC
CACCGTGACCGTCTCCTCCGGAGGCTCCGGAGGCTCCGGAGGCTCCGGAGGCTCCGGAG
GAGAGATCGTGATGACCCAGAGCCCCGGGACCCTCTCCCTGAGCCCCGGGGAGAGGGCT
ACCCTCTCCTGCCGGGCCAGCCAGAGCGTCTCCTCCTCCTACCTGGCCTGGTACCAGCAG
AAGCCGGGGCAGGCCCCTAGGCTCCTGATCTACGGGGCCTCCTCTAGGGCCACCGGCAT
CCCGGACCGCTTCTCCGGGTCCGGGTCCGGGACCGACTTCACCCTCACCATCTCCCGGCT
GGAGCCGGAGGACTTCGCCGTCTACTACTGCCAGCAGTACGGGTCCTCTCCTCCTTACAC
CTTCGGGCAGGGGACCAAGGTGGAGATCAAG.

SEQ ID NO: 110 includes gdT scFv: 1-744; gdT VH: 376-417; gdT Linker: 376-417; gdT VL: 418-744; Free energy: −362.6; gdT CAI: 0.854971378798751; ORF count: 1.

SEQ ID NO: 111 is the sequence name for gd-c V1 HL scFv (72). It is a gdT Targeting scFv construct. It is a DNA sequence. The sequence is:

GAGGTGCAGCTGCTGGAGTCCGGCGGCGGCCTGGTGAAACCCGGCGGCAGCCTGCGGCT
GTCCTGCGCGGCAAGCCGCTTCACGCTGTCCAGCTACGACATGAACTGGGTGCGCCAGG
CACCCGGCAAGGGGCTGGAGTGGGTGTCCAGCATATCCAGCTCGTCAAGCTACATATAC
TACGCGGACAGCGTGAAGGGCCGGTTTACCATCTCGCGGGATAACGCCAAGAACAGCCT
GTACCTGCAGATGAACAGCCTGCGGGCCGAGGACACCGCGGTGTACTACTGCGCAAGGG
ACCGCGGGGTAGGCGGCACGGATTACTACTACTACGGGCTGGACGTGTGGGGCCAGGGG
ACCACCGTGACGGTGTCCTCCGGCGGCTCAGGCGGTTCCGGTGGCTCTGGCGGCTCAGG
CGGCGAGATTGTCATGACGCAGTCACCCGGCACGCTTAGCCTGTCGCCCGGGGAACGCG
CCACGCTGTCCTGCCGGGCCAGCCAGTCGGTGTCCAGCAGCTACCTGGCGTGGTACCAG
CAGAAACCCGGCCAGGCGCCCCGGCTGCTTATCTACGGGGCGTCTAGCCGGGCAACCGG
CATCCCGGACCGCTTCAGCGGGTCGGGCAGCGGGACGGACTTCACGCTGACAATCAGCC
GGCTGGAGCCCGAGGACTTCGCGGTGTACTACTGCCAGCAGTACGGCAGCTCGCCGCCT
TACACGTTTGGCCAGGGCACCAAGGTGGAAATCAAG.

SEQ ID NO: 111 includes gdT scFv: 1-744; gdT VH: 376-417; gdT Linker: 376-417; gdT VL: 418-744; Free energy: −366.6; gdT CAI: 0.761156065773582; ORF count: 4.

SEQ ID NO: 112 is the sequence name for gd-c V1 HL scFv (14). It is a gdT Targeting scFv construct. It is a DNA sequence. The sequence is:

GAGGTCCAGCTGCTGGAGAGCGGCGGCGGCCTGGTCAAACCCGGCGGCTCGCTGCGGCT
GAGCTGCGCCGCCAGCAGGTTCACCCTGAGCTCCTACGACATGAACTGGGTGCGCCAGG
CCCCAGGCAAGGGGCTGGAGTGGGTGAGCTCGATCAGCTCGTCGTCGAGCTACATCTAC
TACGCCGACAGCGTCAAGGGGCGCTTCACCATCTCGCGCGACAACGCCAAGAACTCGCT
CTACCTCCAGATGAACTCGCTGCGGGCCGAGGACACCGCCGTCTACTACTGCGCCCGAG
ATCGCGGGGTTGGCGGCACCGACTACTACTACTACGGGCTCGACGTGTGGGGCCAGGGG
ACCACCGTGACCGTGAGCTCCGGCGGCTCTGGCGGCTCAGGTGGTAGCGGCGGCTCTGG
CGGCGAGATCGTGATGACCCAGAGCCCCGGGACCTTGTCGCTGAGCCCCGGCGAGAGGG
CCACGCTGAGCTGCCGGGCCAGCCAGAGCGTGAGCTCCAGCTACCTGGCCTGGTACCAG
CAGAAACCCGGCCAGGCGCCCCGGCTGTTGATCTACGGGGCCTCATCTCGGGCCACCGG
CATCCCCGACAGGTTCTCGGGGTCGGGGTCGGGGACCGACTTCACCCTGACCATCTCGCG
GCTGGAGCCCGAGGACTTCGCCGTCTACTACTGCCAGCAGTACGGGAGCTCGCCGCCGT
ACACCTTCGGCCAGGGGACCAAGGTCGAGATCAAG.

SEQ ID NO: 112 includes gdT scFv: 1-744; gdT VH: 376-417; gdT Linker: 376-417; gdT VL: 418-744; Free energy: −366.1; gdT CAI: 0.774531811261147; ORF count: 3.

SEQ ID NO: 113 is the sequence name for gd-c V1 HL scFv (22). It is a gdT Targeting scFv construct. It is a DNA sequence. The sequence is:

GAGGTGCAGCTGCTGGAGTCCGGCGGCGGCCTGGTGAAACCCGGCGGCTCCCTGCGGCT
GTCCTGCGCCGCCAGCAGGTTCACCCTCTCCTCCTACGACATGAACTGGGTGCGGCAGGC
CCCAGGGAAGGGCCTGGAGTGGGTGAGCAGCATCAGCAGCAGCAGCAGCTACATCTACT
ACGCCGACAGCGTCAAGGGGCGCTTCACCATCAGCAGGGACAACGCCAAGAACAGCCT
GTACCTGCAGATGAACAGCCTCCGGGCCGAGGACACGGCCGTGTACTACTGCGCCCGTG
ATCGTGGCGTCGGCGGCACCGACTACTACTACTACGGGCTGGACGTGTGGGGCCAGGGG
ACGACCGTGACCGTCTCCTCCGGCGGCTCCGGAGGCTCTGGTGGTTCTGGCGGCTCCGGC
GGCGAGATCGTCATGACCCAGAGCCCCGGGACCCTGTCCCTGAGCCCCGGGGAGAGGGC
CACGCTCTCCTGCCGGGCCAGCCAGTCCGTGTCCTCCTCCTACCTGGCCTGGTACCAGCA
GAAACCCGGCCAGGCGCCCAGGCTGCTGATCTACGGGGCCTCTTCTCGCGCCACGGGCA
TCCCCGACCGCTTCTCCGGGAGCGGCTCCGGGACGGACTTCACCCTGACCATCAGCCGCC
TGGAGCCCGAGGACTTCGCCGTGTACTACTGCCAGCAGTACGGGTCCTCGCCGCCGTAC
ACCTTCGGGCAGGGGACGAAGGTGGAGATCAAG.

SEQ ID NO: 113 includes gdT scFv: 1-744; gdT VH: 376-417; gdT Linker: 376-417; gdT VL: 418-744; Free energy: −359.3; gdT CAI: 0.831881016856322; ORF count: 3.

SEQ ID NO: 114 is the sequence name for gd-c V1 HL scFv (11). It is a gdT Targeting scFv construct. It is a DNA sequence. The sequence is:

GAGGTGCAGCTGCTGGAGAGCGGCGGCGGCCTCGTGAAACCCGGCGGCTCGCTGCGGCT
CTCGTGCGCCGCTTCGCGCTTCACGCTCTCGTCGTACGACATGAACTGGGTGCGGCAGGC
TCCCGGGAAGGGGCTGGAGTGGGTCTCGTCGATCTCGTCGTCGTCGAGCTACATCTACTA
CGCCGACTCCGTGAAGGGGCGCTTCACGATCTCGCGCGACAACGCGAAGAACTCGCTCT
ACCTGCAGATGAACTCGCTGCGCGCCGAGGACACCGCCGTCTACTACTGCGCTCGCGAT
CGCGGAGTCGGCGGCACCGACTACTACTACTACGGGCTCGACGTCTGGGGCCAGGGGAC
GACCGTGACCGTGTCGAGCGGCGGAAGCGGCGGAAGCGGAGGAAGCGGCGGAAGCGGC
GGCGAGATCGTGATGACGCAGTCGCCCGGGACGCTCTCGCTCTCGCCCGGCGAGCGCGC
TACGCTCTCGTGCCGCGCGTCGCAGAGCGTCTCGTCGAGCTACCTCGCGTGGTACCAGCA
GAAACCCGGGCAGGCGCCGCGGCTGCTTATCTACGGCGCTAGCTCTCGCGCGACCGGCA
TCCCCGACCGCTTCTCCGGCTCCGGCTCCGGGACCGACTTCACGCTGACGATCTCGCGGC
TGGAGCCCGAGGACTTCGCCGTCTACTACTGCCAGCAGTACGGCTCGTCGCCGCCGTACA
CGTTCGGGCAGGGGACGAAGGTCGAGATCAAG.

SEQ ID NO: 114 includes gdT scFv: 1-744; gdT VH: 376-417; gdT Linker: 376-417; gdT VL: 418-744; Free energy: −358.3; gdT CAI: 0.705025041042151; ORF count: 1.

SEQ ID NO: 115 is the sequence name for JAML scFv. It is a gdT Targeting scFv construct. It is an AA sequence. The sequence is:

DVQLVESGAELVRPGASKLSCKALAYTFTDYEMHWVKQTPVHGLEWIGIIHPGSGGTVYNQ
KFKGKATLTADKSSSTAYMELSSLTSEDSTVYYCTRRRYYGSSYNWYFDVWGAGNGGSGG
SGGSGGSGGVLTQSPASLAASVGETVTITCRASENIYYSLAWYQQKQGKSPQLLIYNANSLE
DGVPSRFSGSGSGTQYSLKINSMQPEDTATYFCEQTYDVPLTFGAGTKLEL.

SEQ ID NO: 115 includes gdT scFv: 1-234; gdT VH: 117-130; gdT Linker: 117-130; gdT VL: 131-234; Free energy: −328.2; gdT CAI: 0.850906150801958; ORF count: 2.

SEQ ID NO: 116 is the sequence name for JAML scFv. It is a gdT Targeting scFv construct. It is a DNA sequence. The sequence is:

GATGTCCAGCTGGTGGAGTCTGGGGCTGAGCTGGTGAGGCCTGGGGCCAGCAAGCTGTC
CTGCAAGGCCCTGGCCTACACCTTCACAGACTATGAGATGCACTGGGTGAAGCAGACCC
CAGTGCATGGGCTGGAGTGGATTGGGATCATCCATCCAGGCTCTGGTGGCACAGTCTAC
AACCAGAAGTTCAAGGGGAAGGCCACACTCACAGCTGACAAGTCCAGCTCCACAGCCTA
CATGGAGCTGTCCAGCCTGACCTCTGAGGACTCCACAGTCTACTACTGCACCAGGAGGA
GGTACTATGGCTCCAGCTACAACTGGTACTTTGATGTGTGGGGAGCTGGGAATGGTGGCT
CTGGTGGCTCTGGTGGCTCTGGTGGCTCTGGTGGAGTGCTGACCCAGTCCCCAGCCAGCC
TGGCTGCCTCTGTTGGGGAGACAGTCACCATCACCTGCAGGGCCTCTGAGAACATCTACT
ACAGCCTGGCCTGGTACCAGCAGAAGCAGGGGAAGTCTCCCCAGCTGCTGATCTACAAT
GCCAACAGCCTGGAGGATGGGGTGCCCAGCAGGTTCTCTGGGTCTGGGTCTGGCACCCA
GTACTCCCTCAAGATCAACAGCATGCAGCCAGAGGACACAGCCACCTACTTCTGTGAGC
AGACCTATGATGTGCCCCTCACCTTTGGGGCTGGCACCAAGCTGGAGCTC.

SEQ ID NO: 116 includes gdT scFv: 1-702; gdT VH: 349-390; gdT Linker: 349-390; gdT VL: 391-702; Free energy: −302; gdT CAI: 0.868315362715317; ORF count: 10.

SEQ ID NO: 117 is the sequence name for JAML scFv (88). It is a gdT Targeting scFv construct. It is a DNA sequence. The sequence is:

GACGTGCAGCTGGTGGAGTCCGGGGCTGAGCTGGTGAGGCCCGGGGCCTCCAAGCTGAG
CTGCAAGGCCCTGGCCTACACCTTCACCGACTACGAGATGCACTGGGTGAAGCAGACCC
CGGTGCACGGGCTGGAGTGGATCGGGATCATCCATCCCGGGTCCGGCGGCACCGTGTAC
AACCAGAAGTTCAAGGGGAAGGCCACGCTGACCGCCGACAAGTCCAGCTCCACCGCCTA
CATGGAGCTGTCCAGCCTGACCTCCGAGGACTCCACCGTGTACTACTGCACCCGGCGGC
GGTACTACGGGTCCTCCTACAACTGGTACTTCGACGTGTGGGGCGCCGGGAACGGCGGA
TCAGGCGGCTCCGGAGGCTCAGGAGGCTCCGGCGGCGTGCTGACCCAGAGCCCCGCTAG
CCTGGCCGCCTCCGTCGGGGAGACCGTGACCATCACCTGCCGGGCCTCCGAGAACATCT
ACTACTCCCTGGCCTGGTACCAGCAGAAGCAGGGGAAGTCTCCCCAGCTGCTGATCTAC
AACGCCAACTCCCTGGAGGACGGGGTGCCCAGCCGGTTCTCCGGGTCCGGGTCCGGGAC
CCAGTACAGCCTGAAGATCAACAGCATGCAGCCCGAGGACACCGCCACCTACTTCTGCG
AGCAGACCTACGACGTGCCCCTGACCTTCGGGGCCGGGACCAAGCTGGAGCTG.

SEQ ID NO: 117 includes gdT scFv: 1-702; gdT VH: 349-390; gdT Linker: 349-390; gdT VL: 391-702; Free energy: −323.6; gdT CAI: 0.915740281407777; ORF count: 1.

SEQ ID NO: 118 is the sequence name for JAML scFv (84). It is a gdT Targeting scFv construct. It is a DNA sequence. The sequence is:

GATGTCCAGCTCGTGGAGTCCGGGGCTGAGCTCGTGAGGCCCGGGGCCTCGAAGCTGAG
CTGCAAGGCCCTGGCCTACACCTTCACGGACTACGAGATGCACTGGGTGAAGCAGACCC
CGGTGCACGGCCTGGAGTGGATCGGGATCATTCACCCCGGGTCCGGCGGCACCGTGTAC
AACCAGAAGTTCAAGGGCAAGGCCACGCTGACCGCGGACAAGTCCTCGTCCACGGCGTA
CATGGAGCTGAGCTCCCTGACCTCCGAGGACTCCACGGTGTACTACTGCACACGGCGGC
GGTACTACGGGAGCTCCTACAACTGGTACTTCGACGTCTGGGGCGCCGGGAACGGAGGC
TCTGGAGGCTCCGGAGGCTCTGGAGGCTCCGGCGGCGTCCTGACCCAGAGCCCCGCATC
CCTGGCGGCCTCCGTCGGGGAGACCGTGACCATCACCTGCAGGGCCTCGGAGAACATCT
ACTACTCCCTGGCGTGGTACCAGCAGAAGCAGGGGAAGTCCCCGCAGCTCCTGATCTAC
AACGCCAACTCCCTGGAGGACGGGGTGCCCTCGCGGTTCTCGGGGTCCGGGTCCGGGAC
CCAGTACTCCCTGAAGATCAACTCCATGCAGCCCGAGGACACCGCGACGTACTTCTGCG
AGCAGACCTACGACGTGCCCCTGACCTTCGGCGCCGGGACAAAGCTGGAGCTC.

SEQ ID NO: 118 includes gdT scFv: 1-702; gdT VH: 349-390; gdT Linker: 349-390; gdT VL: 391-702; Free energy: −328.2; gdT CAI: 0.850906150801958; ORF count: 2.

SEQ ID NO: 119 is the sequence name for JAML scFv (44). It is a gdT Targeting scFv construct. It is a DNA sequence. The sequence is:

GATGTGCAGCTGGTGGAGTCCGGGGCTGAGCTGGTGAGGCCCGGGGCCTCCAAGCTGTC
CTGCAAGGCCCTGGCCTACACCTTCACCGACTACGAGATGCACTGGGTGAAGCAGACGC
CCGTGCACGGGCTGGAGTGGATCGGGATCATTCACCCCGGGTCCGGCGGCACCGTGTAC
AACCAGAAGTTCAAGGGGAAGGCCACGCTGACCGCCGACAAGTCCTCCTCCACCGCCTA
CATGGAGCTGAGCTCCCTGACCTCCGAGGACTCCACCGTGTACTACTGCACCCGGCGGC
GGTACTACGGGTCCTCCTACAACTGGTACTTCGACGTGTGGGGCGCCGGGAACGGCGGA
TCTGGCGGCTCCGGCGGATCTGGCGGCTCTGGCGGCGTGCTGACCCAGAGCCCCGCTTCC
CTGGCCGCCTCCGTCGGGGAGACCGTGACCATCACCTGCCGGGCCTCCGAGAACATCTA
CTACTCCCTGGCCTGGTACCAGCAGAAGCAGGGGAAGTCTCCCCAGCTGCTGATCTACA
ACGCCAACTCCCTGGAGGACGGGGTGCCCAGCCGGTTCTCCGGGTCCGGGTCCGGGACC
CAGTACTCCCTGAAGATCAACTCCATGCAGCCCGAGGACACCGCCACCTACTTCTGCGA
GCAGACCTACGACGTGCCCCTGACCTTCGGGGCCGGGACCAAGCTGGAGCTG.

SEQ ID NO: 119 includes gdT scFv: 1-702; gdT VH: 349-390; gdT Linker: 349-390; gdT VL: 391-702; Free energy: −318.2; gdT CAI: 0.934766339108284; ORF count: 2.

SEQ ID NO: 120 is the sequence name for JAML scFv (23). It is a gdT Targeting scFv construct. It is a DNA sequence. The sequence is:

GACGTGCAGCTGGTGGAGTCCGGGGCCGAGCTGGTACGGCCAGGCGCCAGCAAGCTGTC
CTGCAAGGCGCTCGCCTACACCTTCACCGACTACGAGATGCACTGGGTGAAGCAGACGC
CCGTGCACGGGCTGGAGTGGATCGGGATCATACACCCCGGGTCCGGCGGCACCGTCTAC
AACCAGAAGTTCAAGGGGAAGGCTACCCTTACGGCCGACAAGTCCTCCTCTACCGCCTA
CATGGAGCTGTCCAGCCTGACGAGCGAGGACTCTACCGTCTACTACTGCACCCGCAGGC
GGTACTACGGGTCCAGCTACAACTGGTACTTCGACGTGTGGGGCGCCGGGAACGGCGGT
AGTGGCGGTAGCGGCGGTAGTGGCGGTAGCGGCGGCGTGCTGACCCAGTCGCCCGCTAG
CCTGGCCGCTAGCGTCGGGGAGACCGTCACCATCACCTGCCGGGCGAGCGAGAACATCT
ACTACTCGCTCGCCTGGTACCAGCAGAAGCAGGGGAAGAGCCCGCAGCTGCTGATCTAT
AACGCCAACAGCCTGGAGGACGGGGTGCCCAGCCGGTTCAGCGGGAGCGGGAGCGGGA
CCCAGTACTCCCTCAAGATCAACAGCATGCAGCCGGAGGATACCGCCACCTACTTCTGC
GAGCAGACGTACGACGTGCCCCTCACGTTCGGGGCCGGCACCAAGCTGGAGCTG.

SEQ ID NO: 120 includes gdT scFv: 1-702; gdT VH: 349-390; gdT Linker: 349-390; gdT VL: 391-702; Free energy: −314.5; gdT CAI: 0.828248130876822; ORF count: 3.

SEQ ID NO: 121 is the sequence name for JAML scFv (78). It is a gdT Targeting scFv construct. It is a DNA sequence. The sequence is:

GACGTGCAGCTCGTGGAGAGCGGCGCAGAGCTTGTGCGCCCCGGGGCCTCCAAGCTGTC
CTGCAAGGCGCTCGCCTACACCTTCACGGACTACGAGATGCACTGGGTGAAGCAGACCC
CTGTGCACGGCCTGGAGTGGATCGGGATCATTCACCCCGGGTCAGGAGGCACCGTGTAC
AACCAGAAGTTCAAGGGGAAGGCCACGCTGACCGCCGACAAGTCCTCCTCCACGGCCTA
CATGGAGCTCTCCTCGCTGACCTCCGAGGACTCCACGGTGTACTACTGCACGCGGAGGA
GGTACTACGGGTCCTCCTACAACTGGTACTTCGACGTGTGGGGCGCGGGGAATGGCGGC
TCAGGAGGCTCAGGAGGCTCAGGAGGCTCAGGCGGCGTGCTCACGCAGAGCCCCGCAA
GCCTCGCGGCGTCCGTCGGGGAGACCGTGACGATCACCTGCAGGGCCTCCGAGAACATC
TACTACTCCCTCGCGTGGTACCAGCAGAAGCAGGGGAAGTCCCCTCAGCTCCTCATCTAC
AACGCGAACTCCCTGGAGGACGGGGTGCCCTCAAGGTTCTCCGGGTCGGGGAGCGGCAC
GCAGTACTCCCTGAAGATCAACTCCATGCAGCCCGAGGACACCGCGACGTACTTCTGCG
AGCAGACGTACGACGTGCCCCTCACCTTCGGGGCCGGCACGAAGCTCGAACTG.

SEQ ID NO: 121 includes gdT scFv: 1-702; gdT VH: 349-390; gdT Linker: 349-390; gdT VL: 391-702; Free energy: −314.4; gdT CAI: 0.837369130789258; ORF count: 3.

SEQ ID NO: 122 is the sequence name for CDXAR ligand. It is a gdT Targeting ligand construct. It is an AA sequence. The sequence is:

LSITTPEEMIEKAKGETAYLPCKFTLSPEDQGPLDIEWLISPADNQKVDQVIILYSGDKIYDDY
YPDLKGRVHFTSNDLKSGDASINVTNLQLSDIGTYQCKVKKAPGVANKKIHLVVLVKPSGA
RCYVDGSEEIGSDFKIKCEPKEGSLPLQYEWQKLSDSQKMPTSWLAGKMCHLQRAVRPLPE
ATSAVIIHPWGPCLLPTWKDIPRLSITKYQVKTLNALLRVRLSHLLR.

SEQ ID NO: 122 includes gdT ligand: 1-233.

SEQ ID NO: 123 is the sequence name for CDXAR ligand. It is a gdT Targeting ligand construct. It is a DNA sequence. The sequence is:

CTGAGCATCACTACCCCTGAGGAGATGATTGAGAAGGCTAAGGGTGAGACAGCCTACCT
GCCCTGCAAGTTCACCCTGAGCCCTGAGGACCAGGGGCCCCTGGACATTGAGTGGCTGA
TCAGCCCAGCTGACAACCAGAAGGTGGACCAGGTCATCATCCTGTACTCAGGGGACAAG
ATCTATGATGACTACTACCCTGACCTGAAGGGCAGGGTGCACTTCACCAGCAATGACCT
GAAGTCAGGGGATGCCAGCATCAATGTGACCAACCTGCAGCTGTCTGACATAGGCACCT
ACCAGTGCAAGGTCAAGAAGGCCCCAGGGGTAGCCAACAAGAAGATCCACCTGGTGGT
GCTGGTCAAGCCCTCAGGGGCCAGGTGCTATGTGGATGGCTCTGAGGAGATAGGCTCTG
ACTTCAAGATCAAGTGTGAGCCCAAAGAGGGCAGCCTGCCCCTGCAGTATGAGTGGCAG
AAGCTGTCTGACAGCCAGAAGATGCCCACTAGCTGGCTGGCTGGCAAGATGTGCCACCT
GCAGAGGGCTGTCAGGCCCCTGCCTGAGGCCACCTCAGCTGTCATCATTCACCCCTGGGG
CCCCTGCCTGCTGCCTACCTGGAAGGACATCCCCAGGCTGAGCATCACTAAGTACCAGGT
CAAGACCCTCAATGCCCTGCTGAGGGTCAGGCTGAGCCACCTGCTGAGG.

SEQ ID NO: 123 includes gdT ligand: 1-233; Free energy: −284.1; gdT CAI: 0.86860152001121; ORF count: 7.

SEQ ID NO: 124 is the sequence name for CDXAR ligand (6). It is a gdT Targeting ligand construct. It is a DNA sequence. The sequence is:

CTGAGCATCACCACGCCCGAGGAGATGATCGAGAAGGCCAAGGGCGAGACCGCCTACCT
GCCCTGCAAGTTCACCCTGAGCCCCGAGGACCAGGGGCCCCTGGACATCGAGTGGCTGA
TCTCGCCCGCGGACAACCAGAAGGTGGACCAGGTGATCATCCTGTACTCCGGGGACAAG
ATCTACGACGACTACTACCCCGACCTGAAGGGGCGCGTGCACTTCACCTCCAACGACCT
GAAGTCCGGGGACGCCAGCATCAACGTGACCAACCTGCAGCTGTCCGACATCGGGACCT
ACCAGTGCAAGGTGAAGAAGGCGCCCGGGGTCGCCAACAAGAAGATCCACCTGGTGGT
GCTGGTGAAGCCCAGCGGGGCCAGGTGCTACGTGGACGGGAGCGAGGAGATCGGGTCC
GACTTCAAGATCAAGTGCGAGCCCAAAGAGGGGTCCCTGCCCCTGCAGTACGAGTGGCA
GAAGCTGTCCGACAGCCAGAAGATGCCGACCTCCTGGCTGGCCGGGAAGATGTGCCACC
TGCAGCGGGCCGTGAGGCCCCTGCCTGAGGCCACCAGCGCCGTGATCATTCACCCCTGG
GGCCCCTGCCTGCTGCCAACATGGAAGGACATCCCGCGGCTGTCCATCACCAAGTACCA
GGTGAAGACCCTGAACGCCCTGCTGCGGGTGCGGCTGTCGCACCTGCTGCGG.

SEQ ID NO: 124 includes gdT ligand: 1-233; Free energy: −291.4; gdT CAI: 0.912300179663014; ORF count: 1.

SEQ ID NO: 125 is the sequence name for CDXAR ligand (57). It is a gdT Targeting ligand construct. It is a DNA sequence. The sequence is:

CTCTCGATCACCACACCCGAGGAGATGATCGAGAAGGCGAAGGGCGAGACCGCCTACTT
GCCCTGCAAGTTCACCCTCTCGCCCGAGGACCAGGGGCCCCTCGATATCGAGTGGCTCAT
CTCGCCCGCGGACAACCAAAAGGTGGACCAGGTGATCATCCTCTATAGTGGGGACAAGA
TCTACGACGACTACTACCCCGATCTCAAGGGGCGCGTCCACTTCACCTCCAACGACCTCA
AGAGCGGGGACGCCTCGATCAACGTGACCAACCTCCAATTGAGCGACATCGGGACCTAC
CAGTGCAAGGTGAAGAAGGCGCCCGGGGTCGCCAACAAGAAGATCCACCTCGTGGTGCT
CGTGAAGCCCAGTGGGGCGCGGTGCTACGTGGATGGGTCGGAGGAGATCGGGAGCGAC
TTCAAGATCAAGTGCGAGCCCAAAGAGGGCTCGCTACCCCTACAATACGAGTGGCAGAA
GTTGAGCGACTCCCAAAAGATGCCCACTAGCTGGCTCGCGGGCAAGATGTGCCACCTCC
AGCGCGCCGTGCGACCCCTACCCGAGGCCACGAGCGCGGTCATCATACACCCCTGGGGC
CCCTGCCTACTCCCCACTTGGAAGGACATCCCGCGCCTCTCGATCACCAAGTACCAGGTG
AAGACGCTCAACGCGCTCTTGCGCGTGCGCCTCTCGCACCTCTTGAGG.

SEQ ID NO: 125 includes gdT ligand: 1-233; Free energy: −290.9; gdT CAI: 0.776501808272779; ORF count: 4.

SEQ ID NO: 126 is the sequence name for CDXAR ligand (56). It is a gdT Targeting ligand construct. It is a DNA sequence. The sequence is:

CTGAGCATCACCACGCCGGAGGAGATGATCGAGAAGGCGAAGGGCGAGACCGCGTACC
TGCCCTGCAAGTTCACCCTGAGCCCGGAGGACCAGGGGCCGCTGGACATCGAGTGGCTG
ATCAGCCCCGCGGACAACCAGAAGGTGGACCAGGTGATCATCCTGTACAGCGGGGACAA
GATCTACGACGACTACTACCCGGACCTGAAGGGGCGCGTGCACTTCACCTCCAACGACC
TGAAGAGCGGGGACGCCAGCATCAACGTGACCAACCTGCAGCTGAGCGACATCGGGAC
CTACCAGTGCAAGGTGAAGAAGGCGCCCGGGGTCGCGAACAAGAAGATCCACCTGGTG
GTGCTGGTGAAGCCCAGCGGGGCCCGGTGCTACGTGGACGGGTCCGAGGAGATCGGGTC
CGACTTCAAGATCAAGTGCGAGCCGAAAGAGGGGTCCCTGCCGCTGCAGTACGAGTGGC
AGAAGCTGAGCGACAGCCAGAAGATGCCGACCAGCTGGCTGGCCGGGAAGATGTGCCA
CCTGCAGCGGGCTGTCCGGCCGCTCCCTGAGGCTACCTCCGCGGTGATCATCCATCCCTG
GGGCCCCTGCCTGCTCCCGACCTGGAAGGACATCCCGCGGCTGAGCATCACCAAGTACC
AGGTGAAGACCCTGAACGCGCTGCTCCGGGTCCGGCTGAGCCACCTGCTCCGG.

SEQ ID NO: 126 includes gdT ligand: 1-233; Free energy: −287.5; gdT CAI: 0.835151871227941; ORF count: 2.

SEQ ID NO: 127 is the sequence name for CDXAR ligand (73). It is a gdT Targeting ligand construct. It is a DNA sequence. The sequence is:

CTCTCGATCACCACGCCCGAGGAGATGATCGAGAAGGCCAAGGGCGAGACCGCCTACTT
GCCCTGCAAGTTCACCCTCTCGCCCGAGGACCAGGGGCCCCTCGACATCGAGTGGCTCAT
CTCGCCCGCGGACAACCAGAAGGTGGACCAGGTGATCATCCTCTACTCGGGCGACAAGA
TCTACGACGACTACTACCCCGACCTCAAGGGGCGCGTGCACTTCACGAGCAACGACCTC
AAGAGCGGGGACGCCTCGATCAACGTGACCAACCTCCAGCTCTCCGACATCGGGACCTA
CCAGTGCAAGGTGAAGAAGGCGCCCGGGGTCGCCAACAAGAAGATCCACCTCGTGGTGT
TGGTGAAGCCCAGCGGGGCGCGGTGCTACGTGGACGGGAGCGAGGAGATCGGGAGCGA
CTTCAAGATCAAGTGCGAGCCCAAAGAGGGGTCGCTCCCGCTCCAGTACGAGTGGCAGA
AGCTGAGCGACTCCCAGAAGATGCCCACGAGCTGGCTCGCGGGGAAGATGTGCCACCTC
CAGCGCGCGGTTCGCCCACTCCCCGAGGCCACGAGCGCGGTGATCATTCACCCATGGGG
CCCCTGCCTCTTGCCCACGTGGAAGGACATCCCGCGCCTCTCGATCACCAAGTACCAGGT
GAAGACCCTCAACGCGCTCCTGCGCGTGCGCCTCTCCCACTTGTTGAGG.

SEQ ID NO: 127 includes gdT ligand: 1-233; Free energy: −287.4; gdT CAI: 0.810082913563651; ORF count: 0.

SEQ ID NO: 128 is the sequence name for CDXAR ligand (63). It is a gdT Targeting ligand construct. It is a DNA sequence. The sequence is:

CTGAGCATCACGACGCCCGAGGAGATGATCGAGAAGGCCAAAGGGGAGACCGCCTACC
TGCCCTGCAAGTTCACCCTGAGCCCCGAGGACCAGGGGCCCCTGGACATCGAGTGGCTG
ATCTCCCCGGCGGACAACCAGAAGGTCGACCAGGTGATCATCCTGTACTCCGGGGACAA
GATCTACGACGACTACTACCCCGACCTGAAGGGCCGGGTGCACTTCACGTCGAACGACC
TGAAGTCCGGGGACGCCTCGATCAACGTGACCAACCTGCAGCTGTCCGACATCGGGACC
TACCAGTGCAAGGTGAAGAAGGCGCCCGGCGTGGCCAACAAGAAGATCCACCTGGTGGT
CCTCGTGAAGCCCAGCGGGGCGCGGTGCTACGTGGACGGCAGCGAGGAGATCGGGTCG
GACTTCAAGATCAAGTGCGAGCCCAAAGAGGGCAGCCTGCCCCTGCAGTACGAGTGGCA
GAAGCTGTCCGACAGCCAGAAGATGCCGACCTCCTGGCTGGCCGGCAAGATGTGCCACC
TGCAGCGCGCCGTGAGGCCACTCCCCGAGGCGACCAGCGCGGTGATCATTCACCCCTGG
GGCCCCTGCCTGCTGCCAACCTGGAAGGACATCCCGCGGCTGTCCATCACGAAGTACCA
GGTGAAGACCCTGAACGCCCTGCTGCGCGTCCGGCTGAGCCACCTGCTGCGC.

SEQ ID NO: 128 includes gdT ligand: 1-233; Free energy: −286; gdT CAI: 0.861258772692867; ORF count: 0.

SEQ ID NO: 129 is the sequence name for CD5 scFv. It is a gdT Targeting scFv construct. It is an AA sequence. The sequence is:

EIQLVQSGGGLVKPGGSVRISCAASGYTFTNYGMNWVRQAPGKGLEWMGWINTHTGEPTY
ADSFKGRFTFSLDDSKNTAYLQINSLRAEDTAVYFCTRRGYDWYFDVWGQGTTVTVSSGGG
GSGGGGSGGGGSDIQMTQSPSSLSASVGDRVTITCRASQDINSYLSWFQQKPGKAPKTLIYRA
NRLESGVPSRFSGSGSGTDYTLTISSLQYEDFGIYYCQQYDESPWTFGGGTKLEIK.

SEQ ID NO: 129 includes gdT scFv: 1-240; gdT VH: 119-133; gdT Linker: 119-133; gdT VL: 134-240.

SEQ ID NO: 130 is the sequence name for CD5 scFv. It is a gdT Targeting scFv construct. It is a DNA sequence. The sequence is:

GAGATCCAGCTGGTGCAGTCTGGGGGGGGGCTGGTGAAGCCTGGGGGGTCAGTGAGGAT
CAGCTGTGCTGCCTCAGGGTACACCTTCACCAACTATGGGATGAACTGGGTGAGGCAGG
CCCCAGGGAAGGGGCTGGAGTGGATGGGGTGGATCAACACCCACACTGGGGAGCCCAC
CTATGCTGACAGCTTCAAGGGGAGGTTCACCTTCAGCCTGGATGACTCCAAGAACACAG
CCTACCTGCAGATCAACTCCCTGAGGGCTGAGGACACAGCTGTGTACTTCTGCACCAGG
AGGGGGTATGACTGGTACTTTGATGTGTGGGGGCAGGGGACCACAGTGACAGTGTCCTC
TGGGGGGGGGGGGTCTGGGGGGGGGGGGTCTGGGGGGGGGGGGTCTGACATCCAGATG
ACCCAGTCCCCCTCCAGCCTGTCTGCCTCAGTGGGGGACAGGGTGACCATCACCTGCAG
GGCCTCCCAGGACATCAACTCCTACCTGTCCTGGTTCCAGCAGAAGCCTGGGAAGGCCC
CCAAGACCCTGATCTACAGGGCCAACAGGCTGGAGTCTGGGGTGCCCTCCAGGTTCTCA
GGGTCTGGGTCTGGGACAGACTACACCCTGACCATCTCCAGCCTGCAGTATGAGGACTTT
GGGATCTACTACTGCCAGCAGTATGATGAGTCCCCCTGGACCTTTGGGGGGGGGACCAA
GCTGGAGATCAAG.

SEQ ID NO: 130 includes gdT scFv: 1-720; gdT VH: 355-399; gdT Linker: 355-399; gdT VL: 400-720; Free energy: −346.2; gdT CAI: 0.943955363575702; ORF count: 8.

SEQ ID NO: 131 is the sequence name for CD5 scFv (11). It is a gdT Targeting scFv construct. It is a DNA sequence. The sequence is:

GAGATCCAGCTGGTGCAGTCCGGCGGCGGCCTGGTGAAACCCGGCGGCTCCGTGCGGAT
CTCCTGCGCCGCCTCCGGGTACACCTTCACCAACTACGGGATGAACTGGGTGCGGCAGG
CTCCCGGGAAGGGGCTGGAGTGGATGGGCTGGATCAACACCCACACCGGGGAGCCCAC
ATACGCCGACTCCTTCAAGGGGCGCTTCACCTTCTCCCTGGACGACTCCAAGAACACCGC
CTACCTGCAGATCAACTCCCTGCGGGCCGAGGACACCGCCGTGTACTTCTGCACCCGGCG
CGGGTACGACTGGTACTTCGACGTGTGGGGCCAGGGGACCACCGTGACCGTGTCCTCTG
GCGGCGGAGGTTCCGGCGGAGGAGGCTCCGGAGGAGGCGGCAGCGACATCCAGATGAC
CCAGAGCCCCAGCAGCCTGTCCGCCTCCGTCGGGGACCGGGTGACCATCACCTGCCGGG
CCAGCCAGGACATCAACAGCTACCTGTCCTGGTTCCAGCAGAAGCCCGGGAAGGCTCCC
AAGACCCTGATCTACCGGGCCAACCGGCTGGAGAGCGGGGTGCCCAGCCGGTTCAGCGG
GTCCGGGTCCGGGACCGACTACACCCTGACCATCAGCTCCCTGCAGTACGAGGACTTCG
GGATCTACTACTGCCAGCAGTACGACGAGTCCCCATGGACCTTCGGCGGCGGCACCAAG
CTGGAGATCAAG.

SEQ ID NO: 131 includes gdT scFv: 1-720; gdT VH: 355-399; gdT Linker: 355-399; gdT VL: 400-720; Free energy: −345.9; gdT CAI: 0.885961949519618; ORF count: 1.

SEQ ID NO: 132 is the sequence name for CD5 scFv (9). It is a gdT Targeting scFv construct. It is a DNA sequence. The sequence is:

GAGATCCAGCTGGTGCAGTCCGGCGGCGGCCTGGTGAAACCCGGCGGCTCCGTGCGGAT
CTCCTGCGCCGCCTCCGGGTACACCTTCACCAACTACGGGATGAACTGGGTGCGGCAGG
CTCCCGGGAAGGGGCTGGAGTGGATGGGCTGGATCAACACCCACACCGGGGAGCCCAC
GTACGCCGACTCCTTCAAGGGGCGCTTCACCTTCTCCCTCGACGACTCCAAGAACACCGC
CTACCTGCAGATCAACTCCCTGCGGGCCGAGGACACCGCCGTGTACTTCTGCACCCGGCG
CGGGTACGACTGGTACTTCGACGTGTGGGGCCAGGGGACCACCGTGACCGTGTCCTCCG
GAGGCGGCGGCTCTGGAGGAGGCGGCTCTGGAGGCGGAGGCTCCGACATCCAGATGAC
CCAGAGCCCCAGCTCCCTCTCCGCCTCCGTCGGGGACCGGGTGACCATCACCTGCCGCGC
CAGCCAGGACATCAACTCCTACCTCTCCTGGTTCCAGCAGAAGCCCGGGAAGGCGCCCA
AGACCCTCATCTACCGCGCCAACCGGCTGGAGTCCGGGGTGCCCAGCCGCTTCTCCGGGT
CCGGGTCCGGGACCGACTACACCCTCACCATCTCCAGCCTGCAGTACGAGGACTTCGGG
ATCTACTACTGCCAGCAGTACGACGAGAGCCCCTGGACCTTCGGCGGCGGCACCAAGCT
GGAGATCAAG.

SEQ ID NO: 132 includes gdT scFv: 1-720; gdT VH: 355-399; gdT Linker: 355-399; gdT VL: 400-720; Free energy: −344.1; gdT CAI: 0.8842887077719; ORF count: 1.

SEQ ID NO: 133 is the sequence name for CD5 scFv (41). It is a gdT Targeting scFv construct. It is a DNA sequence. The sequence is:

GAAATCCAGCTGGTGCAGTCCGGCGGCGGCCTGGTGAAACCCGGCGGCTCCGTGCGGAT
CAGCTGCGCGGCCAGCGGGTACACCTTTACGAACTACGGGATGAACTGGGTGCGCCAGG
CACCCGGCAAGGGGCTCGAATGGATGGGCTGGATCAACACGCACACCGGGGAGCCAAC
CTACGCGGATAGCTTCAAGGGGCGCTTCACGTTCAGCCTGGACGACTCGAAGAACACCG
CCTACCTGCAGATCAACTCGCTGCGCGCCGAGGACACCGCGGTGTACTTCTGCACGCGG
CGCGGGTACGACTGGTACTTCGACGTGTGGGGCCAGGGGACCACCGTGACGGTGTCCTC
CGGAGGAGGCGGCTCCGGAGGAGGCGGCTCAGGAGGCGGCGGATCGGACATCCAGATG
ACCCAGTCGCCCAGCAGCCTGTCGGCCAGCGTGGGCGACCGGGTGACCATCACCTGCCG
GGCGTCCCAGGATATCAACAGCTACCTGTCGTGGTTCCAGCAGAAGCCCGGCAAGGCGC
CCAAAACGCTGATCTACCGGGCCAACCGGCTGGAAAGCGGGGTGCCCAGCCGGTTCAGC
GGGTCGGGCAGCGGGACGGACTACACGCTGACCATCAGCAGCCTGCAGTACGAGGACTT
CGGGATATACTACTGCCAGCAGTACGACGAGTCGCCCTGGACGTTCGGCGGAGGCACCA
AGCTGGAAATCAAG.

SEQ ID NO: 133 includes gdT scFv: 1-720; gdT VH: 355-399; gdT Linker: 355-399; gdT VL: 400-720; Free energy: −342.6; gdT CAI: 0.78445426326926; ORF count: 1.

SEQ ID NO: 134 is the sequence name for CD5 scFv (21). It is a gdT Targeting scFv construct. It is a DNA sequence. The sequence is:

GAGATCCAGCTCGTGCAGTCCGGCGGCGGCCTCGTGAAACCCGGCGGCAGCGTGCGCAT
CTCGTGCGCCGCGTCCGGGTACACGTTCACGAACTACGGCATGAACTGGGTGCGGCAGG
CACCCGGCAAGGGGCTGGAGTGGATGGGCTGGATCAACACGCACACCGGCGAGCCCAC
GTACGCCGACTCGTTCAAGGGGCGCTTCACGTTCAGCCTCGACGACTCCAAGAACACCG
CGTACCTGCAGATCAACAGCCTGCGCGCCGAGGACACCGCCGTGTACTTCTGCACGCGG
CGCGGGTACGACTGGTACTTCGACGTGTGGGGCCAGGGCACGACCGTGACCGTGTCGTC
TGGCGGCGGTGGATCAGGCGGCGGAGGCTCAGGCGGTGGAGGCTCCGACATCCAGATG
ACGCAGTCGCCGTCCAGCCTGTCCGCGTCCGTCGGCGACCGCGTGACGATCACGTGCCG
CGCGTCGCAGGACATCAACTCGTACCTGTCGTGGTTCCAGCAGAAGCCCGGCAAGGCGC
CCAAGACGCTGATCTACCGCGCGAACCGGCTGGAGTCCGGCGTGCCGTCGCGGTTCAGC
GGGTCCGGGTCCGGCACCGACTACACGCTGACGATCAGCAGCCTGCAGTACGAGGACTT
CGGCATCTACTACTGCCAGCAGTACGACGAGTCGCCGTGGACGTTCGGCGGCGGCACGA
AGCTGGAGATCAAG.

SEQ ID NO: 134 includes gdT scFv: 1-720; gdT VH: 355-399; gdT Linker: 355-399; gdT VL: 400-720; Free energy: −338.3; gdT CAI: 0.760359380692982; ORF count: 1.

SEQ ID NO: 135 is the sequence name for CD5 scFv (29). It is a gdT Targeting scFv construct. It is a DNA sequence. The sequence is:

GAGATCCAGCTCGTGCAGTCCGGCGGCGGCCTGGTCAAGCCTGGCGGCTCCGTGCGCAT
CTCCTGCGCGGCCTCGGGGTACACCTTCACGAACTACGGCATGAACTGGGTGCGCCAGG
CCCCGGGGAAAGGCCTGGAGTGGATGGGCTGGATCAACACGCACACCGGGGAGCCCAC
GTACGCCGACTCCTTCAAGGGGCGCTTCACGTTCTCCCTGGACGACTCGAAGAACACGG
CCTACCTGCAGATCAACTCCCTGCGCGCCGAGGACACCGCGGTGTACTTCTGCACGCGCC
GGGGCTACGACTGGTACTTCGACGTCTGGGGCCAGGGGACGACGGTCACCGTGAGCTCA
GGCGGCGGAGGAAGCGGAGGCGGAGGCTCAGGCGGAGGCGGCTCGGACATCCAGATGA
CGCAGAGCCCGTCCTCGCTGAGCGCCTCCGTGGGCGACCGCGTGACGATCACCTGCCGG
GCGTCCCAGGACATCAACTCCTACCTGTCGTGGTTCCAGCAGAAGCCCGGGAAGGCCCC
GAAGACCCTGATCTACCGGGCGAACCGCCTGGAGTCCGGCGTGCCCTCGCGGTTCTCCG
GGTCGGGCTCGGGGACGGACTACACGCTGACCATCTCCTCGCTGCAGTACGAGGACTTC
GGCATCTACTACTGCCAGCAGTACGACGAGTCCCCGTGGACCTTCGGCGGCGGCACGAA
GCTGGAGATCAAG.

SEQ ID NO: 135 includes gdT scFv: 1-720; gdT VH: 355-399; gdT Linker: 355-399; gdT VL: 400-720; Free energy: −334.2; gdT CAI: 0.774216148765874; ORF count: 1.

SEQ ID NO: 136 is the sequence name for IL2r ligand. It is a gdT Targeting ligand construct. It is an AA sequence. The sequence is:

APTSSSTKKTQLQLEHLLLDLQMILNGINNYKNPKLTRMLTFKFYMPKKATELKHLQCLEEE
LKPLEEVLNLAQSKNFHLRPRDLISNINVIVLELKGSETTFMCEYADETATIVEFLNRWITFCQ
SIISTLT.

SEQ ID NO: 136 includes gdT ligand: 1-333.

SEQ ID NO: 137 is the sequence name for IL2r ligand. It is a gdT Targeting ligand construct. It is a DNA sequence. The sequence is:

GCACCAACAAGCAGTAGCACCAAAAAGACGCAGCTTCAGTTAGAGCACCTCCTACTCGA
CCTACAGATGATATTGAATGGTATTAATAACTACAAAAATCCTAAATTGACTCGAATGTT
GACATTTAAATTTTATATGCCCAAAAAGGCAACCGAACTCAAGCATCTGCAGTGCCTGG
AGGAGGAACTCAAGCCACTTGAAGAGGTCCTGAACCTGGCTCAGTCAAAAAATTTTCAT
CTGCGCCCCCGGGACTTAATCAGCAATATCAACGTGATTGTTCTGGAGCTCAAGGGGTCT
GAGACCACTTTTATGTGTGAATACGCTGATGAAACTGCGACAATCGTCGAGTTCCTCAAT
AGATGGATCACTTTCTGTCAATCCATTATTAGCACCCTGACC.

SEQ ID NO: 137 includes gdT ligand: 1-459; Free energy: −92.6; gdT CAI: 0.749692927682812; ORF count: 2.

SEQ ID NO: 138 is the sequence name for IL2r ligand. It is a gdT Targeting ligand construct. It is a DNA sequence. The sequence is:

GCTCCTACCAGCTCCAGCACCAAGAAGACCCAGCTGCAGCTGGAGCACCTGCTGCTGGA
CCTGCAGATGATCCTGAACGGGATCAACAACTACAAGAACCCCAAGCTGACCCGGATGC
TGACCTTCAAGTTCTACATGCCCAAGAAGGCCACCGAGCTGAAGCACCTGCAGTGCCTG
GAGGAGGAGCTGAAGCCCCTGGAGGAGGTGCTGAACCTGGCCCAGAGCAAGAACTTCC
ACCTGCGGCCCCGGGACCTGATCAGCAACATCAACGTGATCGTGCTGGAGCTGAAGGGG
TCCGAGACCACCTTCATGTGCGAGTACGCCGACGAGACCGCCACCATCGTGGAGTTCCT
GAACCGCTGGATCACCTTCTGCCAGAGCATCATCTCCACGCTGACC.

SEQ ID NO: 138 includes gdT ligand: 1-459; Free energy: −140.2; gdT CAI: 0.948946971021626; ORF count: 0.

SEQ ID NO: 139 is the sequence name for IL2r ligand. It is a gdT Targeting ligand construct. It is a DNA sequence. The sequence is:

GCCCCGACCAGCAGCAGCACCAAGAAGACGCAGCTGCAGCTGGAGCACCTGCTGCTGGA
CCTGCAGATGATCCTGAACGGGATCAACAACTACAAGAACCCCAAGCTGACCCGGATGC
TGACCTTCAAGTTCTACATGCCCAAGAAGGCGACCGAGCTGAAGCACCTGCAGTGCCTG
GAGGAGGAGCTGAAGCCCCTGGAGGAGGTGCTGAACCTGGCCCAGTCCAAGAACTTCCA
CCTGCGGCCCCGGGACCTGATCAGCAACATCAACGTGATCGTGCTGGAGCTGAAGGGGT
CCGAGACCACCTTCATGTGCGAGTACGCGGACGAGACCGCCACAATCGTGGAGTTCCTG
AACCGCTGGATCACCTTCTGCCAGTCCATCATCAGCACCCTGACG.

SEQ ID NO: 139 includes gdT ligand: 1-459; Free energy: −142.5; gdT CAI: 0.925399648745741; ORF count: 0.

SEQ ID NO: 140 is the sequence name for IL2r ligand. It is a gdT Targeting ligand construct. It is a DNA sequence. The sequence is:

GCCCCAACCTCCTCCTCCACCAAGAAGACCCAGCTGCAGCTGGAGCACCTGCTGCTGGA
CCTGCAGATGATCCTGAACGGGATCAACAACTACAAGAACCCCAAGCTGACCCGGATGC
TCACCTTCAAGTTCTACATGCCCAAGAAGGCCACCGAGCTGAAGCACCTGCAGTGCCTG
GAGGAGGAGCTGAAGCCCCTGGAGGAGGTGCTGAACCTGGCCCAGTCCAAGAACTTCCA
CCTGCGGCCCCGGGACCTGATCTCCAACATCAACGTGATCGTGCTGGAGCTGAAGGGGT
CCGAGACCACCTTCATGTGCGAGTACGCAGATGAGACGGCTACAATCGTGGAGTTCCTG
AACAGGTGGATCACCTTCTGCCAGTCCATCATCTCCACCTTGACA.

SEQ ID NO: 140 includes gdT ligand: 1-459; Free energy: −139.3; gdT CAI: 0.966990185804835; ORF count: 1.

SEQ ID NO: 141 is the sequence name for IL2r ligand. It is a gdT Targeting ligand construct. It is a DNA sequence. The sequence is:

GCGCCCACGTCCTCCTCCACCAAGAAGACCCAGCTGCAGCTGGAGCACCTGCTGCTGGA
CCTGCAGATGATCCTGAACGGGATCAACAACTACAAGAACCCCAAGCTGACCCGGATGC
TGACCTTCAAGTTCTACATGCCCAAGAAGGCCACCGAGCTGAAGCACCTGCAGTGCCTG
GAGGAGGAGCTGAAGCCCCTGGAGGAGGTGCTGAACCTGGCCCAGTCCAAGAACTTCCA
CCTGCGGCCCCGGGACCTGATCTCCAACATCAACGTGATCGTGCTGGAGCTGAAGGGGT
CCGAGACCACCTTCATGTGCGAGTACGCCGACGAGACCGCCACCATCGTGGAGTTCCTG
AACCGCTGGATCACCTTCTGCCAGAGCATCATCAGCACCCTCACC.

SEQ ID NO: 141 includes gdT ligand: 1-459; Free energy: −136; gdT CAI: 0.957989396711122; ORF count: 0.

SEQ ID NO: 142 is the sequence name for IL2r ligand. It is a gdT Targeting ligand construct. It is a DNA sequence. The sequence is:

GCCCCTACCTCCTCCTCCACCAAGAAGACCCAGCTCCAGCTGGAGCACCTCCTCCTGGAC
CTCCAGATGATCCTCAACGGGATCAACAACTACAAGAACCCCAAGCTCACCCGGATGCT
GACCTTCAAGTTCTACATGCCCAAGAAGGCCACCGAGCTGAAGCACCTCCAGTGCCTGG
AGGAGGAGCTGAAGCCCCTGGAGGAGGTGCTGAACCTGGCCCAGTCCAAGAACTTCCAC
CTCCGGCCCAGGGACCTGATCTCCAACATCAACGTGATCGTCCTGGAGCTGAAGGGGTC
CGAGACCACCTTCATGTGCGAGTACGCCGATGAGACAGCCACCATCGTGGAGTTCCTCA
ACAGGTGGATCACCTTCTGCCAGTCCATCATCAGCACCCTCACC.

SEQ ID NO: 142 includes gdT ligand: 1-459; Free energy: −135.6; gdT CAI: 0.950256495713545; ORF count: 1.

SEQ ID NO: 143 is the sequence name for IL15r ligand. It is a gdT Targeting ligand construct. It is an AA sequence. The sequence is:

NWVNVISDLKKIEDLIQSMHIDATLYTESDVHPSCKVTAMKCFLLELQVISLESGDASIHDTV
ENLIILANNSLSSNGNVTESGCKECEELEEKNIKEFLQSFVHIVQMFINTS.

SEQ ID NO: 143 includes gdT ligand: 1-114.

SEQ ID NO: 144 is the sequence name for IL15r ligand. It is a gdT Targeting ligand construct. It is a DNA sequence. The sequence is:

AACTGGGTTAACGTGATCAGCGATCTGAAGAAGATTGAAGATCTCATACAATCCATGCA
CATCGACGCTACCCTGTATACAGAGTCCGACGTTCACCCTAGCTGTAAGGTGACTGCCAT
GAAGTGCTTTTTACTGGAACTGCAGGTAATCAGTCTGGAGTCTGGTGATGCCTCAATTCA
CGACACGGTAGAGAATCTAATAATCCTTGCCAACAACTCTTTGAGTTCCAATGGCAATGT
GACAGAATCTGGCTGCAAGGAGTGTGAAGAGCTTGAAGAGAAAAACATTAAAGAGTTC
CTGCAATCCTTCGTGCATATAGTGCAGATGTTCATCAACACCTCG.

SEQ ID NO: 144 includes gdT ligand: 1-342; Free energy: −86.8; gdT CAI: 0.773146219024413; ORF count: 3.

SEQ ID NO: 145 is the sequence name for IL15r ligand (12). It is a gdT Targeting ligand construct. It is a DNA sequence. The sequence is:

AACTGGGTGAACGTGATCAGCGATCTGAAGAAGATCGAGGACCTGATCCAGTCCATGCA
CATCGACGCTACCCTGTACACCGAGTCGGACGTTCACCCCAGCTGCAAGGTGACCGCGA
TGAAGTGCTTCCTGCTCGAACTGCAGGTGATCAGCCTGGAGAGCGGGGACGCGAGCATC
CACGATACGGTGGAGAACCTGATCATCCTGGCCAACAACTCGCTCAGCTCGAACGGGAA
CGTGACCGAGAGCGGGTGCAAGGAGTGCGAGGAGCTGGAGGAGAAGAACATCAAGGAG
TTCCTCCAGTCGTTCGTGCACATCGTGCAGATGTTCATCAACACCTCC.

SEQ ID NO: 145 includes gdT ligand: 1-342; Free energy: −125.8; gdT CAI: 0.877487412777548; ORF count: 0.

SEQ ID NO: 146 is the sequence name for IL15r ligand (31). It is a gdT Targeting ligand construct. It is a DNA sequence. The sequence is:

AACTGGGTGAACGTCATCTCCGACCTGAAGAAGATCGAGGATCTGATCCAGTCGATGCA
CATCGACGCGACGCTCTACACCGAGTCGGACGTTCACCCCTCGTGCAAGGTCACGGCGA
TGAAGTGCTTCCTCCTGGAGCTGCAGGTGATCTCCCTGGAGTCGGGCGACGCCTCGATCC
ACGACACGGTCGAGAACCTGATCATCCTCGCGAACAACTCCCTCTCGTCCAACGGGAAC
GTGACCGAGAGCGGGTGCAAGGAGTGCGAGGAGCTGGAGGAGAAGAACATCAAGGAGT
TCCTCCAGTCGTTCGTCCACATCGTCCAGATGTTCATCAACACCTCC.

SEQ ID NO: 146 includes gdT ligand: 1-342; Free energy: −124.3; gdT CAI: 0.830363621275029; ORF count: 0.

SEQ ID NO: 147 is the sequence name for IL15r ligand (29). It is a gdT Targeting ligand construct. It is a DNA sequence. The sequence is:

AACTGGGTGAACGTGATCAGCGACCTGAAGAAGATCGAGGACCTGATCCAGTCCATGCA
CATCGACGCCACGCTGTACACCGAGTCCGACGTGCACCCCAGCTGCAAGGTGACCGCCA
TGAAGTGCTTCCTGCTGGAGCTGCAGGTGATCTCCCTGGAGTCCGGGGACGCCTCCATCC
ACGACACCGTGGAGAACCTGATCATCCTGGCCAACAACTCCCTGTCCTCCAACGGGAAC
GTGACCGAGTCCGGGTGCAAGGAGTGCGAGGAGCTGGAGGAGAAGAACATCAAGGAGT
TCCTGCAGAGCTTCGTGCACATCGTGCAGATGTTCATCAACACGTCG.

SEQ ID NO: 147 includes gdT ligand: 1-342; Free energy: −123.2; gdT CAI: 0.953480352366457; ORF count: 0.

SEQ ID NO: 148 is the sequence name for IL15r ligand (29). It is a gdT Targeting ligand construct. It is a DNA sequence. The sequence is:

AATTGGGTGAACGTCATCTCCGACCTCAAGAAGATCGAGGACCTCATCCAGTCCATGCA
CATCGACGCCACGCTCTACACGGAGTCCGACGTGCACCCGTCCTGCAAGGTGACGGCCA
TGAAGTGCTTCCTGCTGGAGCTGCAGGTCATCTCCTTGGAGTCCGGGGACGCCTCCATCC
ACGACACCGTCGAGAACCTCATCATCCTGGCCAACAACTCCTTGAGCTCCAACGGGAAC
GTGACGGAGTCCGGCTGCAAGGAGTGCGAGGAGCTGGAGGAGAAGAACATCAAGGAGT
TCCTGCAGTCCTTCGTGCACATCGTGCAGATGTTCATCAACACGTCC.

SEQ ID NO: 148 includes gdT ligand: 1-342; Free energy: −122.3; gdT CAI: 0.905077015244385; ORF count: 0.

SEQ ID NO: 149 is the sequence name for IL15r ligand (32). It is a gdT Targeting ligand construct. It is a DNA sequence. The sequence is:

AACTGGGTGAACGTGATCTCCGACCTCAAGAAGATCGAGGACCTCATCCAGTCGATGCA
CATCGACGCGACCCTCTACACGGAGAGCGACGTCCATCCGAGCTGCAAGGTGACCGCGA
TGAAGTGCTTCCTCCTGGAGCTCCAGGTGATCTCCCTGGAGTCCGGGGACGCGAGCATCC
ACGACACCGTCGAGAACCTGATCATCCTCGCGAACAACTCGCTCTCCTCGAACGGGAAC
GTCACCGAGAGCGGCTGCAAGGAGTGCGAGGAGCTCGAAGAGAAGAACATCAAGGAGT
TCCTCCAGAGCTTCGTCCACATCGTCCAGATGTTCATCAACACGTCG.

SEQ ID NO: 149 includes gdT ligand: 1-342; Free energy: −122.1; gdT CAI: 0.814639976789479; ORF count: 0.

SEQ ID NO: 150 is the sequence name for B2m shRNA1. It is a HLA siRNA construct. It is a DNA sequence. The sequence is: GAATGGAGAGAGAATTGAA.

SEQ ID NO: 151 is the sequence name for CIITA shRNA7. It is a HLA siRNA construct. It is a DNA sequence. The sequence is: GCTCAGGCTAAGCTTGTACAA.

SEQ ID NO: 152 is MND promoter. It is a DNA sequence. The sequence is:

CCCCTCCCTTCAGGCCCCGCGCGATTCCGCCCCCAGTTCTGTGCCGGCCAAGATCCCGGC
TAGCGCCGCTATCATTGGTTAGTTCCAAGTTTGCCCGCCCCTCTTCCTCCTCCTTTTTCCG
CCCCCTCCCTCCCGCGGAAGCTGGGGGCGCATGCGTAGAGGTGGACGCTCCCCTCCCCC
GCCCGGGGTAACTGAGGACTCCCGCGCGCGGACTCGCTGCGCCCCACCCTCCCTTTCCCC
GGGGCCGTCCGGAGAGCGGGGGCGAGCTTGAAAGTTCCAGAACGCTGCGGTGAGTGCGT
TATCGTGAGGCGGAGCGCGGTGGGGTGGGTGCGGAAGGGGGCGAGGCCCGAGGAGTGG
AGCCGGGCTTGTGATTGGGTCTTGTAAGGGCAGCCGGGCGTCTATTGGCCGGGGAAGCC
GTAATGGCAGGCAGCAGGGGGGGGCCCCTTCTGGAAGGTTCTAAGATAGGGTATAAGAG
GCAGGGTGGCGGGCGGAAACCGGTCTCATTGAACTCGCCTGCAGCTCTTGGGTTTTTTGT
GGCTTCCTTCGTTATTGGAGCCAGGCCTACACCCCAGGTAAAACCTCTGCTCAAGAGTTG
GGTTG.

SEQ ID NO: 153 is HSPA8 promoter. It is a DNA sequence. The sequence is:

CCCCTCCCTTCAGGCCCCGCGCGATTCCGCCCCCAGTTCTGTGCCGGCCAAGATCCCGGC
TAGCGCCGCTATCATTGGTTAGTTCCAAGTTTGCCCGCCCCTCTTCCTCCTCCTTTTTCCG
CCCCCTCCCTCCCGCGGAAGCTGGGGGCGCATGCGTAGAGGTGGACGCTCCCCTCCCCC
GCCCGGGGTAACTGAGGACTCCCGCGCGCGGACTCGCTGCGCCCCACCCTCCCTTTCCCC
GGGGCCGTCCGGAGAGCGGGGGCGAGCTTGAAAGTTCCAGAACGCTGCGGTGAGTGCGT
TATCGTGAGGCGGAGCGCGGTGGGGTGGGTGCGGAAGGGGGCGAGGCCCGAGGAGTGG
AGCCGGGCTTGTGATTGGGTCTTGTAAGGGCAGCCGGGCGTCTATTGGCCGGGGAAGCC
GTAATGGCAGGCAGCAGGGGGGGGCCCCTTCTGGAAGGTTCTAAGATAGGGTATAAGAG
GCAGGGTGGCGGGCGGAAACCGGTCTCATTGAACTCGCCTGCAGCTCTTGGGTTTTTTGT
GGCTTCCTTCGTTATTGGAGCCAGGCCTACACCCCAGGTAAAACCTCTGCTCAAGAGTTG
GGTTG

SEQ ID NO: 154 is WPREmut. It is a DNA sequence. The sequence is:

ATCAACCTCTGGATTACAAAATTTGTGAAAGATTGACTGATATTCTTAACTATGTTGCTC
CTTTTACGCTGTGTGGATATGCTGCTTTAATGCCTCTGTATCATGCTATTGCTTCCCGTAC
GGCTTTCGTTTTCTCCTCCTTGTATAAATCCTGGTTGCTGTCTCTTTATGAGGAGTTGTGG
CCCGTTGTCCGTCAACGTGGCGTGGTGTGCTCTGTGTTTGCTGACGCAACCCCCACTGGC
TGGGGCATTGCCACCACCTGTCAACTCCTTTCTGGGACTTTCGCTTTCCCCCTCCCGATCG
CCACGGCAGAACTCATCGCCGCCTGCCTTGCCCGCTGCTGGACAGGGGCTAGGTTGCTG
GGCACTGATAATTCCGTGGTGTTGTCGGGGAAATCATCGTCCTTTCCTTGGCTGCTCGCC
TGTGTTGCCAACTGGATCCTGCGCGGGACGTCCTTCTGCTACGTCCCTTCGGCTCTCAATC
CAGCGGACCTCCCTTCCCGAGGCCTTCTGCCGGTTCTGCGGCCTCTCCCGCGTCTTCGCTT
TCGGCCTCCGACGAGTCGGATCTCCCTTTGGGCCGCCTCCCCGCCTG

SEQ ID NO: 155 is 4Gs. It is an AA sequence. The sequence is: GGGGS

SEQ ID NO: 156 is the sequence name for SSTR2-8. It is a Tumor targeting scFv construct. It is an AA sequence. The sequence is:

EIVMTQSPDSLAVSLGERATISCKSSQSLINSRNRKNYLAWYQQNPGQPPKLLIYWASTRESG
VPDRFSGSGSGTDFTLTISSLQAEDVAVYYCKQSYYLWTFGQGTKVEIKGGGGSGGGGSGG
GGSQVQLVESGGRLVQPGGSLRVSCEASGFTFSDYGMAWVRQAPGKGLEWVSFISNLGYSL
YYADSVKGRFTISRDNAKNSLYLQMNSLRAEDTAVYYCARAPYDYDSFDPMDYWGQGTM
VTVS.

SEQ ID NO: 156 includes Target scFv: 1-248; Target VL: 1-112; Target linker: 113-127; Target VH: 128-248.

SEQ ID NO: 157 is the sequence name for SSTR2-8 v1. It is a Tumor targeting scFv construct. It is an expression codon optimized DNA sequence encoding the amino acid of SEQ ID NO: 156. The sequence is:

GAGATCGTGATGACCCAGAGCCCCGACTCCCTGGCCGTTTCCCTCGGGGAGCGGGCCAC
CATCTCCTGCAAGAGCTCCCAGAGCCTGATCAACTCCCGGAACCGGAAGAACTACCTGG
CCTGGTACCAGCAGAACCCCGGGCAGCCGCCGAAGCTGCTGATCTACTGGGCCTCCACG
CGGGAGAGCGGGGTGCCCGATCGCTTCTCCGGCTCCGGCTCCGGGACCGACTTCACCCT
GACCATCTCCAGCCTGCAGGCCGAGGACGTGGCCGTCTACTACTGCAAGCAGAGCTACT
ACCTCTGGACCTTCGGGCAGGGGACCAAGGTGGAGATCAAAGGCGGCGGAGGCTCTGGC
GGAGGAGGCTCTGGAGGCGGCGGCTCCCAGGTGCAGCTGGTGGAGAGCGGCGGAAGGC
TGGTGCAGCCCGGCGGCTCCCTGAGGGTCTCCTGCGAGGCCTCCGGCTTCACCTTCTCCG
ACTACGGGATGGCCTGGGTGAGGCAGGCTCCCGGGAAGGGGCTGGAGTGGGTCTCCTTC
ATCTCCAACCTGGGCTACTCCCTGTACTACGCCGACTCCGTGAAGGGGCGCTTCACCATC
TCCCGGGACAACGCCAAGAACTCCCTCTACCTGCAGATGAACTCCCTGCGGGCCGAGGA
CACCGCCGTCTACTACTGCGCCCGGGCGCCCTACGACTACGACTCCTTCGACCCCATGGA
CTACTGGGGCCAGGGGACCATGGTGACCGTCTCC.

SEQ ID NO: 157 includes Target scFv: 1-744; Target VL: 1-336; Target linker: 337-381; Target VH: 382-744; Free energy: −340.2; gdT CAI: 0.902; ORF count: 1.

SEQ ID NO: 158 is the sequence name for SSTR2-8 v2. It is a Tumor targeting scFv construct. It is an expression codon optimized DNA sequence encoding the amino acid of SEQ ID NO: 156. The sequence is:

GAAATAGTAATGACGCAATCGCCGGACTCGTTGGCCGTGTCCCTGGGCGAGAGGGCCAC
CATCAGCTGCAAGAGCTCCCAGTCCCTGATCAACTCCCGCAACCGCAAGAACTACCTGG
CCTGGTACCAGCAGAACCCCGGCCAGCCTCCCAAGCTGCTGATCTACTGGGCCAGCACC
CGGGAGAGCGGCGTGCCCGACAGGTTCAGCGGCAGCGGCAGCGGCACCGACTTCACCCT
GACCATCAGCTCCCTGCAGGCCGAGGACGTGGCCGTGTACTACTGCAAGCAGAGCTACT
ACCTGTGGACCTTCGGCCAGGGCACCAAGGTGGAGATCAAGGGAGGTGGCGGCAGCGG
CGGAGGTGGCTCTGGCGGTGGCGGCAGCCAGGTGCAGCTGGTGGAGAGCGGCGGCAGG
CTGGTGCAGCCTGGCGGCTCCCTGCGGGTGAGCTGCGAGGCCAGCGGCTTCACCTTCAG
CGACTACGGCATGGCCTGGGTGCGGCAGGCCCCTGGCAAGGGCCTGGAGTGGGTGAGCT
TCATCTCCAACCTGGGCTACAGCCTGTACTACGCCGACAGCGTGAAGGGCCGCTTCACCA
TCTCCCGGGACAACGCCAAGAACTCCCTGTACCTGCAGATGAACTCCCTGCGGGCCGAG
GACACCGCCGTGTACTACTGCGCCCGGGCCCCTTACGACTACGACAGCTTCGACCCCATG
GACTACTGGGGCCAGGGCACTATGGTGACCGTGAGT.

SEQ ID NO: 158 includes Target scFv: 1-744; Target VL: 1-336; Target linker: 337-381; Target VH: 382-744; Free energy: −322.5; gdT CAI: 0.915155531601317; ORF count: 0.

SEQ ID NO: 159 is the sequence name for SSTR2-8 v3. It is a Tumor targeting scFv construct. It is an expression codon optimized DNA sequence encoding SEQ ID NO: 156. The sequence is:

GAAATAGTAATGACGCAATCGCCGGACTCGTTGGCCGTGAGCCTGGGCGAGCGTGCCAC
CATCTCCTGCAAGTCCTCCCAGAGCCTGATCAACTCCCGGAACCGCAAGAACTACCTGGC
CTGGTACCAGCAGAACCCCGGCCAGCCTCCCAAGCTGCTGATCTACTGGGCCTCCACCCG
GGAGTCCGGCGTGCCCGACAGATTCTCCGGCTCCGGCTCCGGCACCGACTTCACCCTGAC
CATCTCCAGCCTGCAGGCCGAGGATGTGGCAGTGTACTACTGCAAGCAGTCCTACTACCT
GTGGACCTTCGGCCAGGGCACCAAGGTGGAGATCAAGGGTGGCGGAGGCTCTGGCGGTG
GCGGTTCCGGAGGCGGAGGCTCCCAGGTGCAGCTGGTGGAGTCCGGCGGCAGGCTGGTG
CAGCCTGGCGGCAGCCTGAGAGTGTCCTGCGAGGCCTCCGGCTTCACCTTCTCCGACTAC
GGCATGGCTTGGGTGCGGCAGGCTCCCGGCAAGGGCCTGGAGTGGGTGTCCTTCATCTC
CAACCTGGGCTACAGCCTGTACTACGCCGACAGCGTGAAGGGCAGGTTCACCATCTCCA
GAGACAATGCCAAGAACAGCCTGTACCTGCAGATGAACAGCCTGAGAGCCGAGGATAC
CGCCGTGTACTACTGCGCCCGCGCCCCTTACGATTACGATAGCTTCGATCCTATGGATTA
CTGGGGCCAGGGCACTATGGTGACCGTGTCG.

SEQ ID NO: 159 includes Target scFv: 1-744; Target VL: 1-336; Target linker: 337-381; Target VH: 382-744; Free energy: −320; gdT CAI: 0.913669760876946; ORF count: 0.

SEQ ID NO: 160 is the sequence name for SSTR2-8 v4. It is a Tumor targeting scFv construct. It is an expression codon optimized DNA sequence encoding SEQ ID NO: 156. The sequence is:

GAAATTGTAATGACACAATCGCCTGACAGTTTGGCCGTCTCCCTCGGGGAGAGGGCCAC
CATCTCCTGCAAGTCCTCCCAGTCCCTCATCAACTCCCGGAATCGGAAGAACTACCTCGC
CTGGTACCAGCAGAACCCCGGCCAGCCTCCCAAGCTCCTCATCTATTGGGCCTCCACCCG
GGAGAGTGGGGTCCCCGATCGGTTCTCGGGGAGTGGGAGTGGGACCGATTTCACCCTCA
CCATCTCTTCCCTCCAGGCCGAGGATGTGGCCGTCTACTATTGCAAGCAGTCCTACTACC
TCTGGACCTTCGGCCAGGGGACCAAGGTGGAGATCAAGGGAGGAGGTGGGAGTGGAGG
AGGTGGGAGTGGAGGAGGTGGGTCCCAGGTCCAGCTCGTGGAGAGTGGTGGGAGACTC
GTCCAGCCCGGAGGGTCCCTCCGGGTCTCCTGCGAGGCCTCGGGGTTCACCTTCTCCGAC
TATGGGATGGCTTGGGTGCGCCAGGCCCCTGGGAAGGGGCTCGAGTGGGTCTCCTTCAT
CTCCAACCTGGGGTACTCCCTCTACTACGCCGACTCCGTGAAGGGGAGATTCACCATCTC
CCGGGACAACGCCAAGAACTCCCTCTACCTCCAGATGAACTCCCTGAGGGCCGAGGACA
CCGCCGTCTACTATTGCGCCCGGGCTCCCTACGACTACGACTCCTTCGACCCCATGGACT
ATTGGGGCCAGGGGACAATGGTGACCGTGAGT.

SEQ ID NO: 160 includes Target scFv: 1-744; Target VL: 1-336; Target linker: 337-381; Target VH: 382-744; Free energy: −319.399993896484; gdT CAI: 0.812998307048897; ORF count: 0.

SEQ ID NO: 161 is the sequence name for GD2-3. It is a Tumor targeting scFv construct. It is an AA sequence. The sequence is:

QVQLVQSGAEVEKPGASVKVSCKASGSSFTGYNMNWVRQAPGQGLEWIGAIDPYYGGTSY
NQKFKGRATLTVDKSISTAYMELSRLRSDDTAVYYCVSGMKYWGQGTLVTVSSGGGGSGG
GGSGGGGSDVVMTQSPLSLPVTLGQPASISCRSSQSLVHRNGNTYLHWYQQRPGQSPRLLIH
KVSNRFSGVPDRFSGSGSGTDFTLKISRVEAEDVGVYYCSQSTHVPPLTFGQGTKLEIK.

SEQ ID NO: 161 includes Target scFv: 1-248; Target VL: 129-241; Target linker: 114-128; Target VH: 1-113.

SEQ ID NO: 162 is the sequence name for GD2-3 v1. It is a Tumor targeting scFv construct. It is an expression codon optimized DNA sequence encoding SEQ ID NO: 161. The sequence is:

CAGGTCCAGCTGGTGCAGTCCGGGGCCGAGGTGGAGAAGCCCGGGGCCTCCGTGAAGGT
GTCCTGCAAGGCCTCCGGGTCCTCCTTCACCGGGTACAACATGAACTGGGTGAGGCAGG
CTCCCGGGCAGGGGCTGGAGTGGATCGGGGCCATCGACCCCTACTACGGCGGCACCTCC
TACAACCAGAAGTTCAAGGGGCGCGCCACGCTGACCGTGGACAAGTCCATCTCCACCGC
CTACATGGAGCTGTCCCGGCTGCGGTCCGACGACACCGCCGTGTACTACTGCGTGTCCGG
GATGAAGTACTGGGGCCAGGGGACCCTGGTGACCGTGTCCTCCGGCGGCGGAGGCTCTG
GAGGAGGCGGCTCTGGCGGCGGAGGCTCTGACGTGGTGATGACCCAGTCGCCCCTGTCC
CTTCCCGTGACCCTGGGGCAGCCCGCGTCCATCTCCTGCCGGTCCAGCCAGTCCCTGGTG
CACCGGAACGGGAACACCTACCTGCACTGGTACCAGCAGCGGCCCGGGCAGTCTCCCAG
GCTGCTGATCCACAAGGTGTCCAACCGCTTCTCCGGGGTGCCCGACCGCTTCTCCGGGTC
CGGGTCCGGGACCGACTTCACCCTGAAGATCTCCCGCGTGGAGGCCGAGGACGTCGGGG
TGTACTACTGCTCCCAGTCCACGCACGTGCCGCCGCTGACCTTCGGGCAGGGGACCAAG
CTGGAGATCAAG.

SEQ ID NO: 162 includes Target scFv: 1-723; Target VL: 385-723; Target linker: 340-384; Target VH: 1-339; Free energy: −335.6; gdT CAI: 0.914; ORF count: 2.

SEQ ID NO: 163 is the sequence name for GD2-3 v2. It is a Tumor targeting scFv construct. It is an expression codon optimized DNA sequence encoding SEQ ID NO: 161. The sequence is:

CAGGTGCAGCTGGTGCAGTCCGGGGCCGAGGTGGAGAAGCCCGGCGCCTCCGTGAAGGT
GAGCTGCAAGGCCAGCGGCTCCTCCTTCACCGGGTACAACATGAACTGGGTGAGACAGG
CTCCCGGGCAGGGGCTGGAGTGGATCGGGGCCATCGACCCCTACTACGGCGGGACCAGC
TACAACCAGAAGTTCAAGGGGAGGGCCACCCTGACAGTGGACAAGTCCATCTCCACCGC
CTACATGGAGCTGTCCCGCCTGCGGTCCGACGACACCGCCGTGTACTACTGCGTGAGCG
GGATGAAGTACTGGGGCCAGGGGACACTGGTGACCGTGTCCTCCGGCGGAGGCGGGTCC
GGCGGAGGCGGGTCCGGCGGAGGCGGGTCCGACGTGGTGATGACCCAGTCTCCCCTGTC
CCTGCCCGTGACCCTCGGCCAGCCCGCCTCCATCTCCTGCCGCTCCTCCCAGTCCCTGGT
GCACAGGAACGGGAACACATACCTGCACTGGTACCAGCAGCGGCCCGGCCAGTCTCCCC
GCCTGCTGATCCACAAGGTGTCCAACCGCTTCTCCGGCGTGCCCGACCGGTTCTCCGGCT
CCGGCAGCGGGACCGACTTCACCCTGAAGATCAGCCGGGTGGAGGCCGAGGACGTGGG
CGTGTACTACTGCTCCCAGTCTACCCACGTGCCTCCCCTGACCTTCGGCCAGGGGACCAA
GCTGGAGATCAAG.

SEQ ID NO: 163 includes Target scFv: 1-723; Target VL: 385-723; Target linker: 340-384; Target VH: 1-339; Free energy: −346.600006103515; gdT CAI: 0.936000252358001; ORF count: 0.

SEQ ID NO: 164 is the sequence name for GD2-3 v3. It is a Tumor targeting scFv construct. It is an expression codon optimized DNA sequence encoding SEQ ID NO: 161. The sequence is:

CAGGTGCAGCTGGTGCAGTCCGGGGCCGAGGTGGAGAAGCCCGGGGCCTCTGTGAAGGT
GTCCTGCAAGGCCTCCGGGTCCTCCTTCACCGGGTACAACATGAACTGGGTGAGACAGG
CTCCCGGGCAGGGGCTGGAGTGGATCGGGGCCATCGACCCCTACTACGGCGGGACCTCC
TACAACCAGAAGTTCAAGGGGAGGGCCACCCTGACAGTGGACAAGTCCATCTCCACCGC
CTACATGGAGCTGTCCAGGCTGAGGTCCGACGACACCGCCGTGTACTACTGCGTGTCCG
GGATGAAGTACTGGGGCCAGGGGACACTGGTGACCGTGTCCTCTGGCGGCGGTGGGTCC
GGCGGTGGCGGGTCTGGCGGCGGTGGGTCCGATGTGGTGATGACACAGTCTCCCCTGTC
TCTGCCCGTGACACTGGGGCAGCCCGCCTCCATCTCCTGCAGGTCCTCCCAGTCTCTGGT
GCACAGGAACGGGAACACATACCTGCACTGGTACCAGCAGAGGCCCGGGCAGAGCCCC
AGGCTGCTGATCCACAAGGTGTCCAACAGGTTCTCCGGGGTGCCCGACAGGTTCTCCGG
GTCCGGGTCCGGGACAGACTTCACCCTGAAGATCTCCAGGGTGGAGGCCGAGGATGTGG
GCGTGTACTACTGCTCCCAGAGCACCCACGTGCCACCCCTGACCTTCGGGCAGGGGACC
AAGCTGGAGATCAAG.

SEQ ID NO: 164 includes Target scFv: 1-723; Target VL: 385-723; Target linker: 340-384; Target VH: 1-339; Free energy: −346.299987792968; gdT CAI: 0.950248810102486; ORF count: 0.

SEQ ID NO: 165 is the sequence name for GD2-3 v4. It is a Tumor targeting scFv construct. It is an expression codon optimized DNA sequence encoding SEQ ID NO: 161. The sequence is:

CAGGTGCAGCTGGTGCAGTCCGGGGCCGAGGTGGAGAAGCCCGGGGCCTCCGTGAAGGT
GTCCTGCAAGGCCTCCGGGTCCTCCTTCACCGGGTACAACATGAACTGGGTGCGGCAGG
CCCCAGGGCAGGGGCTGGAGTGGATCGGGGCCATCGACCCCTACTACGGCGGGACCTCC
TACAACCAGAAGTTCAAGGGGAGGGCCACCCTGACAGTGGACAAGTCCATCTCCACCGC
CTACATGGAGCTGTCCAGGCTGAGGTCCGACGACACCGCCGTGTACTACTGCGTGTCCG
GGATGAAGTACTGGGGCCAGGGCACACTGGTGACCGTGTCCTCCGGCGGTGGCGGGTCC
GGCGGTGGCGGGTCCGGCGGTGGCGGGTCCGATGTGGTGATGACCCAGTCCCCACTGTC
CCTGCCCGTGACACTGGGGCAGCCCGCCTCCATCTCCTGCAGGTCCTCCCAGTCCCTGGT
GCACAGGAACGGGAACACATACCTGCACTGGTACCAGCAGAGGCCCGGGCAGAGCCCC
AGGCTGCTGATCCACAAGGTGTCCAACAGGTTCTCCGGGGTGCCCGATAGGTTCTCCGG
GTCCGGGTCCGGGACCGACTTCACCCTGAAGATCTCCAGGGTGGAGGCCGAGGACGTGG
GCGTGTACTACTGCTCCCAGAGCACCCACGTGCCACCCCTGACCTTCGGGCAGGGGACC
AAGCTGGAGATCAAG.

SEQ ID NO: 165 includes Target scFv: 1-723; Target VL: 385-723; Target linker: 340-384; Target VH: 1-339; Free energy: −340.600006103515; gdT CAI: 0.952480466768884; ORF count: 0.

SEQ ID NO: 166 is the sequence name for PTK7-14. It is a Tumor targeting scFv construct. It is an AA sequence. The sequence is:

EVQLVESGGGLVKPGGSLRLSCAASGFTFSTYLMYWIRQAPGKGLEWVSALGSGGDTYYAD
SVKGRFTISRDNAKNSLYLQMNSLRAEDTAVYYCARGLGYWGQGTLVTVSSGGGGSGGGG
SGGGGSEIVLTQSPGSLSLSPGERATLSCRASQSVSSSYLAWYQQKPGQAPRLLIYGASSRAT
GIPDRFSGSGSGTDFSLTISRLEPEDFAVYYCQQYGSSPMYTFGPGTRVEIK.

SEQ ID NO: 166 includes Target scFv: 1-236; Target VL: 128-236; Target linker: 113-127; Target VH: 1-112.

SEQ ID NO: 167 is the sequence name for PTK7-14 v1. It is a Tumor targeting scFv construct. It is an expression codon optimized DNA sequence encoding SEQ ID NO: 166. The sequence is:

GAGGTGCAGCTGGTGGAGTCTGGCGGCGGCCTGGTGAAGCCCGGAGGCTCCCTGCGGCT
GTCCTGCGCTGCCTCCGGGTTCACCTTCTCCACCTACCTGATGTACTGGATCAGGCAGGC
TCCCGGGAAGGGGCTGGAGTGGGTGTCCGCCCTGGGGTCCGGCGGCGACACCTACTACG
CCGACTCCGTGAAGGGGCGCTTCACCATCTCCCGGGACAACGCCAAGAACTCCCTGTAC
CTGCAGATGAACTCCCTGCGGGCCGAGGACACCGCCGTGTACTACTGCGCCAGGGGCCT
GGGGTACTGGGGCCAGGGGACCCTGGTGACCGTGTCCTCCGGCGGCGGAGGCTCTGGAG
GAGGCGGCTCTGGCGGCGGAGGCTCTGAGATCGTGCTGACCCAGAGCCCCGGGTCCCTG
TCCCTGAGCCCCGGGGAGCGGGCTACCCTGTCCTGCCGGGCCAGCCAGTCCGTGTCCTCC
TCCTACCTGGCCTGGTACCAGCAGAAGCCCGGGCAGGCTCCCAGGCTGCTGATCTACGG
GGCTTCCTCCAGGGCCACCGGCATCCCCGACCGCTTCTCCGGGTCCGGGTCCGGGACCGA
CTTCTCCCTGACCATCTCCCGGCTGGAGCCCGAGGACTTCGCCGTGTACTACTGCCAGCA
GTACGGGTCCAGCCCCATGTACACCTTCGGGCCCGGGACCCGCGTGGAGATCAAG.

SEQ ID NO: 167 includes Target scFv: 1-708; Target VL: 382-708; Target linker: 337-381; Target VH: 1-336; Free energy: −368.700012207031; gdT CAI: 0.910676800487004; ORF count: 2.

SEQ ID NO: 168 is the sequence name for PTK7-14 v2. It is a Tumor targeting scFv construct. It is an expression codon optimized DNA sequence encoding SEQ ID NO: 166. The sequence is:

GAGGTGCAGCTGGTGGAGTCTGGCGGCGGGCTGGTGAAGCCCGGCGGGTCCCTGCGGCT
GTCCTGCGCCGCCTCCGGGTTCACCTTCTCCACATACCTGATGTACTGGATCCGGCAGGC
TCCCGGGAAGGGGCTGGAGTGGGTGTCCGCTCTGGGGTCCGGCGGGGACACCTACTACG
CCGACTCCGTGAAGGGGCGGTTCACCATCTCCAGGGACAACGCCAAGAACTCCCTGTAC
CTGCAGATGAACTCCCTGCGGGCCGAGGACACCGCCGTGTACTACTGCGCCAGGGGCCT
GGGGTACTGGGGCCAGGGGACACTGGTGACCGTGTCCTCTGGCGGCGGTGGGTCCGGCG
GTGGCGGGTCTGGCGGCGGTGGGTCCGAGATCGTGCTGACCCAGAGCCCCGGGTCCCTG
TCCCTGTCACCCGGGGAGCGGGCCACCCTGTCCTGCAGGGCCTCCCAGTCCGTGTCCTCC
AGCTACCTGGCCTGGTACCAGCAGAAGCCCGGGCAGGCTCCCCGGCTGCTGATCTACGG
GGCCTCCTCCAGGGCCACCGGGATCCCCGACCGGTTCTCCGGGTCCGGGTCCGGGACCG
ACTTCTCCCTGACCATCTCCCGGCTGGAGCCCGAGGACTTCGCCGTGTACTACTGCCAGC
AGTACGGGTCCAGCCCCATGTACACCTTCGGGCCCGGGACCAGGGTGGAGATCAAG.

SEQ ID NO: 168 includes Target scFv: 1-708; Target VL: 382-708; Target linker: 337-381; Target VH: 1-336; Free energy: −367; gdT CAI: 0.932214679000254; ORF count: 0.

SEQ ID NO: 169 is the sequence name for PTK7-14 v3. It is a Tumor targeting scFv construct. It is an expression codon optimized DNA sequence encoding SEQ ID NO: 166. The sequence is:

GAGGTGCAGCTGGTGGAGTCTGGCGGCGGGCTGGTGAAGCCCGGCGGGTC
CCTGCGGCTGTCCTGCGCCGCCTCCGGGTTCACCTTTTCCACATACCTGA
TGTACTGGATCAGACAGGCTCCCGGGAAGGGGCTGGAGTGGGTGTCCGCT
CTGGGGTCCGGCGGGGACACCTACTACGCCGACTCCGTGAAGGGGAGGTT
TACAATCTCCAGGGACAACGCCAAGAACTCCCTGTACCTGCAGATGAACT
CCCTGCGGGCCGAGGACACCGCCGTGTACTACTGCGCCCGCGGGCTGGGG
TACTGGGGCCAGGGGACACTGGTGACAGTGTCCTCTGGCGGCGGAGGGTC
CGGCGGAGGCGGGTCTGGCGGCGGAGGGTCCGAGATCGTGCTGACACAGT
CTCCCGGGTCCCTGTCCCTGTCACCCGGGGAGAGGGCCACCCTGTCCTGC
AGGGCCTCCCAGTCCGTGTCCTCCTCTTACCTGGCCTGGTACCAGCAGAA
GCCCGGGCAGGCTCCCAGGCTGCTGATCTACGGGGCCTCTTCCAGGGCCA
CCGGGATCCCCGACAGGTTCTCCGGGTCCGGGTCCGGGACAGACTTCTCC
CTGACAATCTCCAGGCTGGAGCCCGAGGACTTCGCCGTGTACTACTGCCA
GCAGTACGGGTCCTCTCCCATGTACACCTTCGGGCCCGGGACAAGGGTGG
AGATCAAG.

SEQ ID NO: 169 includes Target scFv: 1-708; Target VL: 382-708; Target linker: 337-381; Target VH: 1-336; Free energy: −354.5; gdT CAI: 0.945572669452621; ORF count: 0.

SEQ ID NO: 170 is the sequence name for PTK7-14 v4. It is a Tumor targeting scFv construct. It is an expression codon optimized DNA sequence encoding SEQ ID NO: 166. The sequence is:

GAGGTGCAGCTGGTGGAGTCTGGCGGCGGGCTGGTGAAGCCCGGCGGGTCCCTGCGGCT
GTCCTGCGCCGCCTCCGGCTTCACCTTCTCCACATACCTGATGTACTGGATCCGGCAGGC
TCCCGGGAAGGGGCTGGAGTGGGTGTCCGCCCTCGGGTCCGGCGGGGACACATACTACG
CCGACTCCGTGAAGGGGCGGTTCACCATCTCCCGGGACAACGCCAAGAATTCCCTGTAC
CTGCAGATGAATTCCCTGCGGGCCGAGGACACAGCCGTGTACTACTGCGCCAGAGGCCT
GGGGTACTGGGGCCAGGGGACACTGGTGACAGTGTCCTCCGGCGGAGGCGGGTCCGGCG
GAGGCGGGTCTGGCGGCGGAGGGTCCGAGATCGTCCTGACCCAGTCTCCCGGGTCCCTG
TCCCTGTCTCCCGGGGAGAGGGCCACACTGTCCTGCCGGGCCTCCCAGTCCGTGTCTTCC
TCCTACCTGGCCTGGTACCAGCAGAAGCCCGGGCAGGCCCCACGGCTGCTGATCTACGG
GGCCTCCTCCAGGGCCACAGGCATCCCCGACAGATTCTCCGGGTCCGGGTCCGGGACAG
ACTTCTCCCTGACCATCTCCCGGCTGGAGCCCGAGGACTTCGCCGTGTACTACTGCCAGC
AGTACGGGTCCAGCCCCATGTACACCTTCGGGCCCGGGACAAGGGTGGAGATCAAG.

SEQ ID NO: 170 includes Target scFv: 1-708; Target VL: 382-708; Target linker: 337-381; Target VH: 1-336; Free energy: −352.5; gdT CAI: 0.92852740873711; ORF count: 0.

SEQ ID NO: 171 is the sequence name for PTK7-15. It is a Tumor Targeting scFv construct. It is an AA sequence. The sequence is:

EVQLVESGGGLVKPGGSLRLSCAASGFTFSTYIMYWIRQAPGKGLEWVSAIGSGGDTYYADS
VKGRFTISRDNAKNSLYLQMNSLRAEDTAVYYCARGLGYWGQGTLVTVSSGGGGSGGGGS
GGGGSEIVLTQSPGSLSLSPGERATLSCRASQSVSSSYLAWYQQKPGQAPRLLIYGASSRATGI
PDRFSGSGSGTDFSLTISRLEPEDFAVYYCQQYGSSPMYTFGPGTRVEIK.

SEQ ID NO: 171 includes Target scFv: 1-236; Target VL: 1-112; Target linker: 113-127; Target VH: 128-236.

SEQ ID NO: 172 is the sequence name for PTK7-16. It is a Tumor Targeting scFv construct. It is an AA sequence. The sequence is:

EVQLVESGGGLVKPGGSLRLSCAASGFTFSTYLMYWIRQAPGKGLEWVSA
IGSGGDTYYADSVKGRFTISRDNAKNSLYLQMNSLRAEDTAVYYCARGIG
YWGQGTLVTVSSGGGGSGGGGSGGGGSEIVLTQSPGSLSLSPGERATLSC
RASQSVSSSYLAWYQQKPGQAPRLLIYGASSRATGIPDRFSGSGSGTDFS
LTISRLEPEDFAVYYCQQYGSSPMYTFGPGTRVEIK.

SEQ ID NO: 172 includes Target scFv: 1-236; Target VL: 1-112; Target linker: 113-127; Target VH: 128-236.

SEQ ID NO: 173 is the sequence name for PTK7-17. It is a Tumor Targeting scFv construct. It is an AA sequence. The sequence is:

EVQLVESGGGLVKPGGSLRLSCAASGFTFSTYLMYWIRQAPGKGLEWVSA
IGSGGDTYYADSVKGRFTISRDNAKNSLYLQMNSLRAEDTAVYYCARGLG
YWGQGTLVTVSSGGGGSGGGGSGGGGSEIVLTQSPGSLSLSPGERATLSC
RASQSVSSSYIAWYQQKPGQAPRLLIYGASSRATGIPDRFSGSGSGTDFS
LTISRLEPEDFAVYYCQQYGSSPMYTFGPGTRVEIK.

SEQ ID NO: 173 includes Target scFv: 1-236; Target VL: 1-112; Target linker: 113-127; Target VH: 128-236.

SEQ ID NO: 174 is the sequence name for PTK7-18. It is a Tumor Targeting scFv construct. It is an AA sequence. The sequence is:

EVQLVESGGGLVKPGGSLRLSCAASGFTFSTYLMYWIRQAPGKGLEWVSA
IGSGGDTYYADSVKGRFTISRDNAKNSLYLQMNSLRAEDTAVYYCARGLG
YWGQGTLVTVSSGGGGSGGGGSGGGGSEIVLTQSPGSLSLSPGERATLSC
RASQSVSSSYLAWYQQKPGQAPRLLIYGASSKATGIPDRFSGSGSGTDFS
LTISRLEPEDFAVYYCQQYGSSPMYTFGPGTRVEIK.

SEQ ID NO: 174 includes Target scFv: 1-236; Target VL: 1-112; Target linker: 113-127; Target VH: 128-236.

SEQ ID NO: 175 is the sequence name for PTK7-19. It is a Tumor Targeting scFv construct. It is an AA sequence. The sequence is:

SEQ ID NO: 175
EVQLVESGGGLVKPGGSLRLSCAASGFTFSTYLMYWIRQAPGKGLEWVSA
IGSGGDTYYADSVKGRFTISRDNAKNSLYLQMNSLRAEDTAVYYCARGLG
YWGQGTLVTVSSGGGGSGGGGSGGGGSEIVLTQSPGSLSLSPGERATLSC
RASQSVSSSYLAWYQQKPGQAPRLLIYGASSRATGIPDRFSGSGSGTDFS
LTISRLEPEDFAVYYCQQYGSSPLYTFGPGTRVEIK.

includes Target scFv: 1-236; Target VL: 1-112; Target linker: 113-127; Target VH: 128-236.

SEQ ID NO: 176 is the sequence name for SSTR2-9. It is a Tumor Targeting scFv construct. It is an AA sequence. The sequence is:

EIVMTQSPDSLAVSLGERATISCKSSQSLLNSRNRKNYLAWYQQNPGQPP
KLLIYWASTKESGVPDRFSGSGSGTDFTLTISSLQAEDVAVYYCKQSYYL
WTFGQGTKVEIKGGGGSGGGGSGGGGSQVQLVESGGRLVQPGGSLRVSCE
ASGFTFSDYGMAWVRQAPGKGLEWVSFISNLGYSIYYADSVKGRFTISRD
NAKNSLYLQMNSLRAEDTAVYYCARAPYDYDSFDPMDYWGQGTMVTVS.

SEQ ID NO: 176 includes Target scFv: 1-248; Target VL: 1-112; Target linker: 113-127; Target VH: 128-248.

SEQ ID NO: 177 is the sequence name for SSTR2-10. It is a Tumor Targeting scFv construct. It is an AA sequence. The sequence is:

EIVMTQSPDSLAVSLGERATISCKSSQSLLNSRNRKNYLAWYQQNPGQPP
KLLIYWASTRESGVPDRFSGSGSGTDFTLTISSLQAEDVAVYYCKQSYYI
WTFGQGTKVEIKGGGGSGGGGSGGGGSQVQLVESGGRLVQPGGSLRVSCE
ASGFTFSDYGMAWVRQAPGKGLEWVSFISNLGYSIYYADSVKGRFTISRD
NAKNSLYLQMNSLRAEDTAVYYCARAPYDYDSFDPMDYWGQGTMVTVS.

SEQ ID NO: 177 includes Target scFv: 1-248; Target VL: 1-112; Target linker: 113-127; Target VH: 128-248.

SEQ ID NO: 178 is the sequence name for SSTR2-11. It is a Tumor Targeting scFv construct. It is an AA sequence. The sequence is:

EIVMTQSPDSLAVSLGERATISCKSSQSLLNSRNRKNYLAWYQQNPGQPP
KLLIYWASTRESGVPDRFSGSGSGTDFTLTISSLQAEDVAVYYCKQSYYL
WTFGQGTKVEIKGGGGSGGGGSGGGGSQVQLVESGGRLVQPGGSLRVSCE
ASGFTFSDYGLAWVRQAPGKGLEWVSFISNLGYSIYYADSVKGRFTISRD
NAKNSLYLQMNSLRAEDTAVYYCARAPYDYDSFDPMDYWGQGTMVTVS.

SEQ ID NO: 178 includes Target scFv: 1-248; Target VL: 1-112; Target linker: 113-127; Target VH: 128-248.

SEQ ID NO: 179 is the sequence name for SSTR2-12. It is a Tumor Targeting scFv construct. It is an AA sequence. The sequence is:

EIVMTQSPDSLAVSLGERATISCKSSQSLLNSRNRKNYLAWYQQNPGQPP
KLLIYWASTRESGVPDRFSGSGSGTDFTLTISSLQAEDVAVYYCKQSYYL
WTFGQGTKVEIKGGGGSGGGGSGGGGSQVQLVESGGRLVQPGGSLRVSCE
ASGFTFSDYGMAWVRQAPGKGLEWVSFISNLGYSIYYADSVKGRFTISRD
NAKNSLYLQMNSLRAEDTAVYYCARAPYDYDSFDPMNYWGQGTMVTVS.

SEQ ID NO: 179 includes Target scFv: 1-248; Target VL: 1-112; Target linker: 113-127; Target VH: 128-248.

SEQ ID NO: 180 is the sequence name for SSTR2-13. It is a Tumor Targeting scFv construct. It is an Amino Acid sequence. The sequence is:

EIVMTQSPDSLAVSLGERATISCKSSQSLLNSRNRKNYLAWYQQNPGQPP
KLLIYWASTKESGVPDRFSGSGSGTDFTLTISSLQAEDVAVYYCKQSYYL
WTFGQGTKVEIKGGGGSGGGGSGGGGSQVQLVESGGRLVQPGGSLRVSCE
ASGFTFSDYGMAWVRQAPGKGLEWVSFISNLGYSIYYADSVKGRFTISRD
NAKNSLYLQMNSLRAEDTAVYYCARAPYDYDSFDPMNYWGQGTMVTVS.

SEQ ID NO: 180 includes Target scFv: 1-236; Target VL: 1-112; Target linker: 113-127; Target VH: 128-236.

SEQ ID NO: 181 is the sequence name for SSTR2-16. It is a Tumor Targeting scFv construct. It is an Amino Acid sequence. The sequence is:

EIVMTQSPDSLAVSLGERATISCKSSQSLLNSRNRKNYLAWYQQNPGQPP
KLLIYWASTKESGVPDRFSGSGSGTDFTLTISSLQAEDVAVYYCKQSYYL
WTFGQGTKVEIKGGGGSGGGGSGGGGSQVQLVESGGRLVQPGGSLRVSCE
ASGFTFSDYGLAWVRQAPGKGLEWVSFISNLGYSIYYADSVKGRFTISRD
NAKNSLYLQMNSLRAEDTAVYYCARAPYDYDSFDPMDYWGQGTMVTVS.

SEQ ID NO: 181 includes Target scFv: 1-236; Target VL: 1-112; Target linker: 113-127; Target VH: 128-236.

SEQ ID NO: 182 is the sequence name for PTK7-17. It is a Tumor Targeting scFv construct. It is a DNA sequence. The sequence is:

gaggtgcagctggtggagtctggcggcggcctggtgaagcccggaggctc
cctgcggctgtcctgcgctgcctccgggttcaccttctccacctacctga
tgtactggatcaggcaggctcccgggaaggggctggagtgggtgtccgcc
atcgggtccggcggcgacacctactacgccgactccgtgaaggggcgctt
caccatctcccgggacaacgccaagaactccctgtacctgcagatgaact
ccctgcgggccgaggacaccgccgtgtactactgcgccaggggcctgggg
tactggggccaggggaccctggtgaccgtgtcctccggcggcggaggctc
tggaggaggcggctctggcggcggaggctctgagatcgtgctgacccaga
gccccgggtccctgtccctgagccccggggagcgggctaccctgtcctgc
cgggccagccagtccgtgtcctcctcctacatcgcctggtaccagcagaa
gcccgggcaggctcccaggctgctgatctacggggcttcctccagggcca
ccggcatccccgaccgcttctccgggtccgggtccgggaccgacttctcc
ctgaccatctcccggctggagcccgaggacttcgccgtgtactactgcca
gcagtacgggtccagccccatgtacaccttcgggcccgggacccgcgtgg
agatcaag.

SEQ ID NO: 182 includes Target scFv: 1-708; Target VL: 382-708; Target linker: 337-381; Target VH: 1-336; Free energy: −363.7; gdT CAI: 0.933; ORF count: 1.

SEQ ID NO: 183 is the sequence name for PTK7-19. It is a Tumor Targeting scFv construct. It is a DNA sequence. The sequence is:

gaggtgcagctggtggagtctggcggcggcctggtgaagcccggaggctc
cctgcggctgtcctgcgctgcctccgggttcaccttctccacctacctga
tgtactggatcaggcaggctcccgggaaggggctggagtgggtgtccgcc
atcgggtccggcggcgacacctactacgccgactccgtgaaggggcgctt
caccatctcccgggacaacgccaagaactccctgtacctgcagatgaact
ccctgcgggccgaggacaccgccgtgtactactgcgccaggggcctgggg
tactggggccaggggaccctggtgaccgtgtcctccggcggcggaggctc
tggaggaggcggctctggcggcggaggctctgagatcgtgctgacccaga
gccccgggtccctgtccctgagccccggggagcgggctaccctgtcctgc
cgggccagccagtccgtgtcctcctcctacctggcctggtaccagcagaa
gcccgggcaggctcccaggctgctgatctacggggcttcctccagggcca
ccggcatccccgaccgcttctccgggtccgggtccgggaccgacttctcc
ctgaccatctcccggctggagcccgaggacttcgccgtgtactactgcca
gcagtacgggtccagccccctgtacaccttcgggcccgggacccgcgtgg
agatcaag.

SEQ ID NO: 183 includes Target scFv: 1-708; Target VL: 382-708; Target linker: 337-381; Target VH: 1-336; Free energy: −365.5; gdT CAI: 0.933; ORF count: 1.

SEQ ID NO: 184 is the sequence name for SSTR2-13. It is a Tumor Targeting scFv construct. It is a DNA sequence. The sequence is:

gagatcgtgatgacccagagccccgactccctggccgtttccctcgggga
gcgggccaccatctcctgcaagagctcccagagcctgctgaactcccgga
accggaagaactacctggcctggtaccagcagaaccccgggcagccgccg
aagctgctgatctactgggcctccacgaaggagagcggggtgcccgatcg
cttctccggctccggctccgggaccgacttcaccctgaccatctccagcc
tgcaggccgaggacgtggccgtctactactgcaagcagagctactacctc
tggaccttcgggcaggggaccaaggtggagatcaaaggcggcggaggctc
tggcggaggaggctctggaggcggcggctcccaggtgcagctggtggaga
gcggcggaaggctggtgcagcccggcggctccctgagggtctcctgcgag
gcctccggcttcaccttctccgactacgggatggcctgggtgaggcaggc
tcccgggaaggggctggagtgggtctccttcatctccaacctgggctact
ccatctactacgccgactccgtgaaggggcgcttcaccatctcccgggac
aacgccaagaactccctctacctgcagatgaactccctgcgggccgagga
caccgccgtctactactgcgcccgggcgccctacgactacgactccttcg
accccatgaactactggggccaggggaccatggtgaccgtctcc.

SEQ ID NO: 184 includes Target scFv: 1-744; Target VL: 1-336; Target linker: 337-381; Target VH: 382-744; Free energy: −337.6; gdT CAI: 0.904; ORF count: 0.

SEQ ID NO: 185 is the sequence name for SSTR2-16. It is a Tumor Targeting scFv construct. It is a DNA sequence. The sequence is:

gagatcgtgatgacccagagccccgactccctggccgtttccctcgggga
gcgggccaccatctcctgcaagagctcccagagcctgctgaactcccgga
accggaagaactacctggcctggtaccagcagaaccccgggcagccgccg
aagctgctgatctactgggcctccacgaaggagagcggggtgcccgatcg
cttctccggctccggctccgggaccgacttcaccctgaccatctccagcc
tgcaggccgaggacgtggccgtctactactgcaagcagagctactacctc
tggaccttcgggcaggggaccaaggtggagatcaaaggcggcggaggctc
tggcggaggaggctctggaggcggcggctcccaggtgcagctggtggaga
gcggcggaaggctggtgcagcceggeggctccctgagggtctcctgcgag
gcctccggcttcaccttctccgactacgggCtggcctgggtgaggcaggc
tcccgggaaggggctggagtgggtctccttcatctccaacctgggctact
ccatctactacgccgactccgtgaaggggcgcttcaccatctcccgggac
aacgccaagaactccctctacctgcagatgaactccctgcgggccgagga
caccgccgtctactactgcgcccgggcgccctacgactacgactccttcg
accccatggactactggggccaggggaccatggtgaccgtctcc.

SEQ ID NO: 185 includes Target scFv: 1-744; Target VL: 1-336; Target linker: 337-381; Target VH: 382-744; Free energy: −339.5; gdT CAI: 0.904; ORF count: 0.

In a variation, the present disclosure provides a composition comprising an isolated polynucleotide disclosed herein as any one of SEQ ID NOS: 1 through 185 or encoding an amino acid disclosed herein as any one of SEQ ID NOS: 1 through 185. We also disclose a polynucleotide comprising the nucleotide sequence of the full-length protein of the amino acid sequences disclosed as any one of SED ID NOS: 1 through 185. We also disclose a polynucleotide encoding a protein comprising a fragment of the amino acid sequence of any on of SEQ ID NOS: 1 through 185 having biological activity, the fragment comprising eight contiguous amino acids of any one of SEQ ID NOS: 1 through 185; a polynucleotide which is an allelic variant of a polynucleotide of any of SEQ ID NOS: 1-185 or those disclosed above; a polynucleotide which encodes a species-homologue of the protein encoded by or disclosed as any one of SEQ ID NOS: 1-185 or any of those disclosed above; a polynucleotide that hybridizes under stringent conditions to any one of the polynucleotides specified in or encoded by SEQ ID NOS: 1-185 or any of those disclosed herein; and a polynucleotide that hybridizes under stringent conditions to any one of the polynucleotides specified or encoded by SEQ ID NOS: 1 through 185 and that has a length that is at least 25% of the length of the sequence encoded or encoded by SEQ ID NOS: 1 through 185.

Other systems, methods, features, and advantages of the disclosure will be, or will become, apparent to one with skill in the art upon examination of the following figures and detailed description. It is intended that all such additional systems, methods, features and advantages be included within this description, be within the scope of the invention, and be protected by the following claims.

The invention is not limited to the variations illustrated and described, as it also covers all equivalent implementations insofar as they do not depart form the spirit of the invention. Further, the invention is not yet limited to the combination of features as described herein but may be defined by any other combination of all of the individual features disclosed. Further, the invention is not yet limited to the sequence of method steps as described herein but may be defined by any other combination or order the steps disclosed. Any person skilled in the art of will recognize from the previous detailed description and from the figures and claims that modifications could be made to the disclosed embodiments of the invention without departing from the scope of the invention.

1. A therapeutic agent comprising a single chain antibody variable domain that binds:
PTK7, wherein the single chain antibody variable region that binds PTK7 has an amino acid sequence at least 96% identical to SEQ ID NO: 173, or
SSTR2, wherein the single chain antibody variable region that binds SSTR-2 has an amino acid sequence at least 96% identical to SEQ ID NO: 181.

2. The therapeutic agent of claim 1, further comprising, from N-terminus to C-terminus, a gamma delta T cell optimized signal peptide that is cleaved off prior to secretion, a linker and a T cell binding protein.

3. The therapeutic agent of claim 2, wherein: the linker is SEQ ID NO: 155.

4. The therapeutic agent of claim 2, wherein: the T cell binding protein is SEQ ID NO: 94.

5. The therapeutic agent of claim 1, wherein the single chain antibody variable domain that binds PTK7 is encoded by a nucleic acid sequence at least 96% identical to SEQ ID NO: 182.

6. The therapeutic agent of claim 1, wherein the single chain antibody variable domain that binds SSTR2 is encoded by a nucleic acid sequence at least 96% identical to SEQ ID NO: 185.

7. An engineered gamma delta T cell capable of secreting at least one therapeutic protein, the therapeutic protein comprising, from N-terminus to C-terminus, a gamma delta T cell optimized signal peptide, a tumor cell binding domain, a linker, and a T cell binding domain, wherein the tumor cell binding domain has an amino acid sequence at least 96% identical to SEQ ID NO: 173 or SEQ ID NO: 181.

8. The engineered gamma delta T cell of claim 7, wherein: the linker is SEQ ID NO: 155.

9. The engineered gamma delta T cell of claim 8, wherein: the T cell binding protein is SEQ ID NO: 94.

10. The engineered gamma delta T cell of claim 7, wherein the tumor cell binding domain is encoded by a nucleic acid sequence at least 96% identical to SEQ ID NO: 182.

11. The engineered gamma delta T cell of claim 7, wherein the tumor cell binding domain is encoded by a nucleic acid sequence at least 96% identical to SEQ ID NO: 185.

12. A recombinant viral vector encoding a therapeutic protein capable of biosynthesis and secretion by a gamma delta T cell, the therapeutic protein comprising, from N-terminus to C-terminus, a gamma delta T cell optimized signal peptide that is cleaved off prior to secretion, a tumor cell-binding protein domain, a linker, and a T cell binding protein, wherein the tumor cell-binding protein domain binds:
PTK7, wherein the single chain antibody variable region that binds PTK7 has an amino acid sequence at least 96% identical to SEQ ID NO: 173, or
SSTR2, wherein the single chain antibody variable region that binds SSTR-2 has an amino acid sequence at least 96% identical to SEQ ID NO: 181.

13. The recombinant viral vector of claim 12, wherein:
the linker is SEQ ID NO: 155, and
the T cell binding protein is SEQ ID NO: 94.

14. The recombinant viral vector of claim 12, wherein the tumor cell-binding protein domain that binds SSTR2 is encoded by a nucleic acid sequence at least 96% identical to of SEQ ID NO: 182.

15. The recombinant viral vector of claim 12, wherein the tumor cell-binding protein domain that binds GD2-3 is encoded by a nucleic acid sequence at least 96% identical to of SEQ ID NO: 185.


SEQUENCE LISTING (185 sequences)

SEQ ID NO: 1 - protein, 518 aa, synthetic construct
MYRMQLLSCIALSLALVTNSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - DNA, 1554 bp, synthetic construct
  source 1..1554: mol_type = other DNA
atgtacaggatgcagctgctgtcctgcattgccctgtccctggccctggtgaccaactccgacattcaattgacacaaagcccggcaagcctggctgtgtccctgggccagcgggcgactatttcatgtaaagcaagtcagagcgtagactacgatggtgatagctatctgaactggtatcagcagattccaggacaacctcccaaattgctgatttacgacgcctcgaacctggtcagcgggattccaccacgattctctggaagcggcagtggaaccgattttacgctgaatatacacccagtggaaaaggtggacgctgcgacttatcattgtcagcagtctaccgaggacccatggaccttcggcgggggaacaaagctggagatcaagggtggtggtggaagcggtggtggtggatctggcggtggtggaagtcaggtgcagcttcagcagtccggagccgaactggtgcgccccggcagtagcgtgaagataagctgcaaggctagcggatacgctttctcttcctactggatgaactgggtgaagcagcgccctggccaagggcttgagtggatcggccagatatggcctggtgatggtgacaccaactataatggcaagtttaagggaaaggctacactcactgccgatgaaagttcatccactgcctacatgcagctctccagtctggcaagtgaagactctgctgtctatttctgcgctagacgagagactacaactgtcgggaggtactactatgcaatggattactggggtcagggaaccacagtgacagtgagctcaggaggtggtgggtctgacattaagctacaacagtctggcgccgagttggccaggcctggggccagcgtgaagatgtcttgcaagacctcaggttataccttcacgagatacaccatgcattgggtaaaacagagacctgggcaaggcttggaatggatcggctacatcaaccccagtcgcggatacacaaactacaatcagaaatttaaagacaaggcgactctcaccacagataagtcctcctctaccgcctacatgcagctgtcatctctcacaagcgaagactctgccgtgtattattgcgcaaggtattacgatgaccactattgtttagattattgggggcaaggaactacactcactgtcagctcagttgagggtggaagtgggggatctggtggttcaggcggatctggtggggtcgacgacatccagctgacccagagtcccgccatcatgtcagctagtcccggggagaaagtgactatgacatgcagggcatctagcagcgtttcgtacatgaattggtatcagcagaaatcaggtacctccccgaaacgttggatttatgacacgagcaaggttgcaagcggtgttccataccggttttcgggctccggttccggcacctcctactctctcacgatctccagtatggaagccgaggatgccgcaacctattactgtcagcaatggtcatccaatcccttaaccttcggagccggaacaaaactggagctcaaa

SEQ ID NO: 3 - protein, 19 aa, synthetic construct
MKWVTFISLLFLFSSSSRA

SEQ ID NO: 4 - protein, 25 aa, Homo sapiens
MKKTQTWILTCIYLQLLLFNPLVKT

SEQ ID NO: 5 - DNA, 57 bp, synthetic construct
  source 1..57: mol_type = other DNA
atgaaatgggttacttttattagtttattattcctgttcagctccagctccagggcc

SEQ ID NO: 6 - DNA, 75 bp, Homo sapiens
  source 1..75: mol_type = unassigned DNA
atgaagaaaactcaaacttggatactaacttgcatctacctgcagctgctgctcttcaaccccttggtgaagacg

SEQ ID NO: 7 - DNA, 750 bp, Synthetic construct
  source 1..750: mol_type = other DNA
gacatccagctcacccagagccccgcatccctggccgtgtccctggggcagcgcgcaaccatctcctgcaaggcttcccagtccgtggactacgacggggactcctacctgaactggtaccagcagatccccgggcagccaccgaagctgctgatctacgacgcctccaacctggtgtccgggattcctccgcggttctccgggagcgggtccgggaccgacttcaccctgaacatccatcccgtggagaaggtggacgccgccacctaccactgccagcagtccaccgaggacccctggaccttcggcggcggcaccaagctggagatcaaaggtggcggaggctccggtggcggaggttccggtggcggcggctcccaggtgcagctgcagcagtcaggggccgagctggtgaggcccgggagctccgtgaagatctcctgcaaggcctccgggtacgccttctcctcctactggatgaactgggtgaagcagaggcccgggcaggggctggagtggatcgggcagatctggcccggggacggggacaccaactacaacgggaagttcaaggggaaggctaccctcaccgccgacgagagctcctccaccgcctacatgcagctgagctccctcgcctccgaggactccgccgtgtacttctgcgcccggcgcgagaccaccaccgtggggcgctactactacgccatggactactggggccaggggaccaccgtgaccgtgagctcc

SEQ ID NO: 8 - DNA, 750 bp, synthetic construct
  source 1..750: mol_type = other DNA
gacatccagctgacccagtcgcccgctagcctggcggtctcgctgggccagcgagccaccatcagctgcaaggccagccagtcggtcgactacgacggcgacagctacctcaactggtaccagcagatccccggccagccaccgaagctgctgatctacgacgccagcaacctggtgagcgggataccaccgcggttctcggggtcggggtcggggaccgacttcaccctcaacatccatcccgtcgagaaggtcgacgccgccacctaccactgccagcagtcgaccgaggacccctggaccttcggcggcggcaccaagctggagatcaaaggtggcggtggctccggtggcggtggtagcggtggcggcggctcccaggtccagctccagcagtctggggccgagctggtgcgacccgggagctcggtcaagatcagctgcaaggccagcggctacgccttctccagctactggatgaactgggtcaagcagcgacccggccaggggctggagtggatcggccagatctggcccggcgacggcgacaccaactacaacggcaagttcaagggcaaggccacgctgaccgccgacgagtcgagctcgaccgcctacatgcagctgagctcgctcgccagcgaggactccgcggtctacttctgcgcccggcgcgagaccaccaccgtggggcgctactactacgcgatggactactggggccaggggaccacggtgaccgtgagctcg

SEQ ID NO: 9 - DNA, 750 bp, synthetic construct
  source 1..750: mol_type = other DNA
gacatccagctgacgcagtcgcccgcgtcgctcgccgtgtcgctcgggcaacgcgcgacgatctcgtgcaaggcgtcgcagtccgtcgactacgacggcgactcgtacctgaactggtaccagcagatccccgggcagccgccgaagctgctgatctacgacgcgagcaacctcgtgtccggcattccgccgcggttcagcggcagcggcagcggcaccgacttcacgctgaacattcaccccgtcgagaaggtcgacgccgcgacgtaccactgccagcagtccaccgaggacccgtggacgttcggcggcggcacgaagctggagatcaaaggtggcggcggttccggtggcggtggttccggtggcggcggctcgcaggtgcagctgcagcagtctggcgccgagctcgtgcgacccgggtcgtccgtgaagatctcgtgcaaggcgtccgggtacgcattctcgtcgtactggatgaactgggtgaagcagcgacccgggcaggggctggagtggatcgggcagatctggcccggcgacggcgacacgaactacaacggcaagttcaagggcaaggcgacgctgaccgccgacgagtcgtcgtccaccgcgtacatgcagctgtcgtcgctcgcgagcgaggacagcgccgtgtacttctgcgcgcggcgcgagacgacgaccgtcgggcgctactactacgcgatggactactggggccagggcacgaccgtgaccgtgtccagc

SEQ ID NO: 10 - DNA, 750 bp, synthetic construct
  source 1..750: mol_type = other DNA
gacatccagctgacccagagccccgcgagcctggccgtgagcctggggcagagggccaccatcagctgcaaggcgtcccagagcgtggactacgacggggacagctacctgaactggtaccagcagatccccgggcagccgccgaagctgctgatctacgacgcctccaacctggtgtccgggataccgccgcggttctccgggtccgggtccgggaccgacttcaccctgaacattcaccccgtggagaaggtggacgccgccacctaccactgccagcagagcaccgaggacccctggaccttcggcggcggcaccaagctggagatcaaaggtggcggaggctccggtggcggtggtagcggtggcggcggctcccaggtgcagctgcagcagtctggggccgagctggtgcgacccgggtccagcgtgaagatcagctgcaaggccagcgggtacgccttcagctcctactggatgaactgggtgaagcagaggcccgggcaggggctggagtggatcgggcagatctggcccggggacggggacaccaactacaacgggaagttcaaggggaaggccacgctgaccgccgacgagagcagctccaccgcctacatgcagctgtccagcctggcctccgaggacagcgccgtgtacttctgcgcccggcgcgagaccaccaccgtggggcgctactactacgccatggactactggggccaggggaccaccgtgaccgtgagctcc

SEQ ID NO: 11 - DNA, 750 bp, synthetic construct
  source 1..750: mol_type = other DNA
gacatccagctgacccagtcacccgctagcctggccgtgtccctgggccagcgagccacgatctcctgcaaggccagccagtccgtggactacgacggggactcctacctcaactggtaccagcagatccccggccagccaccgaagctgctgatctacgacgcctccaacctggtgagcgggattccgccgcggttcagcgggtccgggtccgggaccgacttcaccctcaacatccatcccgtggagaaggtggacgccgccacctaccactgccagcagtccaccgaggacccctggaccttcggcggcggcaccaagctggagatcaaaggcggtggtggctccggaggcggtggctctggtggcggcggctcccaggtgcagctccagcagagcggggctgagctggtgaggcccgggtcctccgtgaagatctcctgcaaggcctccgggtacgccttctccagctactggatgaactgggtgaagcagaggcccgggcaggggctggagtggatcgggcagatctggcccggggacggggacaccaactacaacgggaagttcaaggggaaggccacgctgaccgccgacgagagcagctccaccgcctacatgcagctgtccagcctggcctccgaggactccgccgtgtacttctgcgcccggcgcgagaccaccaccgtggggcgctactactacgccatggactactggggccaggggaccaccgtgaccgtgtccagc

SEQ ID NO: 12 - DNA, 729 bp, synthetic construct
  source 1..729: mol_type = other DNA
gatatcaagctccagcagtccggggctgagctggctaggcccggggcctccgtgaagatgtcctgcaagacctccgggtacaccttcaccaggtacaccatgcactgggtgaagcagaggcccgggcaggggctggagtggatcgggtacatcaaccccagccgcgggtacaccaactacaaccagaagttcaaggacaaggccaccttgaccaccgacaagtcctcctccaccgcctacatgcagctgagctccctgacctccgaggactccgccgtctactactgcgccaggtactacgacgaccactactgcctggactactggggccaggggactaccctgaccgtgagctccgtggaaggtggctccggcggctccggaggttccggaggctccggcggcgtggacgacatccagctgacccagtccccagctatcatgtccgccagccccggggagaaggtgaccatgacctgccgggcctcctcctccgtgagctacatgaactggtaccagcagaagtccgggacctctcccaagaggtggatctacgacacctccaaggtggcctccggggtcccctacaggttctccgggtccgggtccgggacctcctactccctgaccatctcctccatggaggccgaggacgccgccacctactactgccagcagtggagctccaatcccctgaccttcggggccgggaccaagctggagctgaag

SEQ ID NO: 13 - DNA, 729 bp, unidentified
  source 1..729: mol_type = other DNA
gacatcaagctccagcagtccggggctgagcttgctcgccccggggccagcgtgaagatgtcctgcaagacctcggggtacaccttcaccaggtacaccatgcactgggtgaagcagcgcccagggcagggcctggagtggatagggtacatcaaccccagccgcgggtacacaaactacaaccagaagttcaaggacaaggccacgctgaccacggacaagtcctcgtccacggcgtacatgcagctgtcctcgctgacctccgaggacagcgcggtgtactactgcgcgcggtactacgacgaccactactgcctggactactggggccaggggacaacgctgaccgtgagcagcgtggaaggcggcagcggcggcagcggaggtagcggaggctccggcggcgtggacgacatccagctcacccagagcccggcgatcatgtccgccagccccggggagaaggtgaccatgacctgccgggcgtcgtcctcggtgagctacatgaactggtaccagcagaagtccgggacctcgcccaagcgctggatctacgacaccagcaaggtggcctccggggtgccctaccgcttctccgggtcggggtcggggacatcgtactccctgaccatctccagcatggaggccgaggacgccgcgacgtactactgccagcagtggtcgtccaacccgctgaccttcggcgcggggacaaagctggagcttaag

SEQ ID NO: 14 - DNA, 729 bp, synthetic construct
  source 1..729: mol_type = other DNA
gacatcaagctgcagcagagcggcgctgagctcgcgcgaccaggcgcgagcgtgaagatgagctgcaagacgagcggctacacgttcacgcgctacacgatgcactgggtgaagcagcgtcccgggcaggggctggagtggatcggctacatcaacccgtcgcgcggctacacgaactacaaccagaagttcaaggacaaggcgacgctgacgaccgacaagagcagcagcaccgcgtacatgcagctgagctcgctgacgagcgaggacagcgccgtgtactactgcgcgcgctactacgacgaccactactgcctcgactactggggccagggcacgacgctgaccgtgagcagcgtcgaaggcggcagcggcggcagcggtggaagcggtggcagcggcggcgtcgacgacatccagctgacgcagtcgcccgcgatcatgagcgcgtcgcccggcgagaaggtgacgatgacgtgccgcgcgagcagcagcgtgtcgtacatgaactggtaccagcagaagagcggcacgtcgccgaagcgctggatctacgacacgtcgaaggtcgcgagcggcgtgccgtaccgcttcagcggcagcggcagcggcacgtcgtactcgctgacgatcagcagcatggaggccgaggacgccgcgacgtactactgccagcagtggagctcgaacccgctgacgttcggcgccggcacgaagctggagctgaag

SEQ ID NO: 15 - DNA, 729 bp, synthetic construct
  source 1..729: mol_type = other DNA
gacatcaagctgcagcagagcggcgcggaattagcgcgccccggggcgtccgtcaagatgtcctgcaagacctccgggtacaccttcacgcggtacaccatgcactgggtgaagcaacgccccgggcagggcctggagtggatcgggtacatcaacccgtcgcggggctacacgaactacaaccagaagttcaaggacaaggcgaccctcacgaccgacaagtcgagcagcacggcgtacatgcagctctcctcgctgaccagcgaggactccgcggtgtactactgcgcgcggtactacgacgaccactactgcctggactactggggccagggcaccacgctgaccgtcagctcggtggaaggcggctccggcggctccggaggttccggaggttccggcggagtggacgacatccagctcacccagagccccgcgattatgagcgcctcgcccggcgagaaggtcaccatgacctgccgggcgagcagctcggtgagctacatgaactggtaccagcagaagagcggcacgtcgcccaagcggtggatctacgataccagcaaggtggcgagcggcgtgccgtaccgcttctcggggagcgggtccggcacgtcgtacagcctgacgatcagcagcatggaggcggaggacgccgcgacgtactactgccagcagtggagcagcaacccgctgaccttcggggccggcacgaagctcgaactgaag

SEQ ID NO: 16 - DNA, 729 bp, synthetic construct
  source 1..729: mol_type = other DNA
gacatcaagctgcagcagtccggagccgagttggcacggcccggggcctccgtgaagatgtcctgcaagacgtccgggtacacgttcacccggtacaccatgcactgggtgaagcagcggcccgggcaggggctggaatggatcgggtacatcaaccccagccgcgggtacaccaactacaaccagaagttcaaggacaaggccacgctgaccaccgacaagtcgtcgtcgaccgcctacatgcagctgagctcgctgaccagcgaggacagcgccgtctactactgcgcccgctactacgacgaccactactgcctggactactggggccaggggaccacgctgacagtgagcagcgtggaaggcggctccggcggctccggaggttccggaggcagcggcggcgtcgacgacatccagctcacccagtccccggccatcatgtcggcgagccccggcgagaaggtgaccatgacgtgccgggcctccagctcggtgtcctacatgaactggtaccagcagaagtcggggaccagccccaagcggtggatctacgacaccagcaaggtggccagcggggtcccctaccgcttctcggggtccgggtccgggacctcgtactcgctgaccatctccagcatggaggccgaggacgccgccacctactactgccagcagtggtcctcgaacccgctcaccttcggggccggcaccaagctggagctgaag

SEQ ID NO: 17 - protein, 236 aa, synthetic construct
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLGYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 18 - DNA, 708 bp, synthetic construct
  source 1..708: mol_type = other DNA
gaggtgcagctggtgcagtccggaggaggtttagttcacccaggaggctcgctgcgactgtcctgtgcagggagcggctttaccttcagcacatatctgatgtactgggtgagacaggcccccggcaagacgctggagtgggtctcagctatcggatcaggtggcgacacctattacgctgatagcgtgaagggccggtttaccataagccgcgacaacgccaaaaatagtctttacctgcagatgaacagcctccgagcagaggatatggccgtctattactgcgctcggggactcgggtattgggggcagggcaccttggtgactgtctcttcagggggaggtggttcaggtggtggaggttctggcggcggcggttccgaaattgtactaacccaatctcctggcacacttagtctgtctcctggagaacgtgcaacgttgagctgccgcgctagccagtccgtgtcttcctcgtacctcgcctggtaccagcaaaagccagggcaagctcccaggttgctgatttacggagcctcaagtagggcgactggaatccctgacagattcagtgggagcgggtccgggactgattttactctcaccatttctagacttgagcccgaagacttcgccgtttattactgtcagcagtatggctcttccccgatgtacacattcggccagggcacaaaactggaaatcaag

SEQ ID NO: 19 - DNA, 708 bp, synthetic construct
  source 1..708: mol_type = other DNA
gaggtgcagctcgtgcagtccggcggaggcctcgtgcacccaggcggctccctgcgcctgtcctgcgccgggtcaggcttcaccttctccacgtacctcatgtactgggtgcggcaggccccagggaagaccctggagtgggtgtccgccatcgggtccggcggcgacacgtactacgccgactccgtgaaggggcgcttcaccatctcccgggacaacgccaagaactccctgtacctgcagatgaactccctgcgggccgaggacatggccgtgtactactgcgccaggggcctggggtactggggccaggggaccctcgtgaccgtgtcctccggcggcggaggctcaggaggaggaggctcaggaggcggcggcagcgagatcgtcctgacgcagagccccgggaccctgtccctgagccccggggagagggcgaccctgtcctgccgggccagccagtccgtgtcctcctcctacctggcctggtaccagcagaagcccgggcaagccccacgcctcctcatctacggggcttcctccagggccacgggcatccccgaccgcttctccgggtccgggtccgggacggacttcaccctgaccatctcccggctggagcccgaggacttcgccgtgtactactgccagcagtacgggtcctcgcccatgtacaccttcgggcaggggacgaagctggagatcaag

SEQ ID NO: 20 - DNA, 708 bp, synthetic construct
  source 1..708: mol_type = other DNA
gaggtccagctggtgcagtccggcggtggcctggtgcacccaggcggcagcctgcggctgtcgtgcgccggcagtgggttcaccttctccacctacctgatgtactgggtgcgccaggccccagggaagaccctggagtgggtgtccgccatcggctccggcggcgacacctactacgccgatagcgtcaaggggcgcttcaccatctcccgcgacaacgccaagaactccctctacctccagatgaactccctgcgggccgaggacatggccgtctactactgcgccaggggcctgggctactggggccaggggaccctggtgaccgtgtcctccggcggcggtggctctggtggtggtggctctggtggcggcggcagcgagatcgtgctgacccagtcgcccgggaccctgtcgctgtcgcccggcgagcgagcgaccctcagctgccgcgcgagccagagcgtgagctccagctacctcgcgtggtaccagcagaagcccgggcaagccccacggctgctgatctacggcgctagctccagggccaccggcatccccgatcgcttcagcggcagcggcagcgggaccgacttcaccctgacgatctcccggctggagcccgaggacttcgccgtctactactgccagcagtacggctccagccccatgtacaccttcggccaggggaccaagctggagatcaag

SEQ ID NO: 21 - DNA, 708 bp, synthetic construct
  source 1..708: mol_type = other DNA
gaggtgcagctggtgcagagcggcggaggcctggtgcacccaggcggcagcctgcggctgagctgcgctggctccggcttcaccttcagcacctacctgatgtactgggtgcggcaggccccaggcaagaccctggagtgggtgtccgccatcggcagcggcggcgacacctactacgccgacagcgtgaaggggcgcttcaccatcagccgggacaacgccaagaacagcctgtacctgcagatgaacagcctgcgggccgaggacatggccgtgtactactgcgccaggggcctgggctactggggccagggcaccctggtgaccgtgagcagcggcggcggtggctctggtggaggaggctctggtggcggcggctccgagatcgtgctgacccagagccccggcaccctgagcctgagccccggggagcgggctaccctgagctgccgggcttcccagagcgtgagcagctcctacctggcctggtaccagcagaagcccgggcaggcaccacggctgctgatctacggggctagcagccgggctaccggcatccccgaccgcttctccggcagcggcagcggcaccgacttcaccctgaccatctccaggctggagcccgaggacttcgccgtgtactactgccagcagtacggcagctctcccatgtacaccttcgggcagggcaccaagctggagatcaag

SEQ ID NO: 22 - DNA, 708 bp, synthetic construct
  source 1..708: mol_type = other DNA
gaggtccagctggtccagtccggcggcggcctggttcatcccggcggcagcctgcggctgagctgcgccgggtctggcttcaccttcagcacctacctgatgtactgggtccggcaggcgcccggcaagaccctggagtgggtgtcggccatcgggtccggcggcgacacctactacgccgactcggtcaagggccggttcaccatcagccgggacaacgccaagaacagcctgtacctgcagatgaacagcctgcgggccgaggacatggccgtctactactgcgcccgcgggctgggttactggggccaggggaccctggtgaccgtcagctccggcggcggcggatctggtggtggcggatctggtggcggcggctccgagatcgtgctgacccagtcgcccgggaccctgagcctgagccccggcgagcgggctaccctgagctgccgggccagccagtcggtcagctccagctacctggcctggtaccagcagaaacccggccaggctccccggctgctgatctacggggctagtagccgggccaccggcatccccgatcggttcagcgggtccgggtccgggaccgacttcaccctgaccatcagccggctggagcccgaggacttcgccgtctactactgccagcagtacgggtccagcccgatgtacaccttcggccaggggaccaagctggagatcaag

SEQ ID NO: 23 - DNA, 708 bp, synthetic construct
  source 1..708: mol_type = other DNA
gaggtgcagctggtgcagagcggcggaggcctggtgcacccaggcggctccctgcggctcagctgcgccggttccgggttcaccttttccacgtatctgatgtactgggtgcgccaggccccggggaagaccctggaatgggtctccgccatcggctccggcggcgacacctactacgccgactccgtgaaggggcgcttcaccatttcccgggacaacgcgaagaattccctgtacctgcagatgaacagtctccgcgccgaggacatggccgtgtactactgtgcccggggcctcggatactggggccaggggaccctggtcaccgtgtccagcggcggcggaggctcaggaggaggaggctcaggaggcggcggctccgagatcgtgttgacccagagccccgggaccctgtccctgagccccggggaacgggccacgctgagctgccgcgcctcgcagagcgtctcctcctcgtacctggcctggtaccagcagaagccggggcaggctcctcggctcctcatctacggggcctcttcccgggccacgggtatccccgacaggttcagcggctccgggtccgggaccgatttcaccttgaccattagccgcctggaacccgaggacttcgccgtgtactattgtcagcagtacgggtcctcgcccatgtacacctttggccaggggaccaagctggagatcaag

SEQ ID NO: 24 - protein, 142 aa, Homo sapiens
EGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSS

SEQ ID NO: 25 - DNA, 426 bp, synthetic construct
  source 1..426: mol_type = other DNA
gaggggatctgcaggaacagggtgaccaacaatgtgaaggatgtgaccaagctggtggccaacctgcccaaggactacatgatcaccctgaagtatgtgccagggatggatgtgctgcccagccactgctggatctctgagatggtggtgcagctgtctgactccctgacagacctgctggacaagttctccaacatctcagaggggctgtccaactactccatcattgacaagctggtgaacatagtggatgacctggtggagtgtgtgaaggagaactcctccaaggacctgaagaagtccttcaagtcccctgagcccaggctgttcacccctgaggagttcttcaggatcttcaacaggtccattgatgccttcaaggactttgtggtggcctctgagacctctgactgtgtggtgtcctca

SEQ ID NO: 26 - DNA, 426 bp, synthetic construct
  source 1..426: mol_type = other DNA
gaggggatctgccggaaccgggtgaccaacaacgtgaaggacgtgaccaagctggtggccaacctgcccaaggactacatgatcaccctgaagtacgtgcccgggatggacgtgctgcccagccactgctggatcagcgagatggtggtgcagctgtccgactccctgaccgacctgctggacaagttctccaacatcagcgaggggctgagcaactactccatcatcgacaagctggtgaacatcgtggacgacctggtggagtgcgtgaaggagaacagcagcaaggacctgaagaagtccttcaagagccccgagccccggctgttcacgcccgaggagttcttccggatcttcaaccggagcatcgacgccttcaaggacttcgtggtggccagcgagaccagcgactgcgtggtgtcctcc

SEQ ID NO: 27 - DNA, 426 bp, synthetic construct
  source 1..426: mol_type = other DNA
gaggggatctgccggaaccgggtgaccaacaatgtgaaggacgtcacaaagttggtcgccaatctgccgaaggactacatgattacgctgaagtacgtccccggaatggatgtgctgcccagccactgctggatttcggagatggtggtgcagctgtccgacagtctgaccgatctgctggacaagttcagcaacatctccgaagggctgtccaactacagcatcatcgataagctggtcaacatcgtcgacgatctggtggagtgcgtcaaagagaacagcagcaaagatctgaagaagtcgttcaaatcgccggagccgcggctgttcacaccggaggagttcttccggatcttcaatcggtcgatcgacgccttcaaagattttgtggtggccagcgaaaccagcgactgcgtcgtcagcagc

SEQ ID NO: 28 - DNA, 426 bp, synthetic construct
  source 1..426: mol_type = other DNA
gaggggatctgccggaaccgcgtgacgaacaacgtgaaggacgtcacgaagctcgtcgcgaacctgccgaaggactacatgatcaccctgaagtacgtccccgggatggacgtgctcccctcgcactgctggatctccgagatggtcgtccagctgtccgactccctgacggacctcctcgacaagttctccaacatctccgaggggctctccaactactcgatcatcgacaagctggtgaacatcgtggacgacctcgtggagtgcgtcaaggagaactcctcgaaggacctcaagaagagcttcaagtcgcccgagccgcggctcttcacgcccgaggagttcttccggatcttcaaccggagcatcgacgccttcaaggacttcgtggtggcctccgagacgtccgactgcgtcgtgtcgagc

SEQ ID NO: 29 - DNA, 426 bp, synthetic construct
  source 1..426: mol_type = other DNA
gaggggatctgccggaaccgggtgaccaacaacgtgaaggacgtcaccaagctggtcgccaacctgcccaaggactacatgatcaccttgaagtacgtgcccggcatggacgtcctgcccagccactgctggatctccgagatggtcgtccagctcagcgactccctgaccgacctcctcgacaagttctccaacatctccgaggggctcagcaactactccatcatcgacaagctcgtgaacatagtggatgacctcgtggagtgcgtgaaggagaacagctccaaggacttgaagaagtccttcaagtccccggagcccaggctgttcacgcccgaggagttcttcaggatcttcaaccgatccattgacgccttcaaggacttcgtggtggcctccgagaccagcgactgcgtggtgtcctcc

SEQ ID NO: 30 - DNA, 426 bp, synthetic construct
  source 1..426: mol_type = other DNA
gaggggatctgccgcaaccgcgtgacgaacaacgtgaaggacgtgacgaagctcgtggcgaacctgcccaaggactacatgatcaccctgaagtacgtccccgggatggacgtgctgcccagccactgctggatctccgagatggtggtgcagctgagcgacagcctgacggacctgctggacaagttcagcaacatctccgaggggctgagcaactacagcatcatcgacaagctggtgaacatcgtggacgacctggtggagtgcgtgaaggagaacagctccaaggacctgaagaagagcttcaagtcgcccgagccccggctgttcacgcccgaggagttcttccggatcttcaaccggagcatcgacgccttcaaggacttcgtcgtggcgagcgagacgtccgactgcgtcgtgtcctcc

SEQ ID NO: 31 - protein, 590 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGS

SEQ ID NO: 32 - DNA, 1770 bp, Homo sapiens
  source 1..1770: mol_type = unassigned DNA
gacgctcataagtccgaggtggcccatcgctttaaggatctgggtgaggaaaattttaaggcgctggtgttgattgcatttgcgcagtatttgcagcagtgtccattcgaggatcacgtgaagttagtgaacgaggtgactgagttcgcaaagacctgtgtcgcggatgagagcgctgagaactgtgataaatcccttcacaccctgttcggcgataaactttgcactgtggccacattgcgcgaaacttacggagagatggccgactgctgcgctaaacaggagcccgagaggaacgagtgcttcttacagcataaggatgacaaccccaatctgccaagactggtcaggcctgaagtggacgtcatgtgtacagccttccatgacaacgaggagacgttcctcaagaaatacttgtacgagatcgctcgacgtcacccttatttctacgcacctgaattgctgttttttgccaagcggtataaagcagctttcacagagtgctgccaggctgcagacaaggctgcctgcttgcttccaaaattggacgagctgagagatgagggcaaggcatcctcagctaaacagcggctgaaatgtgcctctctgcaaaagttcggggagcgggctttcaaggcctgggccgttgcgcggctgagccagagatttcccaaggccgagttcgctgaagtttcaaagctcgtcaccgaccttaccaaggttcatactgaatgttgccatggcgacctgctagaatgcgcagacgacagggcggatctggccaagtacatctgtgagaatcaagactctattagtagcaagctcaaagagtgttgcgagaagccattactcgaaaagtcccattgcatcgcagaggtagaaaacgatgaaatgcctgccgaccttcccagtctcgccgccgactttgtggagagcaaagatgtgtgcaaaaattacgcagaggccaaggatgtattcctggggatgtttctctacgagtatgccagaagacaccccgattatagtgtggtgctccttctgcgcttagccaaaacatacgagaccaccctggagaagtgttgtgcagccgccgatccacacgagtgctatgctaaggtctttgatgaatttaaaccactagtcgaagagccccagaatctcataaagcagaattgcgaactttttgaacagctgggcgaatataaattccaaaatgctctgctagtacgatataccaagaaagtgccccaagtaagtactcctacgttggttgaggtgagccgtaacctggggaaggtgggatcaaagtgctgtaaacaccctgaagcgaaaaggatgccgtgcgcagaagattacctttctgtggtactcaaccagctctgcgtgttacacgagaagactccggtgtctgaccgagttacaaaatgttgcaccgagtcactcgtcaatcgccggccctgcttcagcgctctagaagtcgacgaaacctacgtgcctaaggaattcaacgccgagacgttcacctttcacgccgacatctgtactctctccgagaaggagaggcagattaagaaacaaacagcactggtggaactggttaaacacaaaccaaaggctacaaaggaacagctgaaagcggttatggatgacttcgccgcctttgtcgaaaagtgttgtaaagcagacgataaagaaacatgttttgctgaagaaggtaagaaactggtcgccgcctcgcaggcagctctgggactgggaggtggtgggtct

SEQ ID NO: 33 - DNA, 1770 bp, synthetic construct
  source 1..1770: mol_type = other DNA
gacgcccacaagtccgaggtggcccacaggttcaaggacctgggcgaggagaacttcaaggccctggtgctgatcgccttcgcccagtacctgcagcagtgccccttcgaggaccacgtgaagttggtgaacgaggtgaccgagttcgccaagacctgcgtggccgacgagagcgccgagaactgcgacaagtccctgcacaccctgttcggggacaagctgtgcaccgtggccacgctgcgggagacctacggggagatggccgactgctgcgccaagcaggagcccgagcggaacgagtgcttcctgcagcacaaggacgacaaccccaacctgccccgactggtccggcccgaggtggacgtgatgtgcaccgccttccacgacaacgaggagaccttcctgaagaagtacctgtacgagatcgcccggcggcacccctacttctacgcgcccgagctgctgttcttcgccaagcggtacaaggccgccttcaccgagtgctgccaggccgccgacaaggccgcctgcctgctgcccaagctggacgagctgcgggacgaggggaaggcctccagcgccaagcagcggctgaagtgcgcgtccctgcagaagttcggggagcgggccttcaaggcttgggccgtggcccggctgtcccaacggttccccaaggccgagttcgccgaggtgagcaagttggtgaccgacctgaccaaggtgcacaccgagtgctgccacggggacctgctggagtgcgccgacgaccgggccgatctggccaagtacatctgcgagaaccaggacagcatcagctccaagctgaaggagtgctgcgagaagcccctgctggagaagtcccactgcatcgccgaggtggagaacgacgagatgccggccgatctgccctcgctggccgccgacttcgtggagagcaaggacgtgtgcaagaactacgccgaggccaaggacgtgttcctggggatgttcctgtacgagtacgcccggcggcaccccgactactccgtggtgctgctgctgcggctggccaagacctacgagaccaccttggagaagtgctgcgccgcggccgatccgcacgagtgctacgccaaggtgttcgacgagttcaagcccctggtggaggagccccagaacctgatcaagcagaactgcgagctgttcgagcagctcggggagtacaagttccagaacgccctgctggtgcggtacaccaagaaggtgccccaggtgagcaccccgaccctggtggaggtgagccggaacctggggaaggtcgggtccaagtgctgcaagcaccccgaggccaagcggatgccctgcgccgaggactacctgtccgtggtgctgaaccagctgtgcgtgctgcacgagaagacgcccgtgtccgaccgggtgaccaagtgctgcaccgagagcttggtgaaccggcggccctgcttcagcgccctggaggtggacgagacctacgtgcccaaggagttcaacgccgagaccttcaccttccacgccgacatctgcaccctgagcgagaaggagcggcagatcaagaagcagacggccctggtggagctggtgaagcacaagcccaaggccaccaaggagcagctgaaggccgtgatggacgacttcgccgccttcgtggagaagtgctgcaaggccgacgacaaggagacctgcttcgccgaagagggcaagaagttggtggccgcgtcccaggccgcactgggtctgggaggtggtgggtct

SEQ ID NO: 34 - DNA, 1770 bp, synthetic construct
  source 1..1770: mol_type = other DNA
gacgcccacaagagcgaggtggcgcaccgcttcaaggacctgggcgaggagaacttcaaggcgctggtgctgatcgcgttcgcccagtacctccagcagtgccccttcgaggaccacgtgaagctggtgaacgaggtgaccgagttcgccaagacctgcgtggccgacgagagcgccgagaactgcgacaagagcctccacaccctgttcggggacaagctgtgcaccgtggccacgctgcgcgagacctacggggagatggccgactgctgcgccaagcaggagcccgagcgcaacgagtgcttcctccagcacaaggacgacaaccccaacctaccccgtctggtgcgccccgaggtggacgtgatgtgcaccgcgttccacgacaacgaggagaccttcctcaagaagtacctctacgagatcgcgcggcggcacccctacttctacgcgcccgagctgctgttcttcgccaagcgctacaaggccgcgttcaccgagtgctgccaggccgccgacaaggccgcgtgcctgctccccaagctcgacgagctgcgcgacgaggggaaggcgtcgagcgccaagcagaggctcaagtgcgcctcgctccagaagttcggggagcgcgcgttcaaggcgtgggccgtggcgaggctctcccagcggttccccaaggccgagttcgccgaggtgagcaagctggtgaccgacctcaccaaggtgcacaccgagtgctgccacggggacctgctggagtgcgccgacgaccgcgccgatctcgccaagtacatctgcgagaaccaggacagcatctcctccaagctcaaggagtgctgcgagaagcccctgctggagaagagccactgcatcgccgaggtggagaacgacgagatgccagccgatctcccctcgctcgccgccgacttcgtggagagcaaggacgtgtgcaagaactacgccgaggccaaggacgtgttcctggggatgttcctctacgagtacgcgcggcggcaccccgactacagcgtggtgctgctgctgcggctcgccaagacctacgagaccacgctggagaagtgctgcgccgccgccgatccccacgagtgctacgccaaggtgttcgacgagttcaagcccctggtggaggagccccagaacctgatcaagcagaactgcgagctgttcgagcagctcggggagtacaagttccagaacgcgctgctggtgcgctacaccaagaaggtgccccaggtgagcaccccgaccctggtggaggtgagccgcaacctggggaaggtcgggagcaagtgctgcaagcaccccgaggccaagcggatgccctgcgccgaggactacctctcggtggtgctcaaccagctgtgcgtgctccacgagaagaccccggtgagcgacagggtgaccaagtgctgcaccgagtcgctggtgaaccggaggccctgcttctccgcgctggaggtggacgagacctacgtgcccaaggagttcaacgccgagaccttcaccttccacgccgacatctgcaccctctccgagaaggagcgccagatcaagaagcagaccgcgctggtggagctggtgaagcacaagcccaaggccaccaaggagcagctcaaggcggtgatggacgacttcgccgcgttcgtggagaagtgctgcaaggccgacgacaaggagacctgcttcgccgaagaggggaagaagctggtggccgcgagccaagccgctctggggctgggaggtggtgggtct

SEQ ID NO: 35 - DNA, 1770 bp, synthetic construct
  source 1..1770: mol_type = other DNA
gacgcccacaagtctgaggtcgcccatcggttcaaggatctcggcgaggagaacttcaaggccctcgtgctgatcgccttcgcgcagtacctccagcagtgccccttcgaggaccacgtgaagctggtcaacgaggtgaccgagttcgccaagacctgcgtggccgacgagagcgccgagaactgcgataagagcctgcacaccctgttcggcgacaagctgtgcaccgtggcgaccctcagggagacctacggggagatggccgactgctgcgccaagcaggagcccgagcggaacgagtgcttcctgcagcacaaggatgacaaccccaatctgccccgcttggtgcgccccgaggtggacgtgatgtgcaccgcgttccacgacaacgaggagaccttcctgaagaagtacctctacgagatcgccaggcggcacccctacttctacgctcccgagctgctgttcttcgccaagcggtacaaggcggccttcacggagtgctgccaggcggccgacaaggcggcctgcctgctgcccaagctggacgagctgcgcgacgaggggaaggccagcagcgccaagcagagactgaagtgcgcctctctgcagaagttcggcgagcgggccttcaaggcgtgggccgtcgctcggctctcccagcggttccccaaggcggagttcgccgaggtgtccaagctggtgaccgacctgaccaaggtgcacaccgagtgctgccacggggacctcctggagtgcgccgacgatcgggccgacctggcgaagtacatctgcgagaaccaggatagcatcagctccaagctgaaggagtgctgcgagaagcccctgctggagaagagccactgcatcgccgaggtggagaacgacgagatgcccgcagatctgcccagcctggcggccgacttcgtggagagcaaggatgtctgcaagaattacgccgaggcgaaggacgtgttcctcgggatgttcctctacgagtacgcccggcggcaccccgattacagcgtggtgctcctcctgcggctcgccaagacctacgagacaaccctggagaagtgctgcgctgccgccgatccccacgagtgctacgccaaggtgttcgacgagttcaagcccctcgtggaggagccccagaatctcatcaagcagaactgcgagctgttcgagcagctcggggagtacaagttccagaacgccctgctggtgaggtacaccaagaaggtgccccaggtgagcaccccgaccctcgtggaggtgagccgcaacctggggaaggtcggctctaagtgctgcaagcaccccgaggccaagcggatgccctgcgcggaggactacctgtccgtggtgctcaaccagctgtgcgtgctgcacgagaagacacccgtgtccgaccgggtgaccaagtgctgcacggagtccctggtgaaccggcggccctgcttctccgctctggaggtcgacgagacctacgtgcccaaggagttcaacgccgagacgttcaccttccacgcggatatctgcacgctgagcgagaaggagaggcagatcaagaagcagacggccctcgtggagctcgtgaagcacaagcccaaggccaccaaggagcagctgaaggccgtgatggacgacttcgcggccttcgtggagaagtgctgcaaggcggacgacaaggagacctgcttcgccgaggaaggcaagaagctggtggccgccagtcaggctgccctggggctcggaggtggtgggtct

SEQ ID NO: 36 - DNA, 1770 bp, synthetic construct
  source 1..1770: mol_type = other DNA
gacgcccacaagtccgaggtggcgcaccgcttcaaggacctcggggaagagaacttcaaggccctggtgctgatcgccttcgcgcagtacctgcagcagtgccccttcgaggaccacgtgaagctcgtgaacgaggtcacggagttcgccaagacctgcgtcgccgacgagagcgccgagaactgcgacaagagcctccacaccctgttcggggacaagctctgcaccgtggcgacgctgcgggagacctacggcgagatggcggactgctgcgccaagcaggagcccgagcgcaacgagtgcttcctgcagcacaaggacgacaaccccaacctgccgcggctcgtgaggcccgaggtcgacgtgatgtgcaccgcgttccacgacaacgaggagacgttcctgaagaagtacctctacgagatcgcgaggcggcacccctacttctacgccccggagctgctgttcttcgcgaagcgctacaaggccgccttcacggagtgctgccaggccgccgacaaggccgcgtgcctgctgccgaagctcgacgagctccgggacgagggcaaggcctccagcgccaagcagcgcctcaagtgcgcctcgctgcagaagttcggggagcgggccttcaaggcctgggccgtggcgcggctgtcacagcggttccccaaggccgagttcgccgaggtctcgaagctcgtgacggacctcaccaaggtccacaccgagtgctgccacggggacctcctggagtgcgccgacgacagggccgacctcgcgaagtacatctgcgagaaccaggactccatcagctccaagctcaaggagtgctgcgagaagcccctgctggagaagtcccactgcatcgccgaggtcgagaacgacgagatgcccgcggatctcccgagcctcgccgcggacttcgtggagtccaaggacgtctgcaagaactacgccgaggccaaggacgtgttcctggggatgttcctgtacgagtacgcgaggcggcaccccgactactccgtggtgctgctgctgcggctcgccaagacctacgagacgaccctggagaagtgctgcgccgccgcagatccccacgagtgctacgccaaggtcttcgacgagttcaagcccctggtggaggagccccagaacctcatcaagcagaactgcgagctcttcgagcagctcggggagtacaagttccagaacgcgctgctggtgcggtacaccaagaaggtgccccaggtctcgaccccgaccctggtggaggtgtcccggaacctcgggaaggtcgggtccaagtgctgcaagcaccccgaggcgaagcggatgccctgcgccgaggactacctctccgtggtgctgaaccagctctgcgtgctgcacgagaagaccccggtgtcggaccgcgtcacgaagtgctgcaccgagtccctggtgaaccggaggccctgcttcagcgccctggaggtcgacgagacctacgtccccaaggagttcaacgccgagaccttcacgttccacgccgacatctgcaccctgtccgagaaggagcgccagatcaagaagcagacggccctggtggagctcgtgaagcacaagcccaaggccaccaaggagcagctcaaggccgtgatggacgacttcgcggccttcgtggagaagtgctgcaaggccgacgacaaggagacctgcttcgccgaggaaggcaagaagctcgtggcggcgtcccaggcggctctgggactcggaggtggtgggtct

SEQ ID NO: 37 - DNA, 1770 bp, synthetic construct
  source 1..1770: mol_type = other DNA
gacgcccacaagtccgaggtggcgcaccgcttcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagtccgccgagaactgcgacaagtccctgcacaccctgttcggggacaagctgtgcaccgtggccacgctgcgggagacctacggggagatggccgactgctgcgccaagcaggagcccgagcggaacgagtgcttcctgcagcacaaggacgacaaccccaacctgcccaggctggtgcggcccgaggtggacgtgatgtgcaccgccttccacgacaacgaggagaccttcctgaagaagtacctgtacgagatcgcccggcggcacccctacttctacgcgcccgagctgctgttcttcgccaagcggtacaaggccgccttcaccgagtgctgccaggccgccgacaaggccgcctgcctgctgcccaagctggacgagctgcgggacgaggggaaggcctcctccgccaagcagcggctgaagtgcgccagcctgcagaagttcggggagcgggccttcaaggcttgggccgtggcccggctgtcccagaggttccccaaggccgagttcgccgaggtgtccaagctggtgaccgacctgaccaaggtgcacaccgagtgctgccacggggacctgctggagtgcgccgacgaccgggctgacctggccaagtacatctgcgagaaccaggactccatctcctccaagctgaaggagtgctgcgagaagcccctgctggagaagtcccactgcatcgccgaggtggagaacgacgagatgccggctgacctgcccagcctggccgccgacttcgtggagtccaaggacgtgtgcaagaactacgccgaggccaaggacgtgttcctggggatgttcctgtacgagtacgcccggcggcaccccgactactccgtggtgctgctgctgcggctggccaagacctacgagacgaccctggagaagtgctgcgccgccgccgatccccacgagtgctacgccaaggtgttcgacgagttcaagcccctggtggaggagccccagaacctgatcaagcagaactgcgagctgttcgagcagctgggcgagtacaagttccagaacgccctgctggtgcggtacaccaagaaggtgccccaggtgagcaccccgaccctggtggaggtgtcccggaacctggggaaggtcgggtccaagtgctgcaagcaccccgaggccaagcggatgccctgcgccgaggactacctgtccgtggtgctgaaccagctgtgcgtgctgcacgagaagacgcccgtgtccgaccgggtgaccaagtgctgcaccgagtccctggtgaaccggcggccctgcttcagcgccctggaggtggacgagacctacgtgcccaaggagttcaacgccgagaccttcaccttccacgccgacatctgcaccctgtccgagaaggagcggcagatcaagaagcagacggccctggtggagctggtgaagcacaagcccaaggccaccaaggagcagctgaaggccgtgatggacgacttcgccgccttcgtggagaagtgctgcaaggccgacgacaaggagacctgcttcgccgaagaggggaagaagctggtggccgccagccaggctgccctgggactgggaggtggtgggtct

SEQ ID NO: 38 - protein, 244 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMKYWGQGTSVTVSSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRAD

SEQ ID NO: 39 - DNA, 732 bp, synthetic construct
  source 1..732: mol_type = other DNA
gaagtgcagttgctgcagtctggacccgagctggagaagcctggggcttcagtgatgataagttgtaaagcgagcggctcttcgttcacgggatacaacatgaattgggtaagacagaacatcggaaagtccctcgaatggattggtgcaatcgatccatactatgggggcacaagctataatcagaagtttaaaggcagggccactctgaccgtcgacaaatcctcatccaccgcttatatgcacttaaagagtcttacttctgaagacagcgccgtttattactgcgtgtcaggcatgaagtactggggtcaagggacaagcgtgaccgtcagttccggtggtggtggaagcggtggtggtggatctggcggtggtggaagtgatgtagtgatgacccagactcctctgagtctgcccgttagtttaggtgaccaggccagcatcagctgcaggtccagccagtcattggttcatagaaacggcaatacctacctgcactggtacctgcaaaaacccgggcaatctcctaaacttctcatacacaaggtctctaaccgtttctcgggggtcccggatcggtttagcggatcaggctccggaacagattttacgttgaagatttctcgcgtggaggccgaggaccttggtgtgtatttctgttcccagtccactcatgtgccaccactgacattcggcgctgggaccaaactcgaactaaagcgagcagac

SEQ ID NO: 40 - DNA, 732 bp, synthetic construct
  source 1..732: mol_type = other DNA
gaggtccagctcctccagtccgggcccgagctggagaagcccggggcctccgtgatgatctcctgcaaggcctccgggtcctccttcaccgggtacaacatgaactgggtccgccagaacatcgggaagagcctggagtggatcggggccatcgacccctactacggaggcacctcctacaaccagaagttcaagggccgggccactctgaccgtggacaagtcctcctccaccgcctacatgcacctgaagagcctgacctccgaggactccgccgtctactactgcgtctccgggatgaagtactggggccaggggacctccgtgaccgtctcctccggaggaggaggctccggaggaggaggctccggaggaggaggctccgacgtggtgatgacccagacccctctgagcctgcccgtgagcctcggggaccaggcctccatcagctgcaggagctcccagagcctggtccacaggaacgggaacacctacctccactggtacctccagaagcccgggcagagccccaagctcctgatccacaaggtctccaacaggttctccggggtccccgaccgcttcagcgggtccgggtccgggaccgacttcaccctgaagatctccagagtggaggccgaggacctcggggtctacttctgctcccagagcacccacgtgcctcctctgaccttcggggccgggaccaagctggagctcaagagggccgac

SEQ ID NO: 41 - DNA, 732 bp, synthetic construct
  source 1..732: mol_type = other DNA
gaggtccagctgctgcagtcggggcccgagctggagaagcccggggcctccgtcatgatctcgtgcaaggcctccgggtcctcgttcaccgggtacaacatgaactgggtgcgccagaacatcgggaagtcgctggagtggatcggggccatcgacccctactacggcggcaccagctacaaccagaagttcaaggggcgcgccacgctgacggtggacaagtcgtcgtcgaccgcctacatgcacctgaagtcgctgacgtcggaggactccgccgtctactactgcgtcagcgggatgaagtactggggccaggggacctcggtcaccgtgtcctccggcggcggaggaagcggaggaggaggctccggcggaggaggctccgacgtcgtgatgacgcagaccccgctgtcgctcccggtgtccctcggggaccaggcctccatcagctgccggagctcgcagtccctggtgcaccggaacgggaacacctacctccactggtacctgcagaagccggggcagtcgcccaagctgctgatccacaaggtgtccaacaggttctccggggtccccgaccgcttcagcgggagcgggagcgggaccgacttcaccctgaagatctcccgcgtggaggccgaggacctcggggtctacttctgcagccagagcacccacgtgccaccgctgaccttcggcgccggcaccaagctggagctcaagcgggccgac

SEQ ID NO: 42 - DNA, 732 bp, synthetic construct
  source 1..732: mol_type = other DNA
gaggtccagctgctccagtccgggcccgagctggagaagcccggggcctcggtgatgatcagctgcaaggcctccgggtcgagcttcaccgggtacaacatgaactgggtccggcagaacatcgggaagtcgctggagtggatcggggcgatcgacccctactacggcggcaccagctacaaccagaagttcaaggggcgcgcgaccctgaccgtcgacaagtcgagctcgaccgcctacatgcacctgaagtcgctgacctccgaggactccgcggtctactactgcgtgagcgggatgaagtactggggccaggggacctcggtgaccgtgagctccggcggcggcggatctggtggtggcggttccggcggtggcggttccgacgtggtgatgacccagaccccgctctcgctcccggtctcgctcggggaccaggcctcgatcagctgccggagctcccagtcgctggtccaccggaacgggaacacctacctccactggtacctccagaagcccgggcagtcgcccaagctgctgatccacaaggtgagcaaccgcttctccggggtccccgaccgcttctccgggtccgggtccgggaccgacttcaccctgaagatctcccgggtcgaggccgaggacctcggggtctacttctgctcccagtcgacccacgtgccgccactgaccttcggggccgggaccaagctggagctgaagcgggccgac

SEQ ID NO: 43 - DNA, 732 bp, synthetic construct
  source 1..732: mol_type = other DNA
gaggtgcagctgctgcagtccggccccgagctggagaagcccggggccagcgtgatgatcagctgcaaggccagcgggtccagcttcacggggtacaacatgaactgggtgcggcagaacatcgggaagagcctggagtggatcggggccatcgacccgtactacggcggcacgtcgtacaaccagaagttcaaggggcgcgccacgctgaccgtggacaagtccagcagcacggcctacatgcacctcaagtcgctgaccagcgaggacagcgcggtgtactactgcgtgtccggcatgaagtactggggccaggggaccagcgtgacggtgagcagcggcggtggtggcagcggtggtggcggtagcggcggtggtggcagcgacgtggtgatgacccagacgcccctcagcctgccggtcagcctgggcgaccaggccagcatttcgtgccgcagctcgcagagcctggtgcaccgcaacgggaacacctacctgcactggtacctgcagaagcccgggcagagccccaagctgctgatccacaaggtgtccaaccgcttcagcggggtgcccgaccgcttcagcgggtcgggcagcgggacggacttcaccctgaagatcagccgcgtggaggccgaggacctcggggtgtacttctgctcgcagagcacccacgtgccgccgcttaccttcggggccggcaccaagctggagctcaagcgggccgac

SEQ ID NO: 44 - DNA, 732 bp, synthetic construct
  source 1..732: mol_type = other DNA
gaggtccagctcctccagtccgggcccgagctggagaagcccggggcctccgtgatgatctcctgcaaggcctccgggtcctcgttcaccgggtacaacatgaactgggtgaggcagaacatcgggaagagcctggagtggatcggggccatcgacccctactacggcggaacctcgtacaaccagaagttcaaggggagagccacgctgaccgtggacaagtcctcgtccaccgcgtacatgcacctgaagagcctgacctccgaggactccgccgtctactactgcgtctccgggatgaagtactggggacaggggacctccgtgaccgtctcctccggcggaggaggctcaggcggaggaggctccggcggaggaggctccgacgtggtgatgacccagactcccctctccctgcccgtctccctcggcgaccaggcgagcatctcctgccggtcctctcagtccctcgtccaccggaacgggaacacctacctccactggtacctgcagaagcccgggcagtcgcccaagctgctcatccacaaggtgagcaaccgcttctccggggtccccgaccgcttctccgggtccgggtccgggaccgacttcaccctgaagatctcccgggtcgaggccgaggacctcggcgtctacttctgctcccagtctacccacgttcctccgctgaccttcggcgcggggacgaagctggagctgaagagggccgat

SEQ ID NO: 45 - protein, 241 aa, synthetic construct
EVQLEESGGGLVKPGGSLKLSCAASGFAFSSYDMSWVRQIPEKRLEWVAKVSSGGGSTYYLDTVQGRFTISRDNAKNTLYLQMSSLNSEDTAMYYCARHNYGSFAYWGQGTLVTVSAAKGGGGSGGGGSGGGGSELVMTQTPATLSVTPGDSVSLSCRASQSISNHLHWYQQKSHESPRLLIKYASQSISGIPSRFSGSGSGTDFTLSINSVETEDFGMYFCQQSNSWPHTFGGGTKLEIK

SEQ ID NO: 46 - DNA, 723 bp, synthetic construct
  source 1..723: mol_type = other DNA
gaggtgcagctggaggagtctggaggtggcctggtgaagccaggaggcagcctgaagctgagctgtgctgcctctgggtttgccttcagctcctatgacatgagctgggtgaggcagatccctgagaagaggctggagtgggtagctaaggtgagctctggaggtggcagcacctactacctggacacagtgcagggcaggttcaccatcagcagggacaatgctaagaacaccctgtacctgcagatgagcagcctgaactctgaggacacagctatgtactactgtgccaggcacaactatgggtcctttgcctactggggccaggggaccctggtgacagtgtctgcagctaaaggtggaggtggctctggaggtggaggctctggaggtggaggctctgagctggtgatgacccagaccccagctaccctgagtgtgaccccaggggactctgtgtccctgagctgcagggccagccagtccatctccaaccacctgcactggtaccagcagaagtcccatgagagccccaggctgctgatcaagtatgcctctcagtccatctctggcatccccagcaggttctcagggtcagggtcagggacagacttcaccctgagcatcaactctgtggagacagaggactttgggatgtacttctgccagcagtccaactcctggccccacacctttggaggtggcaccaagctggagatcaag

SEQ ID NO: 47 - DNA, 723 bp, synthetic construct
  source 1..723: mol_type = other DNA
gaggtgcagctggaggagtccggcggcggcttggtgaagcccggcggctccctgaagctgtcctgcgccgcctccgggttcgccttctccagctacgacatgtcctgggtgcggcagatccccgagaagcggctggagtgggtcgccaaggtgtcctccggcggcggctccacctactacctggacaccgtgcaggggcgcttcaccatctcccgggacaacgccaagaacaccctgtacctgcagatgagctccctgaacagcgaggacaccgccatgtactactgcgcccggcacaactacgggtccttcgcctactggggccaggggaccctggtgaccgtgtccgccgccaaaggaggcggcggctccggaggaggtggctcaggcggcggaggcagcgagctggtgatgacccagaccccggccacgctgtccgtgacgcccggggactccgtgtccctgagctgccgggccagccagtccatcagcaaccacctgcactggtaccagcagaagtcccacgagagcccgcggctgctgatcaagtacgccagccagtccatctccggcatccccagccggttctccgggtccgggtccgggaccgacttcaccctgtccatcaactccgtggagaccgaggacttcgggatgtacttctgccagcagagcaacagctggccccacaccttcggcggcggcaccaagctggagatcaag

SEQ ID NO: 48 - DNA, 723 bp, synthetic construct
  source 1..723: mol_type = other DNA
gaagtccagctggaggagagcggcggtggcctcgtgaagcccggcggttcgctcaagctgagctgcgcggccagcgggttcgccttcagctcgtacgacatgagctgggtgcggcagatccccgagaagcgcctggagtgggtcgccaaggtgtcctccggcggcggctcgacgtactacctggacaccgtgcaggggcgcttcacgatcagccgggacaacgcgaagaacaccctgtacctccagatgtcctcgctgaactccgaggacaccgcgatgtactactgcgcgcggcacaactacgggagcttcgcctactggggccagggcaccctggtcaccgtgagcgcggcgaaaggcggtggcggcagtggtggcggaggctctggcggcggtggctccgagctggtgatgacccagaccccggcgacgctctccgtgacgcccggggactccgtgagcctgtcctgccgggcctcgcagagcatctccaaccacctgcactggtaccagcagaagagccacgagtcgccccggctgctcatcaagtacgcctcgcagagcatctccgggattccctcgcggttctccgggagcggctcggggacggacttcacgctgtccatcaactccgtggagaccgaggacttcgggatgtacttctgccagcagagcaactcctggccgcacaccttcggcggcggcaccaagctggagatcaag

SEQ ID NO: 49 - DNA, 723 bp, synthetic construct
  source 1..723: mol_type = other DNA
gaggtccagctggaggaatccggcggcggcctcgtgaagccaggcggcagcctgaagctgtcgtgcgcggcctccgggttcgccttcagctcgtacgacatgagctgggtgcgccagatccccgagaagcgcctggagtgggtcgccaaggtgtcctccggcggcggcagcacgtactacttggacaccgtgcaggggcgcttcacgatctcgcgggacaacgcgaagaacacgctgtacctccagatgtcctcgctgaactcggaggacaccgcgatgtactactgcgcgcggcacaactatgggtccttcgcctactggggccaggggaccctggtgaccgtgagcgcggccaaaggtggcggcggctcaggtggcggaggcagcggtggcggaggcagcgagctcgtgatgacgcagacgccggcgaccctcagtgtgaccccaggggactccgtcagcctgagctgccgggcgtcccagtcgatctcgaaccacctgcactggtaccagcagaagtcccacgagagccccaggttgctcatcaagtacgccagccagtcgatctccgggattccctctcggttctcggggtccggcagcgggaccgacttcacgctgtcgatcaacagcgtggagaccgaggacttcgggatgtacttctgccagcagtccaacagttggccccacacgttcggcggcggcaccaagctggagatcaag

SEQ ID NO: 50 - DNA, 723 bp, synthetic construct
  source 1..723: mol_type = other DNA
gaggtccagctggaggagtccggcggaggcctggtcaagcccggcggcagcctgaagctgtcctgcgccgcctccggcttcgccttcagcagctacgacatgtcctgggtccggcagatcccggagaagcggctggagtgggtcgccaaggtcagctccggcggcggcagcacctactacctggacaccgtccagggccggttcaccatcagccgggacaacgccaagaacaccctgtacctgcagatgtccagcctgaactccgaggacacggccatgtactactgcgcccggcacaactacggcagcttcgcctactggggccaggggaccctggtgaccgtgtcggccgccaaaggcggaggcggctcaggaggcggaggttccggcggcggaggctccgagctggtcatgacccagacgccggccacgctgtccgtgacgcccggcgactccgtcagcctgtcctgccgggccagccagtccatcagcaaccacctgcactggtaccagcagaagtcccacgagtcgccccggctgctgatcaagtacgccagccagtccatctccggcatccccagccggttctccgggtccgggtccgggaccgacttcaccctgtccatcaactccgtcgagaccgaggacttcggcatgtacttctgccagcagtccaacagctggccgcacaccttcggcggcggcaccaagctggagatcaag

SEQ ID NO: 51 - DNA, 723 bp, synthetic construct
  source 1..723: mol_type = other DNA
gaggtccagctggaggagtccggaggaggactcgtgaagcccggaggctccctgaagctctcctgcgccgcctccgggttcgccttctcctcctacgacatgtcctgggtccggcagatccccgagaagaggctggagtgggtcgcgaaggtctcctccggaggaggctcgacctactatctcgacacggtccagggccggttcacgatctcccgggacaacgcgaagaacacgctctacctccagatgtcgagcctgaactccgaggacaccgcgatgtactactgcgcccggcacaactacgggagcttcgcctactggggccaggggactctcgtgaccgtctccgccgcgaaaggcggaggaggctccggaggaggaggctccggaggaggaggctccgagctcgtgatgacccagaccccggcgaccctctccgtgacgcccggggactccgtctccctctcctgtcgcgcgagccagtcgatctcgaaccatctccactggtaccagcagaagagtcacgagtctccccggctcctgatcaagtacgcgagccagtcgatctccgggattccctcgcggttctccgggtccgggtccgggaccgacttcacgctctcgatcaactccgtcgagaccgaggacttcgggatgtacttctgccagcagtcgaactcctggccccacacgttcggcggcggcacgaagctggagatcaag

SEQ ID NO: 52 - protein, 248 aa, synthetic construct
DIVMTQSPDSLAVSLGERATINCKSSQSLLNSRNRKNYLAWYQQKPDQSPKLLIYWASTRESGVPDRFSGSGSGTDFTLTISSLQAEDVAVYYCKQSYYLWTFGGGTKVEIKGGGGSGGGGSGGGGSEVQLVESGGGLVQPGGSLRLSCAASGFTFSDYGMAWFRQAPGKGLEWVSFISNLGYSIYYADSVKGRFTISRDNAKNSLYLQMNSLRAEDTAVYYCARAPYDYDSFDPMDYWGQGTLVTVS

SEQ ID NO: 53 - DNA, 744 bp, synthetic construct
  source 1..744: mol_type = other DNA
gacattgtgatgacccagagcccagactccctggctgtgagcctaggggagagggccaccatcaactgcaagtcctctcagagcctcctcaactccaggaacaggaagaactacctggcctggtaccagcagaagccagaccagagccccaagctgctcatctactgggcctccaccagggagtctggggtgcctgacaggttctctgggtctgggtctgggactgacttcaccctgaccatcagctccctgcaggctgaggatgtggctgtgtactactgcaagcagagctactacctgtggacctttggtggaggcaccaaggtggagatcaaaggaggtggaggctctggtggtggaggctctggtggtggaggctctgaggtgcagctggtggagtctggtggtggcctggtccagccaggaggctccctgaggctgagctgtgctgcctctgggttcaccttctcagactatgggatggcctggttcaggcaggccccagggaagggcctggagtgggtgagcttcatctccaacctgggctactccatctactatgctgactctgtgaagggcaggttcaccatcagcagggacaatgccaagaacagcctgtacctgcagatgaactccctgagggctgaggacacagctgtgtactactgtgccagggccccatatgactatgacagctttgaccccatggactactggggccaggggaccctggtgactgtgagc

SEQ ID NO: 54 - DNA, 744 bp, synthetic construct
  source 1..744: mol_type = other DNA
gacatcgtgatgacccagagccccgactccctggccgtgagcctcggggagagggccacgatcaactgcaagagctcccagagcctgctcaactcccggaaccggaagaactacctggcctggtaccagcagaagcccgaccagtcgcccaagctgctcatctactgggcctccaccagggagagcggggtgcccgaccgcttctccgggagcgggtccgggaccgacttcaccctgaccatctcctctctgcaggccgaggacgtggccgtgtactactgcaagcagagctactacctgtggacattcggcggcggcaccaaggtggagatcaaaggcggcggcggcagtggtggaggaggcagcggcggcggtggctcagaggtgcagctggtggagagcggaggcggcctggtgcagccaggcggctctctgcgcctgtcctgcgccgcctctgggttcaccttctccgactacgggatggcctggttccgccaggccccagggaaggggctggagtgggtgtccttcatctccaacctggggtacagcatctactacgccgactccgtgaagggccggttcaccatcagccgggacaacgccaagaacagcctgtacctgcagatgaactccctgcgcgccgaggacaccgccgtgtactactgcgcccgggctccctacgactacgacagcttcgaccccatggactactggggccaggggacactggtcaccgtgtcc

SEQ ID NO: 55 - DNA, 744 bp, synthetic construct
  source 1..744: mol_type = other DNA
gacatcgtgatgacgcagagccccgactctttggcggtgagcctcggggagagggccaccatcaactgcaagtcgtcgcagagcctcctcaacagccgcaaccggaagaactacctcgcctggtatcagcagaagcccgaccaaagccccaagctgctgatctactgggcctccacacgggagtctggggtgcccgaccgcttcagcggcagcggcagcggcaccgacttcaccctcaccatcagcagcctgcaggcggaggatgtggcggtgtactactgcaaacagtcctactatctgtggaccttcggcggcggcacaaaggtggagatcaaaggcggcggcggcagtggaggaggaggcagcggcggcggaggctctgaggtgcagctggtggagagcggcggcggtttggtgcagcccggcggcagcctccgactcagctgcgccgcctccggcttcaccttctccgactacgggatggcgtggtttcggcaggccccggggaaggggttggagtgggtgagcttcatctccaacttggggtacagcatctactacgccgactctgtgaaggggcgcttcaccatcagccgcgacaacgccaaaaacagcctctatctgcagatgaacagcctccgcgctgaggacacagcggtgtactactgcgcccgcgccccatacgactacgactccttcgaccccatggactactggggtcaggggacgttggtgacggtgtcg

SEQ ID NO: 56 - DNA, 744 bp, synthetic construct
  source 1..744: mol_type = other DNA
gacatcgtcatgacccagtcacctgacagcctggccgtcagcctgggcgaacgggccaccatcaactgtaagtcatctcagagcctgctgaacagccggaaccggaagaactacctggcctggtatcagcagaagcctgatcagtcacctaagctgctgatctactgggcctcaaccagagagtccggcgtgcctgacaggttcagcgggtccgggtccgggaccgacttcaccctgaccatcagcagcctgcaggccgaggacgtggccgtctattactgtaagcagtcttattacctgtggaccttcggcggcggcaccaaggtcgagatcaaaggcggcggcggctcaggtggtggtggctccggcggcggtggctctgaggtccagctggtcgagtccggcggtggcctggtccagccaggcggcagcctgaggctgtcctgcgccgcctccggcttcaccttctctgactacggcatggcctggttcagacaggccccaggcaagggcctggagtgggtcagcttcatcagcaacctgggctacagcatctattacgccgacagcgtcaagggccggttcaccatcagccgggacaacgccaagaacagcctgtacctgcagatgaacagcctgagggccgaggacacggccgtctattactgcgcccgggccccttatgactatgacagctttgaccccatggactactggggccaggggaccctggtgaccgtgtca

SEQ ID NO: 57 - DNA, 744 bp, synthetic construct
  source 1..744: mol_type = other DNA
gacatcgtgatgacccagagccccgactccctggccgtgtccctcggggagagggccaccatcaactgcaagagctcccagtccctgctgaactcccggaaccggaagaactacctggcctggtaccagcagaagcccgaccagagccccaagctgctgatctactgggccagcaccagggagagcggggtgcccgaccgcttctccgggtccgggtccgggaccgacttcaccctgaccatcagctccctgcaggccgaggacgtggccgtgtactactgcaagcagagctactacctgtggaccttcggcggcggcaccaaggtggagatcaaaggcggcggcggctcaggaggaggtggctccggcggcggaggctctgaggtgcagctggtggagagcggcggcggactggtgcagccaggcggctccctgcggctgagctgcgccgcctctgggttcaccttctccgactacgggatggcctggttccggcaggccccagggaaggggctggagtgggtgagcttcatctccaacctggggtactccatctactacgccgactccgtgaaggggcgcttcaccatctcccgggacaacgccaagaactccctgtacctgcagatgaactccctgcgggccgaggacaccgcggtgtactactgcgcccgggccccgtacgactacgacagcttcgaccccatggactactggggccaggggaccctggtgaccgtgagc

SEQ ID NO: 58 - DNA, 744 bp, synthetic construct
  source 1..744: mol_type = other DNA
gatatcgtgatgacccagtccccggactccctggcagtgtccctcggggagcgggccaccatcaactgcaagagctcccagtccctgctgaactcccggaaccggaagaactacctggcctggtaccagcagaagcccgaccagtccccgaagctgctgatctactgggccagcacccgggaatccggggtgcccgaccgcttctccgggtccgggtccgggaccgacttcaccctgaccatcagctccctgcaggccgaggacgtggcagtgtactactgcaagcagtcctactacctgtggaccttcggcggcggcaccaaggtggagatcaaaggcggcggcggctctggaggaggaggctccggcggcggaggttccgaggtgcagctggtggagtccggcggaggactggtgcagcccggcggctccctgcgactgtcctgcgccgcctccgggttcaccttctccgactacgggatggcctggttccggcaggctcccgggaaggggctggagtgggtgtccttcatctccaacctggggtactccatctactacgccgactccgtgaagggccggttcaccatctcccgggacaacgccaagaactccctgtacctgcagatgaactccctgcgggccgaggacaccgcagtgtactactgcgcccgggccccgtacgactacgactccttcgaccccatggactactggggccaggggaccctggtgaccgtgtcc

SEQ ID NO: 59 - protein, 71 aa, synthetic construct
SANSNPAMAPRERKAGCKNFFWKTFTSCGGGGSGGGGSGGGGSSANSNPAMAPRERKAGCKNFFWKTFTSC

SEQ ID NO: 60 - DNA, 258 bp, synthetic construct
  source 1..258: mol_type = other DNA
agcgcgaacagcaacccggcgatggcgccgcgcgaacgcaaagcgggctgcaaaaactttttttggaaaacctttaccagctgcggcggcggcggcagcggcggcggcggcagcggcggcggcggcagcagcgcgaacagcaacccggcgatggcgccgcgcgaacgcaaagcgggctgcaaaaactttttttggaaaacctttaccagctgcggcggcggcggcagcggcggcggcggcagcggcggcggcggcagc

SEQ ID NO: 61 - DNA, 258 bp, synthetic construct
  source 1..258: mol_type = other DNA
tctgccaactccaaccccgctatggctcccagggagcggaaggccgggtgcaagaacttcttctggaagaccttcacctcctgcggaggcggaggctcaggaggcggaggctccggaggaggcggctcctccgccaactccaaccccgctatggctcccagggagcggaaggccgggtgcaagaacttcttctggaagaccttcacctcctgcggaggcggaggctccggaggaggcggctcaggaggcggcggcagc

SEQ ID NO: 62 - DNA, 258 bp, synthetic construct
  source 1..258: mol_type = other DNA
tccgccaactccaaccccgctatggctccccgggagcggaaggctgggtgcaagaacttcttctggaagaccttcacctcctgcggaggcggaggttccggaggcggaggttccggcggaggcggctcctccgccaactccaaccccgcgatggctcccagggagcggaaggccgggtgcaagaacttcttctggaagaccttcacctcctgcggaggcggaggttccggcggaggaggttccggaggcggcggctcc

SEQ ID NO: 63 - DNA, 258 bp, synthetic construct
  source 1..258: mol_type = other DNA
agcgccaactccaaccccgctatggctccccgggagcgcaaggctgggtgcaagaacttcttctggaagaccttcacctcctgcggaggcggaggctctggaggcggaggctctggcggaggcggctcctccgccaactccaaccccgcgatggctcccagggagcgcaaggccgggtgcaagaacttcttctggaagaccttcacctcctgcggaggcggaggctctggcggaggaggctctggaggcggcggctcc

SEQ ID NO: 64 - DNA, 258 bp, synthetic construct
  source 1..258: mol_type = other DNA
tccgccaactccaacccggccatggctccccgggagaggaaggccgggtgcaagaacttcttctggaagaccttcacctcctgcggaggaggaggaagcggaggaggaggaagcggaggaggaggctcctcggccaactccaacccggccatggctccccgggagaggaaggccgggtgcaagaacttcttctggaagaccttcacctcctgcggaggaggaggaagcggaggaggaggaagcggaggaggaggatct

SEQ ID NO: 65 - DNA, 258 bp, synthetic construct
  source 1..258: mol_type = other DNA
tccgccaactccaaccccgctatggctccccgggagcggaaggctgggtgcaagaacttcttctggaagaccttcacctcctgtggcggcggcggatctggcggaggaggctctggcggaggcggctcctccgccaactccaaccccgctatggctcccagggagcggaaggccgggtgcaagaacttcttctggaagaccttcacctcctgtggcggcggcggatctggcggaggaggctctggcggaggcggaagc

SEQ ID NO: 66 - protein, 84 aa, synthetic construct
SANSNPAMAPRERKAGCKNFFWKTFTSCGGSGGSGGSGGSGGSANSNPAMAPRERKAGCKNFFWKTFTSCGGSGGSGGSGGSGG

SEQ ID NO: 67 - DNA, 252 bp, synthetic construct
  source 1..252: mol_type = other DNA
agcgcgaacagcaacccggcgatggcgccgcgcgaacgcaaagcgggctgcaaaaactttttttggaaaacctttaccagctgcggcggcagcggcggcagcggcggcagcggcggcagcggcggcagcgcgaacagcaacccggcgatggcgccgcgcgaacgcaaagcgggctgcaaaaactttttttggaaaacctttaccagctgcggcggcagcggcggcagcggcggcagcggcggcagcggcggc

SEQ ID NO: 68 - DNA, 252 bp, synthetic construct
  source 1..252: mol_type = other DNA
tccgccaactccaaccccgcaatggctccccgggagaggaaggccgggtgcaagaacttcttctggaagaccttcaccagctgcggaggctccggaggctccggaggctccggaggctccggaggctccgccaactccaaccccgcaatggctccccgggagaggaaggccgggtgcaagaacttcttctggaagaccttcaccagctgcggaggctccggaggctccggaggctccggaggctccggaggc

SEQ ID NO: 69 - DNA, 252 bp, synthetic construct
  source 1..252: mol_type = other DNA
tctgccaattccaaccccgcgatggcccctcgggagaggaaggccgggtgcaagaacttcttctggaagaccttcacctcctgcggaggctccggaggctccggaggctccggaggctccggaggctctgccaattccaaccccgcgatggcccctcgggagaggaaggccgggtgcaagaacttcttctggaagaccttcacctcctgcggaggctccggaggctccggaggctccggaggctccggaggc

SEQ ID NO: 70 - DNA, 252 bp, synthetic construct
  source 1..252: mol_type = other DNA
tccgcgaactccaaccccgcgatggctccccgggagaggaaggccgggtgcaagaacttcttctggaagacgttcacgtcctgcggaggctccggaggctccggaggctccggaggctccggaggctccgcgaactccaaccccgcgatggctccccgggagaggaaggccgggtgcaagaacttcttctggaagacgttcacgtcctgcggaggctccggaggctccggaggctccggaggctccggagga

SEQ ID NO: 71 - DNA, 252 bp, synthetic construct
  source 1..252: mol_type = other DNA
agcgccaactccaaccccgcgatggctcccagggagcggaaggccgggtgcaagaacttcttctggaagaccttcacctcctgcggaggctccggaggctccggaggctccggaggctccggaggctccgccaactccaaccccgcgatggctcccagggagcggaaggccgggtgcaagaacttcttctggaagaccttcacctcctgcggaggctccggaggctccggaggctccggaggctccggtggc

SEQ ID NO: 72 - DNA, 252 bp, synthetic construct
  source 1..252: mol_type = other DNA
tccgccaactccaaccccgctatggcccctagggagaggaaggccgggtgcaagaacttcttctggaagaccttcacctcctgcggaggctccggaggctccggaggctccggaggctccggaggctccgccaactccaaccccgctatggcccctagggagaggaaggccgggtgcaagaacttcttctggaagaccttcacctcctgcggaggctccggaggctccggaggctccggaggctccggagga

SEQ ID NO: 73 - protein, 28 aa, synthetic construct
SANSNPAMAPRERKAGCKNFFWKTFTSC

SEQ ID NO: 74 - DNA, 84 bp, synthetic construct
  source 1..84: mol_type = other DNA
tccgccaactccaacccggccatggctccccgggagaggaaggccgggtgcaagaacttcttctggaagaccttcaccagctgc

SEQ ID NO: 75 - DNA, 84 bp, synthetic construct
  source 1..84: mol_type = other DNA
agcgccaacagcaaccccgctatggctcccagggagcgcaaggccgggtgcaagaacttcttctggaagaccttcacctcttgc

SEQ ID NO: 76 - DNA, 84 bp, synthetic construct
  source 1..84: mol_type = other DNA
agcgcgaacagcaacccggccatggcccctcgcgagcgaaaggccgggtgcaagaacttcttctggaagaccttcacctcgtgc

SEQ ID NO: 77 - DNA, 84 bp, synthetic construct
  source 1..84: mol_type = other DNA
agcgccaactccaacccggctatggcgcccagggagaggaaggccggctgcaagaacttcttctggaagaccttcacctcctgc

SEQ ID NO: 78 - DNA, 84 bp, synthetic construct
  source 1..84: mol_type = other DNA
agtgcgaactcgaacccggccatggcgcccagagagcgcaaggccgggtgcaagaactttttctggaaaacgttcacatcgtgc

SEQ ID NO: 79 - DNA, 84 bp, synthetic construct
  source 1..84: mol_type = other DNA
agcgcgaactcgaacccggccatggctccgcgcgagcgtaaggccgggtgcaagaactttttctggaaaaccttcacgagttgt

SEQ ID NO: 80 - protein, 171 aa, synthetic construct
SPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQGRTTAHKDPNAIFLSFQHLLRGKVRFLMLVGGSTLCVRRAPPTTAVPSRTSLVLTLNELG

SEQ ID NO: 81 - DNA, 513 bp, synthetic construct
  source 1..513: mol_type = other DNA
tccccagctccaccagcctgtgacctgagggtgctgagcaagctcctgagggactcccatgtgctgcacagcaggctgagccagtgccctgaggtgcatcccctgccaacccctgtgctgctgccagctgtggacttctcccttggggagtggaagacccagatggaggagaccaaggcccaggacatccttggggctgtgaccctgctgctggaaggggtgatggctgccaggggccagctggggccaacctgcctcagctccctgctggggcagctgtcagggcaggtgaggctgctgctgggagccctgcagtccctgctggggacccagggcaggaccacagcccacaaggaccccaatgccatcttcctgagcttccagcacctgctgaggggcaaggtgaggttcctgatgctggttggaggcagcaccctgtgtgtcaggagagctccaccaaccacagctgtgcccagcaggaccagcctggtgctgaccctgaatgagcttgga

SEQ ID NO: 82 - DNA, 513 bp, synthetic construct
  source 1..513: mol_type = other DNA
agccccgcaccacctgcctgcgacctgcgggtgctgtccaagctgctgcgggacagccacgtgctgcacagcaggctgtcccagtgccccgaggtgcacccactgcccacgcccgtgctgctgcccgctgtggacttctccctgggcgagtggaagacacagatggaggagaccaaggcccaggacatcctgggcgccgtgaccctgctgctggaaggggtgatggccgccagagggcagctggggccaacgtgcctgtcctcactgctggggcagctgtccgggcaggtgcggctgctgctgggcgccctgcagtccctgctgggcacccaggggcgcaccacagctcacaaggaccccaacgccatcttcctgtccttccagcacctgctgcggggcaaggtgcggttcctgatgctggtcggcggcagcaccctgtgcgtgcgcagggcaccaccgaccacagctgtgcccagcaggacctcactggtgctgaccctgaacgagctgggc

SEQ ID NO: 83 - DNA, 513 bp, synthetic construct
  source 1..513: mol_type = other DNA
agcccggctcctccggcctgcgacctgcgcgtgctgagcaagctcctgcgggactcgcacgtgctgcactcgcgcctgagccagtgccccgaggtgcatcccctgcctaccccggtgctcctgcccgcggtggacttctcgctcggggagtggaagacccagatggaggagaccaaggcccaggacatactcggggccgtgaccctgctcctggaaggggtcatggcagctcggggccagctcgggcctacgtgcctgagctccctgctcgggcagctgtccgggcaggtccggctcctgctcggggccctgcagagcctgctcgggacccagggccggaccacggctcacaaggacccgaacgcgatcttcctgagcttccagcacctgctccggggcaaggtcaggttcctgatgctggtcggaggctcgaccctgtgcgtgcgcagggctcctccgaccacggccgtgccctcgcgcacgagcctggtcctgaccctgaacgagctcggg

SEQ ID NO: 84 - DNA, 513 bp, synthetic construct
  source 1..513: mol_type = other DNA
tctccggctccgcctgcctgcgacctgcgggtgctgtcgaagctgctgcgggacagccacgtcctccacagccgcctgagccagtgcccggaggtgcacccgctgcctacgccggtgctgctgccggccgtggacttcagcctcggggagtggaagacgcagatggaggagaccaaggcccaggacatcctcggggccgtgaccctgctcctggaaggggtgatggcagcgcgagggcagctggggcctacctgcctcagctccctgctggggcagctgtcggggcaggtgcggctgctgctcggggccctgcagtccctgctcgggacccagggccggaccacagcccacaaggaccccaacgccatcttcctctccttccagcacctgctccggggcaaggtccggttcctgatgctggtcggcggcagcaccctgtgcgtgagacgggctccgcctaccacggccgtgccctcgcgcacgagcctggtcctgaccctgaacgagctcggg

SEQ ID NO: 85 - DNA, 513 bp, synthetic construct
  source 1..513: mol_type = other DNA
agccccgcgccgccagcgtgcgatctgcgcgtgctgagcaagctgctgcgcgactcgcacgtgctgcactcgcggctctcgcagtgccccgaggtgcacccgctgcccacacccgtgctgctgcccgcggtggacttctcgctcggcgagtggaagacgcagatggaggagacgaaagcgcaggacatcctcggcgcggtgacgctgctgctcgaaggcgtgatggctgctcgcgggcagctcgggcctacgtgcctgagctcgctgctcgggcagctgagcgggcaggtgcggctgctgctcggcgcgctgcagtcgctgctcggcacgcaggggcgcaccacagcgcacaaggacccgaacgcgatcttcctgagcttccagcacctgctgcgcgggaaggtgcgcttcctgatgctcgtcggcggcagcacgctgtgcgtgcgcagagcgcctccgaccaccgcggtgccctcgcgcacctcgctcgtgctcacgctgaacgagctcggc

SEQ ID NO: 86 - DNA, 513 bp, synthetic construct
  source 1..513: mol_type = other DNA
agccccgctccgccagcctgcgacctgcgggtgctgagcaagctgctgcgggacagccacgtgctgcacagccggctgagccagtgccccgaggtgcacccgctgcccacgcccgtgctgctgcccgctgtggacttcagcctgggcgagtggaagacccagatggaggagaccaaggcccaggacatcctgggcgccgtgaccctgctgctggaaggggtgatggccgctaggggccagctggggcctacctgcctgagcagcctgctggggcagctgagcgggcaggtgcggctgctgctgggcgccctgcagagcctgctgggcacccaggggcgcaccacagcccacaaggaccccaacgccatcttcctgagcttccagcacctgctgcggggcaaggtgcgcttcctgatgctggtcggcggcagcaccctgtgcgtgcgcagggctccgcctaccaccgccgtgcccagccgcaccagcctggtgctgaccctgaacgagctcggc

SEQ ID NO: 87 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - DNA, 681 bp, synthetic construct
  source 1..681: mol_type = other DNA
gacaagacacacacctgccccccatgtcccgcgcctgaactcctgggggggccctctgtcttcctgttcccaccgaagccgaaagacacccttatgatatctcggacccctgaggtaacttgtgtagtagttgatgtgtctcacgaagatccagaagtgaagtttaattggtatgtggatggagtggaagttcacaacgctaagaccaagcccagggaggaacaatacaattccacataccgagttgtttcagtgctcactgtgttgcatcaggactggctcaacggcaaggagtataaatgcaaagtcagtaacaaagccctgcccgcccccatcgagaaaaccatctcgaaggcaaaaggccagcctcgcgagccacaggtctacaccctgcctccctcacgtgacgagctgacaaagaatcaggtcagcttgacgtgtttagtgaaaggattttatccaagcgacattgccgtggaatgggagagcaatggccagcctgagaacaattacaaaacaactccacctgtgctcgacagtgatgggtctttcttcttatactccaaactaacggtcgataagagcagatggcagcaaggtaacgtgttttcctgctcagtgatgcatgaggctctgcataaccactatactcagaagtcccttagtctgagcccgggtaag

SEQ ID NO: 89 - DNA, 681 bp, synthetic construct
  source 1..681: mol_type = other DNA
gataagacccacacctgtccgccgtgccccgcacccgagcttctcggcggccccagcgtgttcctgtttccgccgaagcccaaggacaccctgatgatctcccggacgcccgaggtgacctgcgtggtggtggacgtgtcccacgaggaccccgaggtgaagttcaactggtacgtggacggggtcgaggtgcacaacgccaagaccaagccccgcgaggagcagtacaactccacctaccgggtcgtgtccgtgctgaccgtgctgcaccaggactggctgaacgggaaggagtacaagtgcaaggtgtccaacaaggccctgcccgcgcccatcgagaagaccatctccaaggccaaggggcagcccagggagccccaggtgtacaccctgcctccgtcccgggacgagctgaccaagaaccaggtgtccctgacctgcctggtgaaggggttctaccccagcgacatcgccgtggagtgggagtccaacgggcagcccgagaacaactacaagaccacgccgccggtgctggactccgacgggagcttcttcctgtactccaagctgaccgtggacaagtcccggtggcagcaggggaacgtgttcagctgcagcgtgatgcacgaggccctgcacaaccactacacccagaagtccctgagcctgagccccgggaag

SEQ ID NO: 90 - DNA, 681 bp, synthetic construct
  source 1..681: mol_type = other DNA
gacaagacgcacacgtgtccgccgtgccccgcacccgaactgctcggcggccccagcgtgttcctgtttccgccgaagcccaaggacacgctgatgatctcgcgcacgcccgaggtgacgtgcgtcgtcgtcgacgtgtcgcacgaggaccccgaggtgaagttcaactggtacgtcgacggcgtcgaggtgcacaacgccaagacgaagccgcgcgaggagcagtacaacagcacgtaccgcgtcgtgagcgtgctgaccgtgctgcaccaggactggctgaacggcaaggagtacaagtgcaaggtgagcaacaaggcgctgcccgcgccgatcgagaagacgatcagcaaggccaaggggcagccacgcgagccgcaggtgtacacgctgccgccgtcgcgcgacgagctgacgaagaaccaggtgtcgctgacgtgcctcgtgaagggcttctaccccagcgacatcgccgtcgagtgggagagcaacgggcagcccgagaacaactacaagacgacgccgccggtgctcgacagcgacggcagcttcttcctgtacagcaagctgaccgtcgacaagtcgcgctggcagcagggcaacgtgttcagctgcagcgtgatgcacgaggcgctgcacaaccactacacgcagaagtcgctgtcgctgtcgcccggcaag

SEQ ID NO: 91 - DNA, 681 bp, synthetic construct
  source 1..681: mol_type = other DNA
gacaagacgcacacctgtccgccgtgcccggctcccgaactgctcggcggcccgtccgtcttcctgtttccgccgaagccgaaggacaccctgatgatcagccggacgccggaggtgacctgcgtcgtcgtcgacgtcagccacgaggaccccgaggtcaagttcaactggtacgtcgacggcgtcgaggtccacaacgccaagacgaagccccgcgaggagcagtacaactcgacgtaccgggtcgtctccgtcctgaccgtcctccaccaggactggctgaacggcaaggagtacaagtgcaaggtcagcaacaaggccctgccggccccgatcgagaagacgatctcgaaggccaagggccagcctcgggagccccaggtctacacgctgccgccgtcccgggacgagctgaccaagaaccaggtcagcctgacctgcctggtcaagggcttctacccgtccgacatcgccgtcgagtgggagtcgaacggccagcccgagaacaactacaagacgacgccgccggtcctggactccgacggcagcttcttcctgtacagcaagctgaccgtcgacaagtcccggtggcagcagggcaacgtcttcagctgctccgtcatgcacgaggccctgcacaaccactacacccagaagtccctgtccctgtcgcccggcaag

SEQ ID NO: 92 - DNA, 681 bp, synthetic construct
  source 1..681: mol_type = other DNA
gacaagacccatacctgtccgccgtgcccggctccggaactgctcggcggcccgtccgtcttcctgtttccgccgaagccgaaggacaccctgatgatcagccggacgccggaggtgacctgcgtcgtcgtcgacgtcagccacgaggacccggaggtcaagttcaactggtacgtcgacggcgtcgaggtccacaacgccaagaccaagccccgggaagagcagtacaacagcacctaccgggtcgtgtccgtcctgaccgtcctgcaccaggactggctgaacggcaaggagtacaagtgcaaggtcagcaacaaggccctgccggccccgatcgagaagaccatcagcaaggccaagggccagcccagggagccgcaggtctacaccctgccgccgtcccgggacgagctgaccaagaaccaggtcagcctgacctgcctggtcaagggcttctacccgtccgacatcgccgtcgaatgggagtccaacggccagccggagaacaactacaagacgacgccgccggtcctggactccgacggcagcttcttcctgtacagcaagctgaccgtcgacaagtcccggtggcagcagggcaacgtcttcagctgctccgtcatgcacgaggccctgcacaaccactacacccagaagtccctgtccctgtcgcccggcaag

SEQ ID NO: 93 - DNA, 681 bp, synthetic construct
  source 1..681: mol_type = other DNA
gacaagacgcacacgtgtccgccgtgcccagccccggagcttctcggcggcccctcggtgttcctgtttccgccgaagcccaaggacacgctgatgatctcccggaccccggaggtgacctgcgtcgtcgtcgacgtgtcccacgaggaccccgaggtgaagttcaactggtacgtcgacggcgtcgaggtgcacaacgcgaagacgaagccccgcgaggagcagtacaactccacgtaccgcgtcgtctccgtgctcaccgtgctgcaccaggactggctcaacggcaaggagtacaagtgcaaggtgtccaacaaggcgctgcccgcgcccatcgagaagaccatctccaaggccaaggggcagccccgggaaccccaggtgtacacgctgccgccgagccgcgacgagctgaccaagaaccaggtgtccctgacctgcctcgtcaaggggttctacccctcggacatcgccgtggagtgggagtccaacgggcagcccgagaacaactacaagacgacgccgccggtgctcgactccgacgggtccttcttcctctactcgaagctcaccgtggacaagtcccggtggcagcagggcaacgtgttcagctgctccgtgatgcacgaggcgctgcacaaccactacacgcagaagtccctgtccctgagccccgggaag

SEQ ID NO: 94 - protein, 238 aa, synthetic construct
IQLVQSGAEVKKPGASVKVSCKASGYTFTRYTMHWVRQAPGQGLEWIGYINPSRGYTNYNQKFKDRATLTTDKSTSTAYMELSSLRSEDTAVYYCARYYDDHYCLDYWGQGTLVTVSSGGSGGSGGSGGSGGDIQLTQSPSSLSASVGDRVTITCRASSSVSYMNWYQQKPGKAPKRWIYDTSKVASGAPSRFTGSGSGTDYTLTISSLQPEDFATYYCQQWSSNPLTFGGGTKLEIK

SEQ ID NO: 95 - DNA, 714 bp, synthetic construct
  source 1..714: mol_type = other DNA
atccagctggtgcagtccggggccgaggtgaagaagcccggggcctccgtgaaggtgtcctgcaaggcctccgggtacaccttcacccggtacaccatgcactgggtgaggcaggctcccgggcaggggctggagtggatcgggtacatcaaccccagccgcgggtacaccaactacaaccagaagttcaaggaccgggccacgctgaccaccgacaagtccacctccaccgcctacatggagctgagctccctgcggtccgaggacaccgccgtgtactactgcgcccggtactacgacgaccactactgcctggactactggggccaggggaccctggtgaccgtgtcctccggcggctcaggcggctcaggaggctcaggcggctcaggcggcgacatccagctgacccagagccccagctccctgtccgcctccgtcggggaccgggtgaccatcacctgccgggcctcctcctccgtgtcctacatgaactggtaccagcagaagcccgggaaggcgcccaagcggtggatctacgacacctccaaggtggcctccggggctccctcacggttcaccgggtccgggtccgggaccgactacaccctgaccatcagctccctgcagcccgaggacttcgccacctactactgccagcagtggtcctccaacccgctgaccttcggcggcggcaccaagctggagatcaag

SEQ ID NO: 96 - DNA, 714 bp, synthetic construct
  source 1..714: mol_type = other DNA
atccagctcgtgcagagcggggccgaggtgaagaagcccggggcgagcgtgaaggtgtcgtgcaaggcgagcgggtacaccttcacgcgctacaccatgcactgggtgcgccaagctcccgggcaggggctggagtggatcgggtacatcaacccctcgcgcgggtacaccaactacaaccagaagttcaaggaccgcgccacgctcaccaccgacaagtccacctccaccgcctacatggagctctcctcgctgcgctccgaggacaccgcggtgtactactgcgcgcgctactacgacgaccactactgcctcgactactggggccaggggaccctcgtgaccgtgtcgagtggtggtagtggtggtagtggtggtagtggtggtagtggtggcgacatccagctcacccagagccccagctcgttgagcgcgagcgtcggggaccgcgtgaccatcacgtgccgcgcctcgtcgagcgtgagctacatgaactggtaccagcagaagcccgggaaggccccgaagaggtggatctacgacacctccaaggtggcgagcggggctccatcgcgcttcacggggagcgggagcgggaccgactacaccctcaccatctcctcgctccagcccgaggacttcgccacctactactgccagcagtggagctccaacccgctcaccttcggcggcggcaccaagttggagatcaag

SEQ ID NO: 97 - DNA, 714 bp, synthetic construct
  source 1..714: mol_type = other DNA
atccagctggtgcagagcggcgccgaggtgaagaagcccggggcctcggtgaaggtctcctgcaaggcctcggggtacaccttcacccggtacaccatgcactgggtgcgccaagccccagggcagggcctggagtggatcgggtacatcaaccccagccgcgggtacaccaactacaaccagaagttcaaggaccgggccacgctgaccacggacaagtccacgtccacggcgtacatggagctgagctccctgcgctccgaggacaccgcggtgtactactgcgcccggtactacgacgaccactactgcctggactactggggccaggggaccctggtcaccgtgtcctccggaggctccggaggctccggaggctccggaggctccggaggcgacatccagctgacccagagccccagctccctcagcgcctccgtcggggaccgggtgaccatcacctgccgcgcctcgtcctcggtgtcctacatgaactggtaccagcagaagcccgggaaggccccgaagcggtggatctacgacacgtccaaggtcgcctccggggctccgtcgaggttcaccggctccggctccgggacggactacaccctgaccatctcctcgctgcagcccgaggacttcgccacgtactactgccagcagtggtcctcgaatcccctgaccttcggcggcggcacgaagctggagattaag

SEQ ID NO: 98 - DNA, 714 bp, synthetic construct
  source 1..714: mol_type = other DNA
atccagctggtccagtccggggccgaggtgaagaagcccggggcctccgtgaaggtctcctgcaaggcctccgggtacaccttcacccggtacaccatgcactgggtccggcaggctccagggcaggggctggagtggatcgggtacatcaaccccagccgcgggtacaccaactacaaccagaagttcaaggaccgggccacgctgaccaccgacaagtccacctccaccgcctacatggagctgagctccctgcggtccgaggacaccgccgtctactactgcgcccggtactacgacgaccactactgcctggactactggggccaggggaccctggtgaccgtctcctccggaggctccggaggctccggaggctccggaggctccggaggcgacatccagctgacccagagccccagctccctctccgcctccgtcggggaccgggtgaccatcacctgccgggcctcctcctccgtctcctacatgaactggtaccagcagaagcccgggaaggccccgaagcggtggatctacgacacctccaaggtggcctccggggcccctagcaggttcaccgggtccgggtccgggaccgactacaccctgaccatctccagcctccagcccgaggacttcgccacctactactgccagcagtggtcctccaacccgctgaccttcggcggcggcaccaagctggagatcaag

SEQ ID NO: 99 - DNA, 714 bp, synthetic construct
  source 1..714: mol_type = other DNA
atccagctggtgcagtccggggccgaggtgaagaagcccggggcctccgtgaaggtgtcctgcaaggcctccgggtacaccttcacccggtacaccatgcactgggtgaggcaggctcccgggcaggggctggagtggatcgggtacatcaacccctcgcgcgggtacaccaactacaaccagaagttcaaggaccgggccacgctgaccaccgacaagagcacctccaccgcctacatggagctgtccagcctgcggtccgaggacaccgccgtgtactactgcgcccggtactacgacgaccactactgcctggactactggggccaggggaccctggtgaccgtgtccagcggcggctctggcggctctggaggctccggcggctctggaggcgacatccagctgacccagtccccgtccagcctgtccgccagcgtcggggaccgggtgaccatcacctgccgggcctcctccagcgtgtcctacatgaactggtaccagcagaagcccgggaaggcgcccaagcggtggatctacgacacctccaaggtggcctccggggctcccagccgtttcaccgggtccgggtccgggaccgactacaccctgaccatctccagcctgcagcccgaggacttcgccacctactactgccagcagtggtccagcaacccgctgaccttcggcggcggcaccaagctggagatcaag

SEQ ID NO: 100 - DNA, 714 bp, synthetic construct
  source 1..714: mol_type = other DNA
atccagctcgtccagtccggggccgaggtgaagaagcccggggcgtccgtgaaggtgtcctgcaaggcgtccgggtacacgttcacccggtacacgatgcactgggtccgtcaggctcccgggcaggggctggagtggatcgggtacatcaacccgtcccgcgggtacacgaactacaaccagaagttcaaggaccgggcgaccctgacgaccgacaagtccacgtccaccgcgtacatggagctgtcgtccctgcggtccgaggacaccgccgtgtactactgcgcccggtactacgacgaccactactgcctcgactactggggccaggggaccctcgtgaccgtgtcctccggaggttccggcggctccggaggatctggcggctccggcggcgacatccagctgacccagtcgccctcgtccctgtccgcgtccgtcggggaccgggtgacgatcacgtgccgggcgtcctcctccgtgtcctacatgaactggtaccagcagaagcccgggaaggccccgaagaggtggatctacgacacgtccaaggtcgcgtccggggctccatcccggttcaccgggtccgggtccgggaccgactacaccctgacgatctcctcgctccagcccgaggacttcgcgacgtactactgccagcagtggtcctcgaatcccctgacgttcggcggcggcacgaagctggagatcaag

SEQ ID NO: 101 - protein, 244 aa, synthetic construct
QVQLQQSGPGLVKPSQTLSLTCAISGDSVSSNRAAWNWIRQSPSKGLEWLGRTYYRSKWYNEYAASVKSRMSINPDTSKNQFSLQLNSVTPEDTALYYCARDLWELREACDIWGQGTMVTVSSGGSGGSGGSGGSGGDIVMTQSPSFLSTFVGDRVTITCRASQGISSYLAWYQQKPGKVPKLLIYVASTLQSGVPSRFSGSGSGTEFTLTISSLQPEDFATYYCQQLNSYPFTFGPGTKVDIK

SEQ ID NO: 102 - DNA, 732 bp, synthetic construct
  source 1..732: mol_type = other DNA
caggtgcagctgcagcagtccgggcccgggctggtgaagcccagccagaccctgagcctgacctgcgccatcagcggggactccgtcagctccaaccgggccgcctggaactggatcaggcagtccccgtccaaggggctggagtggctcggccggacctactaccgctccaagtggtacaacgagtacgccgcctccgtgaagtcccggatgagcatcaaccccgatacctccaagaaccagttcagcctgcagctgaactccgtgacgcccgaggatacggccctgtactactgcgccagggacctgtgggagctgcgcgaggcctgcgacatctggggccaggggaccatggtgaccgtgtcctccggcggctcaggcggctcaggtggctccggcggatcaggcggcgacatcgtgatgacccagtccccatccttcctgtccaccttcgtcggggaccgggtgaccatcacctgccgggccagccaggggatcagctcctacctggcctggtaccagcagaagcccgggaaggtgcccaagctgctgatctacgtggcctcaaccctgcagtccggggtgcccagccggttcagcgggtccgggtccgggaccgagttcaccctgaccatcagctccctgcagcccgaggacttcgccacctactactgccagcagctgaactcctaccccttcaccttcgggcccgggaccaaggtggacatcaag

SEQ ID NO: 103 - DNA, 732 bp, synthetic construct
  source 1..732: mol_type = other DNA
caggtgcagctgcagcagtccgggcccgggctggtgaagccttcccagaccctctccctcacctgcgccatctccggggattccgtgtcctccaaccgcgccgcctggaactggatcaggcagtccccttccaaggggctggagtggctggggcgcacgtactaccgctccaagtggtacaacgagtacgccgcttccgtgaagtcccgcatgagcatcaaccccgatacctccaagaaccagttctccctgcagctgaactccgtgaccccggaggataccgcgctgtactactgcgcccgggacctgtgggagctgcgggaagcctgcgacatctggggccaggggaccatggtgaccgtgtcctccggaggctccggaggctccggaggttccggaggctccggcggcgacatcgtgatgacgcagtccccttccttcctgagcaccttcgtaggggaccgcgttaccatcacctgcagggcttcccaggggatctcctcctacctcgcatggtaccagcagaagcccgggaaggttcccaagctgctgatctacgtggcttccacgctgcagtccggggtgccttcccggttctccgggagcgggagcgggacggagttcaccctcaccatcagctccctgcagcccgaggacttcgcaacgtactactgccagcagctgaactcctaccccttcaccttcgggcccgggacgaaggtggacatcaag

SEQ ID NO: 104 - DNA, 732 bp, synthetic construct
  source 1..732: mol_type = other DNA
caagtgcagctgcagcagagcgggcccgggctggtgaagccgtcgcagacgctgtcgctgacgtgcgccatcagcggcgacagcgtgagcagcaaccgcgccgcgtggaactggatcaggcagtcgcccagcaaggggctggagtggctggggcgcacgtactaccgcagcaagtggtacaacgagtacgccgccagcgtgaagagccgcatgagcatcaaccccgacaccagcaagaaccagttctcgctgcagctgaacagcgtgacgcccgaggacaccgcgctgtactactgcgcgcgcgacttgtgggagctgcgcgaggcgtgcgacatctggggccagggcaccatggtgaccgtgagcagcggcggcagtggtggcagcggtggtagcggcggtagcggcggcgacatcgtgatgacgcagtcgccgtcgttcttgagcacgttcgtgggcgaccgcgtgaccatcacgtgccgcgcgtcgcagggcatcagcagctacttggcgtggtaccagcagaagcccggcaaggtgcccaagctgctgatctacgtggccagcacgctgcagagcggcgtgccgtcgcgcttcagcggcagcggcagcggcaccgagttcacgctgaccatcagcagcctgcagcccgaggacttcgccacgtactactgccagcagctgaacagctacccgttcacgttcgggcccggcaccaaggtggacatcaag

SEQ ID NO: 105 - DNA, 732 bp, synthetic construct
  source 1..732: mol_type = other DNA
caggtgcagctgcagcagagcggccccgggctcgtgaagccgtcgcagaccctgagcctcacctgcgccatctccggggactccgtgtcctcgaaccgcgccgcgtggaactggattcggcagagccccagcaagggcctggagtggctggggcgcacctactaccgctccaagtggtacaacgagtacgccgcctcggtgaagtcccggatgagcatcaaccccgacacctcgaagaaccagttctcgctgcagctgaactccgtgaccccggaggacacggcgctgtactactgcgcccgggacctctgggagctccgcgaggcctgcgacatctggggccaggggaccatggtcaccgtgtcctccggaggctccggaggctccggaggctccggaggctccggaggcgacatcgtgatgacccagagccccagcttcctgagcaccttcgtcggggaccgggtcaccatcacgtgccgcgcgtcccaggggatctcctcgtacctggcctggtaccagcagaagcccgggaaggtgccgaagctgctgatctacgtggcctcgacgctgcagtccggggtcccgagccgcttcagcggctccgggtccgggaccgagttcaccctgaccatctcgtcgctgcagcccgaggacttcgccacgtactactgccagcagctgaacagctaccccttcaccttcggccccgggaccaaggtggacatcaag

SEQ ID NO: 106 - DNA, 732 bp, synthetic construct
  source 1..732: mol_type = other DNA
caggtgcagctgcagcagagcgggcccgggctggtgaagcccagccagaccctgagcctgacctgcgccatctccggggacagcgtgagcagcaaccgggccgcctggaactggatcaggcagagccccagcaaggggctggagtggctggggcgcacatactaccgctccaagtggtacaatgagtatgccgccagcgtgaagagccgcatgagcatcaaccccgacacctccaagaaccagttcagcctgcagctgaacagcgtgacgcccgaggacactgccctgtactactgcgccagggacctgtgggagctgcgcgaggcctgcgacatctggggccagggcaccatggtgacagtgtcctccggcggctcaggaggctccggaggctctggcggctcaggcggcgacatcgtgatgacccagagccccagcttcctgagcaccttcgtgggcgacagggtgaccatcacctgcagggccagccagggcatcagcagctacctggcctggtaccagcagaagcccggcaaggtgcccaagctgctgatctatgtggccagcaccctgcagagcggggtgcccagccgcttcagcggcagcggcagcggcacagagttcaccctgaccatcagcagcctgcagcccgaggacttcgccacatactactgccagcagctgaacagctaccccttcaccttcgggcccggcaccaaggtggacatcaag

SEQ ID NO: 107 - DNA, 732 bp, synthetic construct
  source 1..732: mol_type = other DNA
caggtgcagctgcagcagagcgggcccgggctcgtgaagccctcgcagaccctctccctcacgtgcgcgatctccggggactccgtgtcctccaaccgcgccgcgtggaactggatacggcagagcccctcgaaggggctggagtggctggggcgcacgtactaccgctccaagtggtacaacgagtacgccgcctccgtgaagtcccgcatgagcatcaaccccgacacctccaagaaccagttctccctgcagctgaactccgtgactcccgaggacaccgcgctgtactactgcgcgcgggacctgtgggagctgcgcgaggcgtgcgacatctggggacaggggaccatggtgaccgtgtcctccggaggaagcggaggaagcggaggaagcggaggaagcggaggagacatcgtgatgacgcagtccccttccttcctctccaccttcgtgggagaccgcgtgaccatcacgtgccgcgcttcccaggggatctcctcctacctcgcgtggtaccagcagaagcccgggaaggtgcccaagctcctcatctacgtggcctccacgctgcagagcggggtgccctcgcgcttctccgggagcgggagcgggacggagttcaccctcaccatctcttccctgcagcccgaggacttcgccacgtactactgccagcagctgaactcctaccccttcaccttcgggcccgggacgaaggtggacatcaag

SEQ ID NO: 108 - protein, 248 aa, synthetic construct
EVQLLESGGGLVKPGGSLRLSCAASRFTLSSYDMNWVRQAPGKGLEWVSSISSSSSYIYYADSVKGRFTISRDNAKNSLYLQMNSLRAEDTAVYYCARDRGVGGTDYYYYGLDVWGQGTTVTVSSGGSGGSGGSGGSGGEIVMTQSPGTLSLSPGERATLSCRASQSVSSSYLAWYQQKPGQAPRLLIYGASSRATGIPDRFSGSGSGTDFTLTISRLEPEDFAVYYCQQYGSSPPYTFGQGTKVEIK

SEQ ID NO: 109 - DNA, 744 bp, synthetic construct
  source 1..744: mol_type = other DNA
gaggtgcagctgctggagagcggcggcggcctggtgaaacccggcggctccctgcggctgtcctgcgccgcctccaggttcaccctgtccagctacgacatgaactgggtgaggcaggctcccgggaaggggctggagtgggtgtcctccatctcctccagctccagctacatctactacgccgattccgtgaaggggagattcaccatctccagggacaacgccaagaactccctgtacctgcagatgaactccctgcgggccgaggacaccgccgtgtactactgcgccagggacaggggcgttggcggcaccgactactactactacgggctggacgtgtggggccaggggaccaccgtgaccgtgtccagcggcggctctggcggctctggaggctctggcggctctggcggcgagatcgtgatgacccagtctcccgggaccctgtccctgtctcccggggagagggctaccctgtcctgcagggccagccagtccgtgtcctccagctacctggcctggtaccagcagaaacccgggcaggctccccggctgctgatctacggggcctcttccagggccaccggcatccccgacaggttctccgggtccgggtccgggaccgacttcaccctgaccatctccaggctggagcccgaggacttcgccgtgtactactgccagcagtacgggtcctctccaccttacaccttcgggcaggggaccaaggtggagatcaag

SEQ ID NO: 110 - DNA, 744 bp, synthetic construct
  source 1..744: mol_type = other DNA
gaggtccagctcctggagagcggaggaggcctggtgaagcccggaggctccctgaggctctcctgcgccgcctccaggttcaccctctcctcctacgacatgaactgggtgaggcaggccccggggaaggggctggagtgggtctcctccatctcctcctcctcctcctacatctactacgccgactccgtgaaggggcgcttcaccatctcccgggacaacgccaagaactccctctacctccagatgaactccctgagggccgaggacacggccgtctactactgcgcccgggaccgaggggtaggaggcaccgactactactactacgggctggacgtctggggccaggggaccaccgtgaccgtctcctccggaggctccggaggctccggaggctccggaggctccggaggagagatcgtgatgacccagagccccgggaccctctccctgagccccggggagagggctaccctctcctgccgggccagccagagcgtctcctcctcctacctggcctggtaccagcagaagccggggcaggcccctaggctcctgatctacggggcctcctctagggccaccggcatcccggaccgcttctccgggtccgggtccgggaccgacttcaccctcaccatctcccggctggagccggaggacttcgccgtctactactgccagcagtacgggtcctctcctccttacaccttcgggcaggggaccaaggtggagatcaag

SEQ ID NO: 111 - DNA, 744 bp, synthetic construct
  source 1..744: mol_type = other DNA
gaggtgcagctgctggagtccggcggcggcctggtgaaacccggcggcagcctgcggctgtcctgcgcggcaagccgcttcacgctgtccagctacgacatgaactgggtgcgccaggcacccggcaaggggctggagtgggtgtccagcatatccagctcgtcaagctacatatactacgcggacagcgtgaagggccggtttaccatctcgcgggataacgccaagaacagcctgtacctgcagatgaacagcctgcgggccgaggacaccgcggtgtactactgcgcaagggaccgcggggtaggcggcacggattactactactacgggctggacgtgtggggccaggggaccaccgtgacggtgtcctccggcggctcaggcggttccggtggctctggcggctcaggcggcgagattgtcatgacgcagtcacccggcacgcttagcctgtcgcccggggaacgcgccacgctgtcctgccgggccagccagtcggtgtccagcagctacctggcgtggtaccagcagaaacccggccaggcgccccggctgcttatctacggggcgtctagccgggcaaccggcatcccggaccgcttcagcgggtcgggcagcgggacggacttcacgctgacaatcagccggctggagcccgaggacttcgcggtgtactactgccagcagtacggcagctcgccgccttacacgtttggccagggcaccaaggtggaaatcaag

SEQ ID NO: 112 - DNA, 744 bp, synthetic construct
  source 1..744: mol_type = other DNA
gaggtccagctgctggagagcggcggcggcctggtcaaacccggcggctcgctgcggctgagctgcgccgccagcaggttcaccctgagctcctacgacatgaactgggtgcgccaggccccaggcaaggggctggagtgggtgagctcgatcagctcgtcgtcgagctacatctactacgccgacagcgtcaaggggcgcttcaccatctcgcgcgacaacgccaagaactcgctctacctccagatgaactcgctgcgggccgaggacaccgccgtctactactgcgcccgagatcgcggggttggcggcaccgactactactactacgggctcgacgtgtggggccaggggaccaccgtgaccgtgagctccggcggctctggcggctcaggtggtagcggcggctctggcggcgagatcgtgatgacccagagccccgggaccttgtcgctgagccccggcgagagggccacgctgagctgccgggccagccagagcgtgagctccagctacctggcctggtaccagcagaaacccggccaggcgccccggctgttgatctacggggcctcatctcgggccaccggcatccccgacaggttctcggggtcggggtcggggaccgacttcaccctgaccatctcgcggctggagcccgaggacttcgccgtctactactgccagcagtacgggagctcgccgccgtacaccttcggccaggggaccaaggtcgagatcaag

SEQ ID NO: 113 - DNA, 744 bp, synthetic construct
  source 1..744: mol_type = other DNA
gaggtgcagctgctggagtccggcggcggcctggtgaaacccggcggctccctgcggctgtcctgcgccgccagcaggttcaccctctcctcctacgacatgaactgggtgcggcaggccccagggaagggcctggagtgggtgagcagcatcagcagcagcagcagctacatctactacgccgacagcgtcaaggggcgcttcaccatcagcagggacaacgccaagaacagcctgtacctgcagatgaacagcctccgggccgaggacacggccgtgtactactgcgcccgtgatcgtggcgtcggcggcaccgactactactactacgggctggacgtgtggggccaggggacgaccgtgaccgtctcctccggcggctccggaggctctggtggttctggcggctccggcggcgagatcgtcatgacccagagccccgggaccctgtccctgagccccggggagagggccacgctctcctgccgggccagccagtccgtgtcctcctcctacctggcctggtaccagcagaaacccggccaggcgcccaggctgctgatctacggggcctcttctcgcgccacgggcatccccgaccgcttctccgggagcggctccgggacggacttcaccctgaccatcagccgcctggagcccgaggacttcgccgtgtactactgccagcagtacgggtcctcgccgccgtacaccttcgggcaggggacgaaggtggagatcaag

SEQ ID NO: 114 - DNA, 744 bp, synthetic construct
  source 1..744: mol_type = other DNA
gaggtgcagctgctggagagcggcggcggcctcgtgaaacccggcggctcgctgcggctctcgtgcgccgcttcgcgcttcacgctctcgtcgtacgacatgaactgggtgcggcaggctcccgggaaggggctggagtgggtctcgtcgatctcgtcgtcgtcgagctacatctactacgccgactccgtgaaggggcgcttcacgatctcgcgcgacaacgcgaagaactcgctctacctgcagatgaactcgctgcgcgccgaggacaccgccgtctactactgcgctcgcgatcgcggagtcggcggcaccgactactactactacgggctcgacgtctggggccaggggacgaccgtgaccgtgtcgagcggcggaagcggcggaagcggaggaagcggcggaagcggcggcgagatcgtgatgacgcagtcgcccgggacgctctcgctctcgcccggcgagcgcgctacgctctcgtgccgcgcgtcgcagagcgtctcgtcgagctacctcgcgtggtaccagcagaaacccgggcaggcgccgcggctgcttatctacggcgctagctctcgcgcgaccggcatccccgaccgcttctccggctccggctccgggaccgacttcacgctgacgatctcgcggctggagcccgaggacttcgccgtctactactgccagcagtacggctcgtcgccgccgtacacgttcgggcaggggacgaaggtcgagatcaag

SEQ ID NO: 115 - protein, 234 aa, synthetic construct
DVQLVESGAELVRPGASKLSCKALAYTFTDYEMHWVKQTPVHGLEWIGIIHPGSGGTVYNQKFKGKATLTADKSSSTAYMELSSLTSEDSTVYYCTRRRYYGSSYNWYFDVWGAGNGGSGGSGGSGGSGGVLTQSPASLAASVGETVTITCRASENIYYSLAWYQQKQGKSPQLLIYNANSLEDGVPSRFSGSGSGTQYSLKINSMQPEDTATYFCEQTYDVPLTFGAGTKLEL

SEQ ID NO: 116 - DNA, 702 bp, synthetic construct
  source 1..702: mol_type = other DNA
gatgtccagctggtggagtctggggctgagctggtgaggcctggggccagcaagctgtcctgcaaggccctggcctacaccttcacagactatgagatgcactgggtgaagcagaccccagtgcatgggctggagtggattgggatcatccatccaggctctggtggcacagtctacaaccagaagttcaaggggaaggccacactcacagctgacaagtccagctccacagcctacatggagctgtccagcctgacctctgaggactccacagtctactactgcaccaggaggaggtactatggctccagctacaactggtactttgatgtgtggggagctgggaatggtggctctggtggctctggtggctctggtggctctggtggagtgctgacccagtccccagccagcctggctgcctctgttggggagacagtcaccatcacctgcagggcctctgagaacatctactacagcctggcctggtaccagcagaagcaggggaagtctccccagctgctgatctacaatgccaacagcctggaggatggggtgcccagcaggttctctgggtctgggtctggcacccagtactccctcaagatcaacagcatgcagccagaggacacagccacctacttctgtgagcagacctatgatgtgcccctcacctttggggctggcaccaagctggagctc

SEQ ID NO: 117 - DNA, 702 bp, synthetic construct
  source 1..702: mol_type = other DNA
gacgtgcagctggtggagtccggggctgagctggtgaggcccggggcctccaagctgagctgcaaggccctggcctacaccttcaccgactacgagatgcactgggtgaagcagaccccggtgcacgggctggagtggatcgggatcatccatcccgggtccggcggcaccgtgtacaaccagaagttcaaggggaaggccacgctgaccgccgacaagtccagctccaccgcctacatggagctgtccagcctgacctccgaggactccaccgtgtactactgcacccggcggcggtactacgggtcctcctacaactggtacttcgacgtgtggggcgccgggaacggcggatcaggcggctccggaggctcaggaggctccggcggcgtgctgacccagagccccgctagcctggccgcctccgtcggggagaccgtgaccatcacctgccgggcctccgagaacatctactactccctggcctggtaccagcagaagcaggggaagtctccccagctgctgatctacaacgccaactccctggaggacggggtgcccagccggttctccgggtccgggtccgggacccagtacagcctgaagatcaacagcatgcagcccgaggacaccgccacctacttctgcgagcagacctacgacgtgcccctgaccttcggggccgggaccaagctggagctg

SEQ ID NO: 118 - DNA, 702 bp, synthetic construct
  source 1..702: mol_type = other DNA
gatgtccagctcgtggagtccggggctgagctcgtgaggcccggggcctcgaagctgagctgcaaggccctggcctacaccttcacggactacgagatgcactgggtgaagcagaccccggtgcacggcctggagtggatcgggatcattcaccccgggtccggcggcaccgtgtacaaccagaagttcaagggcaaggccacgctgaccgcggacaagtcctcgtccacggcgtacatggagctgagctccctgacctccgaggactccacggtgtactactgcacacggcggcggtactacgggagctcctacaactggtacttcgacgtctggggcgccgggaacggaggctctggaggctccggaggctctggaggctccggcggcgtcctgacccagagccccgcatccctggcggcctccgtcggggagaccgtgaccatcacctgcagggcctcggagaacatctactactccctggcgtggtaccagcagaagcaggggaagtccccgcagctcctgatctacaacgccaactccctggaggacggggtgccctcgcggttctcggggtccgggtccgggacccagtactccctgaagatcaactccatgcagcccgaggacaccgcgacgtacttctgcgagcagacctacgacgtgcccctgaccttcggcgccgggacaaagctggagctc

SEQ ID NO: 119 - DNA, 702 bp, synthetic construct
  source 1..702: mol_type = other DNA
gatgtgcagctggtggagtccggggctgagctggtgaggcccggggcctccaagctgtcctgcaaggccctggcctacaccttcaccgactacgagatgcactgggtgaagcagacgcccgtgcacgggctggagtggatcgggatcattcaccccgggtccggcggcaccgtgtacaaccagaagttcaaggggaaggccacgctgaccgccgacaagtcctcctccaccgcctacatggagctgagctccctgacctccgaggactccaccgtgtactactgcacccggcggcggtactacgggtcctcctacaactggtacttcgacgtgtggggcgccgggaacggcggatctggcggctccggcggatctggcggctctggcggcgtgctgacccagagccccgcttccctggccgcctccgtcggggagaccgtgaccatcacctgccgggcctccgagaacatctactactccctggcctggtaccagcagaagcaggggaagtctccccagctgctgatctacaacgccaactccctggaggacggggtgcccagccggttctccgggtccgggtccgggacccagtactccctgaagatcaactccatgcagcccgaggacaccgccacctacttctgcgagcagacctacgacgtgcccctgaccttcggggccgggaccaagctggagctg

SEQ ID NO: 120 - DNA, 702 bp, synthetic construct
  source 1..702: mol_type = other DNA
gacgtgcagctggtggagtccggggccgagctggtacggccaggcgccagcaagctgtcctgcaaggcgctcgcctacaccttcaccgactacgagatgcactgggtgaagcagacgcccgtgcacgggctggagtggatcgggatcatacaccccgggtccggcggcaccgtctacaaccagaagttcaaggggaaggctacccttacggccgacaagtcctcctctaccgcctacatggagctgtccagcctgacgagcgaggactctaccgtctactactgcacccgcaggcggtactacgggtccagctacaactggtacttcgacgtgtggggcgccgggaacggcggtagtggcggtagcggcggtagtggcggtagcggcggcgtgctgacccagtcgcccgctagcctggccgctagcgtcggggagaccgtcaccatcacctgccgggcgagcgagaacatctactactcgctcgcctggtaccagcagaagcaggggaagagcccgcagctgctgatctataacgccaacagcctggaggacggggtgcccagccggttcagcgggagcgggagcgggacccagtactccctcaagatcaacagcatgcagccggaggataccgccacctacttctgcgagcagacgtacgacgtgcccctcacgttcggggccggcaccaagctggagctg

SEQ ID NO: 121 - DNA, 702 bp, synthetic construct
  source 1..702: mol_type = other DNA
gacgtgcagctcgtggagagcggcgcagagcttgtgcgccccggggcctccaagctgtcctgcaaggcgctcgcctacaccttcacggactacgagatgcactgggtgaagcagacccctgtgcacggcctggagtggatcgggatcattcaccccgggtcaggaggcaccgtgtacaaccagaagttcaaggggaaggccacgctgaccgccgacaagtcctcctccacggcctacatggagctctcctcgctgacctccgaggactccacggtgtactactgcacgcggaggaggtactacgggtcctcctacaactggtacttcgacgtgtggggcgcggggaatggcggctcaggaggctcaggaggctcaggaggctcaggcggcgtgctcacgcagagccccgcaagcctcgcggcgtccgtcggggagaccgtgacgatcacctgcagggcctccgagaacatctactactccctcgcgtggtaccagcagaagcaggggaagtcccctcagctcctcatctacaacgcgaactccctggaggacggggtgccctcaaggttctccgggtcggggagcggcacgcagtactccctgaagatcaactccatgcagcccgaggacaccgcgacgtacttctgcgagcagacgtacgacgtgcccctcaccttcggggccggcacgaagctcgaactg

SEQ ID NO: 122 - protein, 233 aa, synthetic construct
LSITTPEEMIEKAKGETAYLPCKFTLSPEDQGPLDIEWLISPADNQKVDQVIILYSGDKIYDDYYPDLKGRVHFTSNDLKSGDASINVTNLQLSDIGTYQCKVKKAPGVANKKIHLVVLVKPSGARCYVDGSEEIGSDFKIKCEPKEGSLPLQYEWQKLSDSQKMPTSWLAGKMCHLQRAVRPLPEATSAVIIHPWGPCLLPTWKDIPRLSITKYQVKTLNALLRVRLSHLLR

SEQ ID NO: 123 - DNA, 699 bp, synthetic construct
  source 1..699: mol_type = other DNA
ctgagcatcactacccctgaggagatgattgagaaggctaagggtgagacagcctacctgccctgcaagttcaccctgagccctgaggaccaggggcccctggacattgagtggctgatcagcccagctgacaaccagaaggtggaccaggtcatcatcctgtactcaggggacaagatctatgatgactactaccctgacctgaagggcagggtgcacttcaccagcaatgacctgaagtcaggggatgccagcatcaatgtgaccaacctgcagctgtctgacataggcacctaccagtgcaaggtcaagaaggccccaggggtagccaacaagaagatccacctggtggtgctggtcaagccctcaggggccaggtgctatgtggatggctctgaggagataggctctgacttcaagatcaagtgtgagcccaaagagggcagcctgcccctgcagtatgagtggcagaagctgtctgacagccagaagatgcccactagctggctggctggcaagatgtgccacctgcagagggctgtcaggcccctgcctgaggccacctcagctgtcatcattcacccctggggcccctgcctgctgcctacctggaaggacatccccaggctgagcatcactaagtaccaggtcaagaccctcaatgccctgctgagggtcaggctgagccacctgctgagg

SEQ ID NO: 124 - DNA, 699 bp, synthetic construct
  source 1..699: mol_type = other DNA
ctgagcatcaccacgcccgaggagatgatcgagaaggccaagggcgagaccgcctacctgccctgcaagttcaccctgagccccgaggaccaggggcccctggacatcgagtggctgatctcgcccgcggacaaccagaaggtggaccaggtgatcatcctgtactccggggacaagatctacgacgactactaccccgacctgaaggggcgcgtgcacttcacctccaacgacctgaagtccggggacgccagcatcaacgtgaccaacctgcagctgtccgacatcgggacctaccagtgcaaggtgaagaaggcgcccggggtcgccaacaagaagatccacctggtggtgctggtgaagcccagcggggccaggtgctacgtggacgggagcgaggagatcgggtccgacttcaagatcaagtgcgagcccaaagaggggtccctgcccctgcagtacgagtggcagaagctgtccgacagccagaagatgccgacctcctggctggccgggaagatgtgccacctgcagcgggccgtgaggcccctgcctgaggccaccagcgccgtgatcattcacccctggggcccctgcctgctgccaacatggaaggacatcccgcggctgtccatcaccaagtaccaggtgaagaccctgaacgccctgctgcgggtgcggctgtcgcacctgctgcgg

SEQ ID NO: 125 - DNA, 699 bp, synthetic construct
  source 1..699: mol_type = other DNA
ctctcgatcaccacacccgaggagatgatcgagaaggcgaagggcgagaccgcctacttgccctgcaagttcaccctctcgcccgaggaccaggggcccctcgatatcgagtggctcatctcgcccgcggacaaccaaaaggtggaccaggtgatcatcctctatagtggggacaagatctacgacgactactaccccgatctcaaggggcgcgtccacttcacctccaacgacctcaagagcggggacgcctcgatcaacgtgaccaacctccaattgagcgacatcgggacctaccagtgcaaggtgaagaaggcgcccggggtcgccaacaagaagatccacctcgtggtgctcgtgaagcccagtggggcgcggtgctacgtggatgggtcggaggagatcgggagcgacttcaagatcaagtgcgagcccaaagagggctcgctacccctacaatacgagtggcagaagttgagcgactcccaaaagatgcccactagctggctcgcgggcaagatgtgccacctccagcgcgccgtgcgacccctacccgaggccacgagcgcggtcatcatacacccctggggcccctgcctactccccacttggaaggacatcccgcgcctctcgatcaccaagtaccaggtgaagacgctcaacgcgctcttgcgcgtgcgcctctcgcacctcttgagg

SEQ ID NO: 126 - DNA, 699 bp, synthetic construct
  source 1..699: mol_type = other DNA
ctgagcatcaccacgccggaggagatgatcgagaaggcgaagggcgagaccgcgtacctgccctgcaagttcaccctgagcccggaggaccaggggccgctggacatcgagtggctgatcagccccgcggacaaccagaaggtggaccaggtgatcatcctgtacagcggggacaagatctacgacgactactacccggacctgaaggggcgcgtgcacttcacctccaacgacctgaagagcggggacgccagcatcaacgtgaccaacctgcagctgagcgacatcgggacctaccagtgcaaggtgaagaaggcgcccggggtcgcgaacaagaagatccacctggtggtgctggtgaagcccagcggggcccggtgctacgtggacgggtccgaggagatcgggtccgacttcaagatcaagtgcgagccgaaagaggggtccctgccgctgcagtacgagtggcagaagctgagcgacagccagaagatgccgaccagctggctggccgggaagatgtgccacctgcagcgggctgtccggccgctccctgaggctacctccgcggtgatcatccatccctggggcccctgcctgctcccgacctggaaggacatcccgcggctgagcatcaccaagtaccaggtgaagaccctgaacgcgctgctccgggtccggctgagccacctgctccgg

SEQ ID NO: 127 - DNA, 699 bp, synthetic construct
  source 1..699: mol_type = other DNA
ctctcgatcaccacgcccgaggagatgatcgagaaggccaagggcgagaccgcctacttgccctgcaagttcaccctctcgcccgaggaccaggggcccctcgacatcgagtggctcatctcgcccgcggacaaccagaaggtggaccaggtgatcatcctctactcgggcgacaagatctacgacgactactaccccgacctcaaggggcgcgtgcacttcacgagcaacgacctcaagagcggggacgcctcgatcaacgtgaccaacctccagctctccgacatcgggacctaccagtgcaaggtgaagaaggcgcccggggtcgccaacaagaagatccacctcgtggtgttggtgaagcccagcggggcgcggtgctacgtggacgggagcgaggagatcgggagcgacttcaagatcaagtgcgagcccaaagaggggtcgctcccgctccagtacgagtggcagaagctgagcgactcccagaagatgcccacgagctggctcgcggggaagatgtgccacctccagcgcgcggttcgcccactccccgaggccacgagcgcggtgatcattcacccatggggcccctgcctcttgcccacgtggaaggacatcccgcgcctctcgatcaccaagtaccaggtgaagaccctcaacgcgctcctgcgcgtgcgcctctcccacttgttgagg

SEQ ID NO: 128 - DNA, 699 bp, synthetic construct
  source 1..699: mol_type = other DNA
ctgagcatcacgacgcccgaggagatgatcgagaaggccaaaggggagaccgcctacctgccctgcaagttcaccctgagccccgaggaccaggggcccctggacatcgagtggctgatctccccggcggacaaccagaaggtcgaccaggtgatcatcctgtactccggggacaagatctacgacgactactaccccgacctgaagggccgggtgcacttcacgtcgaacgacctgaagtccggggacgcctcgatcaacgtgaccaacctgcagctgtccgacatcgggacctaccagtgcaaggtgaagaaggcgcccggcgtggccaacaagaagatccacctggtggtcctcgtgaagcccagcggggcgcggtgctacgtggacggcagcgaggagatcgggtcggacttcaagatcaagtgcgagcccaaagagggcagcctgcccctgcagtacgagtggcagaagctgtccgacagccagaagatgccgacctcctggctggccggcaagatgtgccacctgcagcgcgccgtgaggccactccccgaggcgaccagcgcggtgatcattcacccctggggcccctgcctgctgccaacctggaaggacatcccgcggctgtccatcacgaagtaccaggtgaagaccctgaacgccctgctgcgcgtccggctgagccacctgctgcgc

SEQ ID NO: 129 - protein, 240 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 130 - DNA, 720 bp, synthetic construct
  source 1..720: mol_type = other DNA
gagatccagctggtgcagtctgggggggggctggtgaagcctggggggtcagtgaggatcagctgtgctgcctcagggtacaccttcaccaactatgggatgaactgggtgaggcaggccccagggaaggggctggagtggatggggtggatcaacacccacactggggagcccacctatgctgacagcttcaaggggaggttcaccttcagcctggatgactccaagaacacagcctacctgcagatcaactccctgagggctgaggacacagctgtgtacttctgcaccaggagggggtatgactggtactttgatgtgtgggggcaggggaccacagtgacagtgtcctctggggggggggggtctggggggggggggtctggggggggggggtctgacatccagatgacccagtccccctccagcctgtctgcctcagtgggggacagggtgaccatcacctgcagggcctcccaggacatcaactcctacctgtcctggttccagcagaagcctgggaaggcccccaagaccctgatctacagggccaacaggctggagtctggggtgccctccaggttctcagggtctgggtctgggacagactacaccctgaccatctccagcctgcagtatgaggactttgggatctactactgccagcagtatgatgagtccccctggacctttgggggggggaccaagctggagatcaag

SEQ ID NO: 131 - DNA, 720 bp, synthetic construct
  source 1..720: mol_type = other DNA
gagatccagctggtgcagtccggcggcggcctggtgaaacccggcggctccgtgcggatctcctgcgccgcctccgggtacaccttcaccaactacgggatgaactgggtgcggcaggctcccgggaaggggctggagtggatgggctggatcaacacccacaccggggagcccacatacgccgactccttcaaggggcgcttcaccttctccctggacgactccaagaacaccgcctacctgcagatcaactccctgcgggccgaggacaccgccgtgtacttctgcacccggcgcgggtacgactggtacttcgacgtgtggggccaggggaccaccgtgaccgtgtcctctggcggcggaggttccggcggaggaggctccggaggaggcggcagcgacatccagatgacccagagccccagcagcctgtccgcctccgtcggggaccgggtgaccatcacctgccgggccagccaggacatcaacagctacctgtcctggttccagcagaagcccgggaaggctcccaagaccctgatctaccgggccaaccggctggagagcggggtgcccagccggttcagcgggtccgggtccgggaccgactacaccctgaccatcagctccctgcagtacgaggacttcgggatctactactgccagcagtacgacgagtccccatggaccttcggcggcggcaccaagctggagatcaag

SEQ ID NO: 132 - DNA, 720 bp, synthetic construct
  source 1..720: mol_type = other DNA
gagatccagctggtgcagtccggcggcggcctggtgaaacccggcggctccgtgcggatctcctgcgccgcctccgggtacaccttcaccaactacgggatgaactgggtgcggcaggctcccgggaaggggctggagtggatgggctggatcaacacccacaccggggagcccacgtacgccgactccttcaaggggcgcttcaccttctccctcgacgactccaagaacaccgcctacctgcagatcaactccctgcgggccgaggacaccgccgtgtacttctgcacccggcgcgggtacgactggtacttcgacgtgtggggccaggggaccaccgtgaccgtgtcctccggaggcggcggctctggaggaggcggctctggaggcggaggctccgacatccagatgacccagagccccagctccctctccgcctccgtcggggaccgggtgaccatcacctgccgcgccagccaggacatcaactcctacctctcctggttccagcagaagcccgggaaggcgcccaagaccctcatctaccgcgccaaccggctggagtccggggtgcccagccgcttctccgggtccgggtccgggaccgactacaccctcaccatctccagcctgcagtacgaggacttcgggatctactactgccagcagtacgacgagagcccctggaccttcggcggcggcaccaagctggagatcaag

SEQ ID NO: 133 - DNA, 720 bp, synthetic construct
  source 1..720: mol_type = other DNA
gaaatccagctggtgcagtccggcggcggcctggtgaaacccggcggctccgtgcggatcagctgcgcggccagcgggtacacctttacgaactacgggatgaactgggtgcgccaggcacccggcaaggggctcgaatggatgggctggatcaacacgcacaccggggagccaacctacgcggatagcttcaaggggcgcttcacgttcagcctggacgactcgaagaacaccgcctacctgcagatcaactcgctgcgcgccgaggacaccgcggtgtacttctgcacgcggcgcgggtacgactggtacttcgacgtgtggggccaggggaccaccgtgacggtgtcctccggaggaggcggctccggaggaggcggctcaggaggcggcggatcggacatccagatgacccagtcgcccagcagcctgtcggccagcgtgggcgaccgggtgaccatcacctgccgggcgtcccaggatatcaacagctacctgtcgtggttccagcagaagcccggcaaggcgcccaaaacgctgatctaccgggccaaccggctggaaagcggggtgcccagccggttcagcgggtcgggcagcgggacggactacacgctgaccatcagcagcctgcagtacgaggacttcgggatatactactgccagcagtacgacgagtcgccctggacgttcggcggaggcaccaagctggaaatcaag

SEQ ID NO: 134 - DNA, 720 bp, synthetic construct
  source 1..720: mol_type = other DNA
gagatccagctcgtgcagtccggcggcggcctcgtgaaacccggcggcagcgtgcgcatctcgtgcgccgcgtccgggtacacgttcacgaactacggcatgaactgggtgcggcaggcacccggcaaggggctggagtggatgggctggatcaacacgcacaccggcgagcccacgtacgccgactcgttcaaggggcgcttcacgttcagcctcgacgactccaagaacaccgcgtacctgcagatcaacagcctgcgcgccgaggacaccgccgtgtacttctgcacgcggcgcgggtacgactggtacttcgacgtgtggggccagggcacgaccgtgaccgtgtcgtctggcggcggtggatcaggcggcggaggctcaggcggtggaggctccgacatccagatgacgcagtcgccgtccagcctgtccgcgtccgtcggcgaccgcgtgacgatcacgtgccgcgcgtcgcaggacatcaactcgtacctgtcgtggttccagcagaagcccggcaaggcgcccaagacgctgatctaccgcgcgaaccggctggagtccggcgtgccgtcgcggttcagcgggtccgggtccggcaccgactacacgctgacgatcagcagcctgcagtacgaggacttcggcatctactactgccagcagtacgacgagtcgccgtggacgttcggcggcggcacgaagctggagatcaag

SEQ ID NO: 135 - DNA, 720 bp, synthetic construct
  source 1..720: mol_type = other DNA
gagatccagctcgtgcagtccggcggcggcctggtcaagcctggcggctccgtgcgcatctcctgcgcggcctcggggtacaccttcacgaactacggcatgaactgggtgcgccaggccccggggaaaggcctggagtggatgggctggatcaacacgcacaccggggagcccacgtacgccgactccttcaaggggcgcttcacgttctccctggacgactcgaagaacacggcctacctgcagatcaactccctgcgcgccgaggacaccgcggtgtacttctgcacgcgccggggctacgactggtacttcgacgtctggggccaggggacgacggtcaccgtgagctcaggcggcggaggaagcggaggcggaggctcaggcggaggcggctcggacatccagatgacgcagagcccgtcctcgctgagcgcctccgtgggcgaccgcgtgacgatcacctgccgggcgtcccaggacatcaactcctacctgtcgtggttccagcagaagcccgggaaggccccgaagaccctgatctaccgggcgaaccgcctggagtccggcgtgccctcgcggttctccgggtcgggctcggggacggactacacgctgaccatctcctcgctgcagtacgaggacttcggcatctactactgccagcagtacgacgagtccccgtggaccttcggcggcggcacgaagctggagatcaag

SEQ ID NO: 136 - protein, 133 aa, synthetic construct [sequence omitted]

SEQ ID NO: 137 - DNA, 399 bp, synthetic construct
  source 1..399: mol_type = other DNA
gcaccaacaagcagtagcaccaaaaagacgcagcttcagttagagcacctcctactcgacctacagatgatattgaatggtattaataactacaaaaatcctaaattgactcgaatgttgacatttaaattttatatgcccaaaaaggcaaccgaactcaagcatctgcagtgcctggaggaggaactcaagccacttgaagaggtcctgaacctggctcagtcaaaaaattttcatctgcgcccccgggacttaatcagcaatatcaacgtgattgttctggagctcaaggggtctgagaccacttttatgtgtgaatacgctgatgaaactgcgacaatcgtcgagttcctcaatagatggatcactttctgtcaatccattattagcaccctgacc

SEQ ID NO: 138 - DNA, 399 bp, synthetic construct
  source 1..399: mol_type = other DNA
gctcctaccagctccagcaccaagaagacccagctgcagctggagcacctgctgctggacctgcagatgatcctgaacgggatcaacaactacaagaaccccaagctgacccggatgctgaccttcaagttctacatgcccaagaaggccaccgagctgaagcacctgcagtgcctggaggaggagctgaagcccctggaggaggtgctgaacctggcccagagcaagaacttccacctgcggccccgggacctgatcagcaacatcaacgtgatcgtgctggagctgaaggggtccgagaccaccttcatgtgcgagtacgccgacgagaccgccaccatcgtggagttcctgaaccgctggatcaccttctgccagagcatcatctccacgctgacc

SEQ ID NO: 139 - DNA, 399 bp, synthetic construct
  source 1..399: mol_type = other DNA
gccccgaccagcagcagcaccaagaagacgcagctgcagctggagcacctgctgctggacctgcagatgatcctgaacgggatcaacaactacaagaaccccaagctgacccggatgctgaccttcaagttctacatgcccaagaaggcgaccgagctgaagcacctgcagtgcctggaggaggagctgaagcccctggaggaggtgctgaacctggcccagtccaagaacttccacctgcggccccgggacctgatcagcaacatcaacgtgatcgtgctggagctgaaggggtccgagaccaccttcatgtgcgagtacgcggacgagaccgccacaatcgtggagttcctgaaccgctggatcaccttctgccagtccatcatcagcaccctgacg

SEQ ID NO: 140 - DNA, 399 bp, synthetic construct
  source 1..399: mol_type = other DNA
gccccaacctcctcctccaccaagaagacccagctgcagctggagcacctgctgctggacctgcagatgatcctgaacgggatcaacaactacaagaaccccaagctgacccggatgctcaccttcaagttctacatgcccaagaaggccaccgagctgaagcacctgcagtgcctggaggaggagctgaagcccctggaggaggtgctgaacctggcccagtccaagaacttccacctgcggccccgggacctgatctccaacatcaacgtgatcgtgctggagctgaaggggtccgagaccaccttcatgtgcgagtacgcagatgagacggctacaatcgtggagttcctgaacaggtggatcaccttctgccagtccatcatctccaccttgaca

SEQ ID NO: 141 - DNA, 399 bp, synthetic construct
  source 1..399: mol_type = other DNA
gcgcccacgtcctcctccaccaagaagacccagctgcagctggagcacctgctgctggacctgcagatgatcctgaacgggatcaacaactacaagaaccccaagctgacccggatgctgaccttcaagttctacatgcccaagaaggccaccgagctgaagcacctgcagtgcctggaggaggagctgaagcccctggaggaggtgctgaacctggcccagtccaagaacttccacctgcggccccgggacctgatctccaacatcaacgtgatcgtgctggagctgaaggggtccgagaccaccttcatgtgcgagtacgccgacgagaccgccaccatcgtggagttcctgaaccgctggatcaccttctgccagagcatcatcagcaccctcacc

SEQ ID NO: 142 - DNA, 399 bp, synthetic construct
  source 1..399: mol_type = other DNA
gcccctacctcctcctccaccaagaagacccagctccagctggagcacctcctcctggacctccagatgatcctcaacgggatcaacaactacaagaaccccaagctcacccggatgctgaccttcaagttctacatgcccaagaaggccaccgagctgaagcacctccagtgcctggaggaggagctgaagcccctggaggaggtgctgaacctggcccagtccaagaacttccacctccggcccagggacctgatctccaacatcaacgtgatcgtcctggagctgaaggggtccgagaccaccttcatgtgcgagtacgccgatgagacagccaccatcgtggagttcctcaacaggtggatcaccttctgccagtccatcatcagcaccctcacc

SEQ ID NO: 143 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 144 - DNA, 342 bp, synthetic construct
  source 1..342: mol_type = other DNA
aactgggttaacgtgatcagcgatctgaagaagattgaagatctcatacaatccatgcacatcgacgctaccctgtatacagagtccgacgttcaccctagctgtaaggtgactgccatgaagtgctttttactggaactgcaggtaatcagtctggagtctggtgatgcctcaattcacgacacggtagagaatctaataatccttgccaacaactctttgagttccaatggcaatgtgacagaatctggctgcaaggagtgtgaagagcttgaagagaaaaacattaaagagttcctgcaatccttcgtgcatatagtgcagatgttcatcaacacctcg

SEQ ID NO: 145 - DNA, 342 bp, synthetic construct
  source 1..342: mol_type = other DNA
aactgggtgaacgtgatcagcgatctgaagaagatcgaggacctgatccagtccatgcacatcgacgctaccctgtacaccgagtcggacgttcaccccagctgcaaggtgaccgcgatgaagtgcttcctgctcgaactgcaggtgatcagcctggagagcggggacgcgagcatccacgatacggtggagaacctgatcatcctggccaacaactcgctcagctcgaacgggaacgtgaccgagagcgggtgcaaggagtgcgaggagctggaggagaagaacatcaaggagttcctccagtcgttcgtgcacatcgtgcagatgttcatcaacacctcc

SEQ ID NO: 146 - DNA, 342 bp, synthetic construct
  source 1..342: mol_type = other DNA
aactgggtgaacgtcatctccgacctgaagaagatcgaggatctgatccagtcgatgcacatcgacgcgacgctctacaccgagtcggacgttcacccctcgtgcaaggtcacggcgatgaagtgcttcctcctggagctgcaggtgatctccctggagtcgggcgacgcctcgatccacgacacggtcgagaacctgatcatcctcgcgaacaactccctctcgtccaacgggaacgtgaccgagagcgggtgcaaggagtgcgaggagctggaggagaagaacatcaaggagttcctccagtcgttcgtccacatcgtccagatgttcatcaacacctcc

SEQ ID NO: 147 - DNA, 342 bp, synthetic construct
  source 1..342: mol_type = other DNA
aactgggtgaacgtgatcagcgacctgaagaagatcgaggacctgatccagtccatgcacatcgacgccacgctgtacaccgagtccgacgtgcaccccagctgcaaggtgaccgccatgaagtgcttcctgctggagctgcaggtgatctccctggagtccggggacgcctccatccacgacaccgtggagaacctgatcatcctggccaacaactccctgtcctccaacgggaacgtgaccgagtccgggtgcaaggagtgcgaggagctggaggagaagaacatcaaggagttcctgcagagcttcgtgcacatcgtgcagatgttcatcaacacgtcg

SEQ ID NO: 148 - DNA, 342 bp, synthetic construct
  source 1..342: mol_type = other DNA
aattgggtgaacgtcatctccgacctcaagaagatcgaggacctcatccagtccatgcacatcgacgccacgctctacacggagtccgacgtgcacccgtcctgcaaggtgacggccatgaagtgcttcctgctggagctgcaggtcatctccttggagtccggggacgcctccatccacgacaccgtcgagaacctcatcatcctggccaacaactccttgagctccaacgggaacgtgacggagtccggctgcaaggagtgcgaggagctggaggagaagaacatcaaggagttcctgcagtccttcgtgcacatcgtgcagatgttcatcaacacgtcc

SEQ ID NO: 149 - DNA, 342 bp, synthetic construct
  source 1..342: mol_type = other DNA
aactgggtgaacgtgatctccgacctcaagaagatcgaggacctcatccagtcgatgcacatcgacgcgaccctctacacggagagcgacgtccatccgagctgcaaggtgaccgcgatgaagtgcttcctcctggagctccaggtgatctccctggagtccggggacgcgagcatccacgacaccgtcgagaacctgatcatcctcgcgaacaactcgctctcctcgaacgggaacgtcaccgagagcggctgcaaggagtgcgaggagctcgaagagaagaacatcaaggagttcctccagagcttcgtccacatcgtccagatgttcatcaacacgtcg

SEQ ID NO: 150 - DNA, 19 bp, synthetic construct
  source 1..19: mol_type = other DNA
gaatggagagagaattgaa

SEQ ID NO: 151 - DNA, 21 bp, synthetic construct
  source 1..21: mol_type = other DNA
gctcaggctaagcttgtacaa

SEQ ID NO: 152 - DNA, 600 bp, synthetic construct
  source 1..600: mol_type = other DNA
cccctcccttcaggccccgcgcgattccgcccccagttctgtgccggccaagatcccggctagcgccgctatcattggttagttccaagtttgcccgcccctcttcctcctcctttttccgccccctccctcccgcggaagctgggggcgcatgcgtagaggtggacgctcccctcccccgcccggggtaactgaggactcccgcgcgcggactcgctgcgccccaccctccctttccccggggccgtccggagagcgggggcgagcttgaaagttccagaacgctgcggtgagtgcgttatcgtgaggcggagcgcggtggggtgggtgcggaagggggcgaggcccgaggagtggagccgggcttgtgattgggtcttgtaagggcagccgggcgtctattggccggggaagccgtaatggcaggcagcaggggcgggccccttctggaaggttctaagatagggtataagaggcagggtggcgggcggaaaccggtctcattgaactcgcctgcagctcttgggttttttgtggcttccttcgttattggagccaggcctacaccccaggtaaaacctctgctcaagagttgggttg

SEQ ID NO: 153 - DNA, 600 bp, synthetic construct; identical to SEQ ID NO: 152
  source 1..600: mol_type = other DNA

SEQ ID NO: 154 - DNA, 591 bp, synthetic construct
  source 1..591: mol_type = other DNA
atcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctg

SEQ ID NO: 155 - protein, 5 aa, synthetic construct
GGGGS

SEQ ID NO: 156 - protein, 248 aa, synthetic construct
EIVMTQSPDSLAVSLGERATISCKSSQSLINSRNRKNYLAWYQQNPGQPPKLLIYWASTRESGVPDRFSGSGSGTDFTLTISSLQAEDVAVYYCKQSYYLWTFGQGTKVEIKGGGGSGGGGSGGGGSQVQLVESGGRLVQPGGSLRVSCEASGFTFSDYGMAWVRQAPGKGLEWVSFISNLGYSLYYADSVKGRFTISRDNAKNSLYLQMNSLRAEDTAVYYCARAPYDYDSFDPMDYWGQGTMVTVS

SEQ ID NO: 157 - DNA, 744 bp, synthetic construct
  source 1..744: mol_type = other DNA
gagatcgtgatgacccagagccccgactccctggccgtttccctcggggagcgggccaccatctcctgcaagagctcccagagcctgatcaactcccggaaccggaagaactacctggcctggtaccagcagaaccccgggcagccgccgaagctgctgatctactgggcctccacgcgggagagcggggtgcccgatcgcttctccggctccggctccgggaccgacttcaccctgaccatctccagcctgcaggccgaggacgtggccgtctactactgcaagcagagctactacctctggaccttcgggcaggggaccaaggtggagatcaaaggcggcggaggctctggcggaggaggctctggaggcggcggctcccaggtgcagctggtggagagcggcggaaggctggtgcagcccggcggctccctgagggtctcctgcgaggcctccggcttcaccttctccgactacgggatggcctgggtgaggcaggctcccgggaaggggctggagtgggtctccttcatctccaacctgggctactccctgtactacgccgactccgtgaaggggcgcttcaccatctcccgggacaacgccaagaactccctctacctgcagatgaactccctgcgggccgaggacaccgccgtctactactgcgcccgggcgccctacgactacgactccttcgaccccatggactactggggccaggggaccatggtgaccgtctcc

SEQ ID NO: 158 - DNA, 744 bp, synthetic construct
  source 1..744: mol_type = other DNA
gaaatagtaatgacgcaatcgccggactcgttggccgtgtccctgggcgagagggccaccatcagctgcaagagctcccagtccctgatcaactcccgcaaccgcaagaactacctggcctggtaccagcagaaccccggccagcctcccaagctgctgatctactgggccagcacccgggagagcggcgtgcccgacaggttcagcggcagcggcagcggcaccgacttcaccctgaccatcagctccctgcaggccgaggacgtggccgtgtactactgcaagcagagctactacctgtggaccttcggccagggcaccaaggtggagatcaagggaggtggcggcagcggcggaggtggctctggcggtggcggcagccaggtgcagctggtggagagcggcggcaggctggtgcagcctggcggctccctgcgggtgagctgcgaggccagcggcttcaccttcagcgactacggcatggcctgggtgcggcaggcccctggcaagggcctggagtgggtgagcttcatctccaacctgggctacagcctgtactacgccgacagcgtgaagggccgcttcaccatctcccgggacaacgccaagaactccctgtacctgcagatgaactccctgcgggccgaggacaccgccgtgtactactgcgcccgggccccttacgactacgacagcttcgaccccatggactactggggccagggcactatggtgaccgtgagt

SEQ ID NO: 159 - DNA, 744 bp, synthetic construct
  source 1..744: mol_type = other DNA
gaaatagtaatgacgcaatcgccggactcgttggccgtgagcctgggcgagcgtgccaccatctcctgcaagtcctcccagagcctgatcaactcccggaaccgcaagaactacctggcctggtaccagcagaaccccggccagcctcccaagctgctgatctactgggcctccacccgggagtccggcgtgcccgacagattctccggctccggctccggcaccgacttcaccctgaccatctccagcctgcaggccgaggatgtggcagtgtactactgcaagcagtcctactacctgtggaccttcggccagggcaccaaggtggagatcaagggtggcggaggctctggcggtggcggttccggaggcggaggctcccaggtgcagctggtggagtccggcggcaggctggtgcagcctggcggcagcctgagagtgtcctgcgaggcctccggcttcaccttctccgactacggcatggcttgggtgcggcaggctcccggcaagggcctggagtgggtgtccttcatctccaacctgggctacagcctgtactacgccgacagcgtgaagggcaggttcaccatctccagagacaatgccaagaacagcctgtacctgcagatgaacagcctgagagccgaggataccgccgtgtactactgcgcccgcgccccttacgattacgatagcttcgatcctatggattactggggccagggcactatggtgaccgtgtcg

SEQ ID NO: 160 - DNA, 744 bp, synthetic construct
  source 1..744: mol_type = other DNA
gaaattgtaatgacacaatcgcctgacagtttggccgtctccctcggggagagggccaccatctcctgcaagtcctcccagtccctcatcaactcccggaatcggaagaactacctcgcctggtaccagcagaaccccggccagcctcccaagctcctcatctattgggcctccacccgggagagtggggtccccgatcggttctcggggagtgggagtgggaccgatttcaccctcaccatctcttccctccaggccgaggatgtggccgtctactattgcaagcagtcctactacctctggaccttcggccaggggaccaaggtggagatcaagggaggaggtgggagtggaggaggtgggagtggaggaggtgggtcccaggtccagctcgtggagagtggtgggagactcgtccagcccggagggtccctccgggtctcctgcgaggcctcggggttcaccttctccgactatgggatggcttgggtgcgccaggcccctgggaaggggctcgagtgggtctccttcatctccaacctggggtactccctctactacgccgactccgtgaaggggagattcaccatctcccgggacaacgccaagaactccctctacctccagatgaactccctgagggccgaggacaccgccgtctactattgcgcccgggctccctacgactacgactccttcgaccccatggactattggggccaggggacaatggtgaccgtgagt

SEQ ID NO: 161 - protein, 241 aa, synthetic construct
QVQLVQSGAEVEKPGASVKVSCKASGSSFTGYNMNWVRQAPGQGLEWIGAIDPYYGGTSYNQKFKGRATLTVDKSISTAYMELSRLRSDDTAVYYCVSGMKYWGQGTLVTVSSGGGGSGGGGSGGGGSDVVMTQSPLSLPVTLGQPASISCRSSQSLVHRNGNTYLHWYQQRPGQSPRLLIHKVSNRFSGVPDRFSGSGSGTDFTLKISRVEAEDVGVYYCSQSTHVPPLTFGQGTKLEIK

SEQ ID NO: 162 - DNA, 723 bp, synthetic construct
  source 1..723: mol_type = other DNA
caggtccagctggtgcagtccggggccgaggtggagaagcccggggcctccgtgaaggtgtcctgcaaggcctccgggtcctccttcaccgggtacaacatgaactgggtgaggcaggctcccgggcaggggctggagtggatcggggccatcgacccctactacggcggcacctcctacaaccagaagttcaaggggcgcgccacgctgaccgtggacaagtccatctccaccgcctacatggagctgtcccggctgcggtccgacgacaccgccgtgtactactgcgtgtccgggatgaagtactggggccaggggaccctggtgaccgtgtcctccggcggcggaggctctggaggaggcggctctggcggcggaggctctgacgtggtgatgacccagtcgcccctgtcccttcccgtgaccctggggcagcccgcgtccatctcctgccggtccagccagtccctggtgcaccggaacgggaacacctacctgcactggtaccagcagcggcccgggcagtctcccaggctgctgatccacaaggtgtccaaccgcttctccggggtgcccgaccgcttctccgggtccgggtccgggaccgacttcaccctgaagatctcccgcgtggaggccgaggacgtcggggtgtactactgctcccagtccacgcacgtgccgccgctgaccttcgggcaggggaccaagctggagatcaag

SEQ ID NO: 163 - DNA, 723 bp, synthetic construct
  source 1..723: mol_type = other DNA
caggtgcagctggtgcagtccggggccgaggtggagaagcccggcgcctccgtgaaggtgagctgcaaggccagcggctcctccttcaccgggtacaacatgaactgggtgagacaggctcccgggcaggggctggagtggatcggggccatcgacccctactacggcgggaccagctacaaccagaagttcaaggggagggccaccctgacagtggacaagtccatctccaccgcctacatggagctgtcccgcctgcggtccgacgacaccgccgtgtactactgcgtgagcgggatgaagtactggggccaggggacactggtgaccgtgtcctccggcggaggcgggtccggcggaggcgggtccggcggaggcgggtccgacgtggtgatgacccagtctcccctgtccctgcccgtgaccctcggccagcccgcctccatctcctgccgctcctcccagtccctggtgcacaggaacgggaacacatacctgcactggtaccagcagcggcccggccagtctccccgcctgctgatccacaaggtgtccaaccgcttctccggcgtgcccgaccggttctccggctccggcagcgggaccgacttcaccctgaagatcagccgggtggaggccgaggacgtgggcgtgtactactgctcccagtctacccacgtgcctcccctgaccttcggccaggggaccaagctggagatcaag

SEQ ID NO: 164 - DNA, 723 bp, synthetic construct
  source 1..723: mol_type = other DNA
caggtgcagctggtgcagtccggggccgaggtggagaagcccggggcctctgtgaaggtgtcctgcaaggcctccgggtcctccttcaccgggtacaacatgaactgggtgagacaggctcccgggcaggggctggagtggatcggggccatcgacccctactacggcgggacctcctacaaccagaagttcaaggggagggccaccctgacagtggacaagtccatctccaccgcctacatggagctgtccaggctgaggtccgacgacaccgccgtgtactactgcgtgtccgggatgaagtactggggccaggggacactggtgaccgtgtcctctggcggcggtgggtccggcggtggcgggtctggcggcggtgggtccgatgtggtgatgacacagtctcccctgtctctgcccgtgacactggggcagcccgcctccatctcctgcaggtcctcccagtctctggtgcacaggaacgggaacacatacctgcactggtaccagcagaggcccgggcagagccccaggctgctgatccacaaggtgtccaacaggttctccggggtgcccgacaggttctccgggtccgggtccgggacagacttcaccctgaagatctccagggtggaggccgaggatgtgggcgtgtactactgctcccagagcacccacgtgccacccctgaccttcgggcaggggaccaagctggagatcaag

SEQ ID NO: 165 - DNA, 723 bp, synthetic construct
  source 1..723: mol_type = other DNA
caggtgcagctggtgcagtccggggccgaggtggagaagcccggggcctccgtgaaggtgtcctgcaaggcctccgggtcctccttcaccgggtacaacatgaactgggtgcggcaggccccagggcaggggctggagtggatcggggccatcgacccctactacggcgggacctcctacaaccagaagttcaaggggagggccaccctgacagtggacaagtccatctccaccgcctacatggagctgtccaggctgaggtccgacgacaccgccgtgtactactgcgtgtccgggatgaagtactggggccagggcacactggtgaccgtgtcctccggcggtggcgggtccggcggtggcgggtccggcggtggcgggtccgatgtggtgatgacccagtccccactgtccctgcccgtgacactggggcagcccgcctccatctcctgcaggtcctcccagtccctggtgcacaggaacgggaacacatacctgcactggtaccagcagaggcccgggcagagccccaggctgctgatccacaaggtgtccaacaggttctccggggtgcccgataggttctccgggtccgggtccgggaccgacttcaccctgaagatctccagggtggaggccgaggacgtgggcgtgtactactgctcccagagcacccacgtgccacccctgaccttcgggcaggggaccaagctggagatcaag

SEQ ID NO: 166 - protein, 236 aa, synthetic construct
EVQLVESGGGLVKPGGSLRLSCAASGFTFSTYLMYWIRQAPGKGLEWVSALGSGGDTYYADSVKGRFTISRDNAKNSLYLQMNSLRAEDTAVYYCARGLGYWGQGTLVTVSSGGGGSGGGGSGGGGSEIVLTQSPGSLSLSPGERATLSCRASQSVSSSYLAWYQQKPGQAPRLLIYGASSRATGIPDRFSGSGSGTDFSLTISRLEPEDFAVYYCQQYGSSPMYTFGPGTRVEIK

SEQ ID NO: 167 - DNA, 708 bp, synthetic construct
  source 1..708: mol_type = other DNA
gaggtgcagctggtggagtctggcggcggcctggtgaagcccggaggctccctgcggctgtcctgcgctgcctccgggttcaccttctccacctacctgatgtactggatcaggcaggctcccgggaaggggctggagtgggtgtccgccctggggtccggcggcgacacctactacgccgactccgtgaaggggcgcttcaccatctcccgggacaacgccaagaactccctgtacctgcagatgaactccctgcgggccgaggacaccgccgtgtactactgcgccaggggcctggggtactggggccaggggaccctggtgaccgtgtcctccggcggcggaggctctggaggaggcggctctggcggcggaggctctgagatcgtgctgacccagagccccgggtccctgtccctgagccccggggagcgggctaccctgtcctgccgggccagccagtccgtgtcctcctcctacctggcctggtaccagcagaagcccgggcaggctcccaggctgctgatctacggggcttcctccagggccaccggcatccccgaccgcttctccgggtccgggtccgggaccgacttctccctgaccatctcccggctggagcccgaggacttcgccgtgtactactgccagcagtacgggtccagccccatgtacaccttcgggcccgggacccgcgtggagatcaag

SEQ ID NO: 168 - DNA, 708 bp, synthetic construct
  source 1..708: mol_type = other DNA
gaggtgcagctggtggagtctggcggcgggctggtgaagcccggcgggtccctgcggctgtcctgcgccgcctccgggttcaccttctccacatacctgatgtactggatccggcaggctcccgggaaggggctggagtgggtgtccgctctggggtccggcggggacacctactacgccgactccgtgaaggggcggttcaccatctccagggacaacgccaagaactccctgtacctgcagatgaactccctgcgggccgaggacaccgccgtgtactactgcgccaggggcctggggtactggggccaggggacactggtgaccgtgtcctctggcggcggtgggtccggcggtggcgggtctggcggcggtgggtccgagatcgtgctgacccagagccccgggtccctgtccctgtcacccggggagcgggccaccctgtcctgcagggcctcccagtccgtgtcctccagctacctggcctggtaccagcagaagcccgggcaggctccccggctgctgatctacggggcctcctccagggccaccgggatccccgaccggttctccgggtccgggtccgggaccgacttctccctgaccatctcccggctggagcccgaggacttcgccgtgtactactgccagcagtacgggtccagccccatgtacaccttcgggcccgggaccagggtggagatcaag

SEQ ID NO: 169 - DNA, 708 bp, synthetic construct
  source 1..708: mol_type = other DNA
gaggtgcagctggtggagtctggcggcgggctggtgaagcccggcgggtccctgcggctgtcctgcgccgcctccgggttcaccttttccacatacctgatgtactggatcagacaggctcccgggaaggggctggagtgggtgtccgctctggggtccggcggggacacctactacgccgactccgtgaaggggaggtttacaatctccagggacaacgccaagaactccctgtacctgcagatgaactccctgcgggccgaggacaccgccgtgtactactgcgcccgcgggctggggtactggggccaggggacactggtgacagtgtcctctggcggcggagggtccggcggaggcgggtctggcggcggagggtccgagatcgtgctgacacagtctcccgggtccctgtccctgtcacccggggagagggccaccctgtcctgcagggcctcccagtccgtgtcctcctcttacctggcctggtaccagcagaagcccgggcaggctcccaggctgctgatctacggggcctcttccagggccaccgggatccccgacaggttctccgggtccgggtccgggacagacttctccctgacaatctccaggctggagcccgaggacttcgccgtgtactactgccagcagtacgggtcctctcccatgtacaccttcgggcccgggacaagggtggagatcaag

SEQ ID NO: 170 - DNA, 708 bp, synthetic construct
  source 1..708: mol_type = other DNA
gaggtgcagctggtggagtctggcggcgggctggtgaagcccggcgggtccctgcggctgtcctgcgccgcctccggcttcaccttctccacatacctgatgtactggatccggcaggctcccgggaaggggctggagtgggtgtccgccctcgggtccggcggggacacatactacgccgactccgtgaaggggcggttcaccatctcccgggacaacgccaagaattccctgtacctgcagatgaattccctgcgggccgaggacacagccgtgtactactgcgccagaggcctggggtactggggccaggggacactggtgacagtgtcctccggcggaggcgggtccggcggaggcgggtctggcggcggagggtccgagatcgtcctgacccagtctcccgggtccctgtccctgtctcccggggagagggccacactgtcctgccgggcctcccagtccgtgtcttcctcctacctggcctggtaccagcagaagcccgggcaggccccacggctgctgatctacggggcctcctccagggccacaggcatccccgacagattctccgggtccgggtccgggacagacttctccctgaccatctcccggctggagcccgaggacttcgccgtgtactactgccagcagtacgggtccagccccatgtacaccttcgggcccgggacaagggtggagatcaag

SEQ ID NO: 171 - protein, 236 aa, synthetic construct
EVQLVESGGGLVKPGGSLRLSCAASGFTFSTYIMYWIRQAPGKGLEWVSAIGSGGDTYYADSVKGRFTISRDNAKNSLYLQMNSLRAEDTAVYYCARGLGYWGQGTLVTVSSGGGGSGGGGSGGGGSEIVLTQSPGSLSLSPGERATLSCRASQSVSSSYLAWYQQKPGQAPRLLIYGASSRATGIPDRFSGSGSGTDFSLTISRLEPEDFAVYYCQQYGSSPMYTFGPGTRVEIK

SEQ ID NO: 172 - protein, 236 aa, synthetic construct
EVQLVESGGGLVKPGGSLRLSCAASGFTFSTYLMYWIRQAPGKGLEWVSAIGSGGDTYYADSVKGRFTISRDNAKNSLYLQMNSLRAEDTAVYYCARGIGYWGQGTLVTVSSGGGGSGGGGSGGGGSEIVLTQSPGSLSLSPGERATLSCRASQSVSSSYLAWYQQKPGQAPRLLIYGASSRATGIPDRFSGSGSGTDFSLTISRLEPEDFAVYYCQQYGSSPMYTFGPGTRVEIK

SEQ ID NO: 173 - protein, 236 aa, synthetic construct
EVQLVESGGGLVKPGGSLRLSCAASGFTFSTYLMYWIRQAPGKGLEWVSAIGSGGDTYYADSVKGRFTISRDNAKNSLYLQMNSLRAEDTAVYYCARGLGYWGQGTLVTVSSGGGGSGGGGSGGGGSEIVLTQSPGSLSLSPGERATLSCRASQSVSSSYIAWYQQKPGQAPRLLIYGASSRATGIPDRFSGSGSGTDFSLTISRLEPEDFAVYYCQQYGSSPMYTFGPGTRVEIK

SEQ ID NO: 174 - protein, 236 aa, synthetic construct
EVQLVESGGGLVKPGGSLRLSCAASGFTFSTYLMYWIRQAPGKGLEWVSAIGSGGDTYYADSVKGRFTISRDNAKNSLYLQMNSLRAEDTAVYYCARGLGYWGQGTLVTVSSGGGGSGGGGSGGGGSEIVLTQSPGSLSLSPGERATLSCRASQSVSSSYLAWYQQKPGQAPRLLIYGASSKATGIPDRFSGSGSGTDFSLTISRLEPEDFAVYYCQQYGSSPMYTFGPGTRVEIK

SEQ ID NO: 175 - protein, 236 aa, synthetic construct
EVQLVESGGGLVKPGGSLRLSCAASGFTFSTYLMYWIRQAPGKGLEWVSAIGSGGDTYYADSVKGRFTISRDNAKNSLYLQMNSLRAEDTAVYYCARGLGYWGQGTLVTVSSGGGGSGGGGSGGGGSEIVLTQSPGSLSLSPGERATLSCRASQSVSSSYLAWYQQKPGQAPRLLIYGASSRATGIPDRFSGSGSGTDFSLTISRLEPEDFAVYYCQQYGSSPLYTFGPGTRVEIK

SEQ ID NO: 176 - protein, 248 aa, synthetic construct
EIVMTQSPDSLAVSLGERATISCKSSQSLLNSRNRKNYLAWYQQNPGQPPKLLIYWASTKESGVPDRFSGSGSGTDFTLTISSLQAEDVAVYYCKQSYYLWTFGQGTKVEIKGGGGSGGGGSGGGGSQVQLVESGGRLVQPGGSLRVSCEASGFTFSDYGMAWVRQAPGKGLEWVSFISNLGYSIYYADSVKGRFTISRDNAKNSLYLQMNSLRAEDTAVYYCARAPYDYDSFDPMDYWGQGTMVTVS

SEQ ID NO: 177 - protein, 248 aa, synthetic construct
EIVMTQSPDSLAVSLGERATISCKSSQSLLNSRNRKNYLAWYQQNPGQPPKLLIYWASTRESGVPDRFSGSGSGTDFTLTISSLQAEDVAVYYCKQSYYIWTFGQGTKVEIKGGGGSGGGGSGGGGSQVQLVESGGRLVQPGGSLRVSCEASGFTFSDYGMAWVRQAPGKGLEWVSFISNLGYSIYYADSVKGRFTISRDNAKNSLYLQMNSLRAEDTAVYYCARAPYDYDSFDPMDYWGQGTMVTVS

SEQ ID NO: 178 - protein, 248 aa, synthetic construct
EIVMTQSPDSLAVSLGERATISCKSSQSLLNSRNRKNYLAWYQQNPGQPPKLLIYWASTRESGVPDRFSGSGSGTDFTLTISSLQAEDVAVYYCKQSYYLWTFGQGTKVEIKGGGGSGGGGSGGGGSQVQLVESGGRLVQPGGSLRVSCEASGFTFSDYGLAWVRQAPGKGLEWVSFISNLGYSIYYADSVKGRFTISRDNAKNSLYLQMNSLRAEDTAVYYCARAPYDYDSFDPMDYWGQGTMVTVS

SEQ ID NO: 179 - protein, 248 aa, synthetic construct
EIVMTQSPDSLAVSLGERATISCKSSQSLLNSRNRKNYLAWYQQNPGQPPKLLIYWASTRESGVPDRFSGSGSGTDFTLTISSLQAEDVAVYYCKQSYYLWTFGQGTKVEIKGGGGSGGGGSGGGGSQVQLVESGGRLVQPGGSLRVSCEASGFTFSDYGMAWVRQAPGKGLEWVSFISNLGYSIYYADSVKGRFTISRDNAKNSLYLQMNSLRAEDTAVYYCARAPYDYDSFDPMNYWGQGTMVTVS

SEQ ID NO: 180 - protein, 248 aa, synthetic construct
EIVMTQSPDSLAVSLGERATISCKSSQSLLNSRNRKNYLAWYQQNPGQPPKLLIYWASTKESGVPDRFSGSGSGTDFTLTISSLQAEDVAVYYCKQSYYLWTFGQGTKVEIKGGGGSGGGGSGGGGSQVQLVESGGRLVQPGGSLRVSCEASGFTFSDYGMAWVRQAPGKGLEWVSFISNLGYSIYYADSVKGRFTISRDNAKNSLYLQMNSLRAEDTAVYYCARAPYDYDSFDPMNYWGQGTMVTVS

SEQ ID NO: 181 - protein, 248 aa, synthetic construct
EIVMTQSPDSLAVSLGERATISCKSSQSLLNSRNRKNYLAWYQQNPGQPPKLLIYWASTKESGVPDRFSGSGSGTDFTLTISSLQAEDVAVYYCKQSYYLWTFGQGTKVEIKGGGGSGGGGSGGGGSQVQLVESGGRLVQPGGSLRVSCEASGFTFSDYGLAWVRQAPGKGLEWVSFISNLGYSIYYADSVKGRFTISRDNAKNSLYLQMNSLRAEDTAVYYCARAPYDYDSFDPMDYWGQGTMVTVS

SEQ ID NO: 182 - DNA, 708 bp, synthetic construct
  source 1..708: mol_type = other DNA
gaggtgcagctggtggagtctggcggcggcctggtgaagcccggaggctccctgcggctgtcctgcgctgcctccgggttcaccttctccacctacctgatgtactggatcaggcaggctcccgggaaggggctggagtgggtgtccgccatcgggtccggcggcgacacctactacgccgactccgtgaaggggcgcttcaccatctcccgggacaacgccaagaactccctgtacctgcagatgaactccctgcgggccgaggacaccgccgtgtactactgcgccaggggcctggggtactggggccaggggaccctggtgaccgtgtcctccggcggcggaggctctggaggaggcggctctggcggcggaggctctgagatcgtgctgacccagagccccgggtccctgtccctgagccccggggagcgggctaccctgtcctgccgggccagccagtccgtgtcctcctcctacatcgcctggtaccagcagaagcccgggcaggctcccaggctgctgatctacggggcttcctccagggccaccggcatccccgaccgcttctccgggtccgggtccgggaccgacttctccctgaccatctcccggctggagcccgaggacttcgccgtgtactactgccagcagtacgggtccagccccatgtacaccttcgggcccgggacccgcgtggagatcaag

SEQ ID NO: 183 - DNA, 708 bp, synthetic construct
  source 1..708: mol_type = other DNA
gaggtgcagctggtggagtctggcggcggcctggtgaagcccggaggctccctgcggctgtcctgcgctgcctccgggttcaccttctccacctacctgatgtactggatcaggcaggctcccgggaaggggctggagtgggtgtccgccatcgggtccggcggcgacacctactacgccgactccgtgaaggggcgcttcaccatctcccgggacaacgccaagaactccctgtacctgcagatgaactccctgcgggccgaggacaccgccgtgtactactgcgccaggggcctggggtactggggccaggggaccctggtgaccgtgtcctccggcggcggaggctctggaggaggcggctctggcggcggaggctctgagatcgtgctgacccagagccccgggtccctgtccctgagccccggggagcgggctaccctgtcctgccgggccagccagtccgtgtcctcctcctacctggcctggtaccagcagaagcccgggcaggctcccaggctgctgatctacggggcttcctccagggccaccggcatccccgaccgcttctccgggtccgggtccgggaccgacttctccctgaccatctcccggctggagcccgaggacttcgccgtgtactactgccagcagtacgggtccagccccctgtacaccttcgggcccgggacccgcgtggagatcaag

SEQ ID NO: 184 - DNA, 744 bp, synthetic construct
  source 1..744: mol_type = other DNA
gagatcgtgatgacccagagccccgactccctggccgtttccctcggggagcgggccaccatctcctgcaagagctcccagagcctgctgaactcccggaaccggaagaactacctggcctggtaccagcagaaccccgggcagccgccgaagctgctgatctactgggcctccacgaaggagagcggggtgcccgatcgcttctccggctccggctccgggaccgacttcaccctgaccatctccagcctgcaggccgaggacgtggccgtctactactgcaagcagagctactacctctggaccttcgggcaggggaccaaggtggagatcaaaggcggcggaggctctggcggaggaggctctggaggcggcggctcccaggtgcagctggtggagagcggcggaaggctggtgcagcccggcggctccctgagggtctcctgcgaggcctccggcttcaccttctccgactacgggatggcctgggtgaggcaggctcccgggaaggggctggagtgggtctccttcatctccaacctgggctactccatctactacgccgactccgtgaaggggcgcttcaccatctcccgggacaacgccaagaactccctctacctgcagatgaactccctgcgggccgaggacaccgccgtctactactgcgcccgggcgccctacgactacgactccttcgaccccatgaactactggggccaggggaccatggtgaccgtctcc

SEQ ID NO: 185 - DNA, 744 bp, synthetic construct
  source 1..744: mol_type = other DNA
gagatcgtgatgacccagagccccgactccctggccgtttccctcggggagcgggccaccatctcctgcaagagctcccagagcctgctgaactcccggaaccggaagaactacctggcctggtaccagcagaaccccgggcagccgccgaagctgctgatctactgggcctccacgaaggagagcggggtgcccgatcgcttctccggctccggctccgggaccgacttcaccctgaccatctccagcctgcaggccgaggacgtggccgtctactactgcaagcagagctactacctctggaccttcgggcaggggaccaaggtggagatcaaaggcggcggaggctctggcggaggaggctctggaggcggcggctcccaggtgcagctggtggagagcggcggaaggctggtgcagcccggcggctccctgagggtctcctgcgaggcctccggcttcaccttctccgactacgggctggcctgggtgaggcaggctcccgggaaggggctggagtgggtctccttcatctccaacctgggctactccatctactacgccgactccgtgaaggggcgcttcaccatctcccgggacaacgccaagaactccctctacctgcagatgaactccctgcgggccgaggacaccgccgtctactactgcgcccgggcgccctacgactacgactccttcgaccccatggactactggggccaggggaccatggtgaccgtctcc